(19) United States
(12) Patent Application Publication
(10) Pub. No.: US 2015/0306086 A1
(43) Pub. Date: Oct. 29, 2015

(54) MODULATING CERTAIN TYROSINE KINASES

(71) Applicant: Tesaro, Inc., Waltham, MA (US)

(72) Inventor: Keith M. Wilcoxen, North Reading, MA (US)

(21) Appl. No.: 14/357,884

(22) PCT Filed: Nov. 13, 2012
(86) PCT No.: PCT/US12/64822

§ 371 Date: May 13, 2014

Related U.S. Application Data
(60) Provisional application No. 61/559,592, filed on Nov. 14, 2011.

(51) Int. Cl. A61K 31/454; A61K 45/06; A61K 31/437; A61K 31/541; A61K 31/4545; A61K 31/496; A61K 31/4196; A61K 31/4995; C12Q 1/68; A61K 31/5377

(52) U.S. Cl. CPC A61K 31/454; C12Q 1/6886; A61K 45/06; A61K 31/437; A61K 31/5377; A61K 31/4545; A61K 31/496; A61K 31/4196; A61K 31/4995; A61K 31/541; C12Q 2600/158; C12Q 2600/112

(57) ABSTRACT

The present invention provides therapeutic and diagnostic modalities relevant to treating disorders associated with tyrosine kinase activity.

### CROSS REFERENCE TO RELATED APPLICATIONS

The present application claims the benefit of, and priority to, U.S. Provisional Application Ser. No. 61/559,592, filed Nov. 14, 2011, which is hereby incorporated by reference in its entirety.

### BACKGROUND

Tyrosine kinases regulate a wide range of biological events and activities. Many potent and effective therapeutic agents act by modulating one or more tyrosine kinases. However, certain tyrosine kinases (including, for example, anaplastic lymphoma kinase [ALK]) are known to develop resistance to many therapeutic agents. Furthermore, different tyrosine kinases may have different (although sometimes overlapping) activities; significant effort can be require to identify tyrosine kinase modulators (e.g., inhibitors) with activity and selectivity appropriate to treat any particular disease, disorder, or condition.

### SUMMARY

The present invention encompasses the finding that certain benzimidazole compounds show desirable activity and/or specificity profiles with respect to tyrosine kinases.

For example, the present invention demonstrates that certain such compounds effectively inhibit resistant ALK-associated diseases or disorders. The present invention also demonstrates that certain such compounds inhibit one or more member(s) of the tropomyosin receptor kinase (TRK) and ret proto-oncogene (RET) families.

Among other things, the present invention provides methods comprising administering to a subject suffering from a condition associated with activity of one or more particular tyrosine kinases (e.g., an ALK-inhibitor-resistant tyrosine kinase, a TRK family member, etc) a compound of formula I:

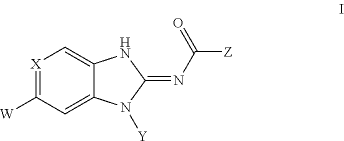

or a pharmaceutically acceptable salt thereof,
wherein W, X, Y and Z are as described herein.

In some embodiments, the present invention provides methods comprising administering to a subject suffering from an ALK-associated condition, and who shows one or more indicia of resistance, a compound of formula I.

In some embodiments, the present invention provides methods comprising administering to a subject suffering from or susceptible to an ALK inhibitor-resistant ALK-associated condition a compound of formula I in combination with one or more additional chemotherapeutic agents.

In some embodiments, the present invention provides methods comprising steps of:
(i) detecting in a subject one or more indicia of resistance (e.g. the presence or level of a resistance-associated marker, progression of disease, etc.); and
(ii) determining, based on the presence of the detected one or more indicia, that the subject is a candidate for therapy with a compound of formula I.

In some embodiments, the present invention provides methods comprising steps of:
(i) detecting in a subject one or more indicia of resistance (e.g. the presence or level of a resistance-associated marker, progression of disease, etc.);
(ii) determining, based on the presence of the detected one or more indicia, that the subject is a candidate for therapy with a compound of formula I, and
(iii) administering to the patient a therapeutically effective amount of a compound of formula I.

In some embodiments, the present invention provides a method of treating an ALK-associated condition, the method comprising administering to a patient in need thereof a compound of formula I, wherein the ALK-associated condition is localized or present in the central nervous system. In some such embodiments, the ALK-associated condition is a cancer of the central nervous system. In some embodiments, the cancer of the central nervous system is a brain cancer or tumor. In some embodiments, the cancer of the central nervous system is a spinal cancer or tumor.

In some embodiments, the present invention provides a method of treating a TRK-mediated condition, the method comprising administering to a patient in need thereof a compound of formula I. In some such embodiments, the TRK-mediated condition is cancer.

In some embodiments, the present invention provides a method of treating a TRK-mediated condition, the method comprising administering to a patient in need thereof a compound of formula I, wherein the TRK-mediated condition is cancer pain.

In some embodiments, the present invention provides a method of preventing or inhibiting the progression of perineural invasion, the method comprising administering to a patient in need thereof a compound of formula I. In some embodiments, the present invention provides a method of preventing or inhibiting cancer metastasis, the method comprising administering to a patient in need thereof a compound of formula I.

### DEFINITIONS

ALK-associated condition: The term “ALK-associated condition” as used herein means any disease or other deleterious condition in which ALK, or a mutant thereof, is known or suspected to play a role. Accordingly, another embodiment of the present invention relates to treating one or more diseases in which ALK, or a mutant thereof, is known or suspected to play a role. Specifically, the present invention relates to a method of treating of a disease or condition selected from a proliferative disorder or an autoimmune disorder, wherein said method comprises administering to a patient in need thereof a compound or composition according to the present invention. In some embodiments, the ALK-associated condition is cancer. In some such embodiments, the ALK-associated condition is lung cancer, neuroblastoma, anaplastic large cell lymphoma, glioblastoma, breast cancer, colon cancer or inflammatory myofibroblastic tumors (IMT).

ALK-associated marker: The term “ALK-associated marker” as used herein means the presence or level of any relevant marker, for example a nucleic acid (i.e. a gene or gene mutation), chemical compound (i.e. a mineral, metal, or small molecule), peptide, or feature whose presence or level correlates with an ALK-associated condition. In some embodiments, an ALK-associated marker is a genetic marker (e.g., a gene or gene sequence, including presence of multiple copies of a gene or gene sequence). In some embodiments, an ALK-associated marker is a fusion gene. In some embodiments, an ALK-associated marker is a protein. In some embodiments, an ALK-associated marker is a fusion protein. In some embodiments, an ALK-associated marker is a level of biological activity (e.g., level of ALK kinase activity).

Combination therapy: The term “combination therapy”, as used herein, refers to those situations in which two or more different pharmaceutical agents are administered in overlapping regimens so that the subject is simultaneously exposed to both agents. For example, a compound of the present invention may be administered with another therapeutic agent simultaneously or sequentially in separate unit dosage forms or together in a single unit dosage form. For purposes of clarity, the term “sequentially” is used herein to mean that a compound of the present invention may be administered before, during or after the administration of another therapeutic agent.

Dosing regimen: A “dosing regimen” (or “therapeutic regimen”), as that term is used herein, is a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regime comprises a plurality of doses and at least two different time periods separating individual doses. In some embodiments, the therapeutic agent is administered continuously over a predetermined period. In some embodiments, the therapeutic agent is administered once a day (QD) or twice a day (BID).

Subject: As used herein, the term “subject” or “patient” refers to any organism upon which embodiments of the invention may be used or administered, e.g., for experimental, diagnostic, prophylactic, and/or therapeutic purposes. Typical subjects include animals (e.g., mammals such as mice, rats, rabbits, non-human primates, and humans; insects; worms; etc.).

Suffering from: An individual who is “suffering from” a disease, disorder, or condition (e.g., cancer) has been diagnosed with and/or exhibits one or more symptoms of the disease, disorder, or condition.

Therapeutic regimen: As used herein, the term “therapeutic regimen” refers to any protocol used to partially or completely alleviate, ameliorate, relieve, inhibit, prevent, delay onset of, reduce severity of and/or reduce incidence of one or more symptoms or features of a particular disease, disorder, and/or condition. In some embodiments, a therapeutic regimen may comprise a treatment or series of treatments whose administration correlates with achievement of a particular result across a relevant population. In some embodiments, a therapeutic regimen involves administration of one or more therapeutic agents, either simultaneously, sequentially or at different times, for the same or different amounts of time. Alternatively, or additionally, the treatment may include exposure to protocols such as radiation, chemotherapeutic agents or surgery. Alternatively or additionally, a “treatment regimen” may include genetic methods such as gene therapy, gene ablation or other methods known to reduce expression of a particular gene or translation of a gene-derived mRNA.

Therapeutic agent: As used herein, the phrase “therapeutic agent” refers to any agent that elicits a desired pharmacological effect when administered to an organism. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, an appropriate population may be defined by a functional assay. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition.

Therapeutically effective amount: As used herein, the term “therapeutically effective amount” refers to an amount of a therapeutic agent whose administration, when viewed in a relevant population, correlates with or is reasonably expected to correlate with achievement of a particular therapeutic effect. The therapeutic effect may be objective (i.e., measurable by some test or marker) or subjective (i.e., subject gives an indication of or feels an effect). A therapeutically effective amount is commonly administered in a dosing regimen that may comprise multiple unit doses. For any particular therapeutic agent, a therapeutically effective amount (and/or an appropriate unit dose within an effective dosing regimen) may vary, for example, depending on route of administration or combination with other pharmaceutical agents. Also, the specific therapeutically effective amount (and/or unit dose) for any particular patient may depend upon a variety of factors including the disorder being treated and the severity of the disorder; the activity of the specific pharmaceutical agent employed; the specific composition employed; the age, body weight, general health, sex and diet of the patient; the time of administration, route of administration, and/or rate of excretion or metabolism of the specific agent employed; the duration of the treatment; and like factors that are well known in the medical arts.

Treatment: As used herein, the term “treatment” (and other grammatical forms thereof, such as “treating”) refers to a therapeutic protocol that alleviates, delays onset of, reduces severity or incidence of, and/or yield prophylaxis of one or more symptoms or aspects of a disease, disorder, or condition. In some embodiments, treatment is administered before, during, and/or after the onset of symptoms. In some embodiments, treatment may be administered to a subject who does not exhibit signs of a disease, disorder, and/or condition. In some embodiments, treatment may be administered to a subject who exhibits only early signs of the disease, disorder, and/or condition, for example for the purpose of decreasing risk of developing pathology associated with the disease, disorder, and/or condition.

TRK-associated condition: The term “TRK-associated condition” as used herein means any disease or other deleterious condition in which at least one TRK receptor, or a mutant thereof, is known or suspected to play a role. In some embodiments, the TRK receptor is selected from TRKA, TRKB, TRKC and p75, or a combination thereof. Alternatively or additionally, a TRK-associated condition is any disease or other deleterious condition whose occurrence, incidence, and/or severity is associated with presence and/or level of TRK activity. As discussed herein, TRK is known or suspected to be involved in a variety of biological conditions or events including, for example, cell proliferation, cancer metastasis and pain. Some of these biological conditions or events may involve other kinases; some may be unique to TRK. In some embodiments, the present invention relates to treating one or more diseases in which TRK, or a mutant thereof, is known or suspected to play a role. For example, in some specific embodiments, the present invention relates to a method of treating a disease or condition selected from a proliferative disorder or pain, wherein said method comprises administering to a patient in need thereof a compound or composition according to the present invention, and particularly a compound or composition that inhibits TRK activity. In some embodiments, the TRK-associated condition is cancer. In some embodiments, the TRK-associated condition is pain.

Tyrosine kinase associated condition: The term “tyrosine-kinase-associated condition” as used herein means any disease or other deleterious condition in which a tyrosine kinase, or a mutant thereof, is known or suspected to play a role. Alternatively or additionally, a tyrosine-kinase-associated condition is any disease or other deleterious condition whose occurrence, incidence, and/or severity is associated with presence and/or level of tyrosine kinase activity.

### DETAILED DESCRIPTION OF CERTAIN EMBODIMENTS

### Tyrosine Kinases

Protein tyrosine kinases are a class of enzymes that catalyze the transfer of a phosphate group from ATP or GTP to a tyrosine residue located on a protein substrate. Receptor tyrosine kinases act to transmit signals from the outside of a cell to the inside by activating secondary messaging effectors via a phosphorylation event. A variety of cellular processes are promoted by these signals, including proliferation, carbohydrate utilization, protein synthesis, angiogenesis, cell growth, and cell survival. Indeed, activating mutations in the tyrosine kinase domain have been identified in patients with a variety of cancers, such as non-small cell lung cancer (Lin, N. U.; Winer, E. P., Breast Cancer Res 6: 204-210, 2004).

Receptor tyrosine kinases are high-affinity cell surface receptors for many polypeptide growth factors, cytokines and hormones. Of the 90 unique tyrosine kinase genes in the human genome, 58 encode receptor tyrosine kinase proteins. Receptor tyrosine kinases have been shown not only to be key regulators of normal cellular processes but also to have a critical role in the development and progression of many types of cancer.

### Anaplastic Lymphoma Kinase

Anaplastic lymphoma kinase (ALK) is a receptor tyrosine kinase which is implicated in a variety of diseases and disorders such as non-small cell lung cancer, neuroblastoma and anaplastic large-cell lymphoma (ALCL). Several known mutations or translocations of ALK result in one or more ALK fusion genes, which promote kinase activity and lead to oncogenic events.

ALK was first described as an oncogene in human cancer in the 1990s, with the description of the nucleophosmin-ALK (NPM-ALK) fusion gene in anaplastic large-cell lymphoma (ALCL), resulting in the acronym ALK. Since then, a large number of ALK translocations in a growing variety of tumor types have been described, in which the uniting theme is the dimerization and inappropriate ligand-independent activation of ALK tyrosine kinase activity by the fusion partner in question. As well as a role in hematological malignancies, ALK translocations are also found in a number of solid tumor types, including NSCLC, squamous cell carcinoma, and more recently thyroid cancer. While initially considered to be rather unusual, the identification of fusions such as TMPRSS2-ERG (transmembrane protease, serine 2-ETS-related gene) in prostate cancer suggest that the occurrence of such fusions in solid tumors has been underestimated.

ALK plays an important role in the development of the brain and exerts its effects on specific neurons in the nervous system. At least two different types of mutations are known to render the ALK gene oncogenic: fusions of any of several other genes, and point or site mutations (e.g., deletions, insertions, inversions, translocations) of ALK gene sequences. Several ALK fusions have been identified in recent years. Typically, such fusions are characterized in that: (i) the partner genes to which ALK is fused are constitutively transcribed in cells in which the fusion occurs and/or (ii) the various fusion partners are able to mediate dimerization (or oligomerization) of the chimeric protein (i.e., the protein produced from the gene fusion). Significantly, all identified ALK fusion proteins show constitutive ALK activity. Because the ALK tyrosine kinase activity is necessary for ALK fusion protein oncogenicity, research has focused on identifying ALK kinase inhibitors. Of particular interest are ALK kinase inhibitors that exhibit activity against any ALK fusion protein harboring the ALK active site.

Certain ALK-inhibitors such as crizotinib cause tumors to shrink or stabilize in the majority of patients harboring an ALK fusion gene. However, despite these remarkable initial responses, cancers eventually develop resistance to ALK inhibitors, including crizotinib, thereby limiting the potential clinical benefit.

NPM-ALK.

The t(2;5)(p23;q35) chromosomal translocation creates a fusion gene composed of the 5′ portion of nucleophosmin (NPM) from chromosome 5 and the 3′ half of anaplastic lymphoma kinase (ALK) gene, derived from chromosome 2. The NPM-ALK chimeric gene encodes a constitutively activated tyrosine kinase that shows potent oncogenic activity. The NPM-ALK kinase, on dimerization, shows phosphotransferase activity and, through its interaction with various ALK-adapter proteins, induces cell transformation and increases cell proliferation in vitro. Thus, the product of the NPM-ALK fusion gene is oncogenic. (Pileri et al., Am. J. Pathology, 1997, 150(4), 1207-1211, hereby incorporated by reference in its entirety).

ELM4-ALK.

The chromosomal rearrangement involving the short arm of chromosome 2 (2p21 and 2p23) generates a fusion gene between EML4 (echinoderm microtubule-associated protein-like 4) and ALK. EML4-ALK undergoes constitutive dimerization through interaction between the coiled-coil domain within the EML4 region of each monomer, thereby activating ALK and generating oncogenic activity. EML4-ALK fusion oncogenes have been reported in approximately 4% of all cases of NSCLC. (Choi et al., New England J. Medicine, 2010, 363(18), 1734-1739; Sasaki et al., European J. Cancer, 2010, 46, 1773-1780, each of which is hereby incorporated by reference in its entirety).

Other ALK Fusion Proteins.

Other constitutively active ALK-fusion proteins have been identified. Recent studies demonstrated that ALK may also be involved in variant translocations, namely, t(1;2)(q25;p23), t(2;3)(p23;q21), t(2;17)(p23;q23) and inv(2)(p23q35), which create the TPM3-ALK, TFG-ALK, CLTC-ALK, and ATIC-ALK fusion genes, respectively. The portion of ALK retained in the ATIC-ALK fusion protein is the same as in NPM-ALK as well as in ALK-fusion proteins TPM3-ALK and TFG-ALK. These X-ALK variant fusion proteins have the same intracytoplasmic region of ALK but lack the NPM nuclear localization domain, which accounts for the restricted cytoplasmic distribution of TPM3-ALK, TFG-ALK, CLTC-ALK, ATIC-ALK and ELM4-ALK fusion proteins. (De Paepe et al., Blood, 2003, 102(7), 2638-2641; Brunangelo et al., Blood, 1999, 94(10), 3509-3515, each of which is hereby incorporated by reference in its entirety).

TPM3-ALK.

The TPM3-ALK fusion protein results from the fusion of the nonmuscular tropomyosin (TPM3) gene with the ALK gene in a t(1;2)(q25;p23) translocation.

TFG-ALK.

The TFG-ALK fusion protein results from the fusion of TRK-fused gene (TFG) with the ALK gene in a t(2;3)(p23;q21). TFG-ALK fusion proteins are associated in cells with the same signaling intermediates used by NPM-ALK for signal transduction, suggesting that different ALK chimeric products likely use similar transforming pathways. (Hernandez et al., Blood, 1999, 94(9), 3265-3268; Hernandez et al., American J. Pathology, 2002, 160(4), 1487-1494, each of which is hereby incorporated by reference in its entirety).

ATIC-ALK.

The ATIC gene (previously named as Pur H) encodes a bifunctional enzyme (5-aminoimidazole-4-carboxamide ribonucleotide transformylase and IMP cyclohydrolase enzymatic activities) that catalyzes the penultimate and final enzymatic activities of the purine nucleotide biosynthetic pathway. Expression of full-length ATIC-ALK cDNA in mouse fibroblasts revealed that the fusion protein (a) possesses constitutive tyrosine kinase activity; (b) forms stable complexes with the signaling proteins Grb2 and Shc; (c) induces tyrosine-phosphorylation of Shc; and (d) provokes oncogenic transformation. (Colleoni et al., Am. J. Pathology, 2000, 156(3), 781-789; Trinei et al., Cancer Res., 2000, 60, 793-798, each of which is hereby incorporated by reference in its entirety).

KIF5B-ALK.

KIF5B is located on the short arm of human chromosome 10 and encodes member 5B of the kinesin family of proteins. KIF5B is a component of a motor protein complex that is associated with microtubules and mediates the transport of organelles within eukaryotic cells. It consists of an amino terminal motor domain followed by a neck region and a stalk region, the latter of which directly mediates homodimerization of KIF5B. Fusion of exons 1 to 24 of KIF5B to exon 20 of ALK would be expected to result in the production of a fusion protein consisting of almost the entire KIF5B sequence ligated to the intracellular region of ALK. It might therefore also be expected that KIF5B-ALK would undergo homodimerization mediated by the stalk region of KIF5B, with consequent activation of the kinase function of ALK, similar to the case of EML4-ALK, in which homo-oligomerization and activation are mediated by the amino terminal coiled-coil domain of EML4. To confirm the identify of the KIF5B-ALK fusion gene, the fusion point of the KIF5B-ALK cDNA was directly amplified by RT-PCR with one primer targeted to exon 24 of KIF5B and the other to exon 22 of ALK (Takeuchi et al., Clinical Cancer Research 2009, 15(9), 3143-3149, hereby incorporated by reference in its entirety). A single PCR product with the expected size of 546 bp was obtained and nucleotide sequencing of the product further confirmed the fusion point of KIF5B-ALK at the cDNA level.

### ALK-Associated Disorders

Anaplastic Large-Cell Lymphoma (ALCL).

Lymphoma is the most common blood cancer. The two main forms of lymphoma are Hodgkin's lymphoma (HL) and non-Hodgkin's lymphoma (NHL). Lymphoma occurs when lymphocytes, a type of white blood cell, grow abnormally. The body has two main types of lymphocytes that can develop into lymphomas: B-lymphocytes (B-cells) and T-lymphocytes (T-cells). Cancerous lymphocytes can travel to many parts of the body, including the lymph nodes, spleen, bone marrow, blood or other organs, and can accumulate to form tumors.

Large cell lymphomas comprise approximately one fourth of all non-Hodgkin's lymphomas in children and young adults. ALCL was first described in 1985 as a previously unrecognized lymphoid tumor in which the neoplastic cells were labeled by the monoclonal antibody Ki-1 (subsequently shown to recognize the CD30 receptor molecule). ALCL is a rare type of NHL, but the second or third most common subtype of T-cell lymphoma. There are three types of ALCL and altogether they comprise about 3 percent of all NHLs in adults and between 10 percent and 30 percent of all NHLs in children. ALCL can present either systemically (in lymph nodes or organs throughout the body) or in the skin.

When ALCL presents in the skin it is called primary cutaneous ALCL and follows a less aggressive course. In almost all cases of primary cutaneous ALCL, the disease is confined to the skin. Despite a tendency to relapse, relapses are usually in the skin only. As long as it is confined to the skin, it is usually managed as an indolent (slow-growing) lymphoma. Approximately 10 percent of the time, primary cutaneous ALCL extends beyond the skin to lymph nodes or organs. If this occurs, it is usually managed like the systemic forms of ALCL.

Occasionally, primary cutaneous ALCL is associated with another rare condition called lymphomatoid papulosis (LyP). LyP is a skin condition with similar features to primary cutaneous ALCL. While LyP is classified as a lymphoma, the skin lesions always go away by themselves, usually over a four to eight week period, and, therefore, do not behave like a malignancy.

The characteristic features of primary cutaneous ALCL include the appearance of solitary or multiple raised red skin lesions, nodules or tumors, which do not go away, have a tendency to ulcerate and may itch. The lesions can appear on any part of the body, often grow very slowly and may be present for a long time before being diagnosed.

Approximately one third of ALCL tumors are positive for the NPM-ALK fusion. Lymphomas with the NPM-ALK fusion typically involve lymph nodes, skin, lung, soft tissue, bone and the gastrointestinal tract, and arise predominantly from activated T lymphocytes. Most tumors with the NPM-ALK fusion are classified as anaplastic large cell non-Hodgkin's lymphomas (A. G. Stansfeld et al., Lancet. 1:292 (1988)). The NPM-ALK 2;5 chromosomal translocation is associated with approximately 60% anaplastic large-cell lymphomas. NPM-ALK protein not only typically accumulates in the cytoplasm but also in the nucleus and nucleolus of lymphoma cells.

Patients with systemic ALCL can be divided into two groups, depending on the expression of ALK. While both lymphomas are treated as aggressive lymphomas, the prognosis for ALCL depends on whether a patient is ALK positive (expresses the protein) or ALK negative (does not express the protein). ALK positive disease responds well to chemotherapy, putting most patients in long-term remission or cure. Most people with ALK negative ALCL respond to chemotherapy, but many will relapse within five years. Because of this, they are sometimes treated more aggressively, often with stem cell transplant. The ALK positive subtype usually affects children and young adults. The ALK negative subtype is more commonly found in older patients over age 60.

Five years after the first description of ALCL, it was noted that tumors carrying the (2;5)(p23;q35) chromosome translocation, a rare cytogenetic abnormality thought initially to be characteristic of malignant histiocytosis, were CD30 (Ki-1)-positive large-cell lymphomas. In 1994, researchers showed that the (2;5) translocation fuses part of the nucleophosmin (NPM) gene on chromosome 5q35 to a portion of the ALK receptor tyrosine kinase gene on chromosome 2p23, resulting in expression of a unique chimeric NPM-ALK protein. Antibodies specific for the ALK kinase have been reported, and the absence of this molecule from normal lymphoid cells means that a positive immunocytochemical reaction for ALK protein is essentially specific for the (2;5) translocation. The major exception is a rare large B-cell lymphoma in which (by an unknown mechanism) full-length ALK protein is expressed.

Non-Small Cell Lung Cancer.

Figures released by the American Cancer Society for 2008 reported 1.6 million new lung cancer cases worldwide. Indeed, lung cancer is the leading cause of cancer death in men and the second leading cause of cancer death in women, with estimated deaths approaching 1.4 million worldwide in 2008. Clinically, primary lung cancer is divided into small-cell lung cancer (SCLC) and non-small cell lung cancer (NSCLC), and patients receive differential therapy based on these criteria. NSCLC is an umbrella term for a number of tumor types that together account for approximately 80% of lung cancers. These include the three main subtypes of squamous-cell lung carcinoma, large-cell lung carcinoma, and adenocarcinoma. Adenocarcinoma accounts for approximately 40% of all NSCLC and is more prevalent among people who have never smoked. For many years, treatment for advanced or metastatic NSCLC has employed chemotherapy regimens for patient care with limited effect. Five-year survival rates for these patients are not encouraging. However, for a subgroup of these patients, there have been radical changes over recent years. Understanding of the basic pathology behind NSCLC at the molecular level has offered up a host of new molecularly targeted therapies, which are revolutionizing this area of cancer care. Activating EGFR (epidermal growth factor receptor) mutations in NSCLC provided the first opportunity to generate molecularly defined treatments such as the inhibitors gefitinib and erlotinib. Results from recent clinical trials provide hope for NSCLC patients harboring oncogenic translocations involving the anaplastic lymphoma kinase (ALK) receptor tyrosine kinase. Just as inhibition of the BCR-ABL (breakpoint cluster region-c-abl oncogene 1, non-receptor tyrosine kinase) complex has changed the face of chronic myeloid leukemia diagnosis, oncogenic ALK fusions offer a step forward in the diagnosis and treatment of ALK-positive NSCLC. Recent advances in drug development, particularly those targeting ALK, have led to significant changes in the way this patient population, and their future therapeutic prospects, are viewed.

The appearance of ALK fusion oncoproteins in NSCLC was first described in 2007 in two independent studies with quite different approaches. Classical tumor DNA library transformation assays were used to identify echinoderm microtubule-associated protein-like 4 (EML4)-ALK. Initial global phosphotyrosine proteomic analyses of NSCLC cell lines further identified a number of oncogenic lesions including EML4-ALK and TRK-fused gene-ALK (TFG-ALK).

Prior to the identification of ALK fusion proteins in NSCLC, the patient population presenting with ALK fusions, such as NPM-ALK in ALCL, was limited. This number changed significantly with the consideration of an estimated 3-13% of NSCLC patients. Calculated at a rate of 5% of ALK translocations and based on 2008 American Cancer Society figures, NSCLC cases amenable to ALK-directed therapies would be predicted to reach in the order of 80,000 new lung cancer patients per year worldwide.

The NSCLC patient group presenting with ALK translocations is somewhat different from the more commonly appreciated smoking-related lung cancer population. It is now recognized that there is an increasing population of ‘non-smoking-associated lung cancer’ NSCLC patients in which aberrations such as EML4-ALK and activating EGFR mutations are enriched. This population is generally predominantly female and tumors are often adenocarcinomas.

The EML4-ALK fusion gene is responsible for approximately 3-5% of non-small-cell lung cancer (NSCLC). The vast majority of cases are adenocarcinomas. On average, ALK lung cancers are found in people who are approximately 10-15 years younger than other lung cancers, and who are also significantly more likely to be non-smokers and somewhat more likely to be light former smokers. To date, multiple EML4-ALK variants have been identified in lung cancer. Although the fusions contain variable truncations of EML4 (occurring at exons 2, 6, 13, 14, 15, 18, and 20), the ALK fusion in all of them starts at a portion encoded by exon 20 of the kinase gene. To date, all EML4-ALK fusions tested biologically demonstrate gain of function properties.

EML4-ALK transcripts are expressed in about 15% of non-tumor lung tissues, which implies that the EML4-ALK rearrangement is not tumor-specific. Moreover, finding that patients expressing the EML4-ALK mRNA in non-tumor lung tissues do not harbor the fusion transcript in the paired tumors raises the question of whether the EML4-ALK rearrangement is directly linked to NSCLC pathogenesis. In fact, the scenarios of EML4-ALK and EGFR1 mutations in lung cancer appear to be quite different. EGFR1 mutations were found in the normal respiratory epithelium of 43% patients with EGFR-mutated lung adenocarcinoma but not in patients with EGFR-mutation-free lung tumors, suggesting a localized field effect phenomenon.

In NSCLC patients carrying the EML4-ALK transcript, only about 2% of tumor cells harbored the corresponding fusion gene, as detected by FISH analysis of paraffin-embedded sections. ALK gene rearrangements, with or without EML4 involvement, were also detected in 9/603 (1.5%) NSCLC samples they studied by FISH in tissue microarrays. The percentage of tumor cells carrying the rearrangement was, however, higher (50% to 100%) than in the present study. Different numbers of cases and techniques in the two studies could, at least in part, account for the discrepancy.

Forced expression of EML4-ALK in lung epithelial cells induced the rapid development of hundreds of lung cancer nodules in mice, and peroral administration of inhibitors of the PTK activity of EML4-ALK was shown to clear such tumors from the lungs, demonstrating the pivotal role of EML4-ALK in the pathogenesis of NSCLC positive for this fusion kinase. This latter observation also supports the clinical application of ALK inhibitors to treat EML4-ALK-positive lung cancer in humans. It should be noted, however, that multiple isoforms of EML4-ALK, generated mainly as a result of diversity in the breakpoint-fusion point within EML4, have been identified in NSCLC specimens. The accurate diagnosis of EML4-ALK-positive tumors will therefore require detection of all in-frame fusions between EML4 and ALK cDNAs, as exemplified by our multiplex reverse transcription- and PCR-based detection system for EML4-ALK. TFG-ALK and KIF5B-ALK fusions have also been implicated in NSCLC tumor samples.

Neuroblastoma.

Neuroblastoma is the most common childhood cancer. Recent study shows that mutation of ALK is linked to 10-15% of neuroblastoma. There is a strong familial association and it was predicted over 30 years ago that there was a genetic element to the disease. ALK mutations have since been identified in neuroblastoma patients. ALK acts as a neuroblastoma predisposition gene, and somatic point mutations occur in sporadic neuroblastoma cases. These mutations promote ALK's kinase activity and can transform cells and display tumorigenic activity in vivo.

ALK-related neuroblastoma susceptibility occurs in individuals who are heterozygous for an ALK mutation and is characterized by an increased risk of developing neuroblastoma, ganglioneuroblastoma, or ganglioneuroma. The risk of tumor development is highest in infancy and decreases by late childhood. Individuals with familial neuroblastoma tend to develop tumors at a younger age (average 9 months) compared to those without familial predisposition (age 2-3 years).

Breast Cancer.

Pleiotrophin (PTN) is expressed in breast cancers and in cell lines derived from human breast cancers; since targeting constitutive PTN signaling by a dominant negative PTN reversed the malignant phenotype of human breast cancer cells in vivo, it is suggested that constitutive PTN signaling contributes to the pathogenesis of advanced breast cancer. Recently, different models to determine the role of inappropriate expression of Ptn in breast cancer were tested and found that inappropriate expression of Ptn that was targeted to breast epithelial cells by the mouse mammary tumor virus (MMTV) promoter does not induce breast cancer in a MMTV-Ptn transgenic mice breast cancer model; however, MMTV-driven PTN signaling cooperated with the oncoprotein polyoma middle T-antigen (PyMT) to promote progression of breast cancer in MMTV-PyMT-Ptn bitransgenic mice. It was furthermore found that secretion of PTN alone through activation of stromal cells and induction of marked remodeling of the microenvironment was sufficient to account for significant features of breast cancer progression, thus, the data supports potentially a very important role of PTN signaling in promoting a more aggressive breast cancer phenotype.

Researchers analyzed the expression of ALK in tissues derived from human breast cancers and demonstrated that ALK is highly expressed in each of the different subtypes of human breast cancer studied. Perez-Pinera et al., Biochem Biophys Res Commun. 2007 Jun. 29; 358(2): 399-403. Further, the cellular location and patterns of ALK expression in the breast cancer cells differs significantly from its pattern of expression in normal breast tissues, which is consistent with the possibility that ALK may be activated through a constitutively activated PTN/RPTPβ/ζ signaling pathway in breast cancers that inappropriately express Ptn.

Other ALK-Associated Conditions.

ALK has been recently implicated in the occurrence of a rare nonlymphoid neoplasm, known as inflammatory myofibroblastic tumor. This tumor is associated with various translocations in which ALK is fused to TPM3, TPM4, CLTC or CARS (encoding the cysteiny1-tRNA synthetase) genes. ALK has also been implicated in glioblastoma, esophageal squamous cell carcinomas and types of breast cancer (Webb et al. Expert Review of Anticancer Therapy, 2009, 9(3); 331-356, which is hereby incorporated by reference in its entirety.

Disorders of the Central Nervous System.

Cancers of the central nervous system (CNS) are considered to be among the most devastating of all cancers. The brain and spinal cord are complex organs that control the CNS, the peripheral nervous system, and many of the voluntary and involuntary systems of the body. The effects can be devastating for the patient and the family when cancer attacks the CNS. It has been found that 20%-40% of all cancers metastasize to the brain. The diagnosis of CNS cancer, with the daunting statistics on median survival of patients with high-grade tumors being less than 12 months, leaves little hope for the patient.

An estimated 12,920 deaths were attributed to primary CNS cancers in 2009. The incidence of CNS tumors is highest in developed, industrialized countries where approximately 6-11 new cases are diagnosed annually per 100,000 population; mortality rates for all types of primary CNS tumors are 3-7 per 100,000. Gliomas comprise 70% of all brain tumors with the most common type, glioblastoma multiforme, also being the most lethal. From 2003 to 2007, the median age of patients at the time of a brain cancer diagnosis was 56 years old. Although the exact incidence of metastatic brain tumors is unknown, estimates range from double to 10 times the number of primary brain tumors, with at least 20%-40% of patients with cancer developing brain metastases at some point in their disease.

### ALK Inhibitor Therapy for ALK-Associated Disorders

Much effort has been invested in the identification and/or characterization of compounds that can inhibit ALK. For example, US patent publication numbers US 2011/0257155, US 2011/0257155, US 2011/0256546, US 2011/0190264, US 2011/0190259, US 2011/0135668, US 2010/0298295, US 2008/0300273, US 2008/0176881, describe various compounds which have demonstrated ALK inhibitory activity.

Recently, one amino pyridine compound, crizotinib, was approved for treatment of non-small cell lung cancer that is locally advanced or has metastasized. Crizotinib acts specifically on tumors that contain the abnormally activated EML4-ALK, which is found in only about 5.5% of patients with NSCLC. Within that small patient population, crizotinib has shown striking activity, eliciting a 57% response rate and an 87% disease control rate at 8 weeks.

Resistance.

Despite the remarkable therapeutic promise of ALK inhibitors such as crizotinib, evidence suggests that resistance develops rapidly, often within one year. For example, at least two de novo mutations within the kinase domain of EML4-ALK are known to confer resistance to multiple ALK inhibitors. Evidence suggests that resistance to crizotinib is a complicated paradigm. Indeed, resistance can manifest as (i) new mutations in the ALK gene known to be associated with resistance or (ii) additional copies of the ALK gene, which can overcome the effects of an ALK inhibitor.

Acquired Resistance can Manifest in Several Ways.

A mutation which coexists with the ALK mutation is one such pathway of acquired resistance. Treatment with an ALK inhibitor does not suppress the biological effects from the second mutation, such as EGFR or KRAS, which develops a competing and overriding activity and conferring resistance.

Conversely, the emergence of a predominance of a new and separate oncogenic mutation may result from the presence of different subsets of cancer cells. Prior to treatment, the cancer is comprised of primarily ALK-positive cancer cells, with a minority of cells harboring another mutation such as KRAS. Treatment with a targeted therapy such as an ALK inhibitor alters the balance of the cancer cells, such that the ALK positive cells die and the cancer continues to grow as a predominantly KRAS positive cancer.

Markers Associated with Resistance.

Development of resistance can be detected, for example, through monitoring disease progression during and after administration of therapy. Any marker whose presence or level correlates with presence, or preferably progression, of an ALK-associated condition can be used to assess development of resistance. Alternatively or additionally, markers associated with ALK level or activity can be used. Markers that specifically correlate with development of resistance (e.g., that reflect or correspond to presence of specific ALK mutations) are of particular interest.

A variety of appropriate markers are available. For example, the most common genetic markers associated with crizotinib resistance are kinase gatekeeper mutations, such as L1196M. Other relevant markers include R1275Q, F1174L, EML4-ALK and NPM-ALK. In some embodiments, an ALK-associated marker is an amplification of ALK activity above a threshold level.

Crizotinib is administered as a 250 mg oral dosage form twice a day (BID). Dosing interruption and/or dose reduction to 200 mg PO BID may be required based on safety and tolerability. The intensity of clinical adverse events is graded by the NCI Common Terminology Criteria for Adverse Events (AE). In such instances, the dose is decreased to 250 mg PO once a day (QD). Hematologic toxicities except lymphopenia (unless associated with clinical events, e.g., opportunistic infections) require dosage modifications: Grade 3 AE (withhold until recovery to Grade ≦2, then resume at the same dose schedule); Grade 4 AE (withhold until recovery to Grade ≦2, then resume at 200 mg PO BID; in case of recurrence, withhold until recovery to Grade ≦2, then resume at 250 mg PO qDay; permanently discontinue in case of Grade 4 recurrence). Non-hematologic toxicities requiring dosage modifications include Grade 3 or 4 alanine aminotransferase (ALT) or aspartate aminotransferase (AST) elevation with Grade ≦1 total bilirubin (withhold until recovery to Grade ≦1 or baseline, then resume at 200 mg PO BID (in case of recurrence, withhold until recovery to Grade ≦1, then resume at 250 mg PO qDay; permanently discontinue in case of further Grade 3 or 4 recurrence)); Grade 3 QTc prolongation (withhold until recovery to Grade ≦1, then resume at 200 mg PO BID). Dosage regimens resulting in Grade 2, 3 or 4 ALT or AST elevation with concurrent Grade 2, 3, or 4 total bilirubin elevation (in absence of cholestasis or hemolysis), any Grade pneumonitis or Grade 4 QTc prolongation mandate that crizotinib be permanently discontinued.

### Treating Resistant ALK-Associated Disorders

In some embodiments, the present invention encompasses the finding that certain compounds of formula I are particularly useful in treating ALK-associated disorders. In some embodiments, the present invention encompasses the recognition that compounds of formula I are useful in treating ALK-associated disorders that are or risk of becoming resistant to an ALK inhibitor. In some embodiments, the present invention provides methods of treating an ALK-associated condition that is susceptible to resistance to an ALK inhibitor comprising administering to a patient in need thereof a compound of formula I. Accordingly, in some embodiments, the present invention provides a method comprising administering to a subject suffering from an ALK-inhibitor-resistant ALK-associated condition a compound of formula I. In some embodiments, the present invention provides a method of treating an ALK-inhibitor-resistant ALK-associated condition, the method comprising administering to a patient in need thereof a compound of formula I.

In some embodiments, the resistance to an ALK inhibitor is crizotinib-resistance. In some such embodiments, compounds of formula I are useful in treating ALK-associated disorders that are or at risk of becoming crizotinib-resistant. Accordingly, in some embodiments, the present invention provides a method of treating a crizotinib-resistant ALK-associated condition comprising administering to a patient in need thereof a compound of formula I.

In some embodiments, the ALK-associated disorder is a cancer. Accordingly, in some embodiments, the present invention provides a method of treating an ALK-inhibitor-resistant cancer, the method comprising administering to a patient in need thereof a compound of formula I. In some embodiments, the ALK-associated condition is selected from NSCLC, neuroblastoma, ALCL, inflammatory myofibroblastic tumor, inflammatory breast cancer, esophageal cancer, gastric cancer and glioblastoma.

In some embodiments, the present invention provides methods of treating an ALK-associated disorder, wherein the ALK-associated disorder manifests in the brain and/or central nervous system. In some such embodiments, the ALK-associated disorder is a cancer. In some embodiments, such cancers are brain cancer and spinal cancer. In some embodiments, the brain cancer is a glioblastoma.

In some embodiments, the present invention provides a method of treating metastatic brain cancer, the method comprising administering to a patient in need thereof a compound of formula I.

In some embodiments, the present invention provides a method of treating an ALK-associated condition, the method comprising administering to a patient in need thereof a compound of formula I, wherein the patient shows one or more indicia of resistance.

In some embodiments, the present invention provides a method comprising administering to a subject suffering from an ALK-associated condition which shows one or more indicia of resistance to an ALK inhibitor a compound of formula I. In some embodiments, the ALK-associated condition is resistant to crizotinib. In some embodiments, the indicia of resistance are selected from L1196M, R1275Q, F1174L, ELM4-ALK, NPM-ALK and combinations thereof.

In some embodiments, the present invention provides a method of detecting in a subject an ALK inhibitor resistance-associated marker and determining that the subject is a candidate for therapy with a compound of formula I. In some such embodiments, the resistance marker is L1196M. In some embodiments, the resistance-associated marker is detected at a level above the threshold correlated with elevated probability of resistance to the ALK inhibitor. In some such embodiments, the ALK inhibitor is crizotinib.

In some embodiments, the present invention provides methods comprising steps of:
(i) detecting in a subject one or more indicia of resistance (e.g. the presence or level of a resistance-associated marker, progression of disease, etc.); and
(ii) determining, based on the presence of the detected one or more indicia, that the subject is a candidate for therapy with a compound of formula I.

In some embodiments, the present invention provides methods comprising steps of:
(i) detecting in a subject one or more indicia of resistance (e.g. the presence or level of a resistance-associated marker, progression of disease, etc.);
(ii) determining, based on the presence of the detected one or more indicia, that the subject is a candidate for therapy with a compound of formula I, and
(iii) administering to the patient a therapeutically effective amount of a compound of formula I.

In some embodiments, the one or more indicia of resistance is an ALK fusion oncoprotein. In some such embodiments, the ALK fusion oncoprotein is selected from NPM-ALK, ELM4-ALK, ATIC-ALK, TPM3-ALK, TFG-ALK, CLTC-ALK and KIF5B-ALK. In some embodiments, the ALK fusion oncoprotein is NPM-ALK. In some embodiments, the ALK fusion oncoprotein is ELM4-ALK. In some embodiments, the one or more indicia of resistance is progression of the disease.

In some embodiments, the compound of formula I is selected from compound I-a and compound I-b.

In some embodiments, the present invention provides methods comprising administering to a subject suffering from or susceptible to a resistant ALK-associated condition a compound of formula I in combination with one or more additional chemotherapeutic agents.

In some embodiments, provided methods comprise administering to a patient in need thereof a compound of formula I and at least one additional chemotherapeutic agent. In some such embodiments, at least one of a compound of formula I and the additional chemotherapeutic agent is administered at a dose lower than when administered as a single agent. In some embodiments, the additional chemotherapeutic agent is selected from the group consisting of docetaxel, pemetrexed, carboplatin, paclitaxel and cisplatin.

### Tropomyosin-Receptor Kinase (TRK)

During discovery of the compounds of formula I, it was surprisingly discovered that compound I-a exhibited comparable activity against the TRK (tropomyosin receptor kinase) family. TRK is a family of tyrosine kinases that regulate survival, development and function of subsets of neurons, especially sensory and sympathetic neurons. TRK receptors affect neuronal survival and differentiation through several signal cascades, including the PI3K/Akt, Ras/MAPK STAT3, and PLCγ pathways. The TRK family consists of TRKA, TRKB, TRKC and p75 with specific ligands for each receptor. Nerve growth factor (NGF) specifically binds TRKA, brain-derived neurotrophic factor (BDNF) and Neurotrophin (NT)-4/5 bind TRKB, and NT3 is known to bind TRKC. The p75 receptor is often referred to as the low-affinity TRK receptor, as much higher concentrations of the neurotrophins are needed to activate its signaling pathways. Therapeutically, most attention has focused on the involvement of NGF and BDNF in pain mechanisms, but increasing attention is being paid to the role of these growth factors, and their cognate receptors, in neurological diseases, behaviors and cancer.

A key feature of malignant cells is their ability to dissociate from the primary tumor and to establish metastatic deposits at distant sites generally through one of the three common routes of metastatic spread; lymphatic, neural and vascular channels. Like vascular and lymphatic routes of metastasis, neural invasion (NI) (sometimes referred to as perineural invasion or PNI) has emerged as a key pathological feature of many cancers, including pancreatic cancer, head and neck cancers, squamous cell carcinoma, prostate cancer, colorectal cancer, breast cancer, biliary tract cancer, stomach cancer and cholangiocarcinoma. The molecular mechanistic drivers of cancers metastasis through PNI are sometimes also contributors to the proliferation of the tumor, as seen in pancreatic, breast and head and neck cancers, to name a few. Studies have demonstrated that PNI involves reciprocal signaling interactions between tumor cells and nerves and invading tumor cells have potentially acquired the ability to respond to proinvasive signals found within the nerve during tumorigenesis. The neurotrophins and their receptors tropomyosin receptor kinases (TRKs) have emerged as key mediators of PNI and the proliferation of some tumor types.

Table 1 lists the cancer types in which perineural invasion has been reported:

TABLE 1

| | Incidence of perineural invasion |
|---|---|
| Cancer Type | (%) |
| Pancreatic Cancer | up to 100 |
| Cholangiocarcinoma | 75-85 |
| Head and Neck Cancers | Up to 80 |
| Prostate Cancer | 75-80 |
| Biliary Tract Cancer | ~80 |
| Stomach Cancer | 50-60 |
| Breast Cancer | 3-38 |
| Colorectal Cancer | 9-33 |
| Squamous Cell carcinoma | 2.5-14 |
| Basal Cell Carcinoma | 0.2-10 |

The first clinical association of TRKs and cancer came with the findings of activating mutations caused by chromosomal rearrangements or mutations in TRKA in papillary and medullary thyroid carcinoma, respectively. However even in these tumor types, the frequency of genetic alterations in TRK genes is low, and such alterations have not been consistently identified in other tumors. Over the years, a number of studies have detailed TRK expression in different tumor types, correlating expression with prognosis or tumor stage. However, there is no clear overarching pattern of association of a particular TRK isoform with prognosis in the literature. In cases where TRKs are not translocated or mutated, there is increasing evidence they have a pathophysiologic role in the biology of tumor cells. Because neurotrophin activation of TRK mediates survival signals and stimulates neuritogenesis (the formation of neuritis) and migration in normal neurons, it is believed these same processes are exploited by tumor cells to survive cytotoxic insults or metastasize via PNI. A number of studies have shown that neurotrophins are important in tumor progression; they initiate mitogenic signals that facilitate tumor growth, prevent apoptosis and regulate angiogenesis, cell spreading and metastasis. In a landmark paper on anoikis (apoptosis resulting from loss of cell matrix interactions), a genome-wide functional screen for metastasis-associated oncogenes was conducted and identified TRKB as a key mediator allowing survival of cells during systemic circulation and resistance to anoikis.

Without wishing to be bound by any particular theory, it is believed that cancers that are susceptible to PNI acquire properties during tumorigenesis that not only allow metastatic spread through neural channels, but also rely on these mechanistic characteristics for proliferation and resistance to cytotoxic therapies and local environmental stress, and exist at the interface of driver oncogenes and tumor supportive mechanisms.

Lung Cancer.

A number of studies have identified a relationship with NT and TRK receptors in lung cancer. While there are contradictory results, the current understanding of the role of NT and TRKs in lung cancer is beginning to be elucidated. There are a number of studies confirming the expression of both NTs and TRK in lung tumors, with NGF and BDNF being the most common NT overexpressed in NSCLC and TRKA and TRKB playing a more significant role than TRKC. Numerous studies have shown that stimulation of TRKA or TRKB signaling increases tumor invasiveness and colony formation in both selected cell lines and lung cancer cells from NSCLC patients. Conversely, NGF has been shown to be anti-proliferative in tumors of neuroendocrine origin, such as SCLC. A general view of the scope of TRK studies in lung cancer does seem to indicate a more important role for TRKB and BDNF in NSCLC and TRKA and NGF in SCLC, but this is not conclusive. It should also be noted that while overexpression and enhanced TRK signaling can be observed in both an autocrine and paracrine manner, there is no conclusive evidence the TRK or NTs are drivers for NSCLC formation. However, it should be noted that p75 is not detected in tumors, perhaps due to its loss during tumor progression. TRKA and TRKB could play a role in NSCLC tumor progression and metastasis, with over-expression being selected as an advantageous phenotype during tumorigenesis. A secondary hypothesis is that expression of NT and TRK within a NSCLC tumor cell population creates a more aggressive tumor phenotype. The positive rate of TRKB in NSCLC varies from 24% to 86.7% in previous studies. A recent study showed that 75.5% and 82.4% of NSCLC samples were positive for TRKB and BDNF, respectively, and TRKB and BDNF were highly expressed in lung cancer tissue. Positive rates and average scores of TRKB and BDNF were highest in LCNEC patients compared to the patients with other histological subtypes. High expression levels of TRKB and BDNF might be involved in neuroendocrine differentiation of LCNEC. This study also showed expression of TRKB alone had a significant inverse correlation with disease free and overall survival and expression of both BDNF and TRKB was associated with worse survival than TRKB expression alone, consistent with the relevance of autocrine signaling.

Head and Neck Cancers.

A number of studies have identified a relationship with BDNF and TRKB receptors in head and neck cancer. The role of BDNF and TRKB in head and neck cancer is only beginning to be elucidated, but collectively the data suggest that the invasiveness of head and neck cancer is influenced by BDNF and TRKB autocrine/paracrine signaling, TRK and the resistance to anoikis exhibited by head and neck cancer cells may be due to TRKB signaling.

Breast Cancer.

The role of neutrophins and their receptors in breast cancer have been studied since 1993. Collectively, there are several points that can be summarized regarding neurotrophins, TRKs and breast cancer. First, NGF is overexpressed in breast cancer and triggers cell survival and proliferation through TRKA and p75. NGF and its precursor are secreted by breast cancer cells and are drivers of invasion through TRKA. BDNF and other TRK ligands can be autocrinally produced and stimulate tumor cell survival and resistance to apoptosis through p75 and TRKB. The targeting of neurotrophins and their receptors has been shown to result in an inhibition of breast tumor growth and metastasis in vivo.

Pancreatic Cancer.

The role of NTs and TRK in pancreatic cancer is preferentially dominated by information related to TRKA and NGF. A number of studies have identified a relationship with NTs and TRK receptors in pancreatic cancer. Multiple laboratories have confirmed the overexpression of NGF and TRKA in pancreatic cancer. In addition, the use of TRK antagonists or anti-NT antibodies has shown the inhibition of cancer cell line growth in vitro and in vivo. There is a significant role for TRKA and NGF in perineural invasion and cancer pain.

Prostate Cancer.

The prostate contains the most abundant source of NGF outside of the nervous system. NGF and NGF-immunoreactive proteins secreted by the prostate are able to stimulate prostatic epithelial growth Immunocytochemistry and mRNA analysis of smooth muscle cells of normal prostate, non-metastatic cells and metastatic cancer cells indicate prostate malignancy involves a switch from paracrine to autocrine control of neurotrophin activity and resultant proliferation and metastasis.

A number of studies have identified a relationship with neurotrophins and the TRK receptors in prostate cancer. Most evidence points to TRKA and TRKB involvement in prostate cancer growth, invasion and metastasis, while the low affinity p75 receptor is considered to be a tumor suppressor (and often lost during prostate cancer progression). Non-clinical studies support the inhibition of TRK expressing prostate tumor growth in vitro and in vivo by NT antibodies and small molecule multikinase inhibitors with TRK inhibitory activity. The most well studied compound is CEP-701 (Lestaurtinib), which did not produce the expected results clinically when assessed for the reduction of PSA in cancer patients. However, Lestaurtinib suffered from poor pharmacokinetics and very high protein binding, with the data indicating the tolerable concentrations may not have been widely achieved in patients.

Cylindroma.

Individuals with germline mutations in the tumor suppressor gene CYLD are at high risk of developing disfiguring cutaneous appendageal tumors, the defining tumor being the highly organised cylindroma. In a recent study, the authors analyzed CYLD mutant tumor genomes by array comparative genomic hybridization and gene expression microarray analysis. CYLD mutant tumors were characterized by an absence of copy-number aberrations apart from LOH chromosome 16q, the genomic location of the CYLD gene. Gene expression profiling of CYLD mutant tumors showed dysregulated tropomyosin receptor kinase (TRK) signaling, with overexpression of TRKB and TRKC in tumors when compared with perilesional skin. Immunohistochemical analysis of a tumor microarray showed strong membranous TRKB and TRKC staining in cylindromas, as well as elevated levels of ERK phosphorylation and BCL2 expression. Membranous TRKC overexpression was also observed in 70% of sporadic basal cell carcinomas of the skins. RNA interference-mediated silencing of TRKB and TRKC, as well as treatment with the small-molecule TRK inhibitor lestaurtinib, reduced colony formation and proliferation in 3D primary cell cultures established from CYLD mutant tumors. These results suggest that TRK inhibition could be used as a strategy to treat tumors with loss of functional CYLD and further investigation of TRK signaling and TRKCi sensitivity of basal cell carcinoma is in order.

TRK in Pain.

The role of NGF in neuronal development has been known for over half a century. NGF plays a critical role in the development of the peripheral nervous system by promoting growth and survival of some neural crest-derived cells in developing embryos, in particular sensory and sympathetic neurons. Loss-of function mutations in the TRKA gene cause congenital insensitivity to pain with anhidrosis (CIPA). CIPA is an autosomal recessive genetic disorder characterized by insensitivity to noxious stimuli, anhidrosis (inability to sweat) and mental retardation due to hyperthermia caused from anhidrosis. Congenital insensitivity to pain with anhidrosis, a human condition in which patients generally have normal proprioception and normal sensation to innocuous pressure but abnormal sensation to thermal stimuli, is caused by a mutation of the TRKA genes that result in a structural neuropathy affecting unmyelinated peripheral nerve fibers. Indeed, genetically modified animals lacking the NGF or TRKA gene are born with virtually no small-caliber primary sensory neurons and are profoundly unresponsive to noxious stimuli. To further support its role in pain, when NGF was administered to AD patients, the most significant side effect was severe reversible back pain.

While there is an increasing interest in the role of NGF/TRK in neurological disease, the inhibition of NGF/TRKA signaling has been most investigated for its role in nociceptor sensitization after injury and cancer related bone pain Immunologic and genetics studies of NGF deprivation during development and maturation demonstrate that NGF has three separate roles-one for survival and development of sensory and sympathetic neurons, the second in maintaining the peptidergic phenotype of primary afferent neurons in the early postnatal period and the third being a key upstream modulator of the expression and sensitization of a variety of neurotransmitter, receptor and ion channels expressed by adult nociceptors. However, whether adult sensory neurons require NGF for maintenance of their phenotype and, if so, how much NGF remains to be determined Preclinical data suggest that reducing or preventing the NGF production that is associated with some types of injury, through the sequestering of NGF or the inhibition of NGF-TRKA signaling is effective in reducing hypersensitivity and nociceptor activation in animal models. Importantly, the studies suggest this approach does not obviously compromise normal nociceptor function or cause the loss of sympathetic or sensory nerve fiber innervation of the skin or bone. For example, NGF blockade does not affect the normal inflammatory response (erythema, heat or swelling) in tissues, and anti-NGF therapy reveals no modification of the biomechanical properties of the femur or histomorphometric indices of bone healing and load bearing. In a model of bone cancer, a novel NGF sequestering antibody demonstrated a profound reduction in both ongoing and movement-evoked bone cancer pain-related behaviors that was greater than that achieved with acute administration of 10 or 30 mg/kg of morphine. This therapy also reduced several neurochemical changes associated with peripheral and central sensitization in the dorsal root ganglion and spinal cord, whereas the therapy did not influence disease progression or markers of sensory or sympathetic innervation in the skin or bone.

There have been over 100 clinical trials specifically involving the addition or blockade of NGF.

It has become increasingly apparent the NGF/TRKA play a significant role in chronic pain and from this has grown three major pharmacological strategies targeting NGF/TRKA signaling. These include sequestration of NGF or inhibiting its binding to TRKA, antagonizing TRKA so as to block NGF from binding to TRKA, and blocking TRKA kinase activity. A number of humanized anti-NGF monoclonal antibodies have entered into clinical trials, and these include RN624 (tanezumab, Pfizer), AMG-403 (fulranumab, Amgen), REGN475 (Regeneron/Sanofi-Aventis), Medi-578 (Medimmune), and ABT-110 (Abbott). Pfizer (Tanezumab) has a rich amount of information regarding human efficacy and safety, with 4 completed phase III clinical trials in osteoarthritis related pain and >10,000 patients treated. Tanezumab has shown impressive results in osteoarthritis pain and chronic back pain. In a study assessing osteoarthritis pain reduction, 450 patients were exposed to increasing doses of tanezumab. The rate of response was 74 to 93% versus 44% with placebo, and the rates of adverse events were 68% and 55% in the tanezumab and placebo groups, respectively. The most common adverse events among patients were headache (9%), upper respiratory infection (%7) and paresthesia (7%).

Cancer Pain.

It has been reported that 75-90% of patients with metastatic or advanced stage cancer will experience significant cancer-induced pain. Chronic pain associated with advanced malignancies has also been shown to be related to NOF signaling. Prostate, and breast cancers, which frequently result in bone metastases, are characterized by severe bone pain. In experimental tumor models in rats, NGF produced by the tumor cells and/or tumor-associated stromal cells has been implicated in the extensive sprouting of sensory neural fibers from the bone tissue and the resulting hyperalgesia. Administration of a TRK selective inhibitor attenuated sarcoma-induced bone cancer pain and significantly blocked the ectopic sprouting of sensory nerve fibers and the formation of neuroma-like structures in the tumor bearing bone.

### Treating TRK-Associated Conditions

In some embodiments, the present invention provides a method of treating a TRK-associated condition, the method comprising administering to a patient in need thereof a compound of formula I.

In some embodiments, the present invention provides a method of inhibiting TRK, the method comprising contacting a cell with a compound of formula I. In some embodiments, the present invention provides a method of treating an ALK-associated disease or disorder comprising administering to a patient in need thereof a therapeutically effective amount of a compound of formula I, wherein the therapeutically effective amount of a compound of formula I is sufficient to treat a TRK-associated condition.

In some embodiments, the present invention provides a method of simultaneously treating an ALK-associated condition and a TRK-associated condition, the method comprising administering to a patient in need thereof a compound of formula I.

In some embodiments, the TRK-associated condition is a TRKA-associated condition. In some embodiments, the TRK-associated condition is a TRKB-associated condition. In some embodiments, the TRK-associated condition is a TRKC-associated condition.

In some embodiments, the TRK-associated condition is cancer. In some such embodiments, the cancer is selected from pancreatic cancer, lung cancer, cholangiocarcinoma, head and neck cancers, prostate cancer, biliary tract cancer, stomach cancer, breast cancer, colorectal cancer, squamous cell cancer, basal cell carcinoma and cylindroma.

In some embodiments, the present invention encompasses the recognition that compounds of formula I, and in particular compounds of formulae I-a and I-b, are useful in treating cancer pain. Accordingly, in some embodiments, the TRK-associated condition is pain. In some such embodiments, the pain is cancer pain. In some embodiments, the pain is bone pain.

In some embodiments, the present invention provides a method of inhibiting TRK, the method comprising administering to a patient in need thereof a compound of formula I.

In some embodiments, the present invention provides a method of treating perineural invasion, the method comprising administering to a patient in need thereof a compound of formula I. In some embodiments, the present invention provides a method of preventing or inhibiting cancer metastasis, the method comprising administering to a patient in need thereof a compound of formula I. In some such embodiments, the compound of formula I is compound selected from I-a and compound I-b.

In some embodiments, the present invention provides a method of preventing or inhibiting perineural invasion-mediated cancer metastasis, the method comprising administering to a patient in need thereof a compound of formula I.

In some embodiments, the present invention provides a method of preventing or inhibiting metastatic spread of cancer through neural channels, the method comprising administering to a patient in need thereof a compound of formula I.

In some embodiments, the present invention provides a method of treating cancer pain, the method comprising administering to a patient in need thereof a compound of formula I. In some embodiments, the present invention provides a method of treating chronic pain associated with advanced malignancies comprising administering to a patient in need thereof a compound of formula I. In some embodiments, the present invention provides a method of treating pain associated with bone metastases comprising administering to a patient in need thereof a compound of formula I. In some embodiments, the present invention provides a method of inhibiting sprouting of sensory neural fibers comprising administering to a patient in need thereof a compound of formula I. In some embodiments, the compound of formula I is selected from the compounds I-a and I-b. In some embodiments, the compound of formula I is the compound I-a. In some embodiments, the compound of formula I is the compound I-b.

In some embodiments, the level of IRK inhibition is a biomarker indicating significant ALK inhibition in a patient. Accordingly, in some embodiments, IRK inhibition is a biomarker for assessing and monitoring ALK inhibition.

In some embodiments, provided methods comprise determining and/or quantifying the level of IRK inhibition in a patient.

Provided Compounds for Use in Accordance with the Present Invention

The present invention provides uses of compounds of formula I, for example in the treatment of tyrosine-kinase-associated disorders and particularly for use in disorders associated with AKL-inhibitor-resistant ALK(s) and/or with TRK(s). Compounds of formula I have the structure:

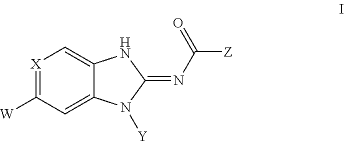

or a pharmaceutically acceptable salt thereof, wherein:
X is selected from CH or N;
Y is selected from a C₃-C₁₂ cycloalkyl or a 3-10 membered heterocyclyl comprising 1, 2, or 3 heteroatoms selected from O, S, or N; wherein the C₃-C₁₂ cycloalkyl and the 3-10 membered heterocyclyl may be monocyclic, bicyclic, or tricyclic, and further wherein the C₃-C₁₂ cycloalkyl and the 3-10 membered heterocyclyl are unsubstituted or are optionally substituted with 1, 2, or 3 substituents independently selected from —R′, —Y′, —SO₂—Y″, —C(═O)—Y″, —SO₂NH—Y″, —C(═O)NH—Y″ or —C(═O)NH—(C₁-C₄)alkylene-Y″; wherein two substituents on a carbon ring member of the Y cycloalkyl or heterocyclyl may join to form a 3-7 membered cycloalkyl group or a 3-7 membered heterocyclyl group that comprises 1 to 3 heteroatoms selected from N, O, or S; and further wherein 1 or 2 carbon atom ring members of the 3-7 membered cycloalkyl or the 3-7 membered heterocyclyl group formed from the two substituents on the carbon ring member of the Y cycloalkyl or heterocyclyl may be double bonded to an O atom;
   Y′ is a C₆-C₁₀ aryl, a 5-10 membered heteroaryl comprising 1, 2, 3, or 4 heteroatoms independently selected from O, S, or N, or a 3-7 membered heterocyclyl comprising 1, 2, or 3 heteroatoms selected from O, S, or N, wherein the C₆-C₁₀ aryl, the 5-10 membered heteroaryl, or the 3-7 membered heterocyclyl Y′ groups are unsubstituted or are optionally substituted with 1, 2, or 3 substituents independently selected from —R′;
   Y″ is selected from a C₃-C₁₀ cycloalkyl; a 3-10 membered heterocyclyl comprising 1, 2, or 3 heteroatoms selected from N, O, and S; a C₆-C₁₀ aryl; or a 5-10 membered heteroaryl comprising 1, 2, 3, or 4 heteroatoms independently selected from N, O, or S; wherein the C₃-C₁₀ cycloalkyl and the 3-10 membered heterocyclyl may be monocyclic or bicyclic, and further wherein the C₃-C₁₀ cycloalkyl, the 3-10 membered heterocyclyl, the C₆-C₁₀ aryl, or the 5-10 membered heteroaryl Y″ groups are unsubstituted or are optionally substituted with 1, 2, or 3 substituents independently selected from —R′;
R′ is —F, —Cl, —Br, —I, —C≡N, —NO₂, —OH, —O—(C₁-C₆)alkyl, —SH, —S—(C₁-C₆)alkyl, —OCF₃, —OCHF₂, —CF₃ (C₁-C₆)alkyl, —(C₂-C₆)alkenyl, —(C₂-C₆)alkynyl, —NH₂, —NH((C₁-C₄)alkyl)—N((C₁-C₄)alkyl)₂, —NHSO₂—(C₁-C₆)alkyl, —NHC(═O)—(C₁-C₆)alkyl, —C(═O)NH₂, —C(═O)NH((C₁-C₆)alkyl), —C(═O)NH—(C₁-C₄)alkylene-CF₃, —C(═O)NH—(C₁-C₄)alkylene-F, —C(═O)NH—(C₂-C₄)alkenyl, —C(═O)N((C₁-C₆)alkyl)₂, —C(═O)NH—OH, —C(═O)NH—O—(C₁-C₆)alkyl, —C(═O)—(C₁-C₄)alkylene-CF₃, —C(═O)N—(C₁-C₄)alkylene-F, —C(═O)—(C₂-C₄)alkenyl, —C(═O)—(C₁-C₄)alkylene-NH₂, —C(═O)—(C₁-C₄)alkylene-NH((C₁-C₄)alkyl), —C(═O)—(C₁-C₄)alkylene-N(((C₁-C₄)alkyl)₂, —C(═O)NH—(C₁-C₄)alkylene-OH, —C(═O)NH—(C₁-C₄)alkylene-O—(C₁-C₆)alkyl, —C(═O)—(C₁-C₆)alkyl, —CO₂H, —C(═O)—O—(C₁-C₆)alkyl, —C(═O)NH—(C₁-C₄)alkylene-NH₂, —C(═O)NH—(C₁-C₄)alkylene-NH((C₁-C₆)alkyl), —C(═O)NH—(C₁-C₄)alkylene-N((C₁-C₆)alkyl)₂, —SO₂NH₂, —SO₂NH((C₁-C₆)alkyl), —SO₂N((C₁-C₆)alkyl)₂, —SO₂NH((C₂-C₄)alkenyl), —SO₂NH((C₂-C₄)alkynyl), —SO₂NH—(C₁-C₄)alkylene-OH, —SO₂NH—(C₁-C₄)alkylene-O—(C₁-C₄)alkyl, —SO₂—(C₁-C₆)alkyl, —SO—(C₁-C₆)alkyl, —(C₁-C₄)alkylene-NH—C(═O)—(C₁-C₆)alkyl, —(C₁-C₄)alkylene-NH₂, —(C₁-C₄)alkylene-NH—(C₁-C₆)alkyl, —(C₁-C₄)alkylene-N((C₁-C₆)alkyl)₂, —(C₁-C₄)alkylene-NH—(C₁-C₄)alkylene-CF₃, —CH(CF₃)(OH), —SO₃H, —(C₁-C₄)alkylene-OH, —(C₁-C₄)alkylene-O—(C₁-C₆)alkyl, —(C₁-C₄)alkylene-C(═O)—(C₁-C₆)alkyl, —(C₁-C₄)alkylene-C(═O)—O—(C₁-C₆)alkyl, or —(C₁-C₄)alkylene-C(═O)—OH;
W is selected from —H, —F, —Cl, —Br, —I, —(C₁-C₆)alkyl, —(CR^{a}R^{a′})_{q}—OH, —(CR^{a}R^{a′})_{q}—O—(C₁-C₆)alkyl, —(CR^{a}R^{a′})_{q}—O—W′, —O—(CR^{a}R^{a′})_{q}—W′, —O—(CR^{a}R^{a′})_{q}—OH, —O—(CR^{a}R^{a′})_{q}—O—(C₁-C₆)alkyl, —(CR^{a}R^{a′})_{q}—O—(CR^{a}R^{a′})_{q}— OH, —(CR^{a}R^{a′})_{q}—O—(CR^{a}R^{a′})_{q}—O—(C₁-C₆)alkyl, —(CR^{a}R^{a′})_{q}—SH, —(CR^{a}R^{a′})_{q}—S—(C₁-C₆)alkyl, —(CR^{a}R^{a′})_{q}—S—W′, —S—(CR^{a}R^{a′})_{q}—W′, —(CR^{a}R^{a′})_{q}—S(O)₂, —(C₁-C₆)alkyl, —(CR^{a}R^{a′})_{q}—S(O)₂—W′, —S(O)₂—(CR^{a}R^{a′})_{q}—W′, —(CR^{a}R^{a′})_{q}—NH₂, —(CR^{a}R^{a′})_{q}—NH—(C₁-C₆)alkyl, —(CR^{a}R^{a′})_{q}—N—((C₁-C₆)alkyl)₂, —(CR^{a}R^{a′})_{q}—N⁺—((C₁-C₆)alkyl)₃, —(CR^{a}R^{a′})_{q}—NH—W′, —(CR^{a}R^{a′})_{q}—NH— (CR^{a}R^{a′})_{q}—OH, —NH—(CR^{a}R^{a′})_{q}—W′, or —(CR^{a}R^{a′})_{q}—W′;
   W′ is selected from a C₃-C₁₀ cycloalkyl; a 3-10 membered heterocyclyl comprising 1, 2, or 3 heteroatoms selected from N, O, and S; a C₆-C₁₀ aryl; or a 5-10 membered heteroaryl comprising 1, 2, 3, or 4 heteroatoms independently selected from N, O, or S; wherein the C₃-C₁₀ cycloalkyl and the 3-10 membered heterocyclyl may be monocyclic or bicyclic, and further wherein the C₃-C₁₀ cycloalkyl, the 3-10 membered heterocyclyl, the C₆-C₁₀ aryl, or the 5-10 membered heteroaryl W′ groups are unsubstituted or are optionally substituted with 1, 2, 3, or 4 substituents independently selected from —R′ or —C(═O)—W″; and further wherein W′ may include 0, 1, or 2 ═O groups when W′ is a C₃-C₁₀ cycloalkyl or a 3-10 membered heterocyclyl, and further wherein the ═O groups may be bonded to a ring carbon atom or a ring S atom;
      W″ is selected from a C₃-C₁₀ cycloalkyl; a 3-10 membered heterocyclyl comprising 1, 2, or 3 heteroatoms selected from N, O, and S; a C₆-C₁₀ aryl; or a 5-10 membered heteroaryl comprising 1, 2, 3, or 4 heteroatoms independently selected from N, O, or S; wherein the C₃-C₁₀ cycloalkyl and the 3-10 membered heterocyclyl may be monocyclic or bicyclic, and further wherein the C₃-C₁₀ cycloalkyl, the 3-10 membered heterocyclyl, the C₆-C₁₀ aryl, or the 5-10 membered heteroaryl W″ groups are unsubstituted or are optionally substituted with 1, 2, 3, or 4 substituents independently selected from —R′; and further wherein W″ may include 0, 1, or 2 ═O groups when W″ is a C₃-C₁₀ cycloalkyl or a 3-10 membered heterocyclyl, and further wherein the ═O groups may be bonded to a ring carbon atom or a ring S atom;
q is, in each instance, independently selected from 0, 1, 2, 3, or 4;
R^{a} and R^{a′} are, in each instance, independently selected from —H, —CH₃, —CH₂CH₃, —F, —CF₃, or —C≡N; or:
   R^{a} and R^{a′} may join to form a cyclopropyl ring together with the carbon atom to which they are attached;
Z is selected from a C₆-C₁₀ aryl; a 5-10 membered heteroaryl comprising 1, 2, 3 or 4 heteroatoms independently selected from O, S, or N; a 4-7 membered heterocyclyl comprising 1, 2, or 3 heteroatoms selected from O, S, or N; a C₃-C₇ cycloalkyl; a —N(H)-heterocyclyl, wherein the heterocyclyl of —N(H)-heterocyclyl is a 4-7 membered heterocyclyl comprising 1, 2, or 3 heteroatoms selected from O, S, or N; a —N(H)—(C₃-C₇)cycloalkyl; or Z is a —O—(C₁-C₆)alkyl; wherein the C₆-C₁₀ aryl, the 5-10 membered heteroaryl, the 4-7 membered heterocyclyl, the C₃-C₇ cycloalkyl; the —N(H)-heterocyclyl, and the —N(H)—(C₃-C₇)cycloalkyl are unsubstituted or are optionally substituted with 1, 2, or 3 substituents independently selected from —R′;
W is not —H, —F, —Cl, —Br, —I, or unsubstituted —(C₁-C₆)alkyl if X is CH;
Y is not unsubstituted cyclopropyl, cyclobutyl, or cyclopentyl if W is —H, —F, —Cl, —Br, —I, or —(C₁-C₆)alkyl;
W is not —CH₂OH or —CH₂O(C₁-C₄alkyl) if Y is a group of formula

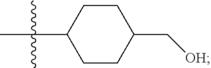 [SMILES: *C1CCC(CO)CC1]

and
W is not —SH, —OH, —S—(C₁-C₆)alkyl), or —S—(C₁-C₆)alkyl) if Z is —O—(C₁-C₆)alkyl);
wherein the symbol [symbol] , when drawn across a bond, indicates the point of attachment to the rest of the molecule.

In some embodiments, X is N.

In some embodiments, X is CH.

In some embodiments, Z is selected from —OMe or —NH-cyclohexyl; or an unsubstituted or substituted phenyl, pyridyl, benzothiophenyl, thiazolyl, pyradizinyl, pyrimidinyl, indolyl, cyclohexyl, morpholinyl, pyrrolidinyl, piperazinyl, piperidinyl, isothiazolyl, or thiomorpholinyl group. In some such embodiments, Z is selected from —OMe or —NH-cyclohexyl; or an unsubstituted or substituted phenyl, pyridyl, benzothiophenyl, thiazolyl, pyradizinyl, pyrimidinyl, indolyl, cyclohexyl, morpholinyl, pyrrolidinyl, piperazinyl, or piperidinyl.

In some embodiments, Z is an unsubstituted or substituted phenyl, pyridyl, benzothiophenyl, thiazolyl, pyradizinyl, pyrimidinyl, indolyl, cyclohexyl, morpholinyl, pyrrolidinyl, piperazinyl, piperidinyl, isothiazolyl, or thiomorpholinyl group. In some embodiments, Z is an unsubstituted or substituted phenyl, pyridyl, benzothiophenyl, thiazolyl, pyradizinyl, pyrimidinyl, indolyl, cyclohexyl, morpholinyl, pyrrolidinyl, piperazinyl, or piperidinyl group. In some such embodiments, Z is a substituted phenyl, pyridyl, benzothiophenyl, thiazolyl, pyradizinyl, pyrimidinyl, indolyl, cyclohexyl, morpholinyl, pyrrolidinyl, piperazinyl, or piperidinyl group.

In some embodiments, Z is an unsubstituted or substituted phenyl or pyridyl. In some such embodiments, Z is a substituted phenyl or pyridyl. In some other such embodiments, Z is a substituted phenyl.

In some embodiments, Z is selected from

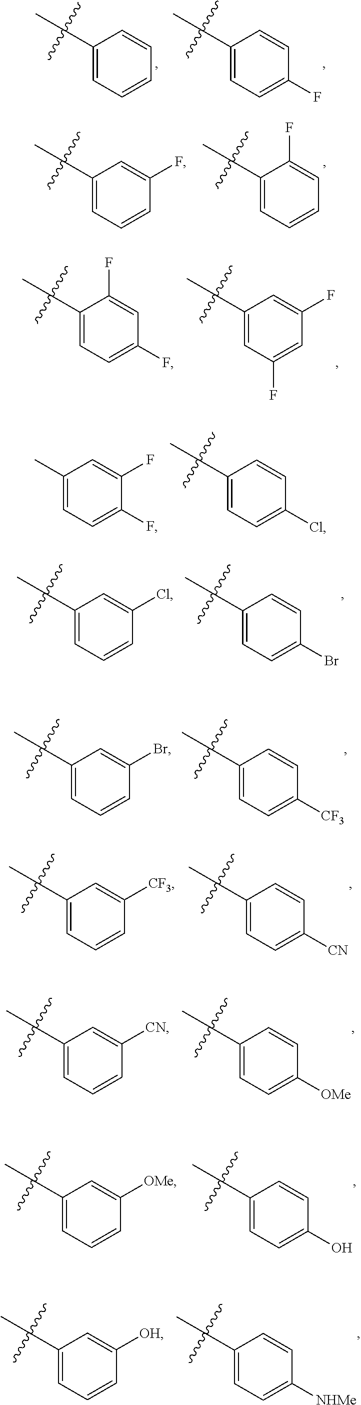

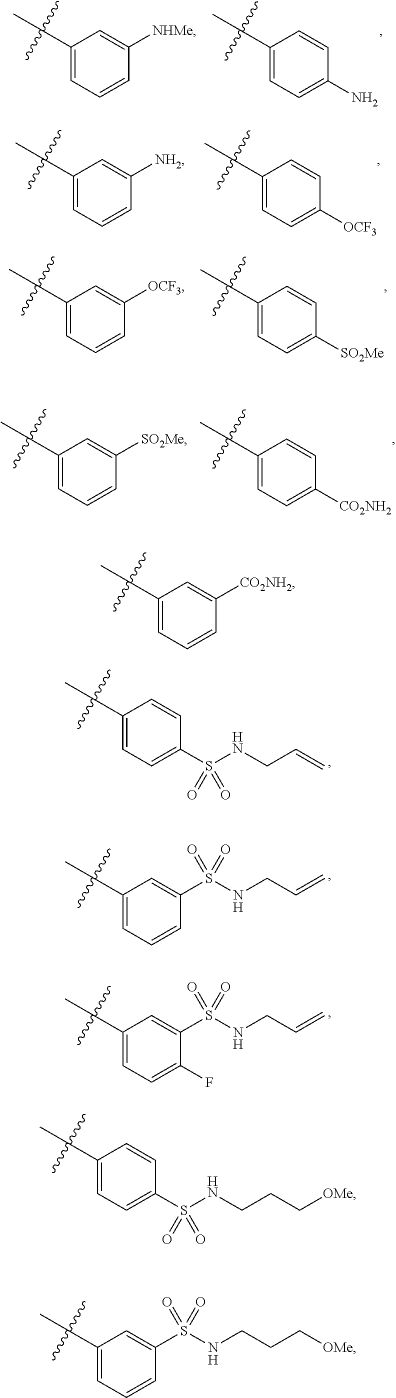

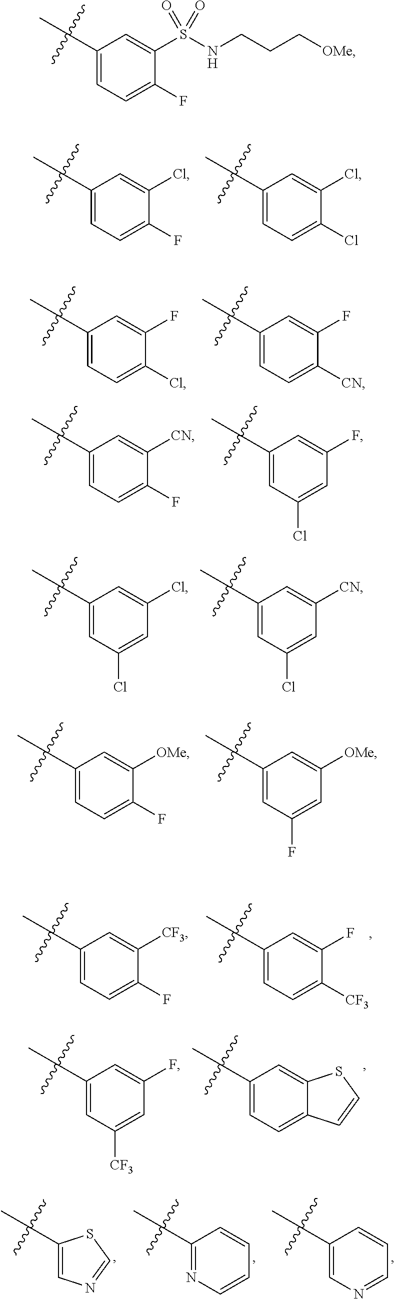

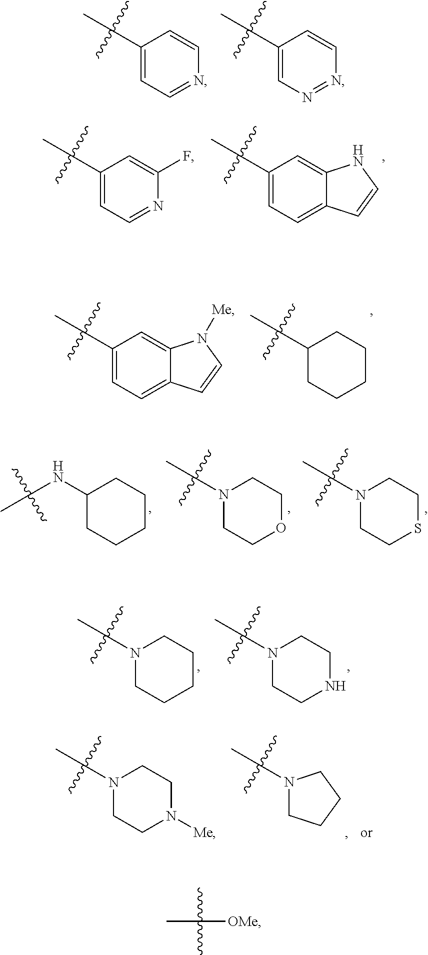

wherein the symbol [symbol] , when drawn across a bond, indicates the point of attachment to the rest of the molecule.

In some embodiments, Z is selected from

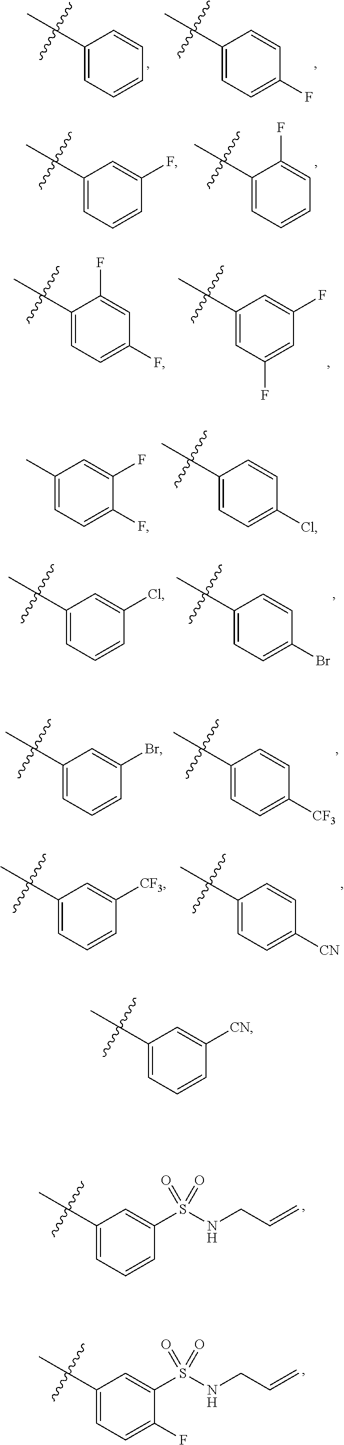

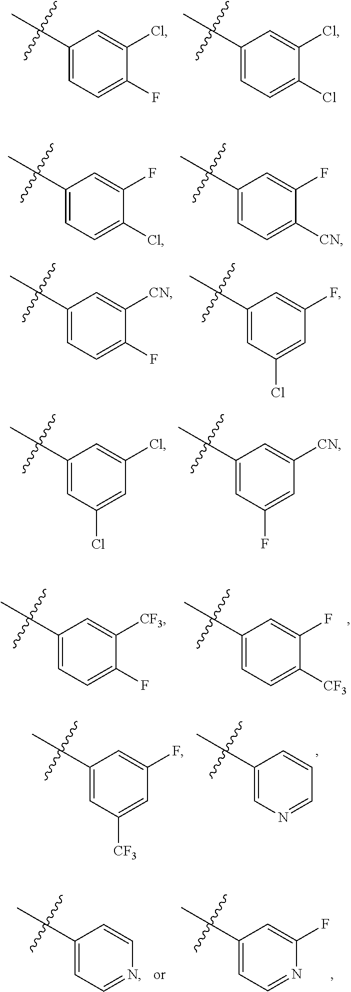

wherein the symbol [symbol] , when drawn across a bond, indicates the point of attachment to the rest of the molecule.

In some embodiments, Y is an unsubstituted or substituted cycloheptyl, cyclohexyl, cyclopentyl, cyclobutyl, piperidinyl, pyrrolidinyl, azetidinyl, adamantyl, bicyclo[2.2.2]octyl, bicyclo[3.2.1]octyl, bicyclo[4.1.1]octyl, bicyclo[2.2.1]heptyl, bicyclo[3.1.1]heptyl, or bicyclo[2.1.1]hexyl. In some such embodiments, Y is a substituted cycloheptyl, cyclohexyl, cyclopentyl, cyclobutyl, piperidinyl, pyrrolidinyl, adamantyl, bicyclo[2.2.2]octyl, bicyclo[3.2.1]octyl, bicyclo[4.1.1]octyl, bicyclo[2.2.1]heptyl, bicyclo[3.1.1]heptyl, or bicyclo[2.1.1]hexyl group. In some such embodiments, Y is an unsubstituted or substituted cyclohexyl. In some such embodiments, Y is a substituted cyclohexyl. In other such embodiments, Y is an unsubstituted or substituted adamantyl. In some such embodiments, Y is an unsubstituted adamantyl. In other embodiments, Y is a substituted adamantyl. In other such embodiments, Y is an unsubstituted or substituted cyclobutyl. In some such embodiments, Y is a substituted cyclobutyl. In still other embodiments, Y is an unsubstituted or substituted cyclopentyl or cycloheptyl. In some such embodiments Y is a substituted cyclopentyl or cycloheptyl. In still other embodiments, Y is an unsubstituted or substituted piperidinyl. In some such embodiments, Y is a substituted piperidinyl. In some embodiments where Y is substituted, Y is substituted with a group that includes a carbonyl (C═O) functional group. Examples include, but are not limited to ketones, esters, and amides.

In some embodiments, Y is selected from

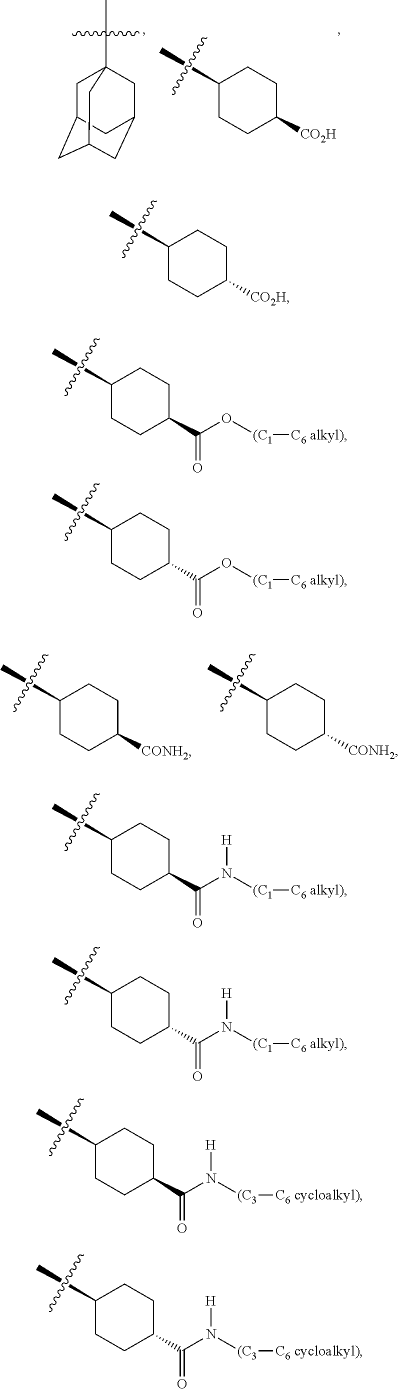

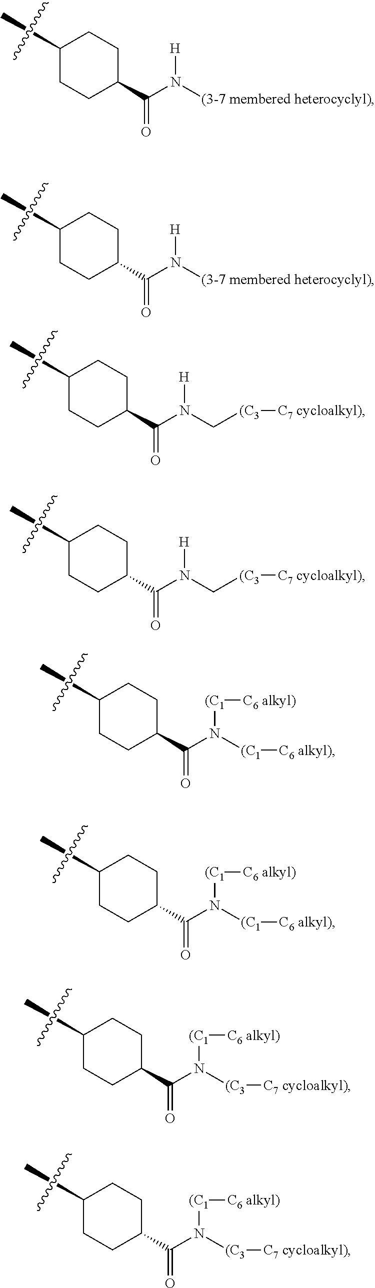

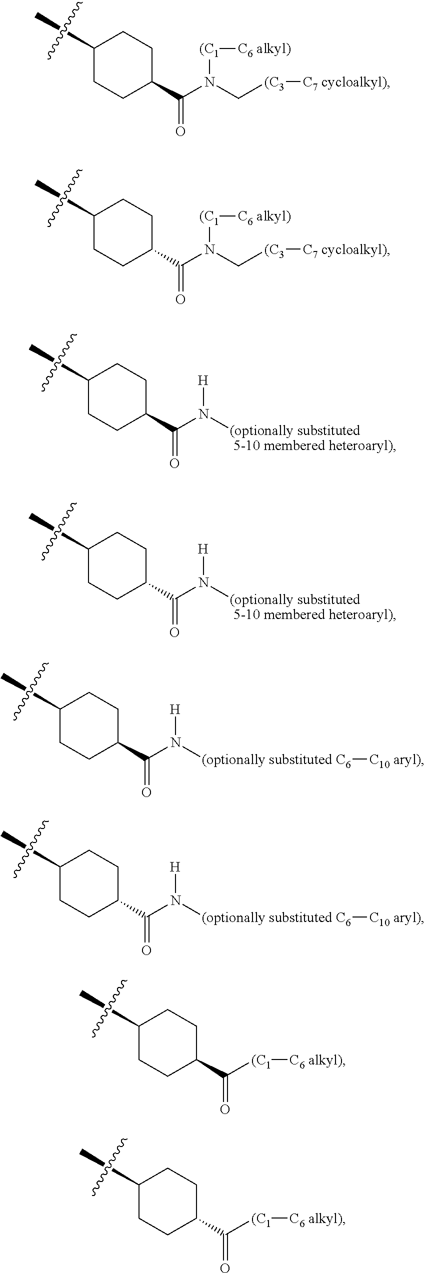

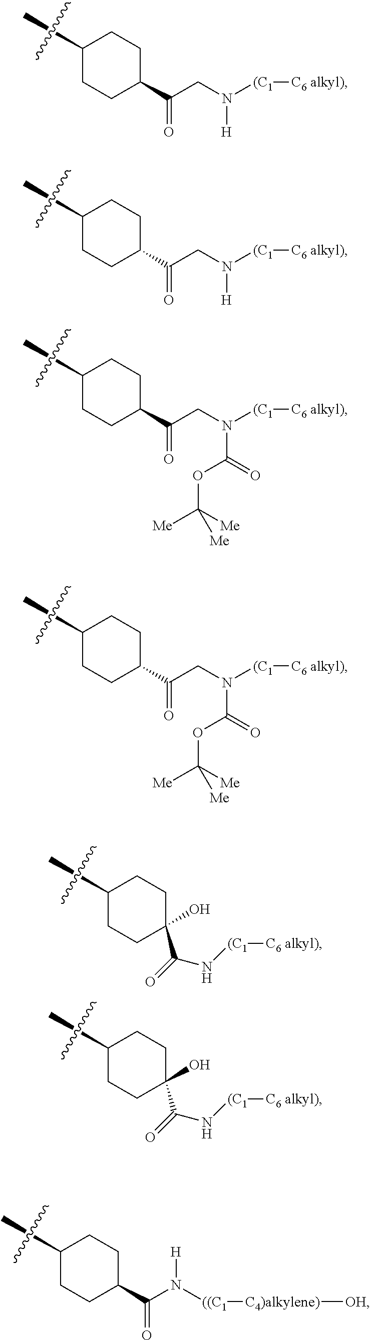

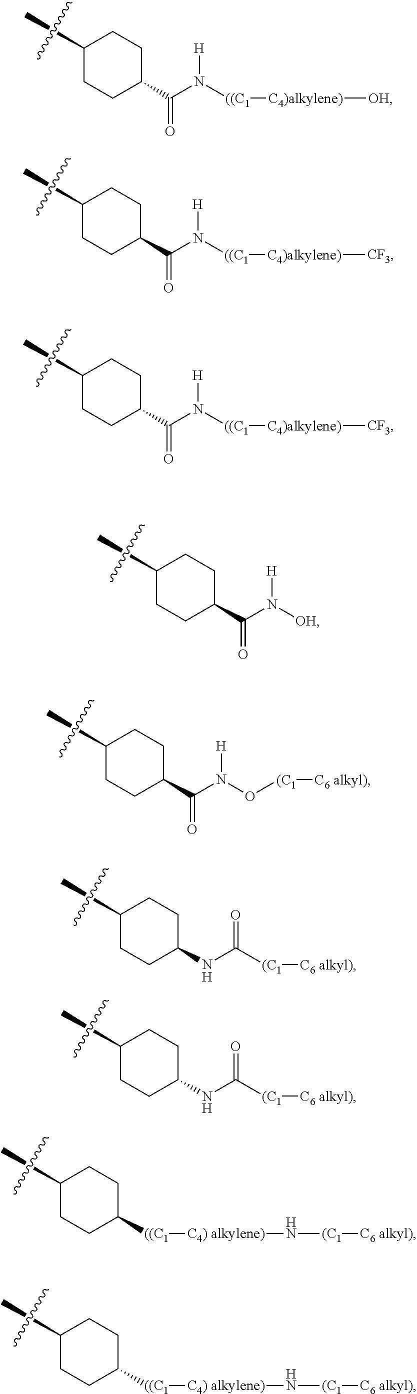

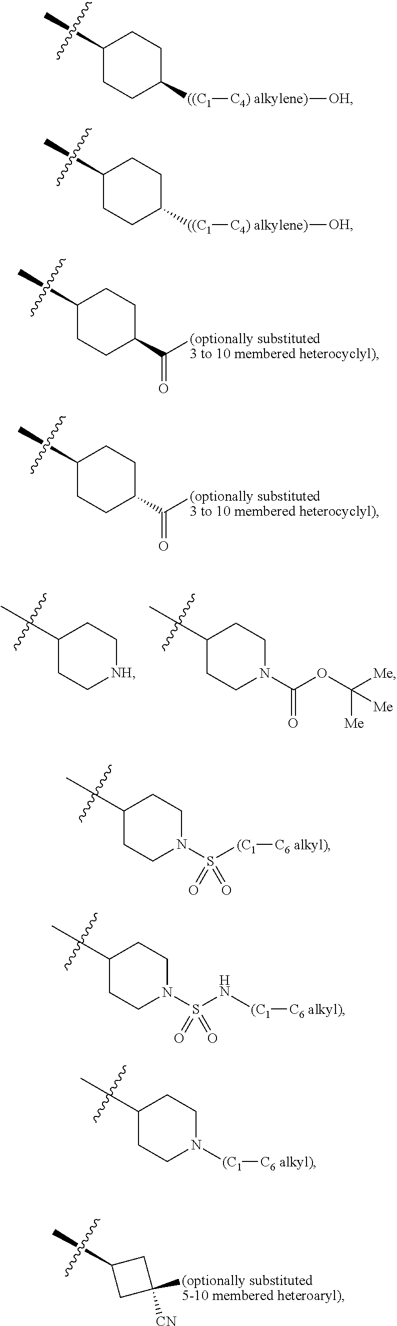

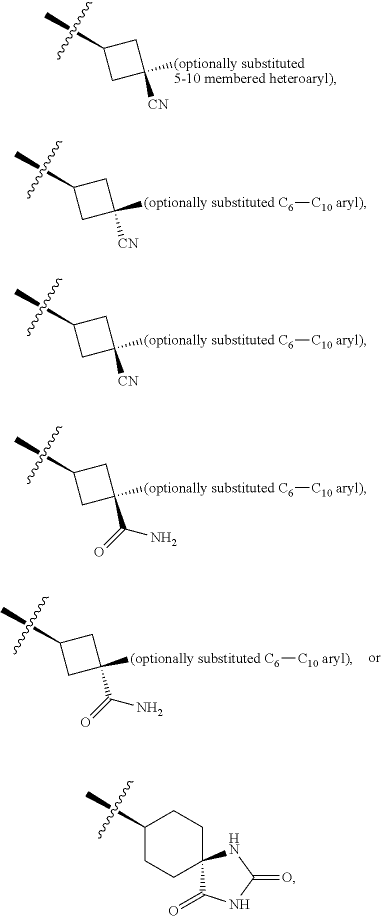

wherein the symbol [symbol] , when drawn across a bond, indicates the point of attachment to the rest of the molecule.

In some embodiments, Y is selected from

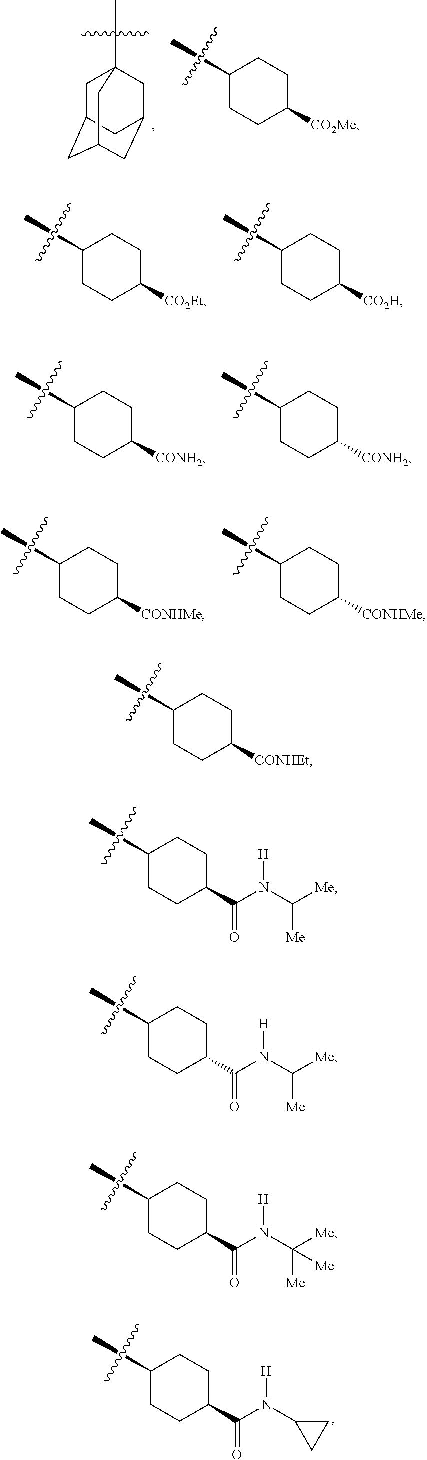

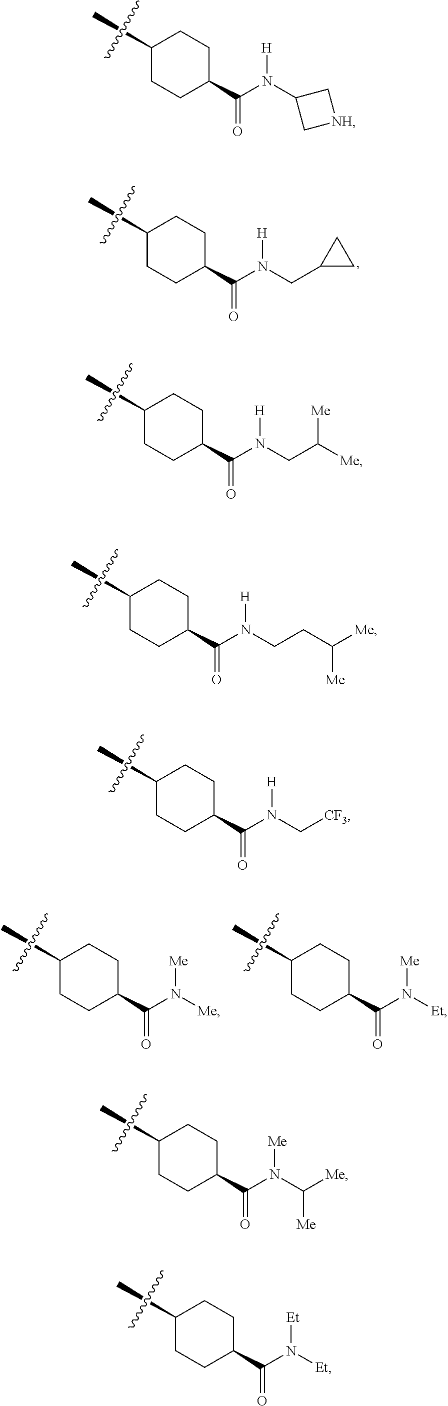

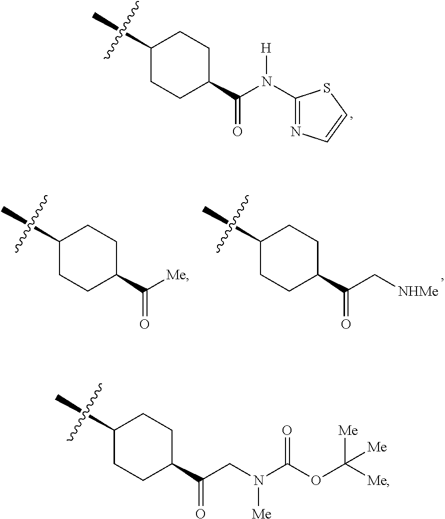

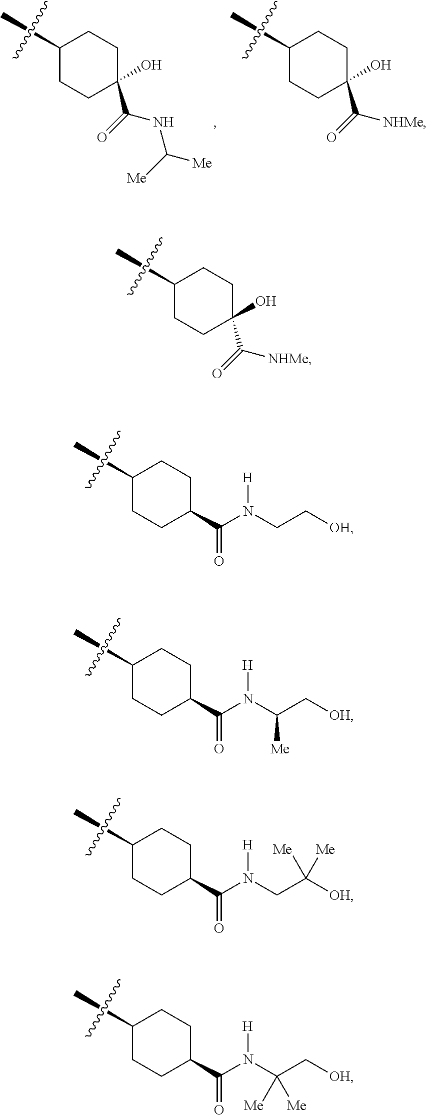

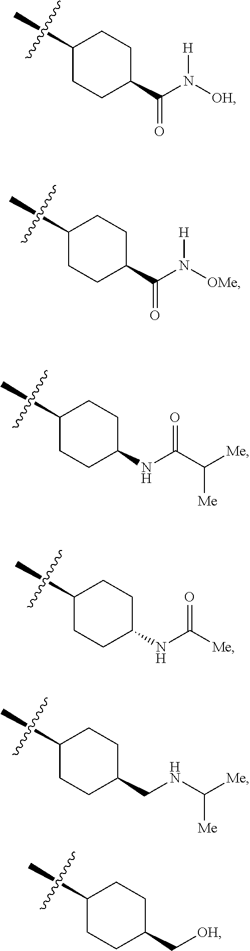

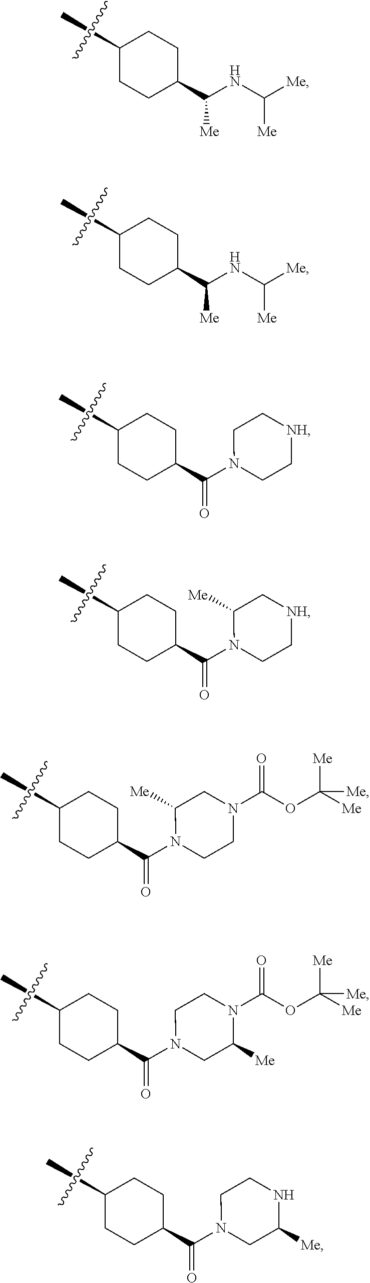

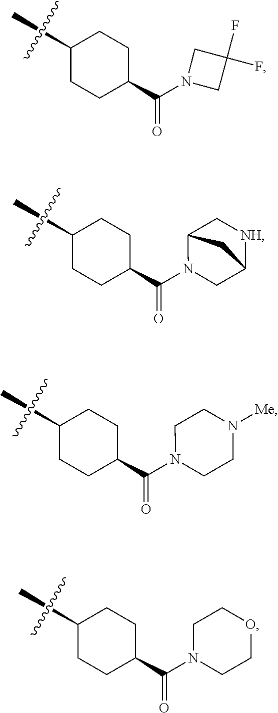

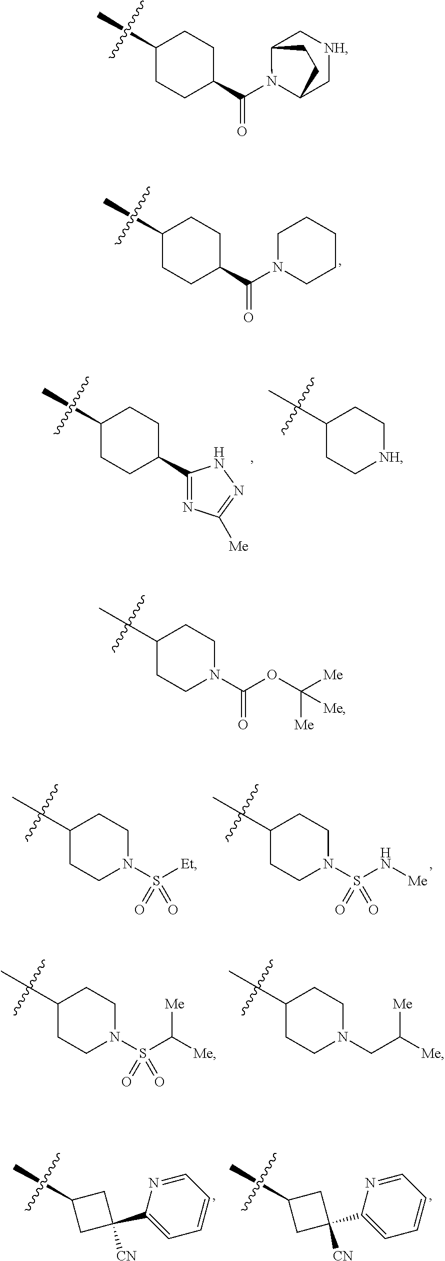

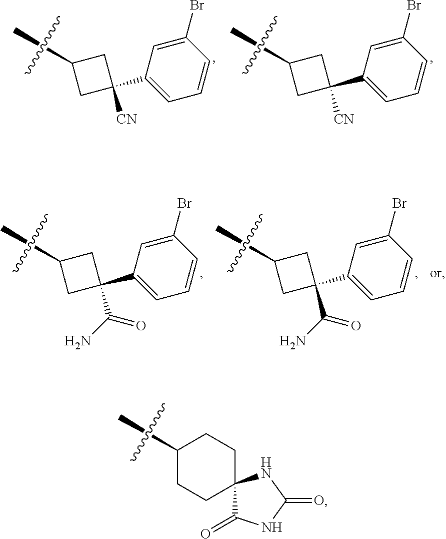

wherein the symbol [symbol] , when drawn across a bond, indicates the point of attachment to the rest of the molecule.

In some embodiments, Y is selected from

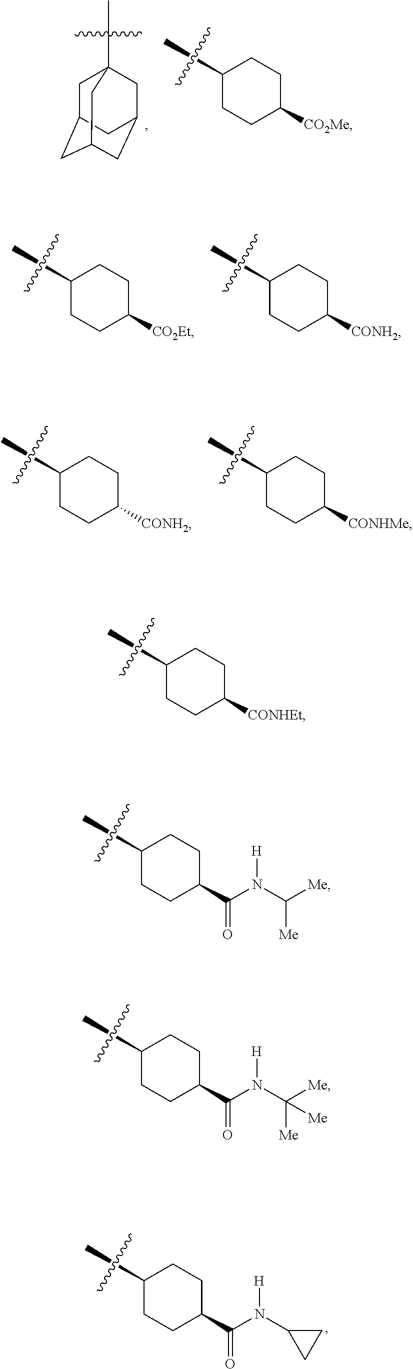

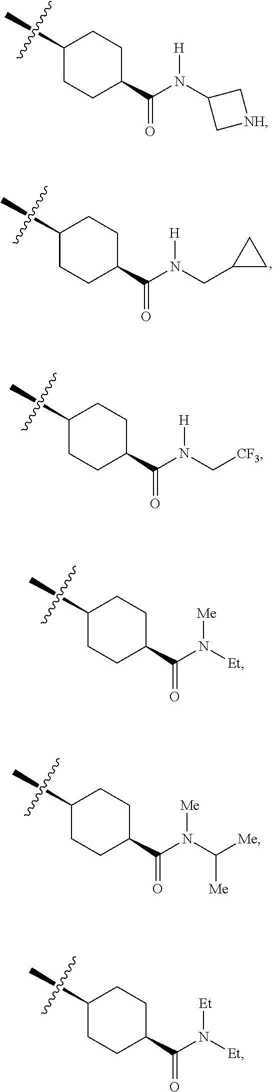

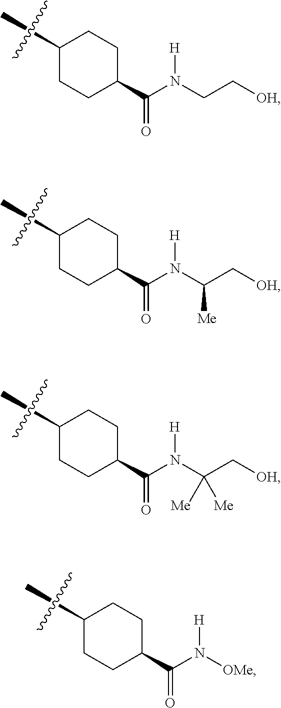

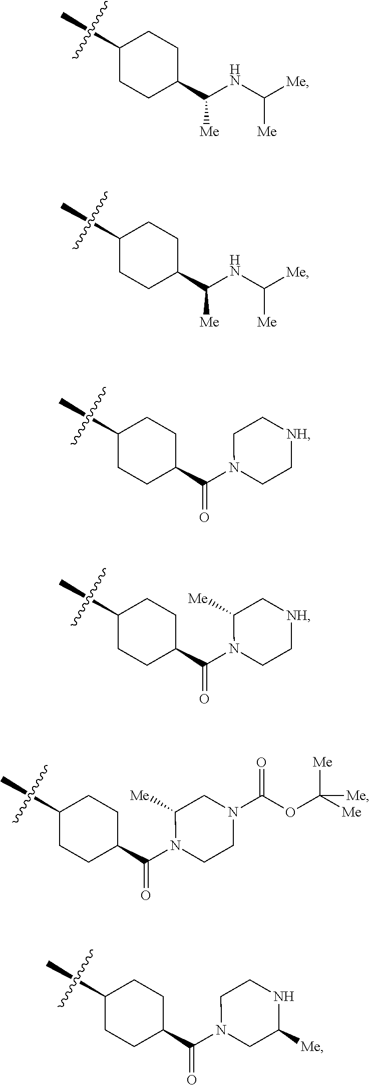

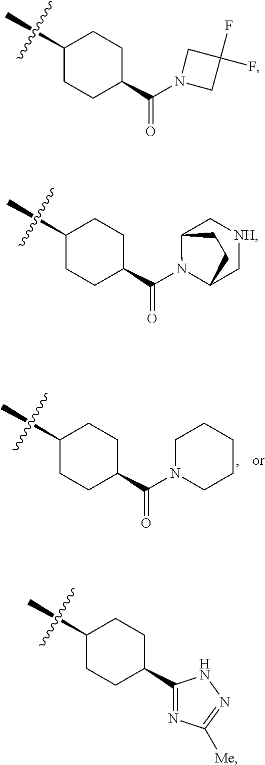

wherein the symbol [symbol] , when drawn across a bond, indicates the point of attachment to the rest of the molecule.

In some embodiments, Y is

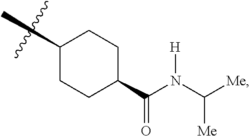

wherein the symbol [symbol] , when drawn across a bond, indicates the point of attachment to the rest of the molecule.

In some embodiments, W is selected from —CH₂OH, —CH₂OCH₃, —CH₂OCH₂CH₂OH, —CH₂OCH₂CH₂OCH₃, —OCH₂CH₂OH, —OCH₂CH₂OMe, —W′, —CH₂W′, —OW′, —OCH₂W′, —OCH₂CH₂W′, —OCH₂CH₂CH₂W′, —NHW′, —NHCH₂W′, —NHCH₂CH₂W′, —NHCH₂CH₂CH₂W′, or —W′—C(═O)—W″; wherein W′, if present, is selected from a 3-10 membered heterocyclyl comprising 1 or 2 heteroatoms selected from N, O, and S; a C₆-C₁₀ aryl; or a 5-10 membered heteroaryl comprising 1, 2, 3, or 4 heteroatoms independently selected from N, O, or S; wherein the 3-10 membered heterocyclyl W′ group may be monocyclic or bicyclic, and further wherein the 3-10 membered heterocyclyl, the C₆-C₁₀ aryl, or the 5-10 membered heteroaryl W′ groups are unsubstituted or are substituted with 1, 2, 3, or 4 substituents independently selected from —F, —Cl, —Br, —(C₁-C₆)alkyl, —CH(CF₃)(OH), —(C₁-C₄)alkylene-NH₂, —(C₁-C₄)alkylene-NH—(C₁-C₄)alkylene-CF₃, —C(═O)NH₂, —SO₂— (C₁-C₆)alkyl, —CF₃, —CO₂H, —(C₁-C₄)alkylene-C(═O)—(C₁-C₄)alkyl, —(C₁-C₄)alkylene-C(═O)—O— (C₁-C₄)alkyl, —(C₁-C₄)alkylene-C(═O)— OH, —(C₁-C₄)alkylene-OH, —OH, —O—(C₁-C₆)alkyl, or —SO₃H; and further wherein W′ may include 0, 1, or 2 ═O groups when W′ is a 3-10 membered heterocyclyl, and further wherein the ═O groups may be bonded to a ring carbon atom or a ring S atom; and further wherein W″, if present, is a 3-10 membered heterocyclyl comprising 1, 2, or 3 heteroatoms selected from N, O, and S, wherein the 3-10 membered heterocyclyl W″ group may be monocyclic or bicyclic, and further wherein the 3-10 membered heterocyclyl W″ group is unsubstituted or is optionally substituted with 1, 2, 3, or 4 substituents independently selected from —F, —Cl, —Br, —I, —C≡N, —NO₂, —(C₁-C₆)alkyl, —(C₂-C₆)alkenyl, —(C₂-C₆)alkynyl, —OH, —NH₂, —NH((C₁-C₄)alkyl), —N((C₁-C₄)alkyl)₂, —CF₃, —CO₂H, —C(═O)—O— (C₁-C₄)alkyl, —SH, —S—(C₁-C₆)alkyl, —OCF₃, or —OCHF₂, or a pharmaceutically acceptable salt thereof, tautomer thereof, a pharmaceutically acceptable salt of the tautomer, a stereoisomer of any of the foregoing, or a mixture thereof.

In some embodiments, W is selected from —W′, —CH₂W′, —OW′, —OCH₂W′, —OCH₂CH₂W′, —OCH₂CH₂CH₂W′, —NHW′, —NHCH₂W′, —NHCH₂CH₂W′, —NHCH₂CH₂CH₂W′, or —W′—C(═O)—W″; wherein W′, is selected from a 3-10 membered heterocyclyl comprising 1 or 2 heteroatoms selected from N, O, and S; a C₆-C₁₀ aryl; or a 5-10 membered heteroaryl comprising 1, 2, 3, or 4 heteroatoms independently selected from N, O, or S; wherein the 3-10 membered heterocyclyl W′ group may be monocyclic or bicyclic, and further wherein the 3-10 membered heterocyclyl, the C₆-C₁₀ aryl, or the 5-10 membered heteroaryl W′ groups are unsubstituted or are substituted with 1, 2, 3, or 4 substituents independently selected from —F, —Cl, —Br, —(C₁-C₆)alkyl, —CH(CF₃)(OH), —(C₁-C₄)alkylene-NH₂, —(C₁-C₄)alkylene-NH—(C₁-C₄)alkylene-CF₃, —C(═O)NH₂, —SO₂— (C₁-C₆)alkyl, —CF₃, —CO₂H, —(C₁-C₄)alkylene-C(═O)—(C₁-C₄)alkyl, —(C₁-C₄)alkylene-C(═O)—O— (C₁-C₄)alkyl, —(C₁-C₄)alkylene-C(═O)— OH, —(C₁-C₄)alkylene-OH, —OH, —O—(C₁-C₆)alkyl, or —SO₃H; and further wherein W′ may include 0, 1, or 2 ═O groups when W′ is a 3-10 membered heterocyclyl, and further wherein the ═O groups may be bonded to a ring carbon atom or a ring S atom; and further wherein W″, if present, is a 3-10 membered heterocyclyl comprising 1, 2, or 3 heteroatoms selected from N, O, and S, wherein the 3-10 membered heterocyclyl W″ group may be monocyclic or bicyclic, and further wherein the 3-10 membered heterocyclyl W″ group is unsubstituted or is optionally substituted with 1, 2, 3, or 4 substituents independently selected from —F, —Cl, —Br, —I, —C≡N, —NO₂, —(C₁-C₆)alkyl, —(C₂-C₆)alkenyl, —(C₂-C₆)alkynyl, —OH, —NH₂, —NH((C₁-C₄)alkyl), —N((C₁-C₄)alkyl)₂, —CF₃, —CO₂H, —C(═O)—O—(C₁-C₄)alkyl, —SH, —S—(C₁-C₆)alkyl, —OCF₃, or —OCHF₂.

In some embodiments, W is selected from —H, —F, —Cl, —OH, —OMe, —SO₂Me, —CH₂OH, —CH₂OMe, —OCH₂CH₂OH, —OCH₂CH₂OMe, or a group selected from

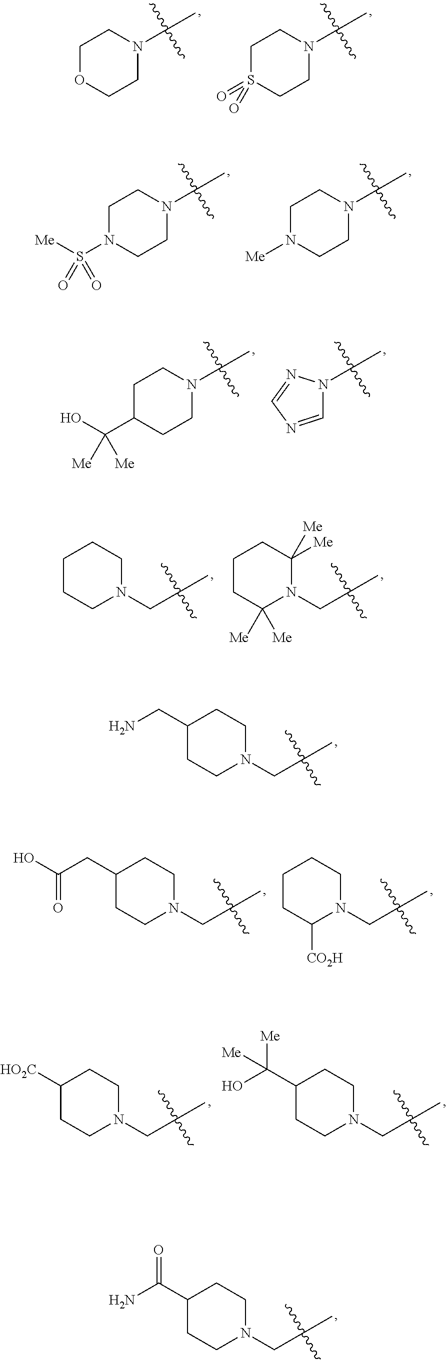

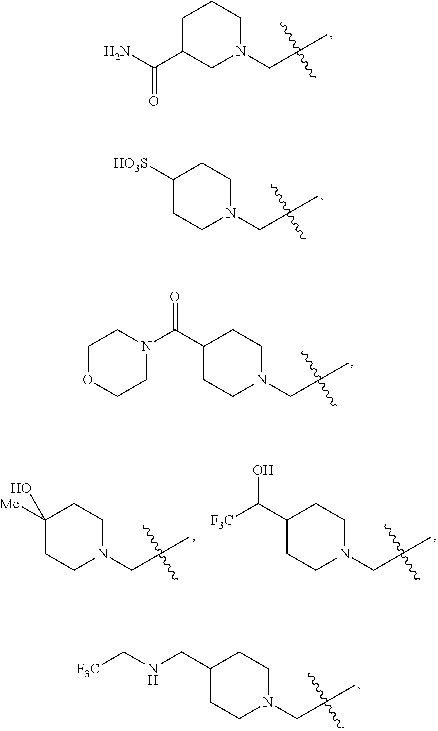 [SMILES: *CN1CCCC(C(N)=O)C1 ; *CN1CCC(S(=O)(=O)O)CC1 ; *CN1CCC(C(=O)N2CCOCC2)CC1 ; *CN1CCC(C)(O)CC1 ; *CN1CCC(C(O)C(F)(F)F)CC1 ; *CN1CCC(CNCC(F)(F)F)CC1]

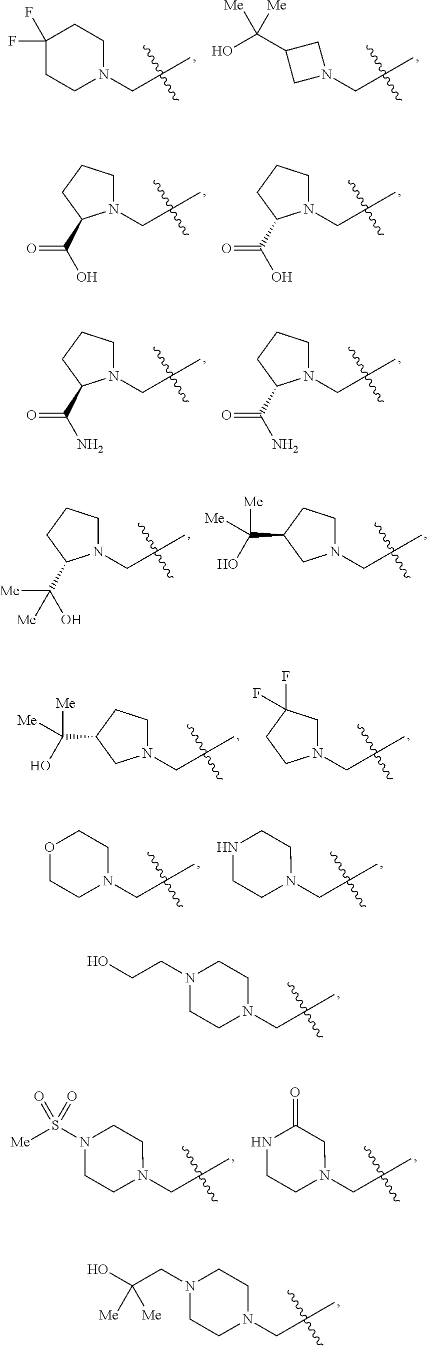

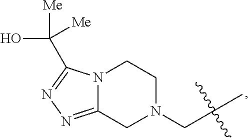 [SMILES: *CN1CCn2c(nnc2C(C)(C)O)C1]

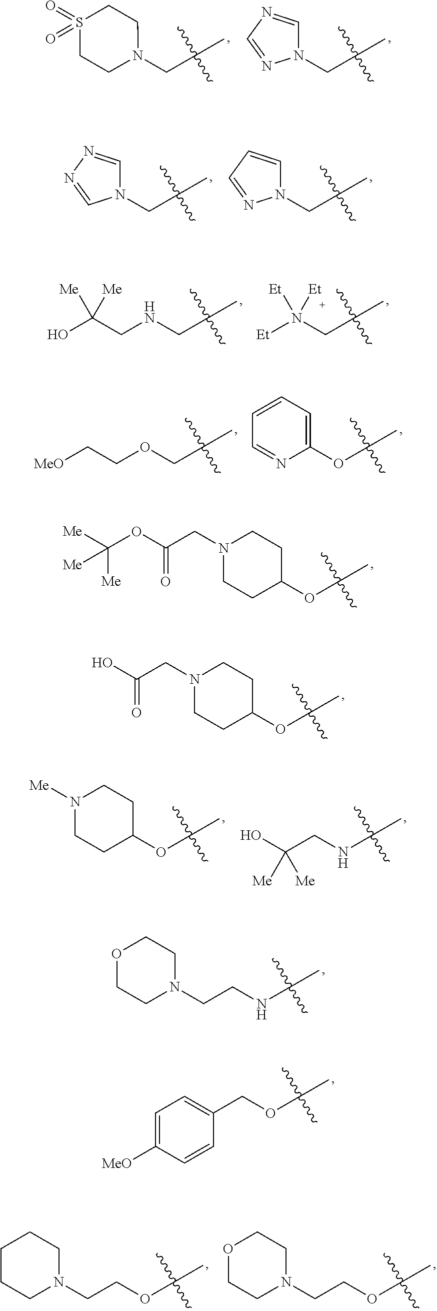

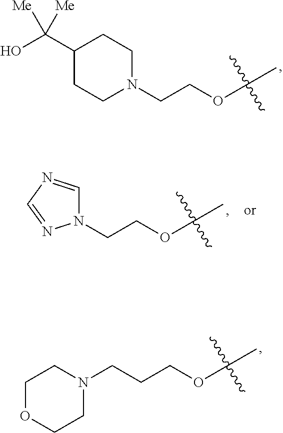 [SMILES: *OCCN1CCC(C(C)(C)O)CC1 ; *OCCn1cncn1 ; *OCCCN1CCOCC1]

wherein the symbol [symbol] , when drawn across a bond, indicates the point of attachment to the rest of the molecule.

In some embodiments, W is not —H, —F, —Cl, —OH, or —OMe.

In some embodiments, W is selected from —OH, —SO₂Me, —CH₂OH, —CH₂OMe, —OCH₂CH₂OH, —OCH₂CH₂OMe, or a group selected from

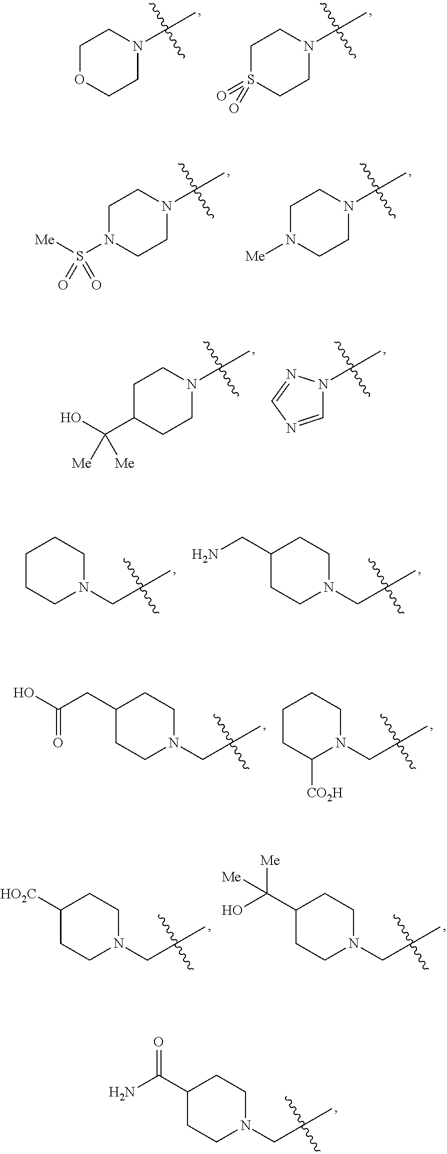

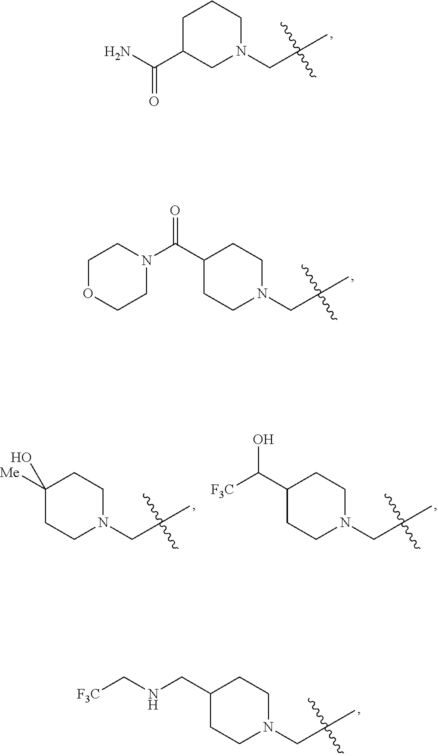 [SMILES: *CN1CCCC(C(N)=O)C1 ; *CN1CCC(C(=O)N2CCOCC2)CC1 ; *CN1CCC(C)(O)CC1 ; *CN1CCC(C(O)C(F)(F)F)CC1 ; *CN1CCC(CNCC(F)(F)F)CC1]

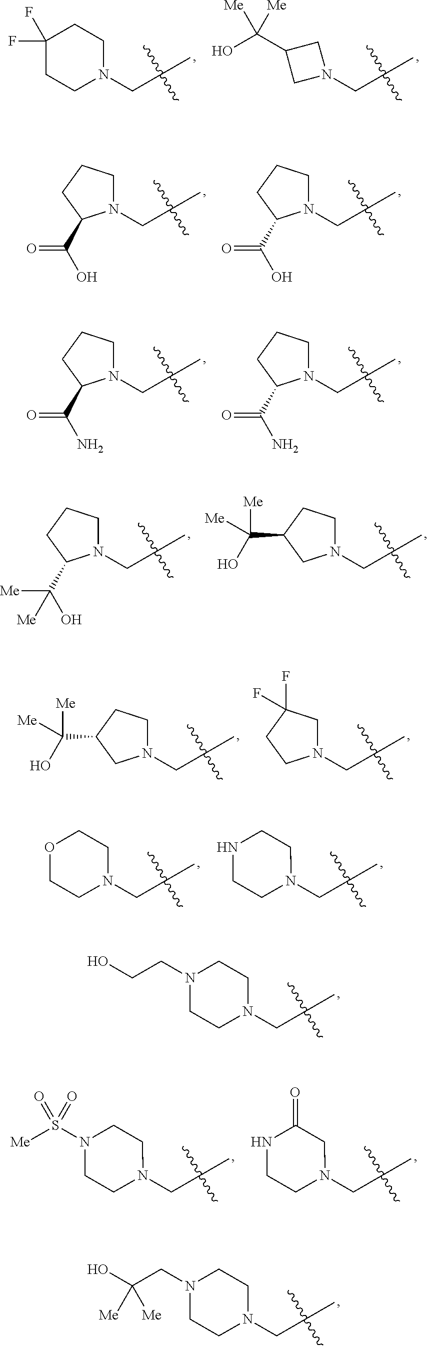

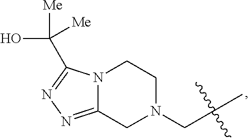 [SMILES: *CN1CCn2c(nnc2C(C)(C)O)C1]

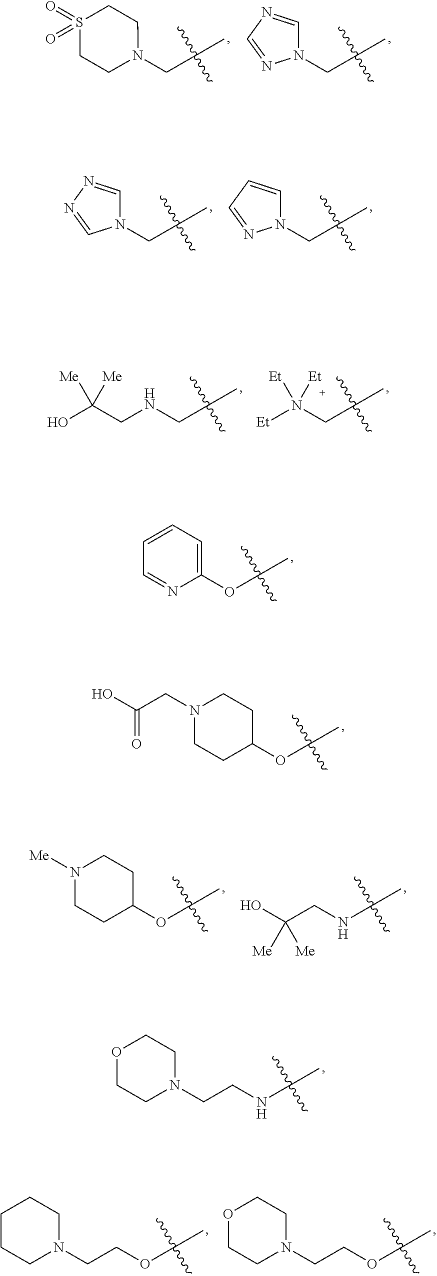

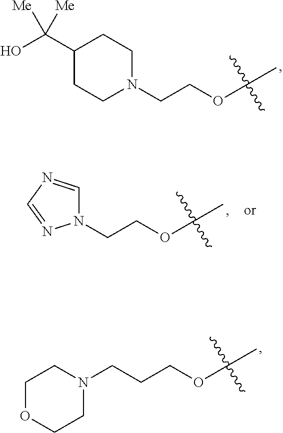 [SMILES: *OCCN1CCC(C(C)(C)O)CC1 ; *OCCn1cncn1 ; *OCCCN1CCOCC1]

wherein the symbol [symbol] , when drawn across a bond, indicates the point of attachment to the rest of the molecule.

In some embodiments, W is selected from

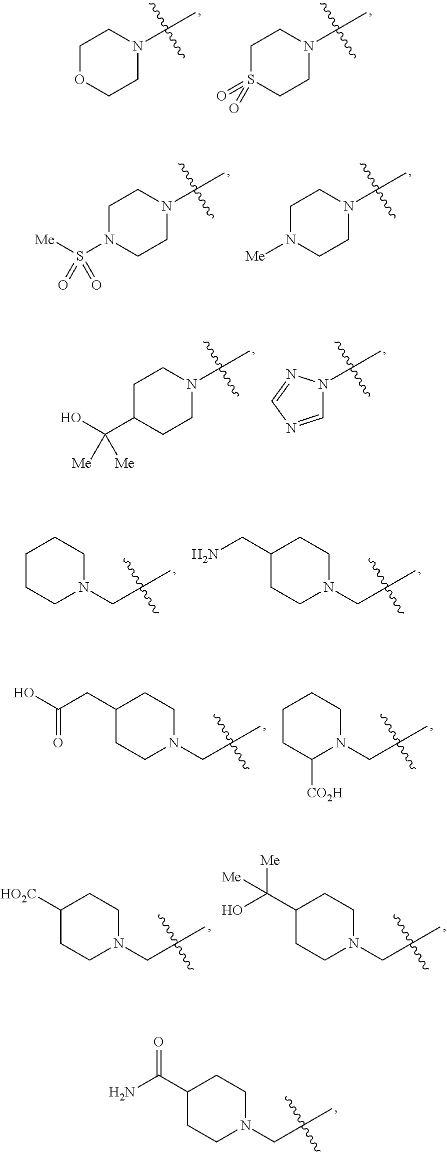

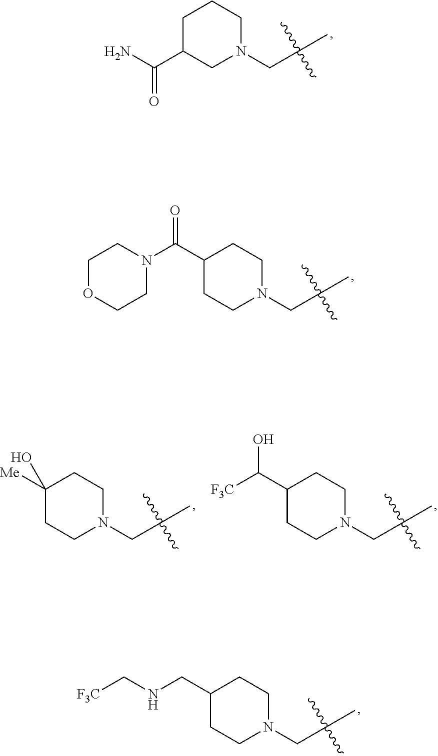 [SMILES: *CN1CCCC(C(N)=O)C1 ; *CN1CCC(C(=O)N2CCOCC2)CC1 ; *CN1CCC(C)(O)CC1 ; *CN1CCC(C(O)C(F)(F)F)CC1 ; *CN1CCC(CNCC(F)(F)F)CC1]

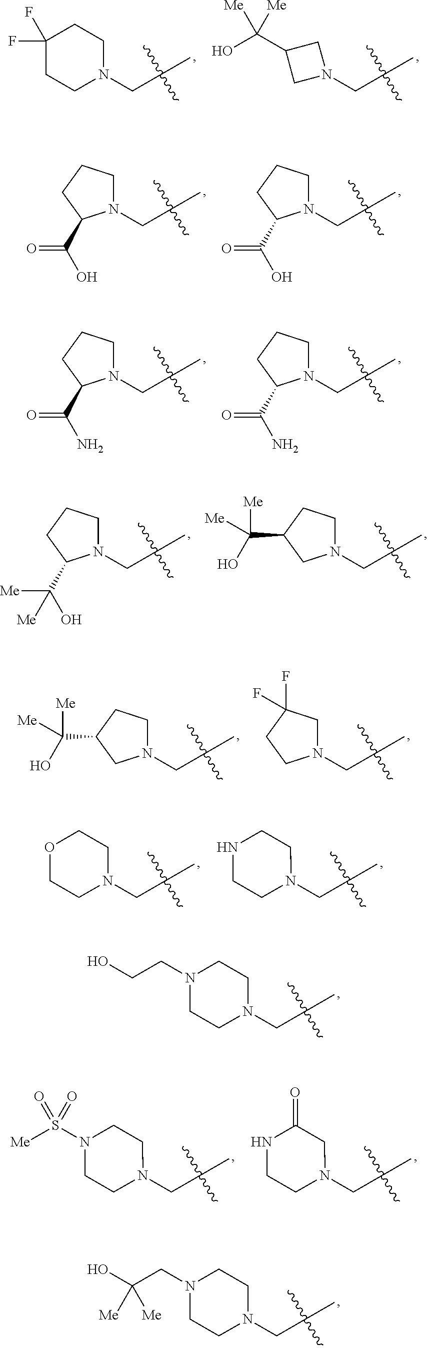

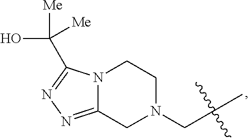 [SMILES: *CN1CCn2c(nnc2C(C)(C)O)C1]

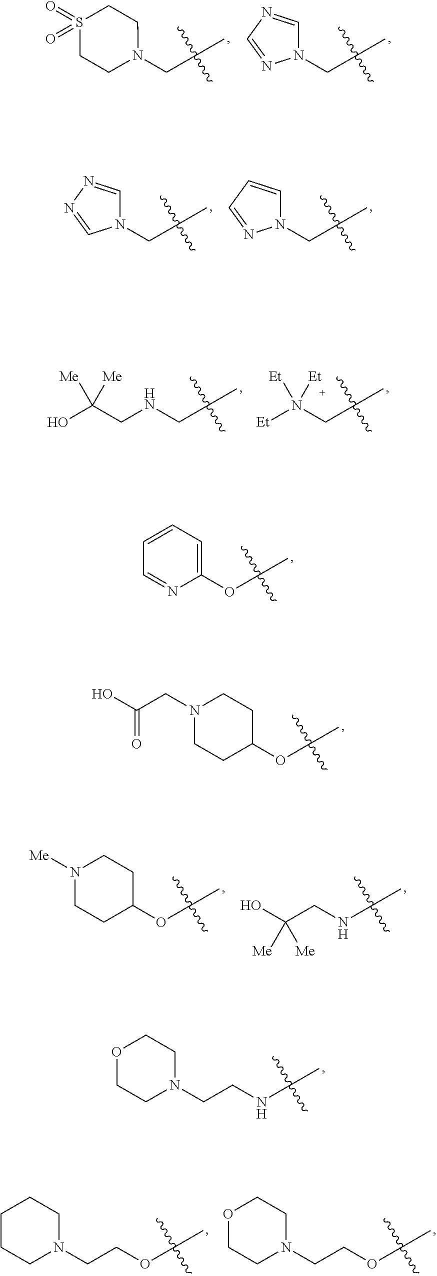

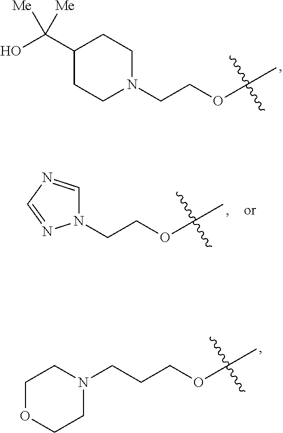 [SMILES: *OCCN1CCC(C(C)(C)O)CC1 ; *OCCn1cncn1 ; *OCCCN1CCOCC1]

wherein the symbol [symbol] , when drawn across a bond, indicates the point of attachment to the rest of the molecule.

Compounds of the invention may exist in multiple tautomeric forms. These forms are illustrated below as “Tautomer A” and “Tautomer B”:

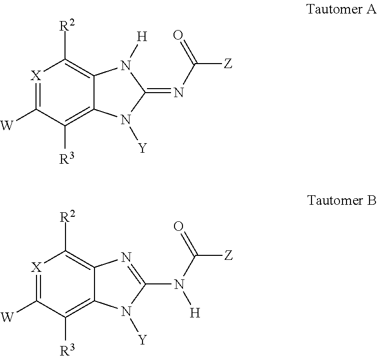

The present invention provides compounds in either tautomeric form (e.g., substantially free of the other form), or as a combination of forms (whether in equal or unequal amounts). Those skilled in the art will appreciate that, where a single tautomeric form is depicted, in some embodiments, the other form or a combination of forms may be utilized. Those skilled in the art will further appreciate that this same principle may, in some embodiments, apply to different salt and/or stereoisomeric forms of a depicted structure.

Compounds of the present disclosure include, but are not limited to, compounds of Formula I and all pharmaceutically acceptable forms thereof. Pharmaceutically acceptable forms of the compounds recited herein include pharmaceutically acceptable salts, solvates, crystal forms (including polymorphs and clathrates), chelates, non-covalent complexes, prodrugs, and mixtures thereof. In certain embodiments, the compounds described herein are in the form of pharmaceutically acceptable salts. As used herein, the term “compound” encompasses not only the compound itself, but also a pharmaceutically acceptable salt thereof, a solvate thereof, a chelate thereof, a non-covalent complex thereof, a prodrug thereof, and mixtures of any of the foregoing. In some embodiments, the term “compound” encompasses the compound itself, pharmaceutically acceptable salts thereof, tautomers of the compound, pharmaceutically acceptable salts of the tautomers, and ester prodrugs such as (C₁-C₄)alkyl esters. In other embodiments, the term “compound” encompasses the compound itself, pharmaceutically acceptable salts thereof, tautomers of the compound, pharmaceutically acceptable salts of the tautomers.

In some embodiments, the compound of formula I has the structure:

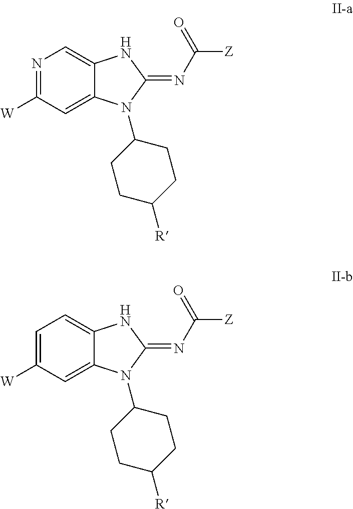

wherein W, Z and R′ are as defined above and described herein.

In some embodiments, a compound of formula I has the structure:

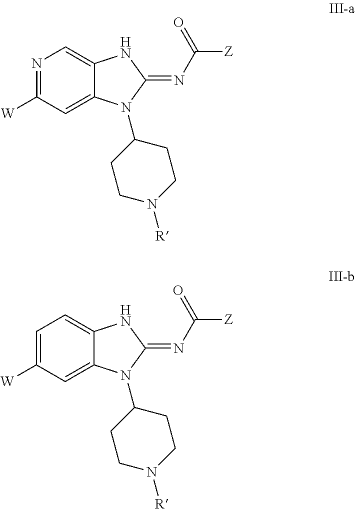

wherein W, Z and R′ are as defined above and described herein.

In some embodiments, a compound of formula I has the structure:

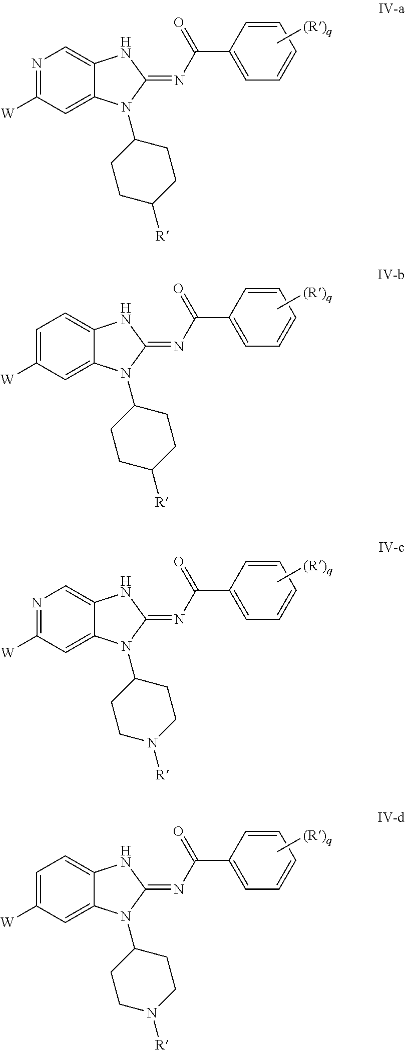

wherein W, Z, R′ and q are as defined above and described herein.

In some embodiments, the present invention encompasses the finding that compounds of formula I, and in particular compounds of formulae I-a and I-b, cross the blood-brain barrier (BBB). Accordingly, the present invention provides a method of treating a disease, disorder or condition localized or present in the central nervous system, the method comprising administering to a patient in need thereof a compound of formula I. In some such embodiments, the compound of formula I is selected from compound I-a and I-b. In some embodiments, the disease, disorder or condition localized or present in the central nervous system is cancer. In some embodiments, the disease, disorder or condition localized or present in the central nervous system is brain cancer. In some embodiments, the disease, disorder or condition localized or present in the central nervous system is spinal cancer.

In some embodiments, the compound is selected from

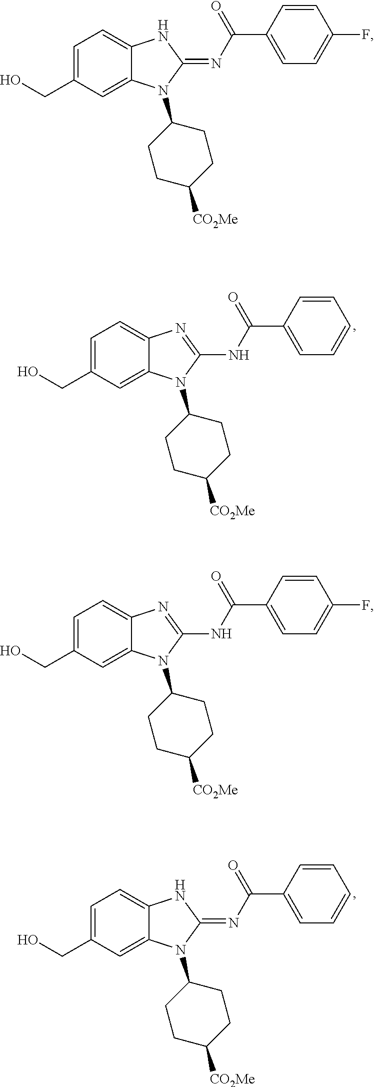 [SMILES: COC(=O)[C@H]1CC[C@@H](n2/c(=N/C(=O)c3ccc(F)cc3)[nH]c3ccc(CO)cc32)CC1 ; COC(=O)[C@H]1CC[C@@H](n2c(NC(=O)c3ccccc3)nc3ccc(CO)cc32)CC1 ; COC(=O)[C@H]1CC[C@@H](n2c(NC(=O)c3ccc(F)cc3)nc3ccc(CO)cc32)CC1 ; COC(=O)[C@H]1CC[C@@H](n2/c(=N/C(=O)c3ccccc3)[nH]c3ccc(CO)cc32)CC1]

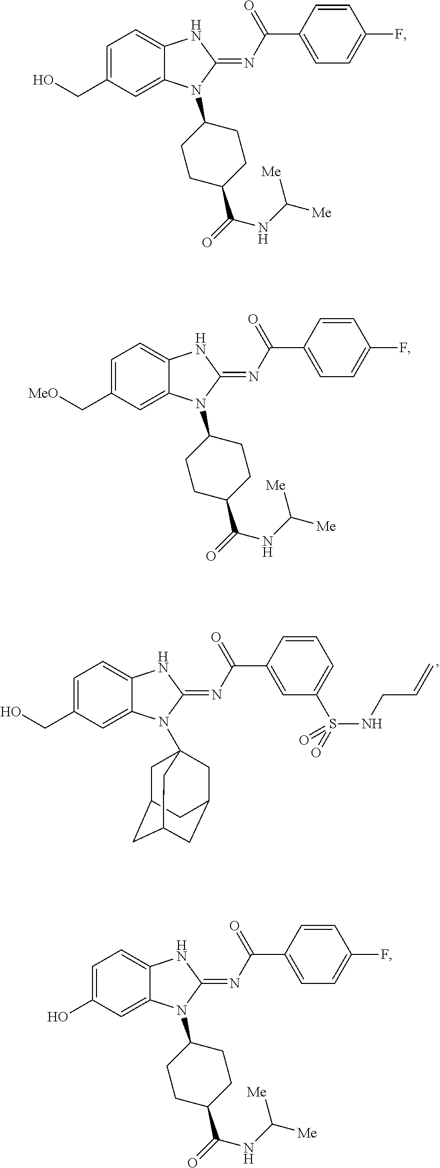 [SMILES: CC(C)NC(=O)[C@H]1CC[C@@H](n2/c(=N/C(=O)c3ccc(F)cc3)[nH]c3ccc(CO)cc32)CC1 ; COCc1ccc2[nH]/c(=N\C(=O)c3ccc(F)cc3)n([C@H]3CC[C@@H](C(=O)NC(C)C)CC3)c2c1 ; C=CCNS(=O)(=O)c1cccc(C(=O)/N=c2\[nH]c3ccc(CO)cc3n2C23CC4CC(CC(C4)C2)C3)c1 ; CC(C)NC(=O)[C@H]1CC[C@@H](n2/c(=N/C(=O)c3ccc(F)cc3)[nH]c3ccc(O)cc32)CC1]

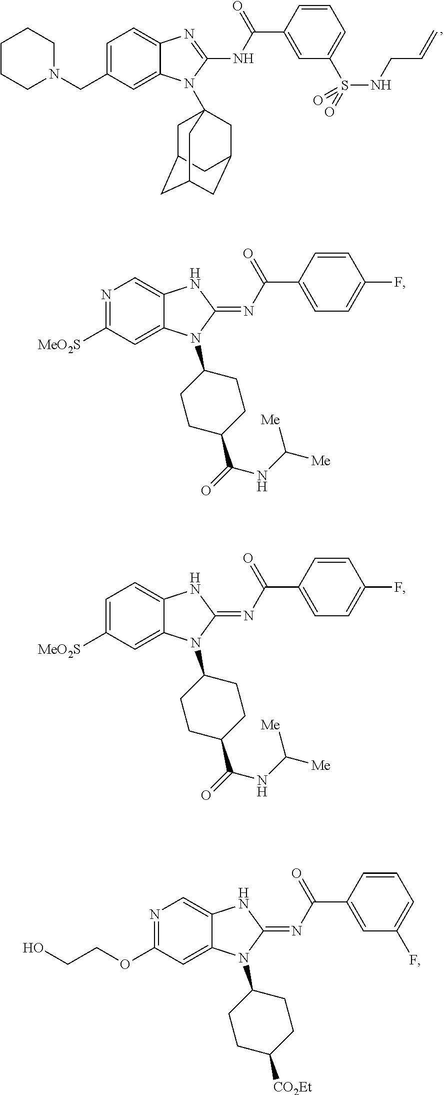 [SMILES: C=CCNS(=O)(=O)c1cccc(C(=O)Nc2nc3ccc(CN4CCCCC4)cc3n2C23CC4CC(CC(C4)C2)C3)c1 ; CC(C)NC(=O)[C@H]1CC[C@@H](n2/c(=N/C(=O)c3ccc(F)cc3)[nH]c3cnc(S(C)(=O)=O)cc32)CC1 ; CC(C)NC(=O)[C@H]1CC[C@@H](n2/c(=N/C(=O)c3ccc(F)cc3)[nH]c3ccc(S(C)(=O)=O)cc32)CC1 ; CCOC(=O)[C@H]1CC[C@@H](n2/c(=N/C(=O)c3cccc(F)c3)[nH]c3cnc(OCCO)cc32)CC1]

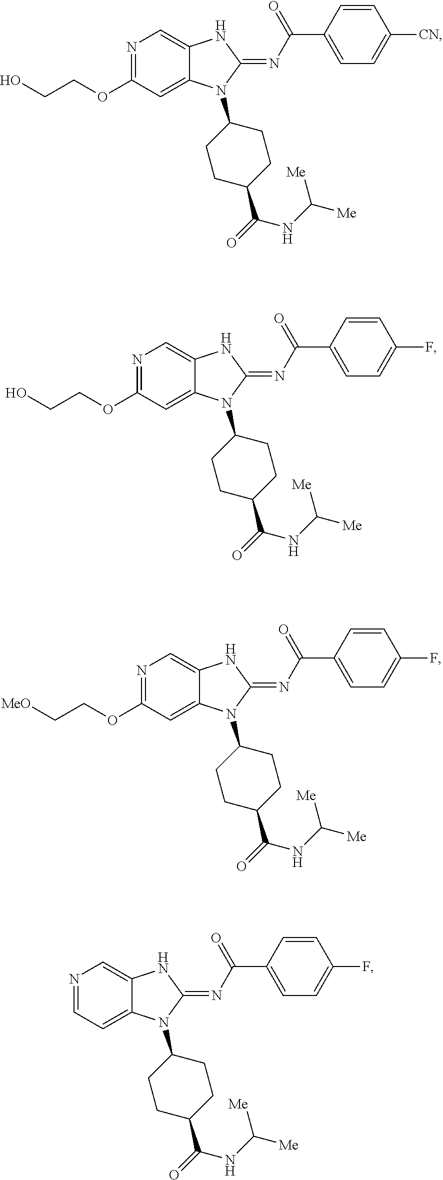 [SMILES: CC(C)NC(=O)[C@H]1CC[C@@H](n2/c(=N/C(=O)c3ccc(C#N)cc3)[nH]c3cnc(OCCO)cc32)CC1 ; CC(C)NC(=O)[C@H]1CC[C@@H](n2/c(=N/C(=O)c3ccc(F)cc3)[nH]c3cnc(OCCO)cc32)CC1 ; COCCOc1cc2c(cn1)[nH]/c(=N\C(=O)c1ccc(F)cc1)n2[C@H]1CC[C@@H](C(=O)NC(C)C)CC1 ; CC(C)NC(=O)[C@H]1CC[C@@H](n2/c(=N/C(=O)c3ccc(F)cc3)[nH]c3cnccc32)CC1]

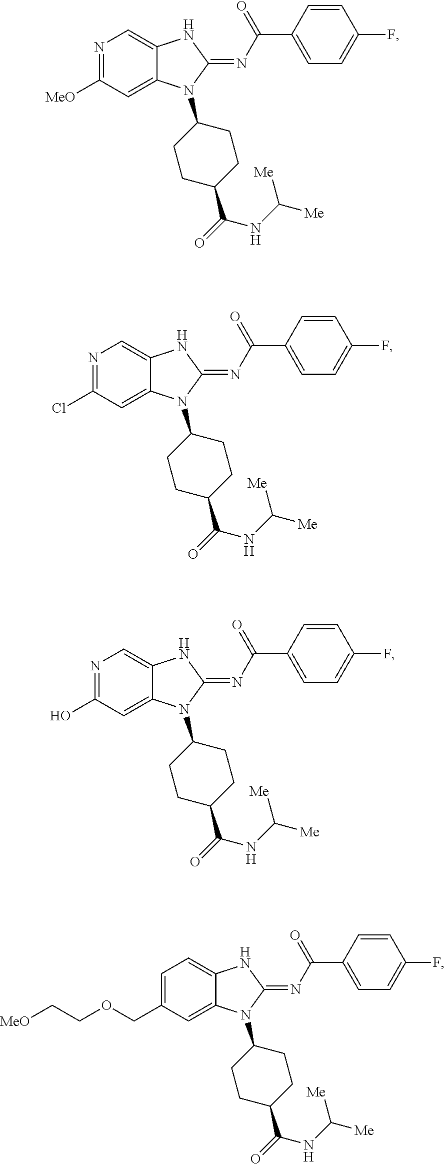 [SMILES: COc1cc2c(cn1)[nH]/c(=N\C(=O)c1ccc(F)cc1)n2[C@H]1CC[C@@H](C(=O)NC(C)C)CC1 ; CC(C)NC(=O)[C@H]1CC[C@@H](n2/c(=N/C(=O)c3ccc(F)cc3)[nH]c3cnc(Cl)cc32)CC1 ; CC(C)NC(=O)[C@H]1CC[C@@H](n2/c(=N/C(=O)c3ccc(F)cc3)[nH]c3cnc(O)cc32)CC1 ; COCCOCc1ccc2[nH]/c(=N\C(=O)c3ccc(F)cc3)n([C@H]3CC[C@@H](C(=O)NC(C)C)CC3)c2c1]

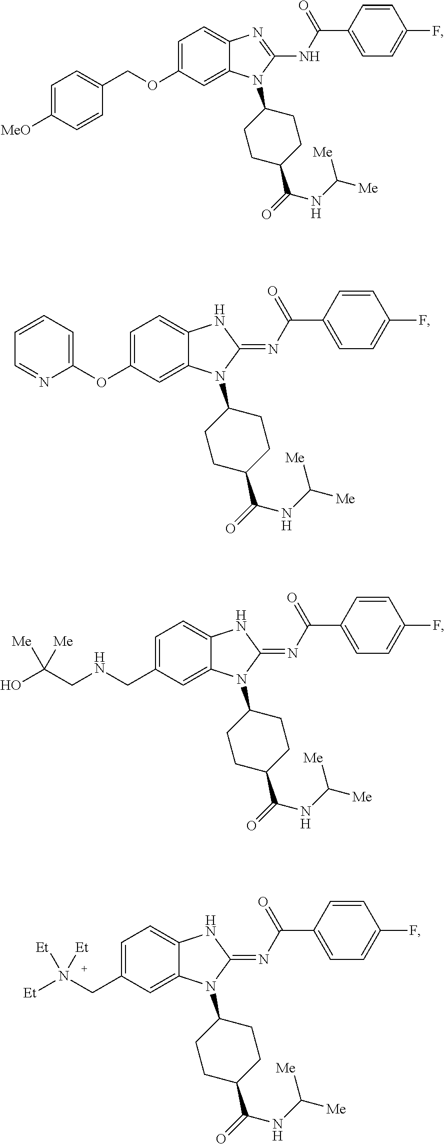

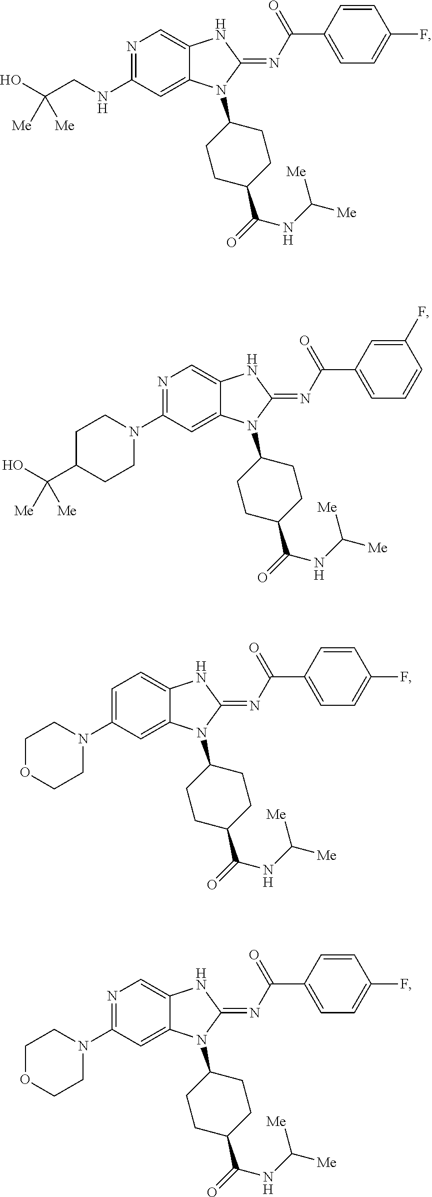 [SMILES: CC(C)NC(=O)[C@H]1CC[C@@H](n2/c(=N/C(=O)c3ccc(F)cc3)[nH]c3cnc(NCC(C)(C)O)cc32)CC1 ; CC(C)NC(=O)[C@H]1CC[C@@H](n2/c(=N/C(=O)c3cccc(F)c3)[nH]c3cnc(N4CCC(C(C)(C)O)CC4)cc32)CC1 ; CC(C)NC(=O)[C@H]1CC[C@@H](n2/c(=N/C(=O)c3ccc(F)cc3)[nH]c3ccc(N4CCOCC4)cc32)CC1 ; CC(C)NC(=O)[C@H]1CC[C@@H](n2/c(=N/C(=O)c3ccc(F)cc3)[nH]c3cnc(N4CCOCC4)cc32)CC1]

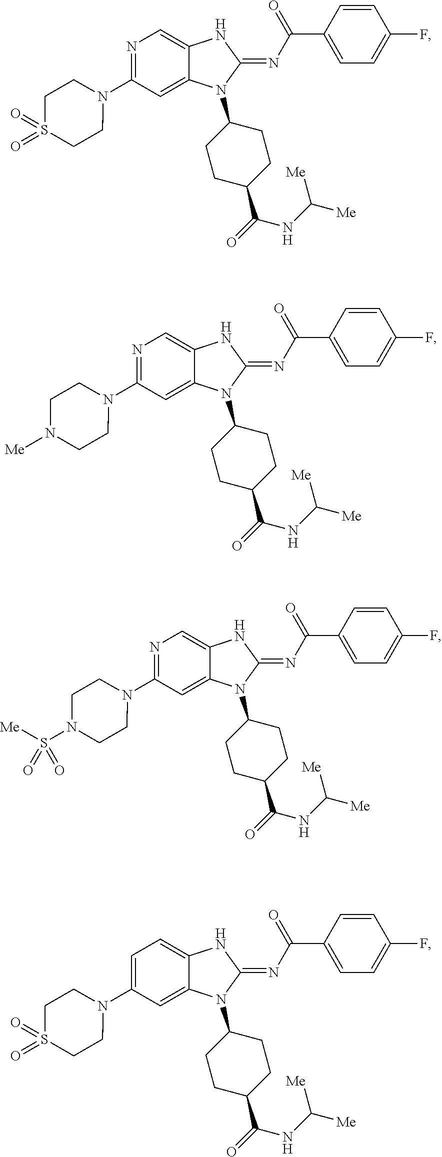 [SMILES: CC(C)NC(=O)[C@H]1CC[C@@H](n2/c(=N/C(=O)c3ccc(F)cc3)[nH]c3cnc(N4CCS(=O)(=O)CC4)cc32)CC1 ; CC(C)NC(=O)[C@H]1CC[C@@H](n2/c(=N/C(=O)c3ccc(F)cc3)[nH]c3cnc(N4CCN(C)CC4)cc32)CC1 ; CC(C)NC(=O)[C@H]1CC[C@@H](n2/c(=N/C(=O)c3ccc(F)cc3)[nH]c3cnc(N4CCN(S(C)(=O)=O)CC4)cc32)CC1 ; CC(C)NC(=O)[C@H]1CC[C@@H](n2/c(=N/C(=O)c3ccc(F)cc3)[nH]c3ccc(N4CCS(=O)(=O)CC4)cc32)CC1]

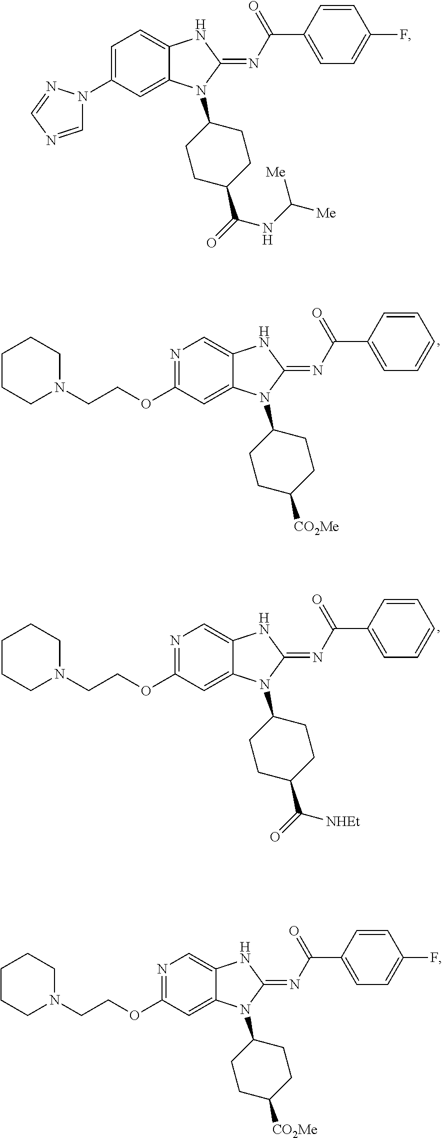

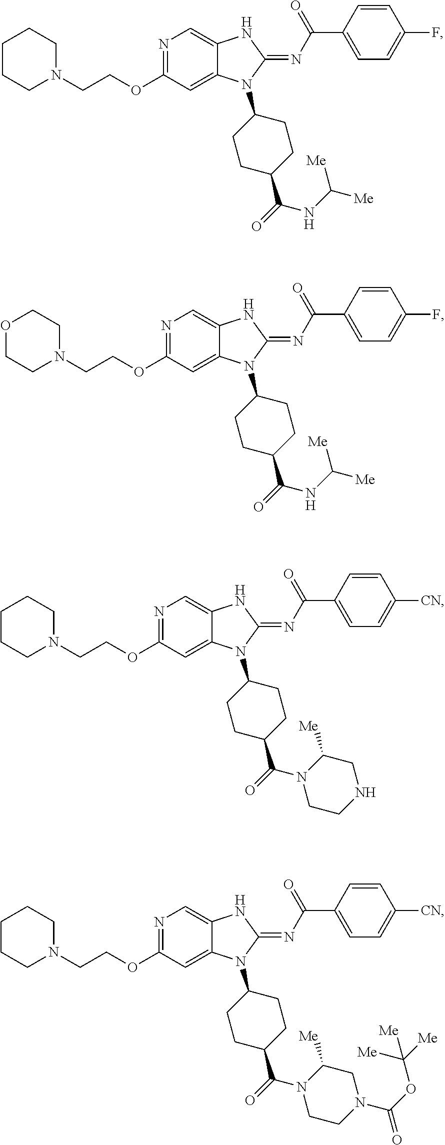

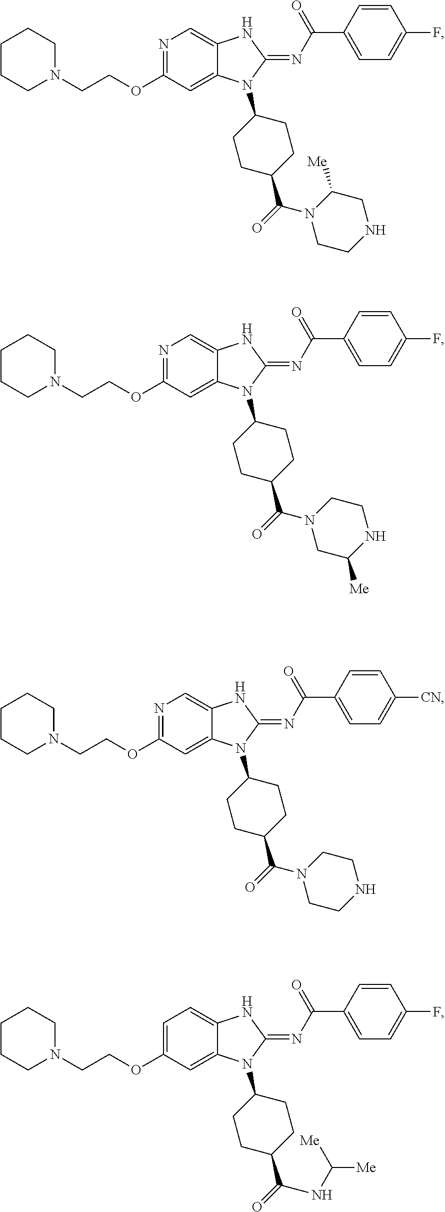

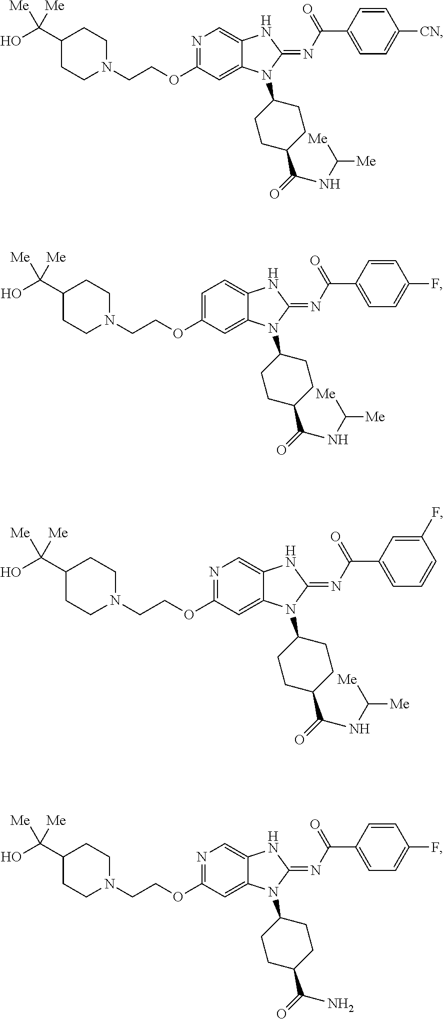 [SMILES: CC(C)NC(=O)[C@H]1CC[C@@H](n2/c(=N/C(=O)c3ccc(C#N)cc3)[nH]c3cnc(OCCN4CCC(C(C)(C)O)CC4)cc32)CC1 ; CC(C)NC(=O)[C@H]1CC[C@@H](n2/c(=N/C(=O)c3ccc(F)cc3)[nH]c3ccc(OCCN4CCC(C(C)(C)O)CC4)cc32)CC1 ; CC(C)NC(=O)[C@H]1CC[C@@H](n2/c(=N/C(=O)c3cccc(F)c3)[nH]c3cnc(OCCN4CCC(C(C)(C)O)CC4)cc32)CC1 ; CC(C)(O)C1CCN(CCOc2cc3c(cn2)[nH]/c(=N\C(=O)c2ccc(F)cc2)n3[C@H]2CC[C@@H](C(N)=O)CC2)CC1]

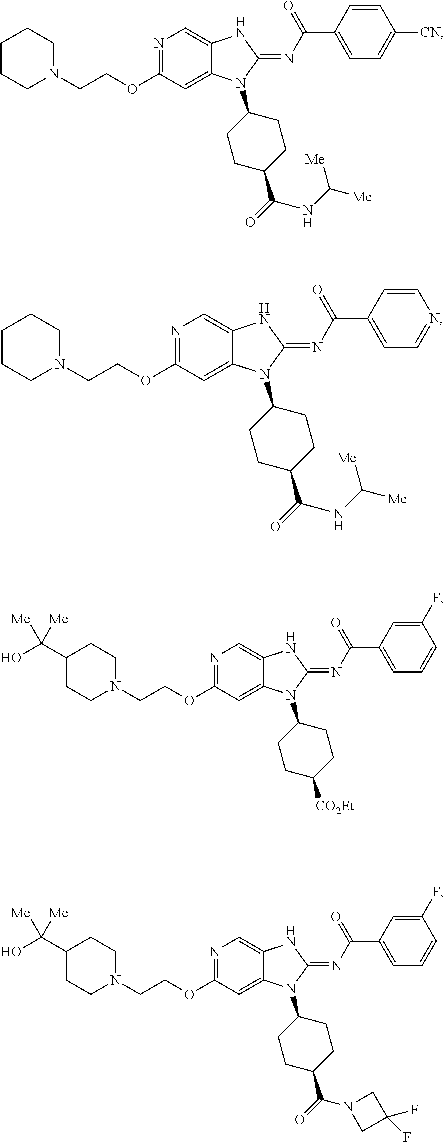 [SMILES: CC(C)NC(=O)[C@H]1CC[C@@H](n2/c(=N/C(=O)c3ccc(C#N)cc3)[nH]c3cnc(OCCN4CCCCC4)cc32)CC1 ; CC(C)NC(=O)[C@H]1CC[C@@H](n2/c(=N/C(=O)c3ccncc3)[nH]c3cnc(OCCN4CCCCC4)cc32)CC1 ; CCOC(=O)[C@H]1CC[C@@H](n2/c(=N/C(=O)c3cccc(F)c3)[nH]c3cnc(OCCN4CCC(C(C)(C)O)CC4)cc32)CC1 ; CC(C)(O)C1CCN(CCOc2cc3c(cn2)[nH]/c(=N\C(=O)c2cccc(F)c2)n3[C@H]2CC[C@@H](C(=O)N3CC(F)(F)C3)CC2)CC1]

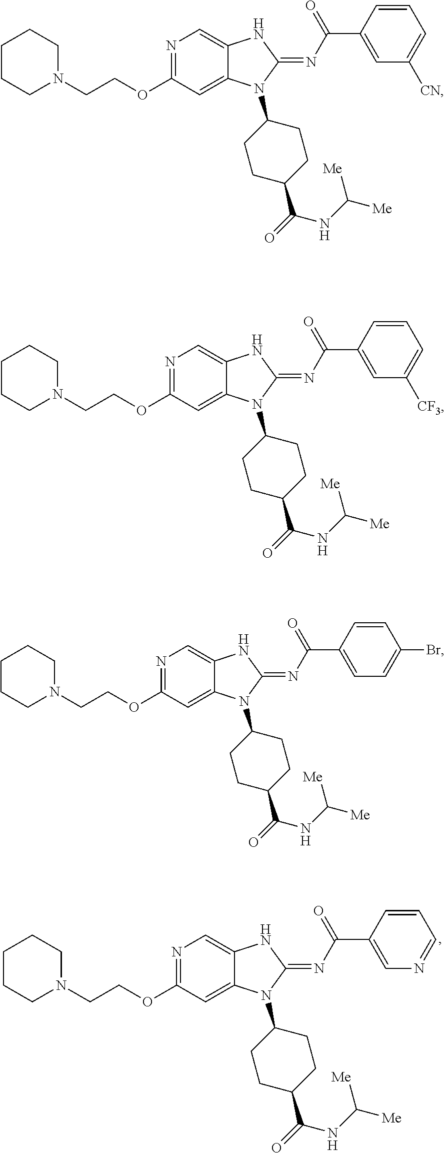 [SMILES: CC(C)NC(=O)[C@H]1CC[C@@H](n2/c(=N/C(=O)c3cccc(C#N)c3)[nH]c3cnc(OCCN4CCCCC4)cc32)CC1 ; CC(C)NC(=O)[C@H]1CC[C@@H](n2/c(=N/C(=O)c3cccc(C(F)(F)F)c3)[nH]c3cnc(OCCN4CCCCC4)cc32)CC1 ; CC(C)NC(=O)[C@H]1CC[C@@H](n2/c(=N/C(=O)c3ccc(Br)cc3)[nH]c3cnc(OCCN4CCCCC4)cc32)CC1 ; CC(C)NC(=O)[C@H]1CC[C@@H](n2/c(=N/C(=O)c3cccnc3)[nH]c3cnc(OCCN4CCCCC4)cc32)CC1]

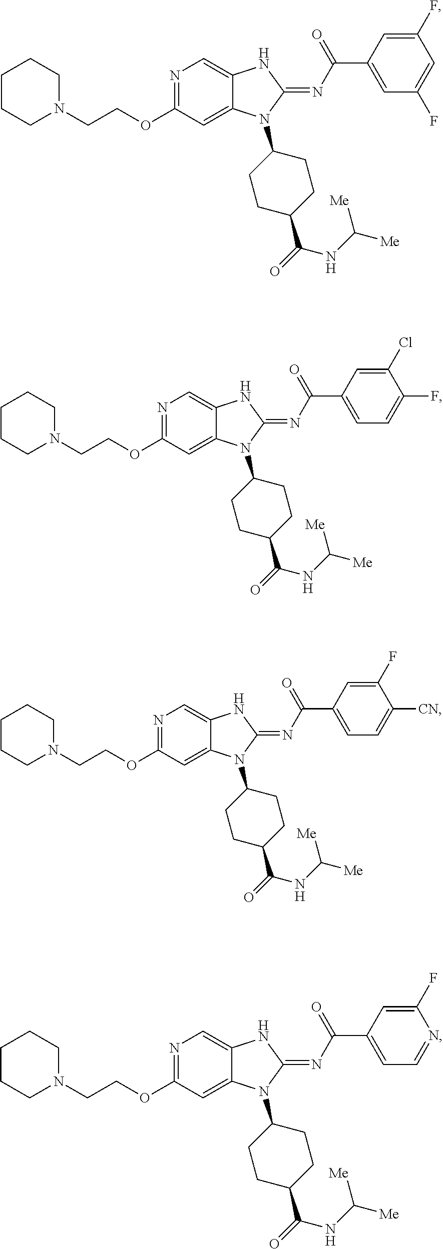 [SMILES: CC(C)NC(=O)[C@H]1CC[C@@H](n2/c(=N/C(=O)c3cc(F)cc(F)c3)[nH]c3cnc(OCCN4CCCCC4)cc32)CC1 ; CC(C)NC(=O)[C@H]1CC[C@@H](n2/c(=N/C(=O)c3ccc(F)c(Cl)c3)[nH]c3cnc(OCCN4CCCCC4)cc32)CC1 ; CC(C)NC(=O)[C@H]1CC[C@@H](n2/c(=N/C(=O)c3ccc(C#N)c(F)c3)[nH]c3cnc(OCCN4CCCCC4)cc32)CC1 ; CC(C)NC(=O)[C@H]1CC[C@@H](n2/c(=N/C(=O)c3ccnc(F)c3)[nH]c3cnc(OCCN4CCCCC4)cc32)CC1]

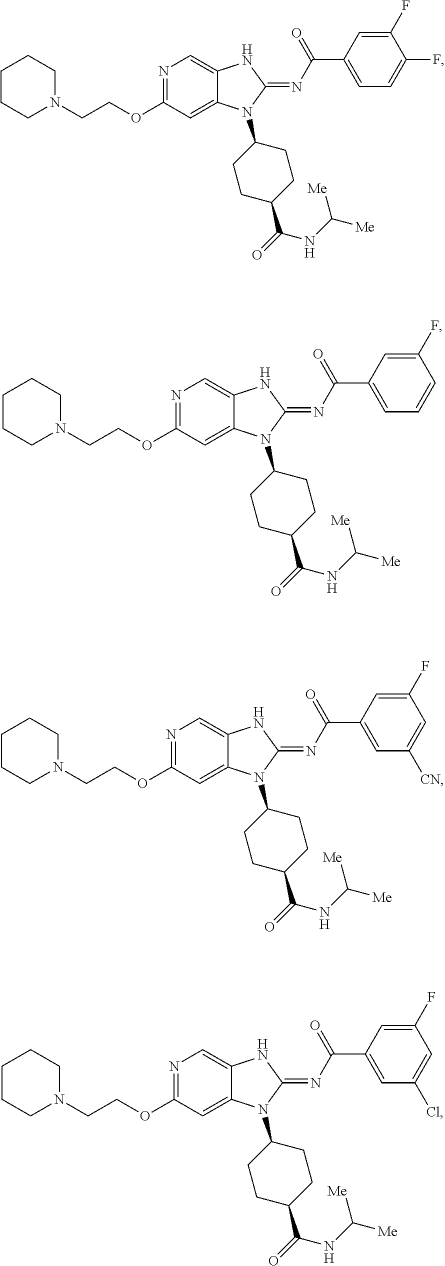 [SMILES: CC(C)NC(=O)[C@H]1CC[C@@H](n2/c(=N/C(=O)c3ccc(F)c(F)c3)[nH]c3cnc(OCCN4CCCCC4)cc32)CC1 ; CC(C)NC(=O)[C@H]1CC[C@@H](n2/c(=N/C(=O)c3cccc(F)c3)[nH]c3cnc(OCCN4CCCCC4)cc32)CC1 ; CC(C)NC(=O)[C@H]1CC[C@@H](n2/c(=N/C(=O)c3cc(F)cc(C#N)c3)[nH]c3cnc(OCCN4CCCCC4)cc32)CC1 ; CC(C)NC(=O)[C@H]1CC[C@@H](n2/c(=N/C(=O)c3cc(F)cc(Cl)c3)[nH]c3cnc(OCCN4CCCCC4)cc32)CC1]

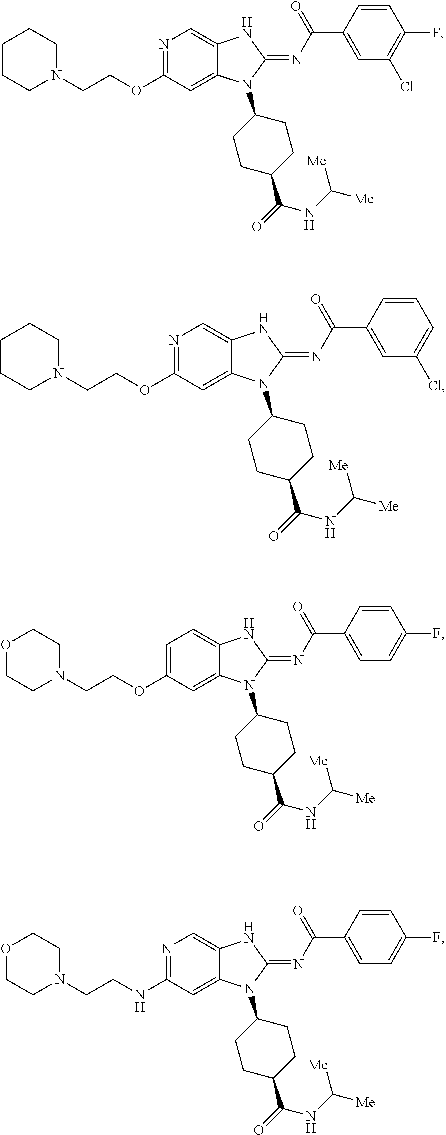 [SMILES: CC(C)NC(=O)[C@H]1CC[C@@H](n2/c(=N/C(=O)c3ccc(F)c(Cl)c3)[nH]c3cnc(OCCN4CCCCC4)cc32)CC1 ; CC(C)NC(=O)[C@H]1CC[C@@H](n2/c(=N/C(=O)c3cccc(Cl)c3)[nH]c3cnc(OCCN4CCCCC4)cc32)CC1 ; CC(C)NC(=O)[C@H]1CC[C@@H](n2/c(=N/C(=O)c3ccc(F)cc3)[nH]c3ccc(OCCN4CCOCC4)cc32)CC1 ; CC(C)NC(=O)[C@H]1CC[C@@H](n2/c(=N/C(=O)c3ccc(F)cc3)[nH]c3cnc(NCCN4CCOCC4)cc32)CC1]

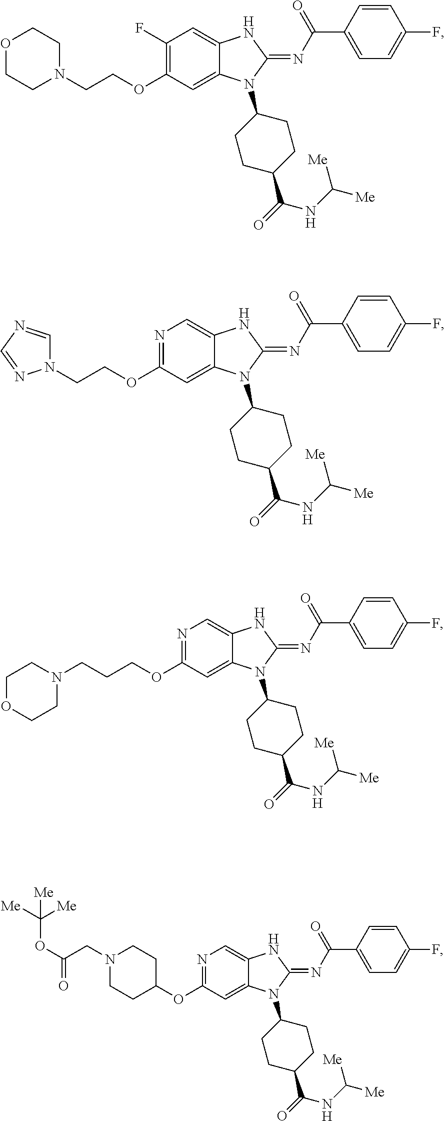 [SMILES: CC(C)NC(=O)[C@H]1CC[C@@H](n2/c(=N/C(=O)c3ccc(F)cc3)[nH]c3cc(F)c(OCCN4CCOCC4)cc32)CC1 ; CC(C)NC(=O)[C@H]1CC[C@@H](n2/c(=N/C(=O)c3ccc(F)cc3)[nH]c3cnc(OCCn4cncn4)cc32)CC1 ; CC(C)NC(=O)[C@H]1CC[C@@H](n2/c(=N/C(=O)c3ccc(F)cc3)[nH]c3cnc(OCCCN4CCOCC4)cc32)CC1 ; CC(C)NC(=O)[C@H]1CC[C@@H](n2/c(=N/C(=O)c3ccc(F)cc3)[nH]c3cnc(OC4CCN(CC(=O)OC(C)(C)C)CC4)cc32)CC1]

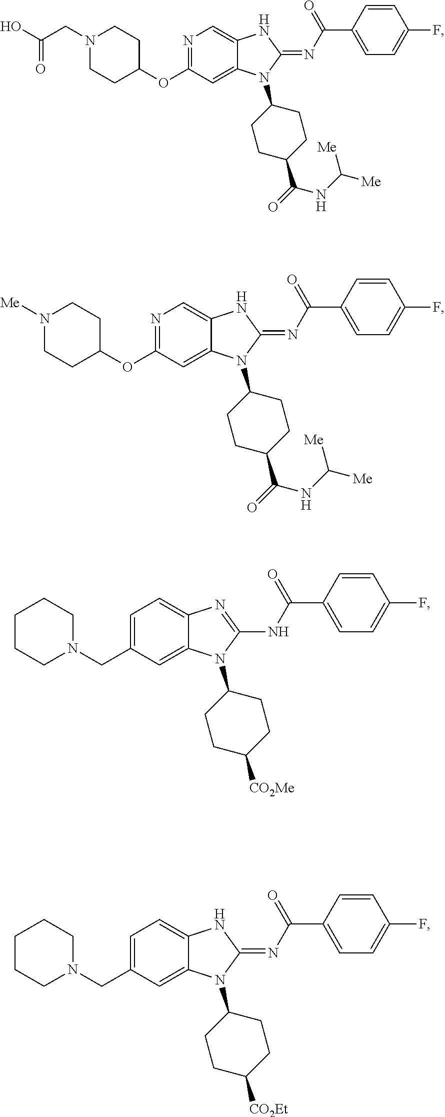 [SMILES: CC(C)NC(=O)[C@H]1CC[C@@H](n2/c(=N/C(=O)c3ccc(F)cc3)[nH]c3cnc(OC4CCN(CC(=O)O)CC4)cc32)CC1 ; CC(C)NC(=O)[C@H]1CC[C@@H](n2/c(=N/C(=O)c3ccc(F)cc3)[nH]c3cnc(OC4CCN(C)CC4)cc32)CC1 ; COC(=O)[C@H]1CC[C@@H](n2c(NC(=O)c3ccc(F)cc3)nc3ccc(CN4CCCCC4)cc32)CC1 ; CCOC(=O)[C@H]1CC[C@@H](n2/c(=N/C(=O)c3ccc(F)cc3)[nH]c3ccc(CN4CCCCC4)cc32)CC1]

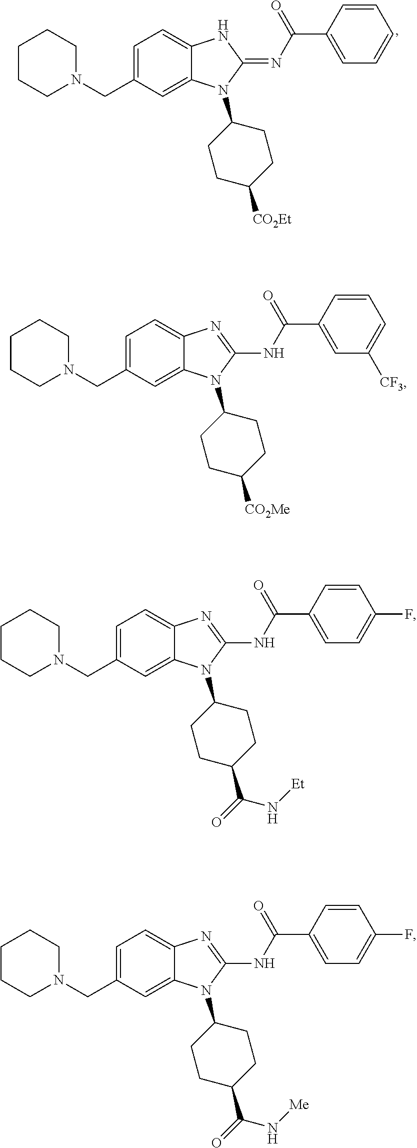 [SMILES: CCOC(=O)[C@H]1CC[C@@H](n2/c(=N/C(=O)c3ccccc3)[nH]c3ccc(CN4CCCCC4)cc32)CC1 ; COC(=O)[C@H]1CC[C@@H](n2c(NC(=O)c3cccc(C(F)(F)F)c3)nc3ccc(CN4CCCCC4)cc32)CC1 ; CCNC(=O)[C@H]1CC[C@@H](n2c(NC(=O)c3ccc(F)cc3)nc3ccc(CN4CCCCC4)cc32)CC1 ; CNC(=O)[C@H]1CC[C@@H](n2c(NC(=O)c3ccc(F)cc3)nc3ccc(CN4CCCCC4)cc32)CC1]

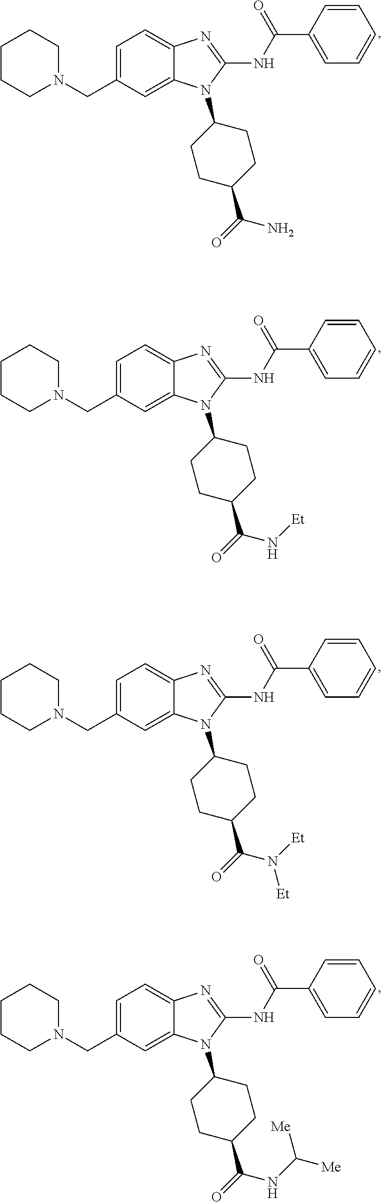 [SMILES: NC(=O)[C@H]1CC[C@@H](n2c(NC(=O)c3ccccc3)nc3ccc(CN4CCCCC4)cc32)CC1 ; CCNC(=O)[C@H]1CC[C@@H](n2c(NC(=O)c3ccccc3)nc3ccc(CN4CCCCC4)cc32)CC1 ; CCN(CC)C(=O)[C@H]1CC[C@@H](n2c(NC(=O)c3ccccc3)nc3ccc(CN4CCCCC4)cc32)CC1 ; CC(C)NC(=O)[C@H]1CC[C@@H](n2c(NC(=O)c3ccccc3)nc3ccc(CN4CCCCC4)cc32)CC1]

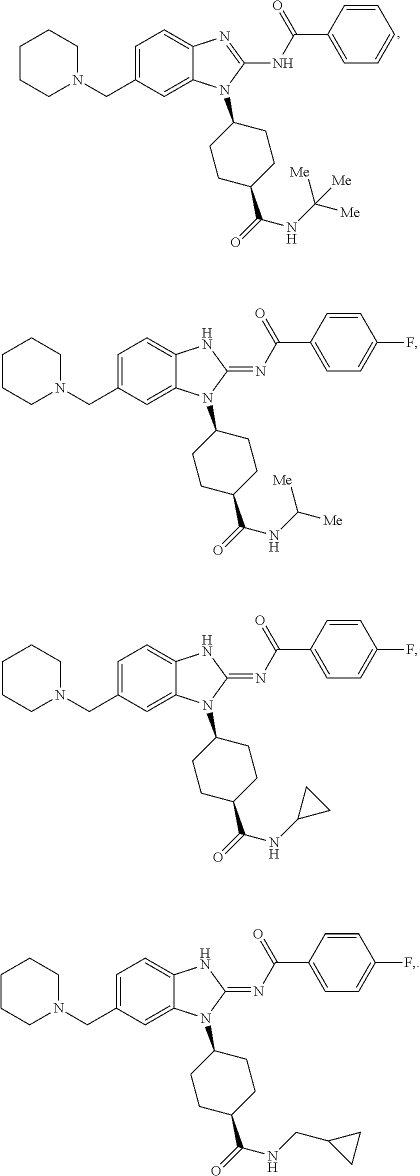 [SMILES: CC(C)(C)NC(=O)[C@H]1CC[C@@H](n2c(NC(=O)c3ccccc3)nc3ccc(CN4CCCCC4)cc32)CC1 ; CC(C)NC(=O)[C@H]1CC[C@@H](n2/c(=N/C(=O)c3ccc(F)cc3)[nH]c3ccc(CN4CCCCC4)cc32)CC1 ; O=C(/N=c1\[nH]c2ccc(CN3CCCCC3)cc2n1[C@H]1CC[C@@H](C(=O)NC2CC2)CC1)c1ccc(F)cc1 ; O=C(/N=c1\[nH]c2ccc(CN3CCCCC3)cc2n1[C@H]1CC[C@@H](C(=O)NCC2CC2)CC1)c1ccc(F)cc1]

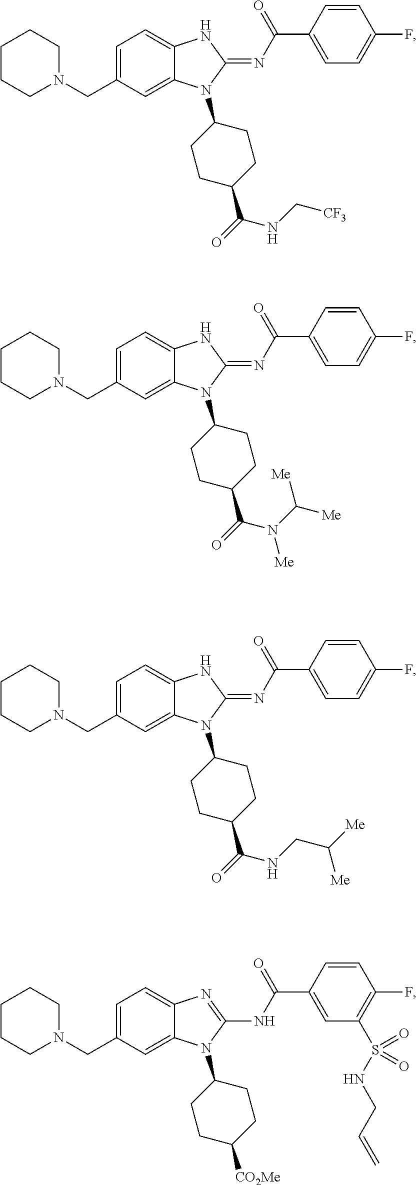 [SMILES: O=C(/N=c1\[nH]c2ccc(CN3CCCCC3)cc2n1[C@H]1CC[C@@H](C(=O)NCC(F)(F)F)CC1)c1ccc(F)cc1 ; CC(C)N(C)C(=O)[C@H]1CC[C@@H](n2/c(=N/C(=O)c3ccc(F)cc3)[nH]c3ccc(CN4CCCCC4)cc32)CC1 ; CC(C)CNC(=O)[C@H]1CC[C@@H](n2/c(=N/C(=O)c3ccc(F)cc3)[nH]c3ccc(CN4CCCCC4)cc32)CC1 ; C=CCNS(=O)(=O)c1cc(C(=O)Nc2nc3ccc(CN4CCCCC4)cc3n2[C@H]2CC[C@@H](C(=O)OC)CC2)ccc1F]

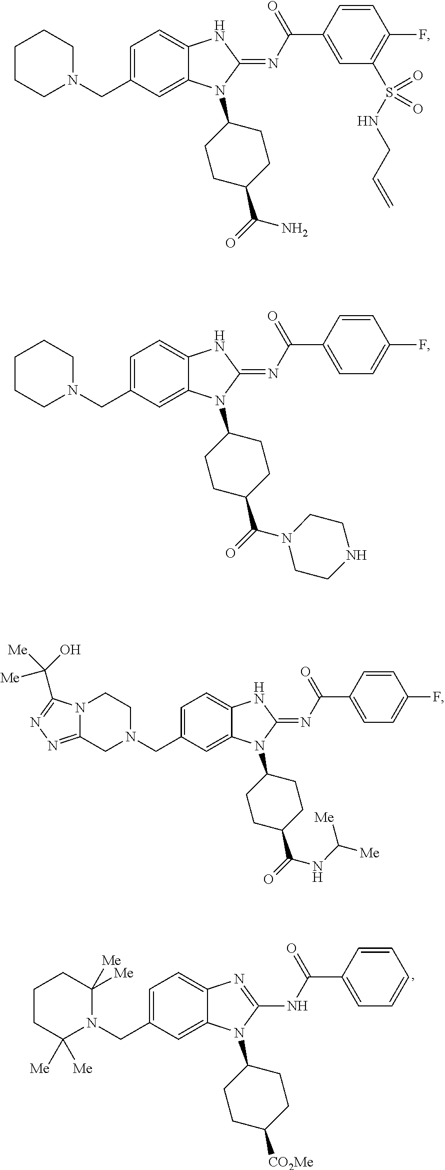 [SMILES: C=CCNS(=O)(=O)c1cc(C(=O)/N=c2\[nH]c3ccc(CN4CCCCC4)cc3n2[C@H]2CC[C@@H](C(N)=O)CC2)ccc1F ; O=C(/N=c1\[nH]c2ccc(CN3CCCCC3)cc2n1[C@H]1CC[C@@H](C(=O)N2CCNCC2)CC1)c1ccc(F)cc1 ; CC(C)NC(=O)[C@H]1CC[C@@H](n2/c(=N/C(=O)c3ccc(F)cc3)[nH]c3ccc(CN4CCn5c(nnc5C(C)(C)O)C4)cc32)CC1 ; COC(=O)[C@H]1CC[C@@H](n2c(NC(=O)c3ccccc3)nc3ccc(CN4C(C)(C)CCCC4(C)C)cc32)CC1]

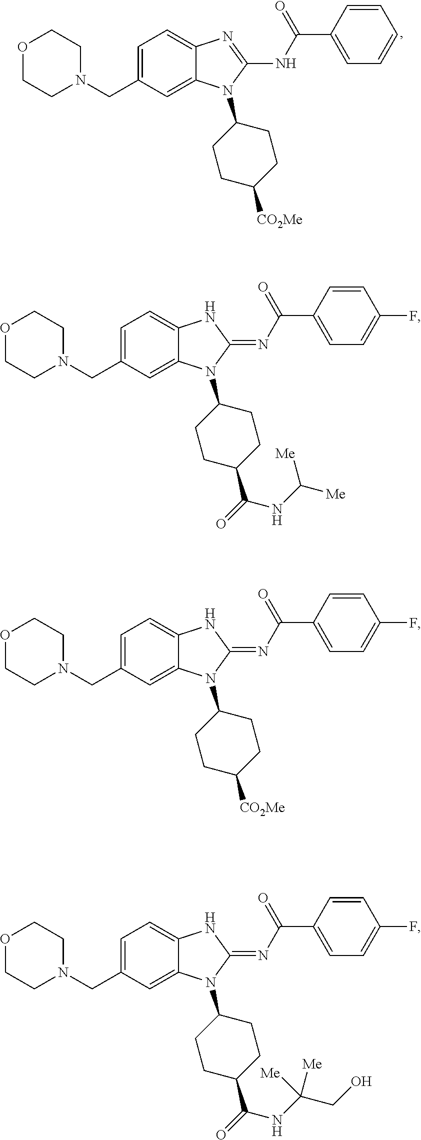 [SMILES: COC(=O)[C@H]1CC[C@@H](n2c(NC(=O)c3ccccc3)nc3ccc(CN4CCOCC4)cc32)CC1 ; CC(C)NC(=O)[C@H]1CC[C@@H](n2/c(=N/C(=O)c3ccc(F)cc3)[nH]c3ccc(CN4CCOCC4)cc32)CC1 ; COC(=O)[C@H]1CC[C@@H](n2/c(=N/C(=O)c3ccc(F)cc3)[nH]c3ccc(CN4CCOCC4)cc32)CC1 ; CC(C)(CO)NC(=O)[C@H]1CC[C@@H](n2/c(=N/C(=O)c3ccc(F)cc3)[nH]c3ccc(CN4CCOCC4)cc32)CC1]

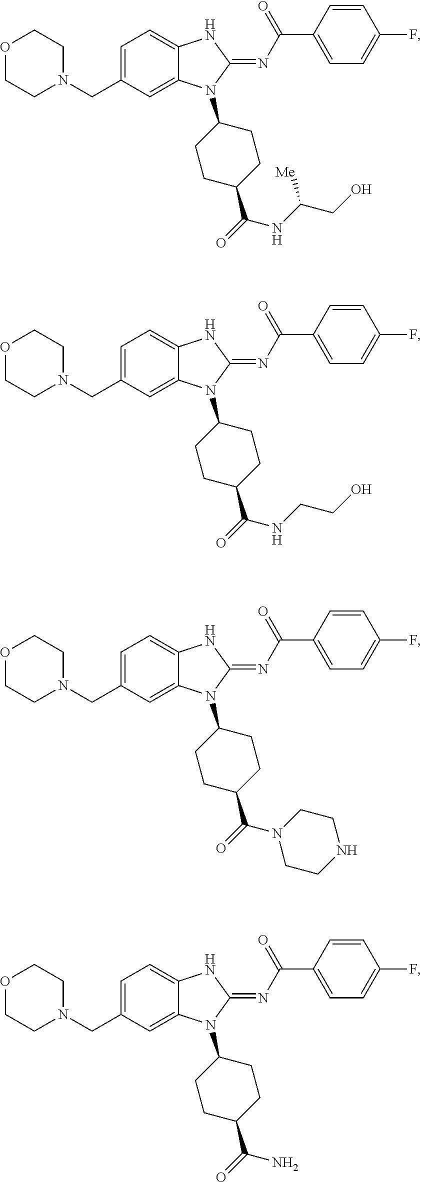 [SMILES: C[C@H](CO)NC(=O)[C@H]1CC[C@@H](n2/c(=N/C(=O)c3ccc(F)cc3)[nH]c3ccc(CN4CCOCC4)cc32)CC1 ; O=C(/N=c1\[nH]c2ccc(CN3CCOCC3)cc2n1[C@H]1CC[C@@H](C(=O)NCCO)CC1)c1ccc(F)cc1 ; O=C(/N=c1\[nH]c2ccc(CN3CCOCC3)cc2n1[C@H]1CC[C@@H](C(=O)N2CCNCC2)CC1)c1ccc(F)cc1 ; NC(=O)[C@H]1CC[C@@H](n2/c(=N/C(=O)c3ccc(F)cc3)[nH]c3ccc(CN4CCOCC4)cc32)CC1]

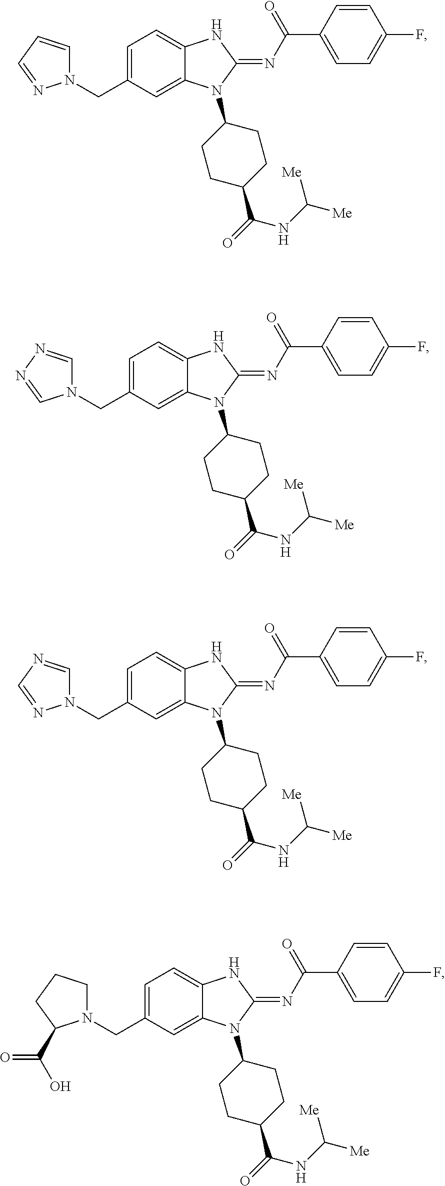 [SMILES: CC(C)NC(=O)[C@H]1CC[C@@H](n2/c(=N/C(=O)c3ccc(F)cc3)[nH]c3ccc(Cn4cccn4)cc32)CC1 ; CC(C)NC(=O)[C@H]1CC[C@@H](n2/c(=N/C(=O)c3ccc(F)cc3)[nH]c3ccc(Cn4cnnc4)cc32)CC1 ; CC(C)NC(=O)[C@H]1CC[C@@H](n2/c(=N/C(=O)c3ccc(F)cc3)[nH]c3ccc(Cn4cncn4)cc32)CC1 ; CC(C)NC(=O)[C@H]1CC[C@@H](n2/c(=N/C(=O)c3ccc(F)cc3)[nH]c3ccc(CN4CCC[C@@H]4C(=O)O)cc32)CC1]

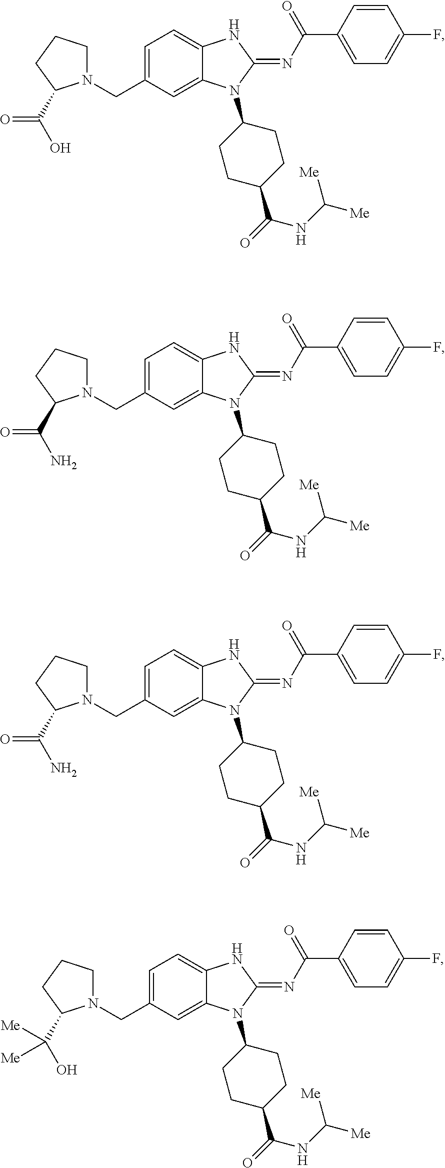 [SMILES: CC(C)NC(=O)[C@H]1CC[C@@H](n2/c(=N/C(=O)c3ccc(F)cc3)[nH]c3ccc(CN4CCC[C@H]4C(=O)O)cc32)CC1 ; CC(C)NC(=O)[C@H]1CC[C@@H](n2/c(=N/C(=O)c3ccc(F)cc3)[nH]c3ccc(CN4CCC[C@@H]4C(N)=O)cc32)CC1 ; CC(C)NC(=O)[C@H]1CC[C@@H](n2/c(=N/C(=O)c3ccc(F)cc3)[nH]c3ccc(CN4CCC[C@H]4C(N)=O)cc32)CC1 ; CC(C)NC(=O)[C@H]1CC[C@@H](n2/c(=N/C(=O)c3ccc(F)cc3)[nH]c3ccc(CN4CCC[C@H]4C(C)(C)O)cc32)CC1]

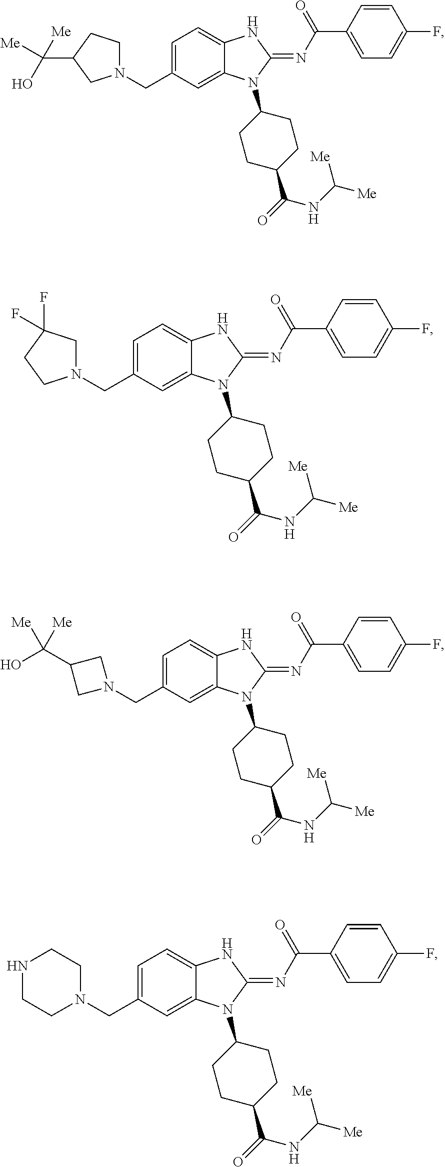 [SMILES: CC(C)NC(=O)[C@H]1CC[C@@H](n2/c(=N/C(=O)c3ccc(F)cc3)[nH]c3ccc(CN4CCC(C(C)(C)O)C4)cc32)CC1 ; CC(C)NC(=O)[C@H]1CC[C@@H](n2/c(=N/C(=O)c3ccc(F)cc3)[nH]c3ccc(CN4CCC(F)(F)C4)cc32)CC1 ; CC(C)NC(=O)[C@H]1CC[C@@H](n2/c(=N/C(=O)c3ccc(F)cc3)[nH]c3ccc(CN4CC(C(C)(C)O)C4)cc32)CC1 ; CC(C)NC(=O)[C@H]1CC[C@@H](n2/c(=N/C(=O)c3ccc(F)cc3)[nH]c3ccc(CN4CCNCC4)cc32)CC1]

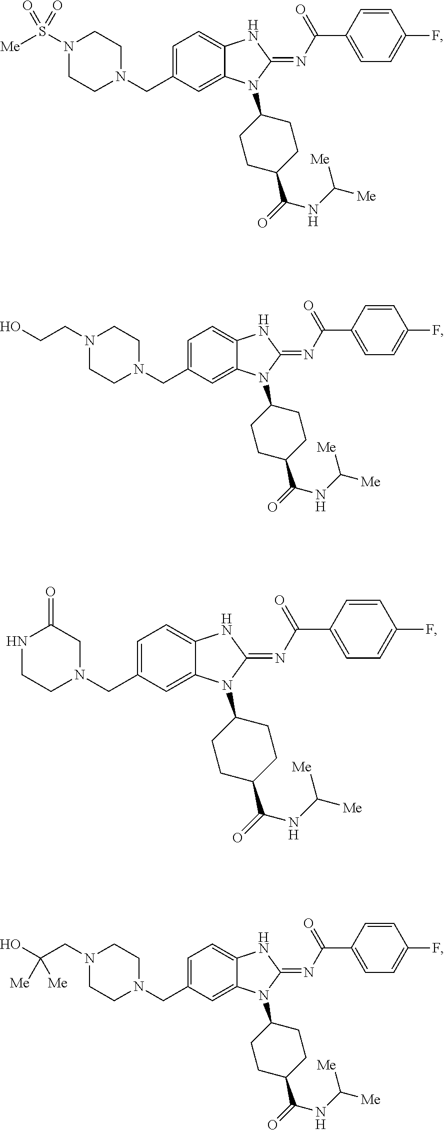 [SMILES: CC(C)NC(=O)[C@H]1CC[C@@H](n2/c(=N/C(=O)c3ccc(F)cc3)[nH]c3ccc(CN4CCN(S(C)(=O)=O)CC4)cc32)CC1 ; CC(C)NC(=O)[C@H]1CC[C@@H](n2/c(=N/C(=O)c3ccc(F)cc3)[nH]c3ccc(CN4CCN(CCO)CC4)cc32)CC1 ; CC(C)NC(=O)[C@H]1CC[C@@H](n2/c(=N/C(=O)c3ccc(F)cc3)[nH]c3ccc(CN4CCNC(=O)C4)cc32)CC1 ; CC(C)NC(=O)[C@H]1CC[C@@H](n2/c(=N/C(=O)c3ccc(F)cc3)[nH]c3ccc(CN4CCN(CC(C)(C)O)CC4)cc32)CC1]

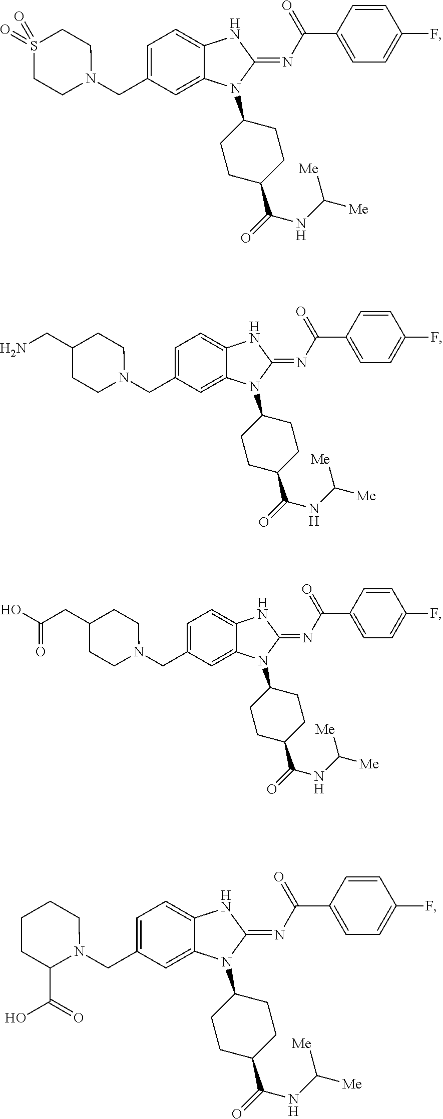 [SMILES: CC(C)NC(=O)[C@H]1CC[C@@H](n2/c(=N/C(=O)c3ccc(F)cc3)[nH]c3ccc(CN4CCS(=O)(=O)CC4)cc32)CC1 ; CC(C)NC(=O)[C@H]1CC[C@@H](n2/c(=N/C(=O)c3ccc(F)cc3)[nH]c3ccc(CN4CCC(CN)CC4)cc32)CC1 ; CC(C)NC(=O)[C@H]1CC[C@@H](n2/c(=N/C(=O)c3ccc(F)cc3)[nH]c3ccc(CN4CCC(CC(=O)O)CC4)cc32)CC1 ; CC(C)NC(=O)[C@H]1CC[C@@H](n2/c(=N/C(=O)c3ccc(F)cc3)[nH]c3ccc(CN4CCCCC4C(=O)O)cc32)CC1]

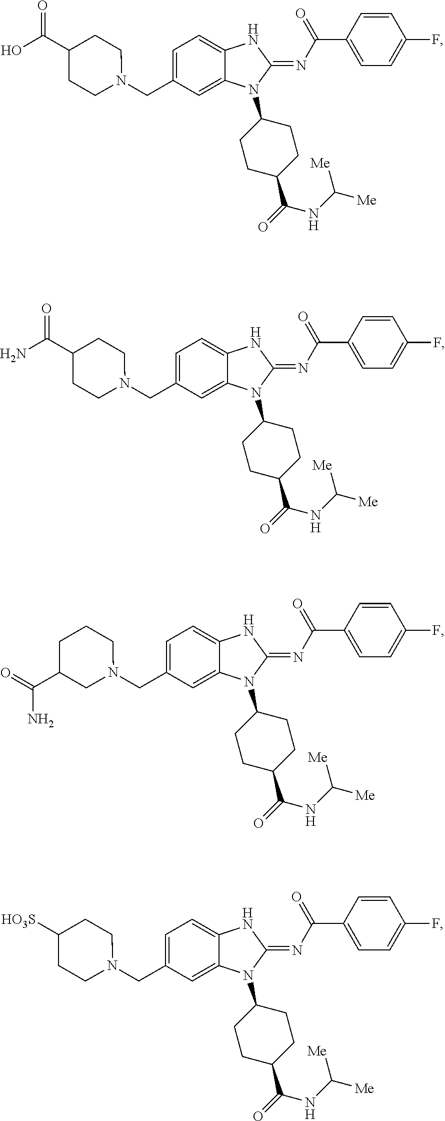 [SMILES: CC(C)NC(=O)[C@H]1CC[C@@H](n2/c(=N/C(=O)c3ccc(F)cc3)[nH]c3ccc(CN4CCC(C(=O)O)CC4)cc32)CC1 ; CC(C)NC(=O)[C@H]1CC[C@@H](n2/c(=N/C(=O)c3ccc(F)cc3)[nH]c3ccc(CN4CCC(C(N)=O)CC4)cc32)CC1 ; CC(C)NC(=O)[C@H]1CC[C@@H](n2/c(=N/C(=O)c3ccc(F)cc3)[nH]c3ccc(CN4CCCC(C(N)=O)C4)cc32)CC1 ; CC(C)NC(=O)[C@H]1CC[C@@H](n2/c(=N/C(=O)c3ccc(F)cc3)[nH]c3ccc(CN4CCC(S(=O)(=O)O)CC4)cc32)CC1]

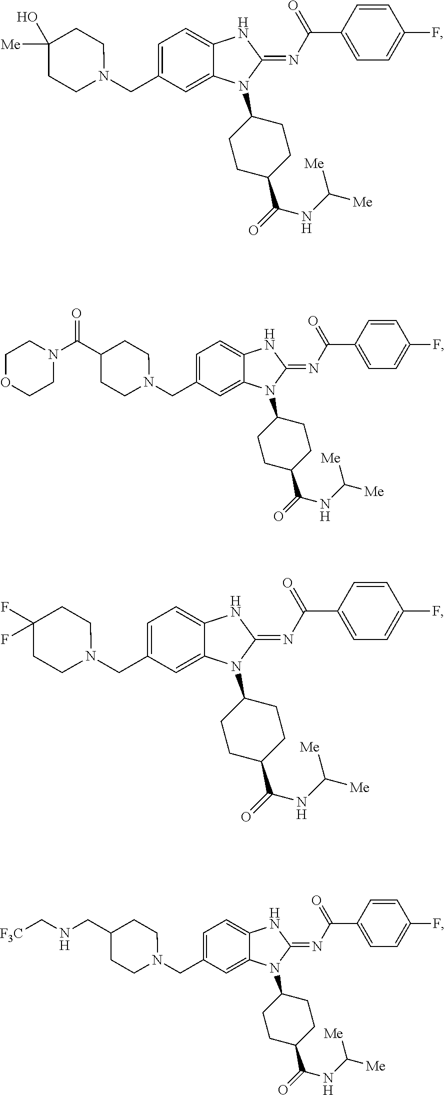 [SMILES: CC(C)NC(=O)[C@H]1CC[C@@H](n2/c(=N/C(=O)c3ccc(F)cc3)[nH]c3ccc(CN4CCC(C)(O)CC4)cc32)CC1 ; CC(C)NC(=O)[C@H]1CC[C@@H](n2/c(=N/C(=O)c3ccc(F)cc3)[nH]c3ccc(CN4CCC(C(=O)N5CCOCC5)CC4)cc32)CC1 ; CC(C)NC(=O)[C@H]1CC[C@@H](n2/c(=N/C(=O)c3ccc(F)cc3)[nH]c3ccc(CN4CCC(F)(F)CC4)cc32)CC1 ; CC(C)NC(=O)[C@H]1CC[C@@H](n2/c(=N/C(=O)c3ccc(F)cc3)[nH]c3ccc(CN4CCC(CNCC(F)(F)F)CC4)cc32)CC1]

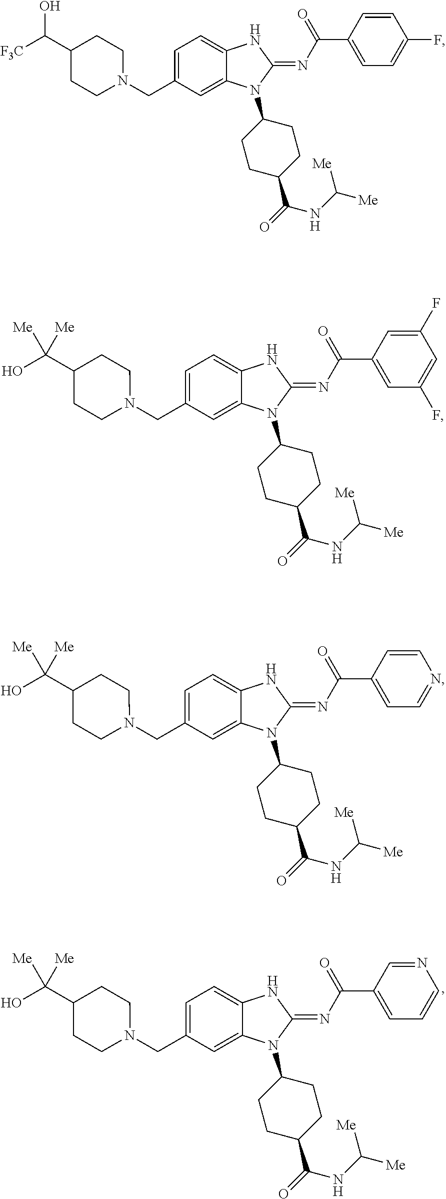 [SMILES: CC(C)NC(=O)[C@H]1CC[C@@H](n2/c(=N/C(=O)c3ccc(F)cc3)[nH]c3ccc(CN4CCC(C(O)C(F)(F)F)CC4)cc32)CC1 ; CC(C)NC(=O)[C@H]1CC[C@@H](n2/c(=N/C(=O)c3cc(F)cc(F)c3)[nH]c3ccc(CN4CCC(C(C)(C)O)CC4)cc32)CC1 ; CC(C)NC(=O)[C@H]1CC[C@@H](n2/c(=N/C(=O)c3ccncc3)[nH]c3ccc(CN4CCC(C(C)(C)O)CC4)cc32)CC1 ; CC(C)NC(=O)[C@H]1CC[C@@H](n2/c(=N/C(=O)c3cccnc3)[nH]c3ccc(CN4CCC(C(C)(C)O)CC4)cc32)CC1]

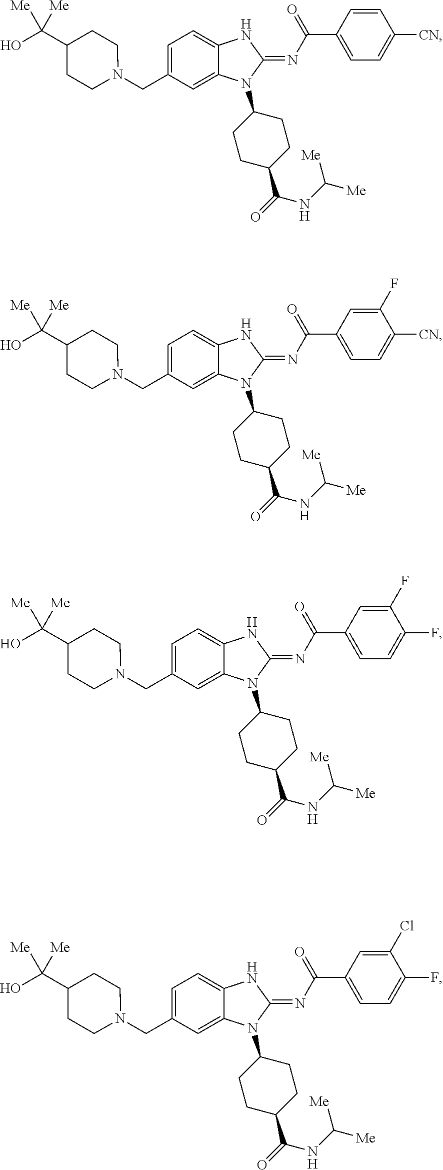 [SMILES: CC(C)NC(=O)[C@H]1CC[C@@H](n2/c(=N/C(=O)c3ccc(C#N)cc3)[nH]c3ccc(CN4CCC(C(C)(C)O)CC4)cc32)CC1 ; CC(C)NC(=O)[C@H]1CC[C@@H](n2/c(=N/C(=O)c3ccc(C#N)c(F)c3)[nH]c3ccc(CN4CCC(C(C)(C)O)CC4)cc32)CC1 ; CC(C)NC(=O)[C@H]1CC[C@@H](n2/c(=N/C(=O)c3ccc(F)c(F)c3)[nH]c3ccc(CN4CCC(C(C)(C)O)CC4)cc32)CC1 ; CC(C)NC(=O)[C@H]1CC[C@@H](n2/c(=N/C(=O)c3ccc(F)c(Cl)c3)[nH]c3ccc(CN4CCC(C(C)(C)O)CC4)cc32)CC1]

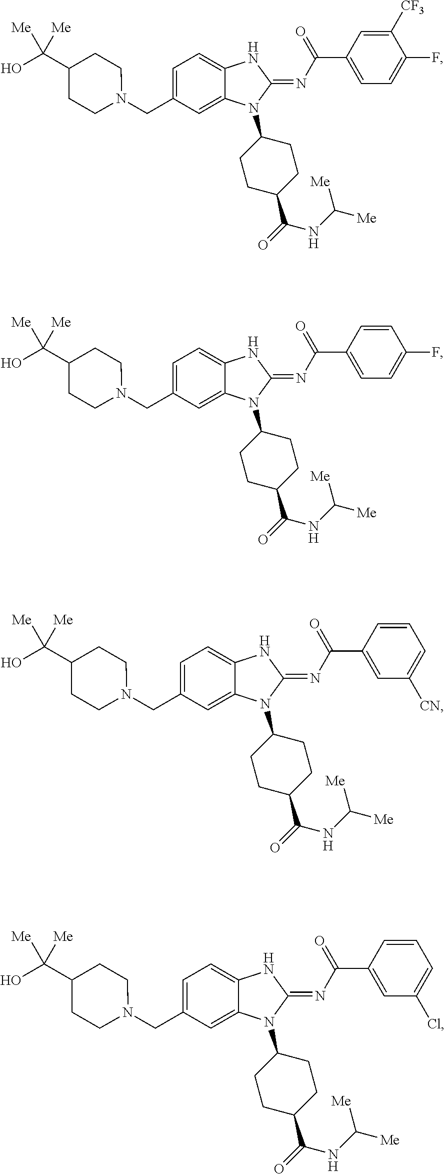 [SMILES: CC(C)NC(=O)[C@H]1CC[C@@H](n2/c(=N/C(=O)c3ccc(F)c(C(F)(F)F)c3)[nH]c3ccc(CN4CCC(C(C)(C)O)CC4)cc32)CC1 ; CC(C)NC(=O)[C@H]1CC[C@@H](n2/c(=N/C(=O)c3ccc(F)cc3)[nH]c3ccc(CN4CCC(C(C)(C)O)CC4)cc32)CC1 ; CC(C)NC(=O)[C@H]1CC[C@@H](n2/c(=N/C(=O)c3cccc(C#N)c3)[nH]c3ccc(CN4CCC(C(C)(C)O)CC4)cc32)CC1 ; CC(C)NC(=O)[C@H]1CC[C@@H](n2/c(=N/C(=O)c3cccc(Cl)c3)[nH]c3ccc(CN4CCC(C(C)(C)O)CC4)cc32)CC1]

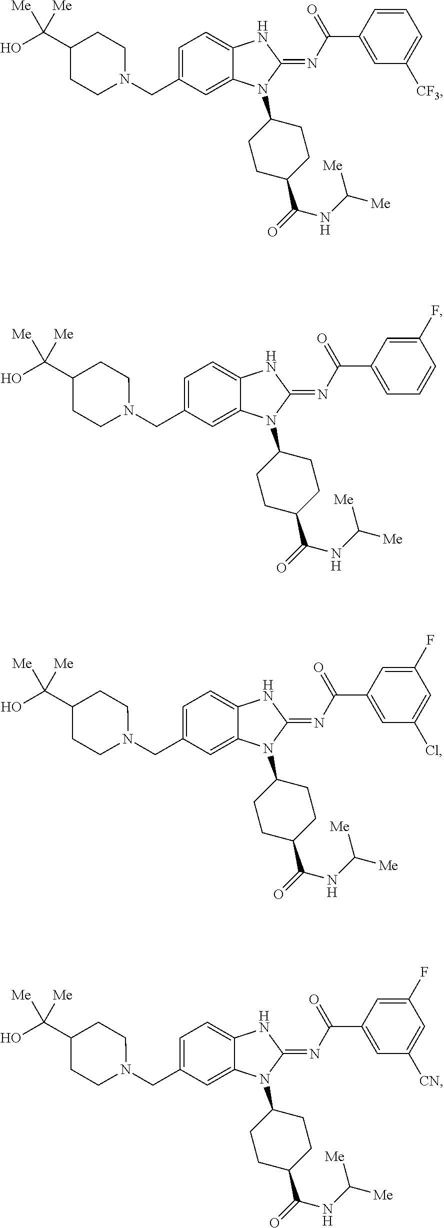 [SMILES: CC(C)NC(=O)[C@H]1CC[C@@H](n2/c(=N/C(=O)c3cccc(C(F)(F)F)c3)[nH]c3ccc(CN4CCC(C(C)(C)O)CC4)cc32)CC1 ; CC(C)NC(=O)[C@H]1CC[C@@H](n2/c(=N/C(=O)c3cccc(F)c3)[nH]c3ccc(CN4CCC(C(C)(C)O)CC4)cc32)CC1 ; CC(C)NC(=O)[C@H]1CC[C@@H](n2/c(=N/C(=O)c3cc(F)cc(Cl)c3)[nH]c3ccc(CN4CCC(C(C)(C)O)CC4)cc32)CC1 ; CC(C)NC(=O)[C@H]1CC[C@@H](n2/c(=N/C(=O)c3cc(F)cc(C#N)c3)[nH]c3ccc(CN4CCC(C(C)(C)O)CC4)cc32)CC1]

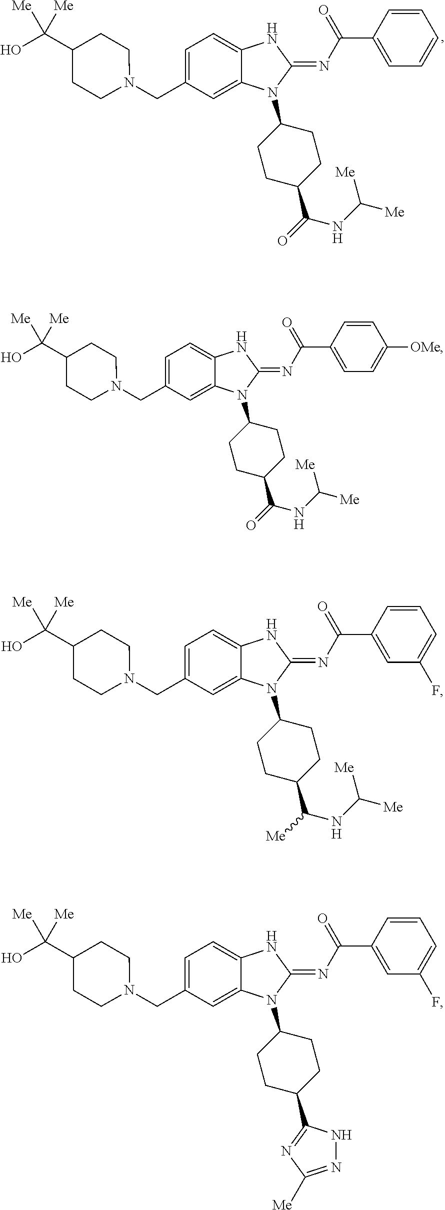 [SMILES: CC(C)NC(=O)[C@H]1CC[C@@H](n2/c(=N/C(=O)c3ccccc3)[nH]c3ccc(CN4CCC(C(C)(C)O)CC4)cc32)CC1 ; COc1ccc(C(=O)/N=c2\[nH]c3ccc(CN4CCC(C(C)(C)O)CC4)cc3n2[C@H]2CC[C@@H](C(=O)NC(C)C)CC2)cc1 ; CC(C)NC(C)[C@H]1CC[C@@H](n2/c(=N/C(=O)c3cccc(F)c3)[nH]c3ccc(CN4CCC(C(C)(C)O)CC4)cc32)CC1 ; Cc1n[nH]c([C@H]2CC[C@@H](n3/c(=N/C(=O)c4cccc(F)c4)[nH]c4ccc(CN5CCC(C(C)(C)O)CC5)cc43)CC2)n1]

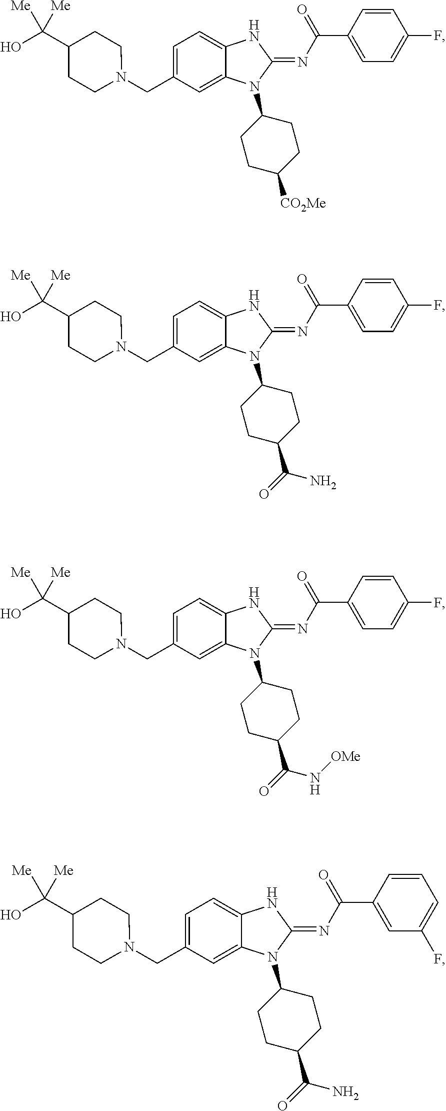 [SMILES: COC(=O)[C@H]1CC[C@@H](n2/c(=N/C(=O)c3ccc(F)cc3)[nH]c3ccc(CN4CCC(C(C)(C)O)CC4)cc32)CC1 ; CC(C)(O)C1CCN(Cc2ccc3[nH]/c(=N\C(=O)c4ccc(F)cc4)n([C@H]4CC[C@@H](C(N)=O)CC4)c3c2)CC1 ; CONC(=O)[C@H]1CC[C@@H](n2/c(=N/C(=O)c3ccc(F)cc3)[nH]c3ccc(CN4CCC(C(C)(C)O)CC4)cc32)CC1 ; CC(C)(O)C1CCN(Cc2ccc3[nH]/c(=N\C(=O)c4cccc(F)c4)n([C@H]4CC[C@@H](C(N)=O)CC4)c3c2)CC1]

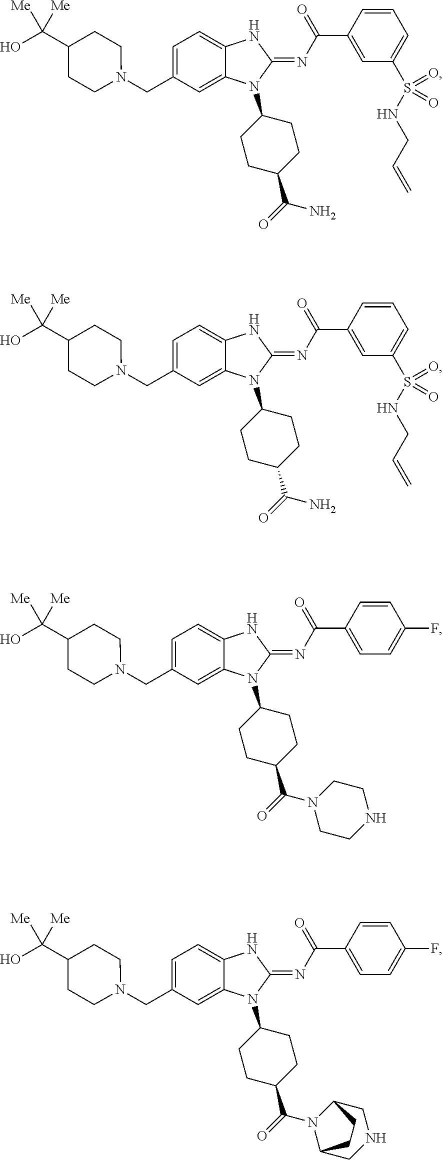 [SMILES: C=CCNS(=O)(=O)c1cccc(C(=O)/N=c2\[nH]c3ccc(CN4CCC(C(C)(C)O)CC4)cc3n2[C@H]2CC[C@@H](C(N)=O)CC2)c1 ; C=CCNS(=O)(=O)c1cccc(C(=O)/N=c2\[nH]c3ccc(CN4CCC(C(C)(C)O)CC4)cc3n2[C@H]2CC[C@H](C(N)=O)CC2)c1 ; CC(C)(O)C1CCN(Cc2ccc3[nH]/c(=N\C(=O)c4ccc(F)cc4)n([C@H]4CC[C@@H](C(=O)N5CCNCC5)CC4)c3c2)CC1 ; CC(C)(O)C1CCN(Cc2ccc3[nH]/c(=N\C(=O)c4ccc(F)cc4)n([C@H]4CC[C@@H](C(=O)N5[C@@H]6CC[C@H]5CNC6)CC4)c3c2)CC1]

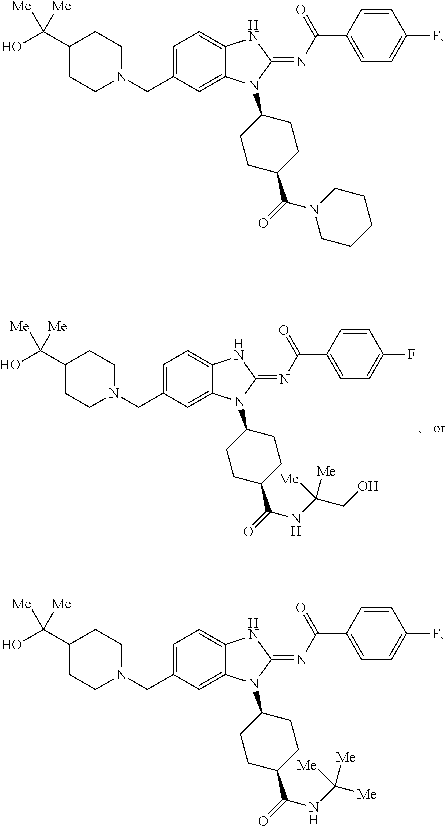 [SMILES: CC(C)(O)C1CCN(Cc2ccc3[nH]/c(=N\C(=O)c4ccc(F)cc4)n([C@H]4CC[C@@H](C(=O)N5CCCCC5)CC4)c3c2)CC1 ; CC(C)(CO)NC(=O)[C@H]1CC[C@@H](n2/c(=N/C(=O)c3ccc(F)cc3)[nH]c3ccc(CN4CCC(C(C)(C)O)CC4)cc32)CC1 ; CC(C)(C)NC(=O)[C@H]1CC[C@@H](n2/c(=N/C(=O)c3ccc(F)cc3)[nH]c3ccc(CN4CCC(C(C)(C)O)CC4)cc32)CC1]

or a pharmaceutically acceptable salt thereof, tautomer thereof, a pharmaceutically acceptable salt of the tautomer, a stereoisomer of any of the foregoing, or a mixture thereof.

In some embodiments, the compound of formula I is selected from

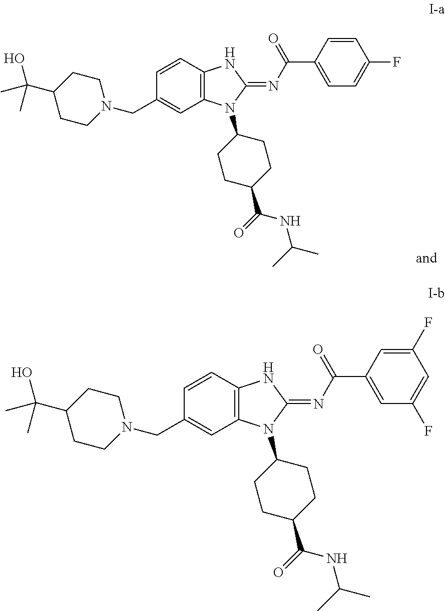 [SMILES: CC(C)NC(=O)[C@H]1CC[C@@H](n2/c(=N/C(=O)c3ccc(F)cc3)[nH]c3ccc(CN4CCC(C(C)(C)O)CC4)cc32)CC1 ; CC(C)NC(=O)[C@H]1CC[C@@H](n2/c(=N/C(=O)c3cc(F)cc(F)c3)[nH]c3ccc(CN4CCC(C(C)(C)O)CC4)cc32)CC1]

In some embodiments of any of those described above, a compound of formula I is provided as a pharmaceutically acceptable salt. In some embodiments, a compound of formula I is a single stereoisomer whereas in other embodiments, a compound of formula I is a mixture of enantiomers or is a mixture of stereoisomers and such a mixture may include equal or unequal amount of specific stereoisomers. In some embodiments a compound of formula I is a racemic mixture of stereoisomers.

In some embodiments, a compound of formula I is provided as a salt. Such salts may be anhydrous or associated with water as a hydrate.

In some embodiments, provided compounds are characterized in that they show inhibitory activity toward one or more tyrosine kinase(s). In some such embodiments, provided compounds show inhibitory activity (i.e., IC₅₀) within the range of about 0.05 μM to about 5 μM, or about 0.05 μM to about 1 μM, or about 0.05 μM to about 0.1 μM toward ALK. In some embodiments, provided compounds show inhibitory activity (i.e., IC₅₀) within the range of less than 1 nM, or about 1 nM to about 100 nM toward any of TRKA, TRKB or TRKC. In some embodiments, provided compounds show inhibitory activity (i.e., IC₅₀) within the range of less than 1 nM toward TRKA.

In some such embodiments, the one or more tyrosine kinases is/are selected from the group consisting of ALK, TRKA, TRKB, TRKC, p75 and RET.

In some embodiments, provided compounds are characterized in that they show relative inhibitory activity toward certain tyrosine kinases such as TRKA>TRKB>TRKC. In some embodiments, provided compounds are characterized in that they show relative inhibitory activity toward certain tyrosine kinases such as ALK>TRKA>TRKB>TRKC. In some embodiments, provided compounds are characterized in that they show relative inhibitory activity toward certain tyrosine kinases such as ALK>TRKA>TRKB>TRKC>RET.

Also provided are pharmaceutical formulations that include at least one pharmaceutically acceptable carrier, excipient or diluent and a therapeutically effective amount of the compound of any of the embodiments described herein. In some such embodiments, the compound is present in an amount effective for the treatment of cancer.

Further provided are pharmaceutical formulations that include at least one pharmaceutically acceptable carrier, and a therapeutically effective amount of the composition of matter of any of the embodiments described herein in combination with at least one additional compound such as a cytotoxic agent or a compound that inhibits another kinase.

In some embodiments, the present invention provides pharmaceutical formulations that include at least one pharmaceutically acceptable carrier, and a therapeutically effective amount of the composition of matter of any of the embodiments described herein in combination with at least one additional chemotherapeutic agent

In some embodiments, the one or more additional chemotherapeutic agent is an antimetabolite antineoplastic agent selected from 5-FU-fibrinogen, acanthifolic acid, aminothiadiazole, brequinar sodium, carmofur, Ciba-Geigy CGP-30694, cyclopentyl cytosine, cytarabine phosphate stearate, cytarabine conjugates, Lilly DATHF, Merrel Dow DDFC, dezaguanine, dideoxycytidine, dideoxyguanosine, didox, Yoshitomi DMDC, doxifluridine, Wellcome EHNA, Merck & Co. EX-015, fazarabine, floxuridine, fludarabine phosphate, 5-fluorouracil, N-(2′-furanidyl)-5-fluorouracil, Daiichi Seiyaku FO-152, isopropyl pyrrolizine, Lilly LY-188011, Lilly LY-264618, methobenzaprim, methotrexate, Wellcome MZPES, norspermidine, NCI NSC-127716, NCI NSC-264880, NCI NSC-39661, NCI NSC-612567, Warner-Lambert PALA, pentostatin, piritrexim, plicamycin, Asahi Chemical PL-AC, Takeda TAC-788, thioguanine, tiazofurin, Erbamont TIF, trimetrexate, tyrosine kinase inhibitors, Taiho UFT and uricytin.

In some embodiments, the one or more additional chemotherapeutic agent is an alkylating-type antineoplastic agent selected from Shionogi 254-S, aldo-phosphamide analogues, altretamine, anaxirone, Boehringer Mannheim BBR-2207, bestrabucil, budotitane, Wakunaga CA-102, carboplatin, carmustine, Chinoin-139, Chinoin-153, chlorambucil, cisplatin, cyclophosphamide, American Cyanamid CL-286558, Sanofi CY-233, cyplatate, Degussa D-19-384, Sumimoto DACHP(Myr)2, diphenylspiromustine, diplatinum cytostatic, Erba distamycin derivatives, Chugai DWA-2114R, ITI E09, elmustine, Erbamont FCE-24517, estramustine phosphate sodium, fotemustine, Unimed G-6-M, Chinoin GYKI-17230, hepsul-fam, ifosfamide, iproplatin, lomustine, mafosfamide, mitolactol, Nippon Kayaku NK-121, NCI NSC-264395, NCI NSC-342215, oxaliplatin, Upjohn PCNU, prednimustine, Proter PTT-119, ranimustine, semustine, SmithKline SK&F-101772, Yakult Honsha SN-22, spiromus-tine, Tanabe Seiyaku TA-077, tauromustine, temozolomide, teroxirone, tetraplatin and trimelamol.

In some embodiments, the one or more additional chemotherapeutic agent is an antibiotic-type antineoplastic agent selected from Taiho 4181-A, aclarubicin, actinomycin D, actinoplanone, Erbamont ADR-456, aeroplysinin derivative, Ajinomoto AN-201-II, Ajinomoto AN-3, Nippon Soda anisomycins, anthracycline, azino-mycin-A, bisucaberin, Bristol-Myers BL-6859, Bristol-Myers BMY-25067, Bristol-Myers BMY-25551, Bristol-Myers BMY-26605, Bristol-Myers BMY-27557, Bristol-Myers BMY-28438, bleomycin sulfate, bryostatin-1, Taiho C-1027, calichemycin, chromoximycin, dactinomycin, daunorubicin, Kyowa Hakko DC-102, Kyowa Hakko DC-79, Kyowa Hakko DC-88A, Kyowa Hakko DC89-A1, Kyowa Hakko DC92-B, ditrisarubicin B, Shionogi DOB-41, doxorubicin, doxorubicin-fibrinogen, elsamicin-A, epirubicin, erbstatin, esorubicin, esperamicin-A1, esperamicin-Alb, Erbamont FCE-21954, Fujisawa FK-973, fostriecin, Fujisawa FR-900482, glidobactin, gregatin-A, grincamycin, herbimycin, idarubicin, illudins, kazusamycin, kesarirhodins, Kyowa Hakko KM-5539, Kirin Brewery KRN-8602, Kyowa Hakko KT-5432, Kyowa Hakko KT-5594, Kyowa Hakko KT-6149, American Cyanamid LL-D49194, Meiji Seika ME 2303, menogaril, mitomycin, mitoxantrone, SmithKline M-TAG, neoenactin, Nippon Kayaku NK-313, Nippon Kayaku NKT-01, SRI International NSC-357704, oxalysine, oxaunomycin, peplomycin, pilatin, pirarubicin, porothramycin, pyrindanycin A, Tobishi RA-I, rapamycin, rhizoxin, rodorubicin, sibanomicin, siwenmycin, Sumitomo SM-5887, Snow Brand SN-706, Snow Brand SN-07, sorangicin-A, sparsomycin, SS Pharmaceutical SS-21020, SS Pharmaceutical SS-7313B, SS Pharmaceutical SS-9816B, steffimycin B, Taiho 4181-2, talisomycin, Takeda TAN-868A, terpentecin, thrazine, tricrozarin A, Upjohn U-73975, Kyowa Hakko UCN-10028A, Fujisawa WF-3405, Yoshitomi Y-25024, and zorubicin.

In some embodiments, the one or more additional chemotherapeutic agent is an antineoplastic agent selected from tubulin interacting agents, topoisomerase II inhibitors, topoisomerase I inhibitors and hormonal agents, selected from, but not limited to, the group consisting of α-carotene, α-difluoromethyl-arginine, acitretin, Biotec AD-5, Kyorin AHC-52, alstonine, amonafide, amphethinile, amsacrine, Angiostat, ankinomycin, anti-neoplaston A10, antineoplaston A2, antineoplaston A3, antineoplaston A5, antineoplaston AS2-1, Henkel APD, aphidicolin glycinate, asparaginase, Avarol, baccharin, batracylin, benfluron, benzotript, Ipsen-Beaufour BIM-23015, bisantrene, Bristol-Myers BMY-40481, Vestar boron-10, bromofosfamide, Wellcome BW-502, Wellcome BW-773, caracemide, carmethizole hydrochloride, Ajinomoto CDAF, chlorsulfaquinoxalone, Chemes CHX-2053, Chemex CHX-100, Warner-Lambert CI-921, Warner-Lambert CI-937, Warner-Lambert CI-941, Warner-Lambert CI-958, clanfenur, claviridenone, ICN compound 1259, ICN compound 4711, Contracan, Yakult Honsha CPT-11, crisnatol, curaderm, cytochalasin B, cytarabine, cytocytin, Merz D-609, DABIS maleate, dacarbazine, datelliptinium, didemnin-B, dihaematoporphyrin ether, dihydrolenperone, dinaline, distamycin, Toyo Pharmar DM-341, Toyo Pharmar DM-75, Daiichi Seiyaku DN-9693, docetaxel elliprabin, elliptinium acetate, Tsumura EPMTC, the epothilones, ergotamine, etoposide, etretinate, fenretinide, Fujisawa FR-57704, gallium nitrate, genkwadaphnin, Chugai GLA-43, Glaxo GR-63178, grifolan NMF-5N, hexadecylphosphocholine, Green Cross HO-221, homoharringtonine, hydroxyurea, BTG ICRF-187, ilmofosine, isoglutamine, isotretinoin, Otsuka JI-36, Ramot K-477, Otsuak K-76COONa, Kureha Chemical K-AM, MECT Corp KI-8110, American Cyanamid L-623, leukoregulin, lonidamine, Lundbeck LU-23-112, Lilly LY-186641, NCI (US) MAP, marycin, Merrel Dow MDL-27048, Medco MEDR-340, merbarone, merocyanlne derivatives, methylanilinoacridine, Molecular Genetics MGI-136, minactivin, mitonafide, mitoquidone mopidamol, motretinide, Zenyaku Kogyo MST-16, N-(retinoyl)amino acids, Nisshin Flour Milling N-021, N-acylated-dehydroalanines, nafazatrom, Taisho NCU-190, nocodazole derivative, Normosang, NCI NSC-145813, NCI NSC-361456, NCI NSC-604782, NCI NSC-95580, ocreotide, Ono ONO-112, oquizanocine, Akzo Org-10172, paclitaxel, pancratistatin, pazelliptine, Warner-Lambert PD-111707, Warner-Lambert PD-115934, Warner-Lambert PD-131141, Pierre Fabre PE-1001, ICRT peptide D, piroxantrone, polyhaematoporphyrin, polypreic acid, Efamol porphyrin, probimane, procarbazine, proglumide, Invitron protease nexin I, Tobishi RA-700, razoxane, Sapporo Breweries RBS, restrictin-P, retelliptine, retinoic acid, Rhone-Poulenc RP-49532, Rhone-Poulenc RP-56976, SmithKline SK&F-104864, Sumitomo SM-108, Kuraray SMANCS, SeaPharm SP-10094, spatol, spirocyclopropane derivatives, spirogermanium, Unimed, SS Pharmaceutical SS-554, strypoldinone, Stypoldione, Suntory SUN 0237, Suntory SUN 2071, superoxide dismutase, Toyama T-506, Toyama T-680, taxol, Teijin TEI-0303, teniposide, thaliblastine, Eastman Kodak TJB-29, tocotrienol, topotecan, Topostin, Teijin TT-82, Kyowa Hakko UCN-01, Kyowa Hakko UCN-1028, ukrain, Eastman Kodak USB-006, vinblastine sulfate, vincristine, vindesine, vinestramide, vinorelbine, vintriptol, vinzolidine, withanolides, and Yamanouchi YM-534.

In some embodiments, the one or more additional chemotherapeutic agent is selected from acemannan, aclarubicin, aldesleukin, alemtuzumab, alitretinoin, altretamine, amifostine, aminolevulinic acid, amrubicin, amsacrine, anagrelide, anastrozole, ANCER, ancestim, ARGLABIN, arsenic trioxide, BAM 002 (Novelos), bexarotene, bicalutamide, broxuridine, capecitabine, celmoleukin, cetrorelix, cladribine, clotrimazole, cytarabine ocfosfate, DA 3030 (Dong-A), daclizumab, denileukin diftitox, deslorelin, dexrazoxane, dilazep, docetaxel, docosanol, doxercalciferol, doxifluridine, doxorubicin, bromocriptine, carmustine, cytarabine, fluorouracil, HIT diclofenac, interferon alfa, daunorubicin, doxorubicin, tretinoin, edelfosine, edrecolomab, eflornithine, emitefur, epirubicin, epoetin beta, etoposide phosphate, exemestane, exisulind, fadrozole, filgrastim, finasteride, fludarabine phosphate, formestane, fotemustine, gallium nitrate, gemcitabine, gemtuzumab zogamicin, gimeracil/oteracil/tegafur combination, glycopine, goserelin, heptaplatin, human chorionic gonadotropin, human fetal alpha fetoprotein, ibandronic acid, idarubicin, (imiquimod, interferon alfa, interferon alfa, natural, interferon alfa-2, interferon alfa-2a, interferon alfa-2b, interferon alfa-N1, interferon alfa-n3, interferon alfacon-1, interferon alpha, natural, interferon beta, interferon beta-1a, interferon beta-1b, interferon gamma, natural interferon gamma-1a, interferon gamma-1b, interleukin-1 beta, iobenguane, irinotecan, irsogladine, lanreotide, LC 9018 (Yakult), leflunomide, lenograstim, lentinan sulfate, letrozole, leukocyte alpha interferon, leuprorelin, levamisole+fluorouracil, liarozole, lobaplatin, lonidamine, lovastatin, masoprocol, melarsoprol, metoclopramide, mifepristone, miltefosine, mirimostim, mismatched double stranded RNA, mitoguazone, mitolactol, mitoxantrone, molgramostim, nafarelin, naloxone+pentazocine, nartograstim, nedaplatin, nilutamide, noscapine, novel erythropoiesis stimulating protein, NSC 631570 octreotide, oprelvekin, osaterone, oxaliplatin, paclitaxel, pamidronic acid, pegaspargase, peginterferon alfa-2b, pentosan polysulfate sodium, pentostatin, picibanil, pirarubicin, rabbit antithymocyte polyclonal antibody, polyethylene glycol interferon alfa-2a, porfimer sodium, raloxifene, raltitrexed, rasburicase, rhenium Re 186 etidronate, RH retinamide, rituximab, romurtide, samarium (153 Sm) lexidronam, sargramostim, sizofiran, sobuzoxane, sonermin, strontium-89 chloride, suramin, tasonermin, tazarotene, tegafur, temoporfin, temozolomide, teniposide, tetrachlorodecaoxide, thalidomide, thymalfasin, thyrotropin alfa, topotecan, toremifene, tositumomab-iodine 131, trastuzumab, treosulfan, tretinoin, trilostane, trimetrexate, triptorelin, tumor necrosis factor alpha, natural, ubenimex, bladder cancer vaccine, Maruyama vaccine, melanoma lysate vaccine, valrubicin, verteporfin, vinorelbine, VIRULIZIN, zinostatin stimalamer, or zoledronic acid; abarelix; AE 941 (Aeterna), ambamustine, antisense oligonucleotide, bcl-2 (Genta), APC 8015 (Dendreon), cetuximab, decitabine, dexaminoglutethimide, diaziquone, EL 532 (Elan), EM 800 (Endorecherche), eniluracil, etanidazole, fenretinide, filgrastim SD01 (Amgen), fulvestrant, galocitabine, gastrin 17 immunogen, HLA-B7 gene therapy (Vical), granulocyte macrophage colony stimulating factor, histamine dihydrochloride, ibritumomab tiuxetan, ilomastat, IM 862 (Cytran), interleukin-2, iproxifene, LDI 200 (Milkhaus), leridistim, lintuzumab, CA 125 MAb (Biomira), cancer MAb (Japan Pharmaceutical Development), HER-2 and Fc MAb (Medarex), idiotypic 105AD7 MAb (CRC Technology), idiotypic CEA MAb (Trilex), LYM-1-iodine 131 MAb (Techniclone), polymorphic epithelial mucin-yttrium 90 MAb (Antisoma), marimastat, menogaril, mitumomab, motexafin gadolinium, MX 6 (Galderma), nelarabine, nolatrexed, P 30 protein, pegvisomant, pemetrexed, porfiromycin, prinomastat, RL 0903 (Shire), rubitecan, satraplatin, sodium phenylacetate, sparfosic acid, SRL 172 (SR Pharma), SU 5416 (SUGEN), TA 077 (Tanabe), tetrathiomolybdate, thaliblastine, thrombopoietin, tin ethyl etiopurpurin, tirapazamine, cancer vaccine (Biomira), melanoma vaccine (New York University), melanoma vaccine (Sloan Kettering Institute), melanoma oncolysate vaccine (New York Medical College), viral melanoma cell lysates vaccine (Royal Newcastle Hospital), or valspodar.

In some embodiments, the one or more additional chemotherapeutic agent is selected from HERCEPTIN™ (trastuzumab), RITUXAN™ (rituximab), ZEVALIN™ (ibritumomab tiuxetan), and LYMPHOCIDE™ (epratuzumab), GLEEVEC™ (imatinib), BEXXAR™ (iodine 131 tositumomab), ERBITUX™ (IMC-C225), AVASTIN™ (Bevacizumab) or VEGF-TRAP™ (aflibercept), ABX-EGF (panitumumab), IRESSA™ (gefitinib) and TARCEVA™ (erlotinib).

Provided compounds can be prepared using the general synthetic routes shown below in Scheme 1, Scheme 2, and Scheme 3.

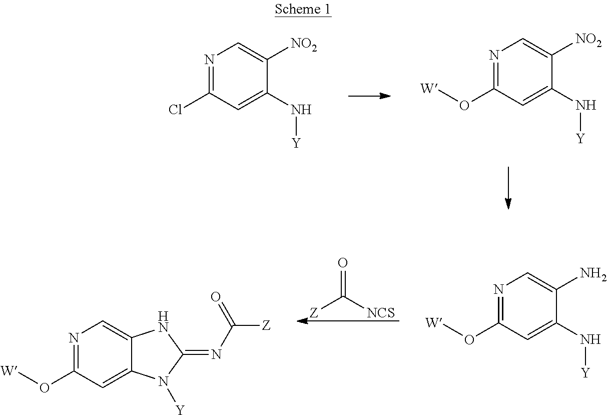

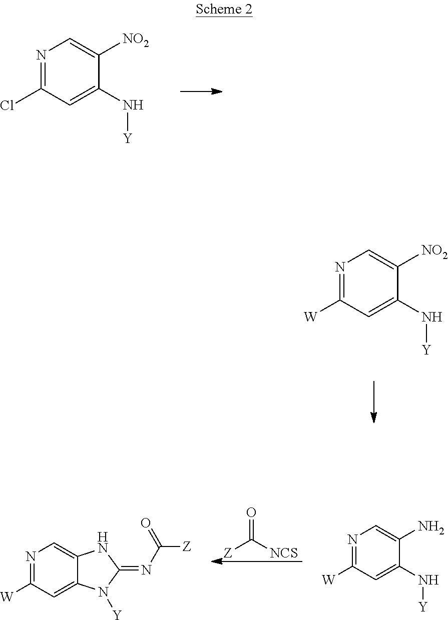

As shown in Schemes 1 and 2, Y-substituted 4-amino-2-chloro-5-nitropyridines provide excellent access to compounds of Formula I where X is N. As shown in Schemes 1 and 2, nucleophiles, such as 2-(piperidin-1-yl)ethanol or morpholine can be reacted with Y-substituted 4-amino-2-chloro-5-nitropyridine compounds to displace the chlorine group and form the appropriate bond to a selected W group. Reduction of the nitro group to an amine using hydrogenation conditions followed by reaction with a selected isothiocyanate such as 4-fluorobenzoyl isothiocyanate to form the five-membered ring and provide the appropriate Z group allows ready access to the compounds of Formula I where X is N.

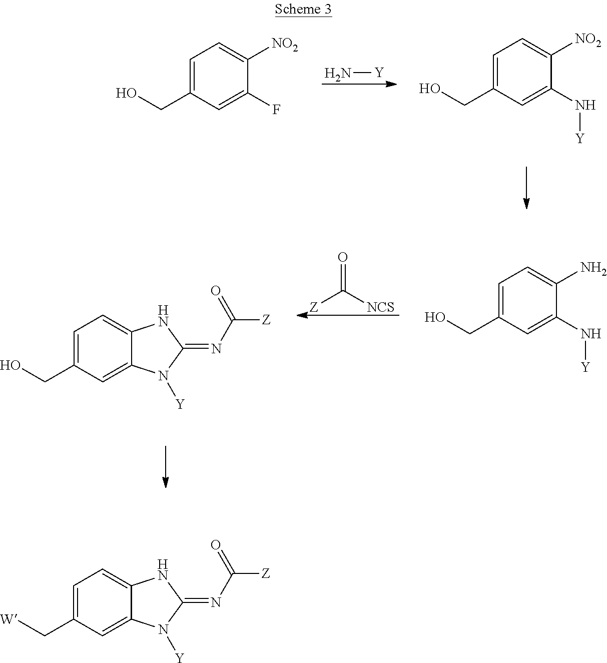

As shown in Scheme 3, (3-fluoro-4-nitrophenyl)methanol provides a convenient starting material for the preparation of various compounds of Formula I where X is C. For example, an amine nucleophile bearing a selected Y group such as N-(cis-4-aminocyclohexyl)isobutyramide may be reacted with (3-fluoro-4-nitrophenyl)methanol to form the Y substituted (3-amino-4-nitrophenyl)methanol compound. Reduction of the nitro group to form the amino compound followed by reaction with an appropriate isothiocyanate such as 4-benzoyl isothiocyanate forms the five-membered ring and adds the desired Z group to provide a useful hydroxymethyl compound that can be readily converted to various compounds of Formula I. For example, the hydroxymethyl compound may be converted to a reactive chloromethyl intermediate by reaction with thionyl chloride. The chloromethyl intermediate may then be reacted with an appropriate nucleophile such as 2-(piperidin-4-yl)propan-2-ol to obtain the compound of Formula I.

Modification of the above Schemes can be used to synthesize numerous compounds of the invention as will be apparent to those skilled in the art.

In some embodiments, a compound of formula I is administered once, twice, three times or four times a day. In some embodiments, a compound of formula I is administered in a dose of from about 1 mg to about 20 mg, from about 5 mg to about 20 mg, from about 10 mg to about 30 mg, from about 20 mg to about 50 mg, from about 50 mg to about 1200 mg, from about 300 mg to about 1200 mg, from about 600 mg to about 1200 mg, from about 800 mg to about 1200 mg, from about 1000 mg to about 1200 mg, from about 50 mg to about 500 mg, from about 100 mg to about 500 mg, or from about 300 mg to about 500 mg. In some embodiments, a compound of formula I is administered in a dose of about 1 mg, 5 mg, 10 mg, 15 mg, 20 mg, 25 mg, 30 mg, 35 mg, 40 mg, 45 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1025 mg, 1050 mg, 1075 mg, 1100 mg, 1125 mg, 1150 mg, 1175 mg or 1200 mg.

In certain embodiments, compounds of formula I, or a pharmaceutically acceptable composition thereof, are administered in combination with an antiproliferative or chemotherapeutic agent selected from any one or more of abarelix, aldesleukin, alemtuzumab, alitretinoin, allopurinol, altretamine, amifostine, anastrozole, arsenic trioxide, asparaginase, azacitidine, BCG Live, bevacuzimab, fluorouracil, bexarotene, bleomycin, bortezomib, busulfan, calusterone, capecitabine, camptothecin, carboplatin, carmustine, celecoxib, cetuximab, chlorambucil, cladribine, clofarabine, cyclophosphamide, cytarabine, dactinomycin, darbepoetin alfa, daunorubicin, denileukin, dexrazoxane, docetaxel, doxorubicin (neutral), doxorubicin hydrochloride, dromostanolone propionate, epirubicin, epoetin alfa, erlotinib, estramustine, etoposide phosphate, etoposide, exemestane, filgrastim, floxuridine fludarabine, fulvestrant, gefitinib, gemcitabine, gemtuzumab, goserelin acetate, histrelin acetate, hydroxyurea, ibritumomab, idarubicin, ifosfamide, imatinib mesylate, interferon alfa-2a, interferon alfa-2b, irinotecan, lenalidomide, letrozole, leucovorin, leuprolide acetate, levamisole, lomustine, megestrol acetate, melphalan, mercaptopurine, 6-MP, mesna, methotrexate, methoxsalen, mitomycin C, mitotane, mitoxantrone, nandrolone, nelarabine, nofetumomab, oprelvekin, oxaliplatin, paclitaxel, palifermin, pamidronate, pegademase, pegaspargase, pegfilgrastim, pemetrexed disodium, pentostatin, pipobroman, plicamycin, porfimer sodium, procarbazine, quinacrine, rasburicase, rituximab, sargramostim, sorafenib, streptozocin, sunitinib maleate, talc, tamoxifen, temozolomide, teniposide, VM-26, testolactone, thioguanine, 6-TG, thiotepa, topotecan, toremifene, tositumomab, trastuzumab, tretinoin, ATRA, uracil mustard, valrubicin, vinblastine, vincristine, vinorelbine, zoledronate, or zoledronic acid.

Other examples of agents the inhibitors of this invention may also be combined with include, without limitation: treatments for Alzheimer's Disease such as donepezil hydrochloride (Aricept®) and rivastigmine (Exelon®); treatments for Parkinson's Disease such as L-DOPA/carbidopa, entacapone, ropinrole, pramipexole, bromocriptine, pergolide, trihexephendyl, and amantadine; agents for treating Multiple Sclerosis (MS) such as beta interferon (e.g., Avonex® and Rebif®), glatiramer acetate (Copaxone®), and mitoxantrone; treatments for asthma such as albuterol and montelukast (Singulair®); agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; and agents for treating immunodeficiency disorders such as gamma globulin.

In certain embodiments, compounds of formula I, or a pharmaceutically acceptable composition thereof, are administered in combination with a monoclonal antibody or an siRNA therapeutic.

Those additional agents may be administered separately from a composition comprising a compound of formula I as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

The amount of both a compound of formula I and additional therapeutic agent (in those compositions which comprise an additional therapeutic agent as described above)) that may be combined with the carrier materials to produce a single dosage form will vary depending upon the host treated and the particular mode of administration. Preferably, compositions of this invention should be formulated so that a dosage of between 0.01-100 mg/kg body weight/day of a compound of formula I can be administered.

In those compositions which comprise an additional therapeutic agent, that additional therapeutic agent and the compound of formula I may act synergistically. Therefore, the amount of additional therapeutic agent in such compositions will be less than that required in a monotherapy utilizing only that therapeutic agent. In such compositions a dosage of between 0.01-1,000 μg/kg body weight/day of the additional therapeutic agent can be administered.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

Compounds of formula I, or pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor Implantable devices coated with a compound of this invention are another embodiment of the present invention.

### EXEMPLIFICATION

### Example 1

The IC₅₀ values for crizotinib, compound I-a and compound I-b were determined using a Mobility Shift Assay platform. Each of the test compounds was dissolved in and serially diluted with dimethylsulfoxide (DMSO) and assay buffer to achieve final concentrations of 1.0, 0.3, 0.1, 0.03, 0.01, 0.003, 0.001, 0.0003, 0.0001 and 0.00003 Crizotinib, I-a and I-b were tested against a 19 kinase panel consisting of ALK, ALK [F1174L], ALK [L1196M], ALK [R1275Q], EML4-ALK, EGFR [T790M], FAK, FLT1, FLT3, FLT4, KDR, RET, RET [G691S], RET [M918T], RET [S891A], RET [Y791F], TRKA, TRKB and TRKC.

Off-Chip Mobility Assay.

The 5 μL of ×4 compound solution, 5 μL of ×4 Substrate/ATP/Metal solution, and 10 μL of ×2 kinase solution were prepared with assay buffer (20 mM HEPES, 0.01% Triton X-100, 2 mM DTT, pH 7.5) and mixed and incubated in a well of polypropylene 384 well microplate for 1 or 5 hour(s)* at room temperature. (*; depend on kinase). 60 μL of Termination Buffer (QuickScout Screening Assist MSA; Cama Biosciences) was added to the well. The reaction mixture was applied to LabChip3000 system (Caliper Life Science), and the product and substrate peptide peaks were separated and quantitated. The kinase reaction was evaluated by the product ratio calculated from peak heights of product(P) and substrate(S) peptides (P/(P+S)).

Assay reaction conditions are given in Table 2, below. Calculated IC₅₀ values are set forth in Table 3.

TABLE 2
Assay Reaction Conditions

| | Substrate | | ATP (μM) | | Metal | | |
|---|---|---|---|---|---|---|---|
| Kinase | Name | (nM) | Km | Assay | Name | (mM) | Positive control |
| ALK | Srctide | 1000 | 57 | 50 | Mg | 5 | Staurosporine |
| ALK [F1174L] | Srctide | 1000 | 49 | 50 | Mg | 5 | Staurosporine |
| ALK [L1196M] | Srctide | 1000 | 63 | 75 | Mg | 5 | Staurosporine |
| ALK [R1275Q] | Srctide | 1000 | 84 | 100 | Mg | 5 | Staurosporine |
| EML4-ALK* | Srctide | 1000 | 43 | 50 | Mg | 5 | Staurosporine |
| EGFR [T790M] | Srctide | 1000 | 0.9 | 1 | Mg + Mn | 5 + 1 | Staurosporine |
| FAK | Blk/Lyntide | 1000 | 25 | 25 | Mg | 5 | Staurosporine |
| FLT1* | CSKtide | 1000 | 140 | 150 | Mg | 5 | Staurosporine |
| FLT3 | Srctide | 1000 | 94 | 100 | Mg | 5 | Staurosporine |
| FLT4 | CSKtide | 1000 | 72 | 75 | Mg | 5 | Staurosporine |
| KDR | CSKtide | 1000 | 74 | 75 | Mg | 5 | Staurosporine |
| RET | CSKtide | 1000 | 7.5 | 10 | Mg | 5 | Staurosporine |
| RET [G691S] | CSKtide | 1000 | 13 | 10 | Mg | 5 | Staurosporine |
| RET [M918T] | CSKtide | 1000 | 4.2 | 5 | Mg | 5 | Staurosporine |
| RET [S891A] | CSKtide | 1000 | 11 | 10 | Mg | 5 | Staurosporine |
| RET [Y791F] | CSKtide | 1000 | 29 | 25 | Mg | 5 | Staurosporine |
| TRKA | CSKtide | 1000 | 65 | 75 | Mg | 5 | Staurosporine |
| TRKB | Srctide | 1000 | 80 | 75 | Mg | 5 | Staurosporine |
| TRKC | Srctide | 1000 | 47 | 50 | Mg | 5 | Staurosporine |

*Reaction time is 5 hours

TABLE 3
IC₅₀ (nM)

| Kinase | Crizotinib | I-a | I-b | Staurosporine |
|---|---|---|---|---|
| ALK | B | A | A | B |
| ALK(F1174L) | B | B | B | B |
| ALK(L1196M) | B | A | A | B |
| ALK(R1275Q) | B | A | A | B |
| EML4-ALK | B | A | A | B |
| EGFR(T790M) | E | E | E | B |
| FAK | B | B | C | B |
| FLT1 | D | E | E | B |
| FLT3 | D | B | B | A |
| FLT4 | E | E | E | A |
| KDR | E | E | E | B |
| RET | D | B | E | B |
| RET(G691S) | E | B | E | B |
| RET(M918T) | E | B | E | B |
| RET(S 891A) | C | B | C | A |
| RET(Y791F) | E | B | E | B |
| TRKA | B | A | B | A |
| TRKB | B | B | B | A |
| TRKC | B | B | B | A |

A = <1 nM;
B = 1-100 nM;
C = 101-500 nM;
D = 501-1000 nM;
E = >1000 nM

### Example 2

### ALK Inhibition in Enzyme Assay

The cytoplasmic domain (amino acids 1058-1620) of wild-type human ALK was expressed in SF9 cells as an N-terminal GST fusion protein. Kinase activity of the purified protein was assessed using a Lance® TR-FRET assay. The kinase reaction was performed in a 384-well microtiter plate using 2 nM enzyme in 20 mM HEPES (pH 7.5), 0.05% BSA, 2 mM DTT, 10 mM MgCl₂, 1 μM peptide substrate (Biotin-Ahx-EQEDEPEGIYGVLF-OH)(SEQ ID NO: 1), and ATP at 40 μM (the Km apparent). The reaction was allowed to proceed for 90 minutes at room temperature and was then terminated with 20 mM EDTA in 50 mM Tris (pH 7.5), 100 mM NaCl, 0.05% BSA, and 0.1% Tween-20. Phosphorylation of the peptide substrate was detected using the Lance® detection reagents streptavidin-allophycocyanin (SA-APC) and Eu-W1024 anti-phosphotyrosine antibody (PT66) from Perkin Elmer Life Sciences (Waltham, Mass.). The plates were read on a RUBY star plate reader (BMG LABTECH, Cary, N.C.) with an excitation wavelength of 320 nm. Emission was monitored at 615 nm and 665 nm, with increased emission at 665 nm indicative of peptide phosphorylation. Compound IC₅₀ values were calculated from the magnitude of signal in the 655 nm emission channel and were expressed as the mean of three replicates.

Table 4 sets forth the ALK IC₅₀ values obtained using the procedure set forth above for the Example compounds described herein.

TABLE 4
ALK IC₅₀ values of Example Compounds

| Example # in | ALK IC₅₀ | |
|---|---|---|
| WO 2012/018668 | (μM)^{a} | Structure |
| 1 | ++++ | 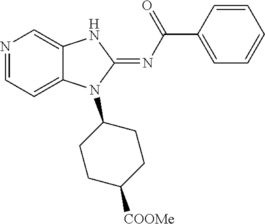 [SMILES: COC(=O)[C@H]1CC[C@@H](n2/c(=N/C(=O)c3ccccc3)[nH]c3cnccc32)CC1] |
| 2 | ++++++ | 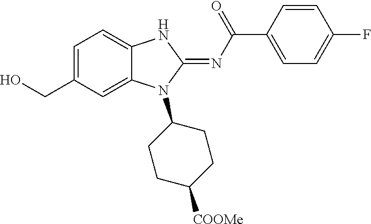 [SMILES: COC(=O)[C@H]1CC[C@@H](n2/c(=N/C(=O)c3ccc(F)cc3)[nH]c3ccc(CO)cc32)CC1] |
| 3 | ++++++ | 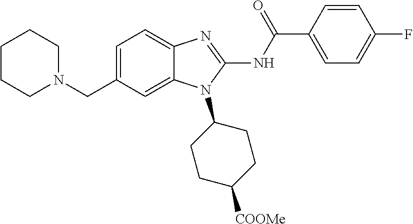 [SMILES: COC(=O)[C@H]1CC[C@@H](n2c(NC(=O)c3ccc(F)cc3)nc3ccc(CN4CCCCC4)cc32)CC1] |
| 4 | ++++++ | 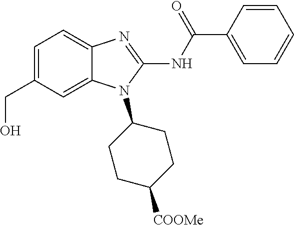 [SMILES: COC(=O)[C@H]1CC[C@@H](n2c(NC(=O)c3ccccc3)nc3ccc(CO)cc32)CC1] |
| 5 | ++++++ | 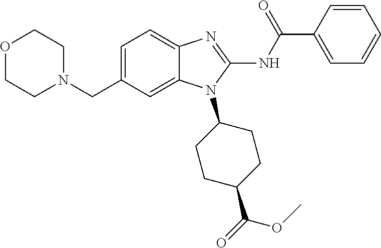 [SMILES: COC(=O)[C@H]1CC[C@@H](n2c(NC(=O)c3ccccc3)nc3ccc(CN4CCOCC4)cc32)CC1] |
| 6 | ++++++ | 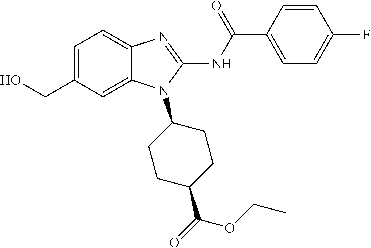 [SMILES: CCOC(=O)[C@H]1CC[C@@H](n2c(NC(=O)c3ccc(F)cc3)nc3ccc(CO)cc32)CC1] |
| 7 | ++++++ | 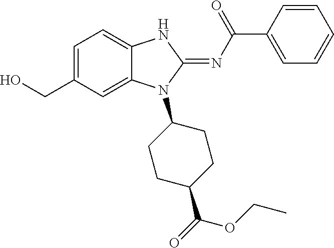 [SMILES: CCOC(=O)[C@H]1CC[C@@H](n2/c(=N/C(=O)c3ccccc3)[nH]c3ccc(CO)cc32)CC1] |
| 8 | ++++++ | 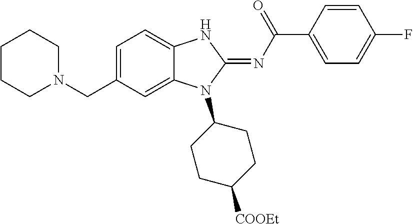 [SMILES: CCOC(=O)[C@H]1CC[C@@H](n2/c(=N/C(=O)c3ccc(F)cc3)[nH]c3ccc(CN4CCCCC4)cc32)CC1] |
| 9 | ++++++ | 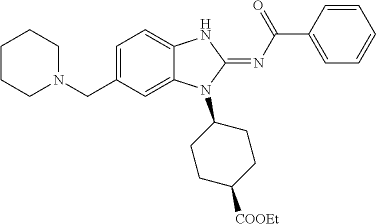 [SMILES: CCOC(=O)[C@H]1CC[C@@H](n2/c(=N/C(=O)c3ccccc3)[nH]c3ccc(CN4CCCCC4)cc32)CC1] |
| 10 | ++++ | 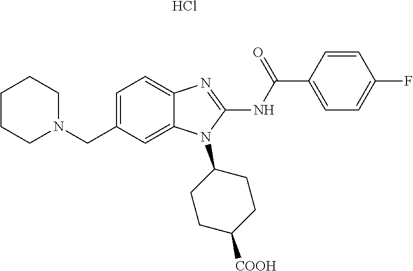 [SMILES: Cl ; O=C(Nc1nc2ccc(CN3CCCCC3)cc2n1[C@H]1CC[C@@H](C(=O)O)CC1)c1ccc(F)cc1] |
| 11 | ++++++ | 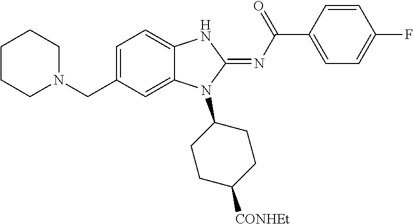 |
| 12 | ++++ | 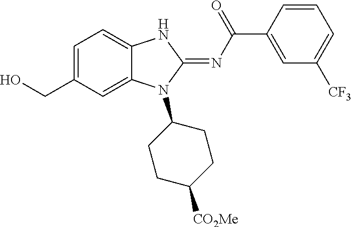 [SMILES: COC(=O)[C@H]1CC[C@@H](n2/c(=N/C(=O)c3cccc(C(F)(F)F)c3)[nH]c3ccc(CO)cc32)CC1] |
| 13 | ++++ | 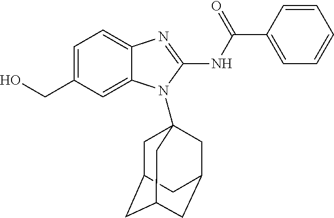 [SMILES: O=C(Nc1nc2ccc(CO)cc2n1C12CC3CC(CC(C3)C1)C2)c1ccccc1] |
| 14 | ++++++ | 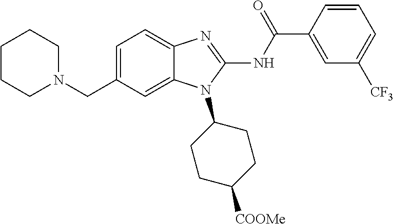 [SMILES: COC(=O)[C@H]1CC[C@@H](n2c(NC(=O)c3cccc(C(F)(F)F)c3)nc3ccc(CN4CCCCC4)cc32)CC1] |
| 15 | ++++++ | 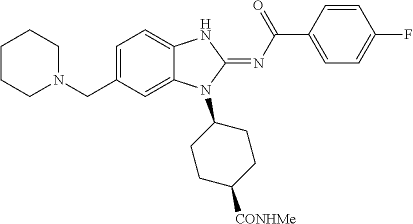 |
| 16 | +++++ | 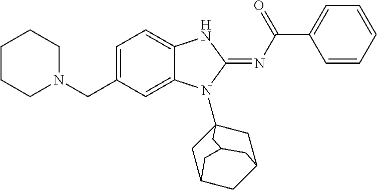 [SMILES: O=C(/N=c1\[nH]c2ccc(CN3CCCCC3)cc2n1C12CC3CC(CC(C3)C1)C2)c1ccccc1] |
| 17 | ++++++ | 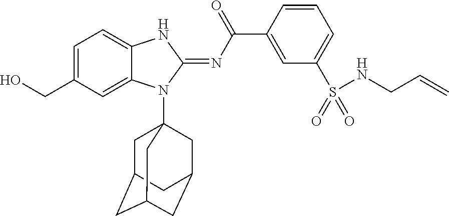 [SMILES: C=CCNS(=O)(=O)c1cccc(C(=O)/N=c2\[nH]c3ccc(CO)cc3n2C23CC4CC(CC(C4)C2)C3)c1] |
| 18 | ++++++ | 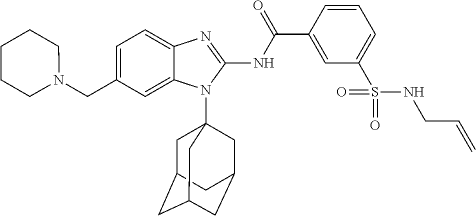 [SMILES: C=CCNS(=O)(=O)c1cccc(C(=O)Nc2nc3ccc(CN4CCCCC4)cc3n2C23CC4CC(CC(C4)C2)C3)c1] |
| 19 | ++++++ | 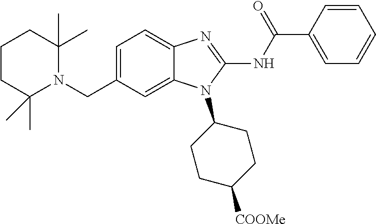 [SMILES: COC(=O)[C@H]1CC[C@@H](n2c(NC(=O)c3ccccc3)nc3ccc(CN4C(C)(C)CCCC4(C)C)cc32)CC1] |
| 20 | ++++++ | 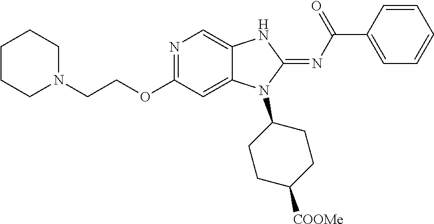 [SMILES: COC(=O)[C@H]1CC[C@@H](n2/c(=N/C(=O)c3ccccc3)[nH]c3cnc(OCCN4CCCCC4)cc32)CC1] |
| 21 | ++++++ | 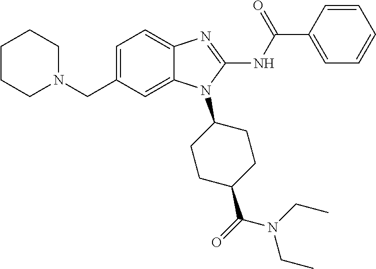 [SMILES: CCN(CC)C(=O)[C@H]1CC[C@@H](n2c(NC(=O)c3ccccc3)nc3ccc(CN4CCCCC4)cc32)CC1] |
| 22 | ++++++ | 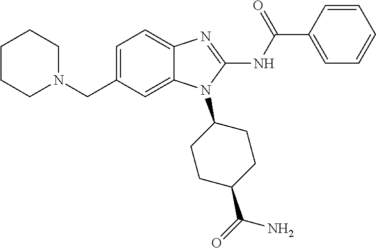 [SMILES: NC(=O)[C@H]1CC[C@@H](n2c(NC(=O)c3ccccc3)nc3ccc(CN4CCCCC4)cc32)CC1] |
| 23 | ++++++ | 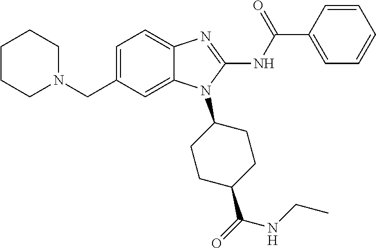 [SMILES: CCNC(=O)[C@H]1CC[C@@H](n2c(NC(=O)c3ccccc3)nc3ccc(CN4CCCCC4)cc32)CC1] |
| 24 | ++++++ | 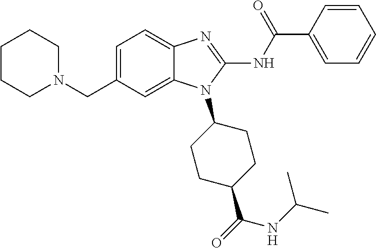 [SMILES: CC(C)NC(=O)[C@H]1CC[C@@H](n2c(NC(=O)c3ccccc3)nc3ccc(CN4CCCCC4)cc32)CC1] |
| 25 | +++ | 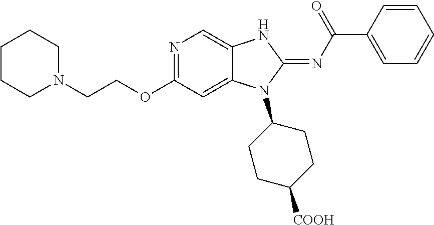 [SMILES: O=C(/N=c1\[nH]c2cnc(OCCN3CCCCC3)cc2n1[C@H]1CC[C@@H](C(=O)O)CC1)c1ccccc1] |
| 26 | ++++++ | 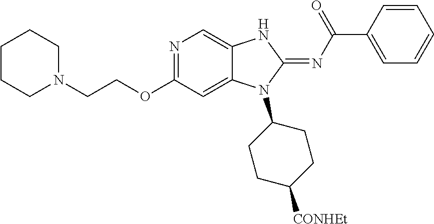 |
| 27 | ++++ | 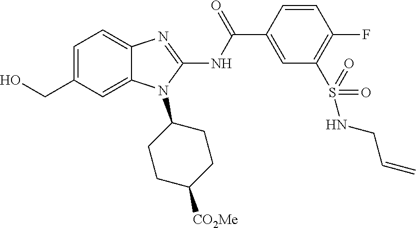 [SMILES: C=CCNS(=O)(=O)c1cc(C(=O)Nc2nc3ccc(CO)cc3n2[C@H]2CC[C@@H](C(=O)OC)CC2)ccc1F] |
| 28 | ++++ | 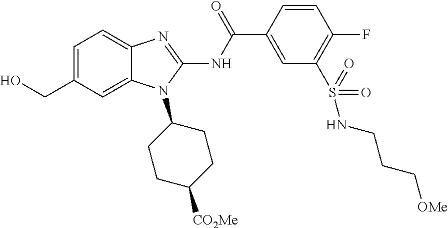 [SMILES: COCCCNS(=O)(=O)c1cc(C(=O)Nc2nc3ccc(CO)cc3n2[C@H]2CC[C@@H](C(=O)OC)CC2)ccc1F] |
| 29 | ++++++ | 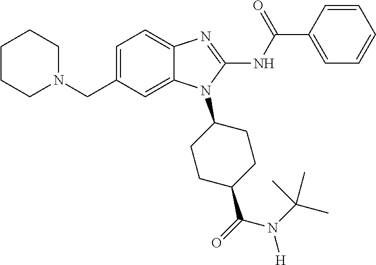 [SMILES: CC(C)(C)NC(=O)[C@H]1CC[C@@H](n2c(NC(=O)c3ccccc3)nc3ccc(CN4CCCCC4)cc32)CC1] |
| 30 | ++++++ | 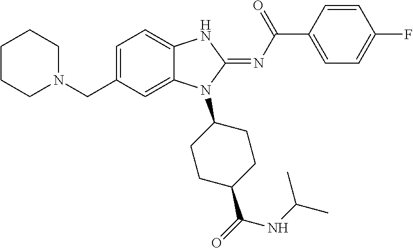 [SMILES: CC(C)NC(=O)[C@H]1CC[C@@H](n2/c(=N/C(=O)c3ccc(F)cc3)[nH]c3ccc(CN4CCCCC4)cc32)CC1] |
| 31 | ++++++ | 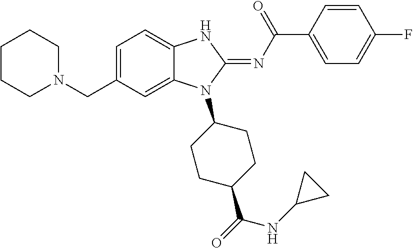 [SMILES: O=C(/N=c1\[nH]c2ccc(CN3CCCCC3)cc2n1[C@H]1CC[C@@H](C(=O)NC2CC2)CC1)c1ccc(F)cc1] |
| 32 | ++++++ | 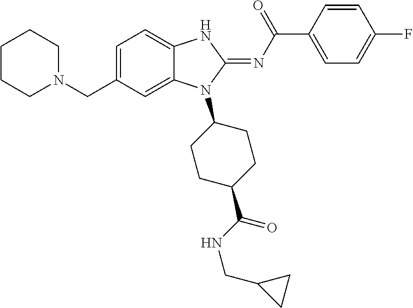 [SMILES: O=C(/N=c1\[nH]c2ccc(CN3CCCCC3)cc2n1[C@H]1CC[C@@H](C(=O)NCC2CC2)CC1)c1ccc(F)cc1] |
| 33 | ++++++ | 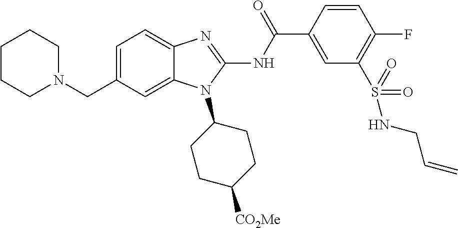 [SMILES: C=CCNS(=O)(=O)c1cc(C(=O)Nc2nc3ccc(CN4CCCCC4)cc3n2[C@H]2CC[C@@H](C(=O)OC)CC2)ccc1F] |
| 34 | ++++ | 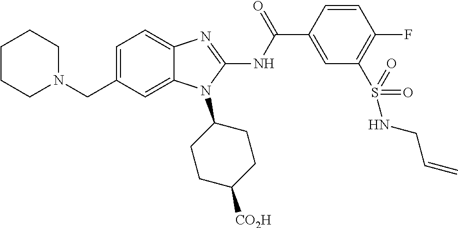 [SMILES: C=CCNS(=O)(=O)c1cc(C(=O)Nc2nc3ccc(CN4CCCCC4)cc3n2[C@H]2CC[C@@H](C(=O)O)CC2)ccc1F] |
| 35 | ++++++ | 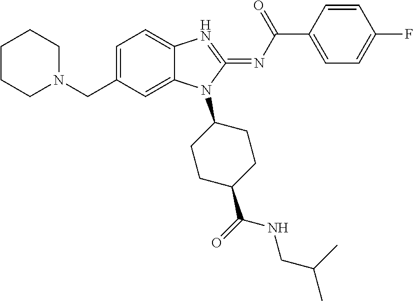 |
| 36 | ++++ | 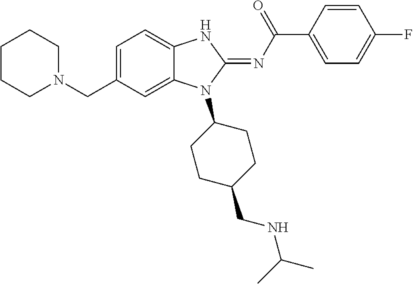 [SMILES: CC(C)NC[C@H]1CC[C@@H](n2/c(=N/C(=O)c3ccc(F)cc3)[nH]c3ccc(CN4CCCCC4)cc32)CC1] |
| 37 | ++++ | 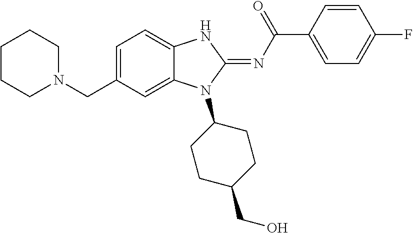 [SMILES: O=C(/N=c1\[nH]c2ccc(CN3CCCCC3)cc2n1[C@H]1CC[C@@H](CO)CC1)c1ccc(F)cc1] |
| 38 | ++++++ | 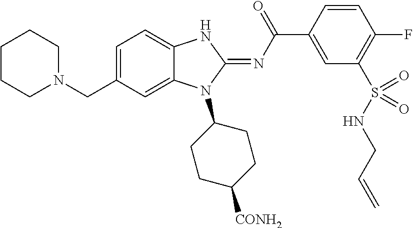 [SMILES: C=CCNS(=O)(=O)c1cc(C(=O)/N=c2\[nH]c3ccc(CN4CCCCC4)cc3n2[C@H]2CC[C@@H](C(N)=O)CC2)ccc1F] |
| 39 | ++++++ | 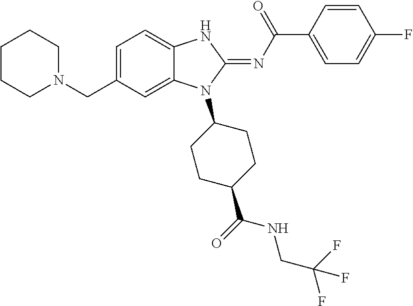 [SMILES: O=C(/N=c1\[nH]c2ccc(CN3CCCCC3)cc2n1[C@H]1CC[C@@H](C(=O)NCC(F)(F)F)CC1)c1ccc(F)cc1] |
| 40 | +++++ | 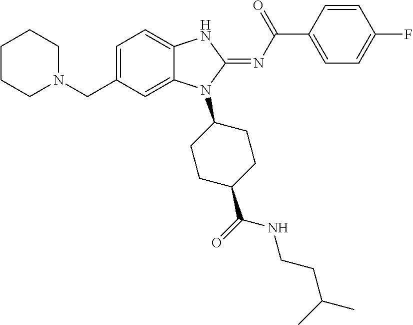 [SMILES: CC(C)CCNC(=O)[C@H]1CC[C@@H](n2/c(=N/C(=O)c3ccc(F)cc3)[nH]c3ccc(CN4CCCCC4)cc32)CC1] |
| 41 | ++++++ | 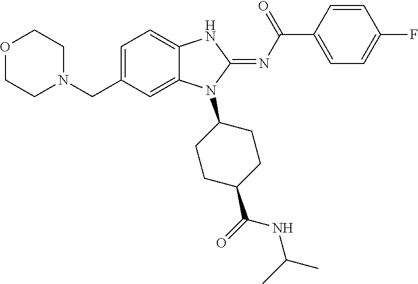 [SMILES: CC(C)NC(=O)[C@H]1CC[C@@H](n2/c(=N/C(=O)c3ccc(F)cc3)[nH]c3ccc(CN4CCOCC4)cc32)CC1] |
| 42 | +++ | 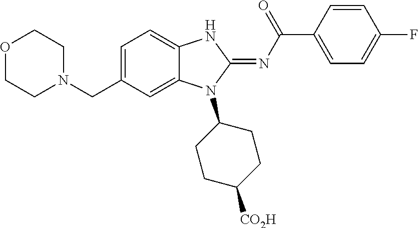 [SMILES: O=C(/N=c1\[nH]c2ccc(CN3CCOCC3)cc2n1[C@H]1CC[C@@H](C(=O)O)CC1)c1ccc(F)cc1] |
| 43 | ++++++ | 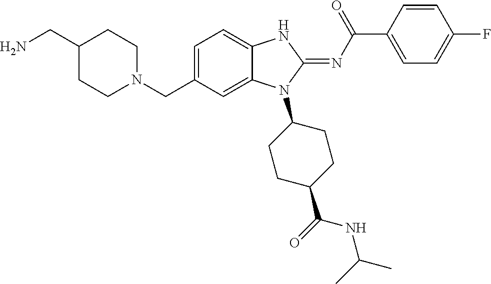 [SMILES: CC(C)NC(=O)[C@H]1CC[C@@H](n2/c(=N/C(=O)c3ccc(F)cc3)[nH]c3ccc(CN4CCC(CN)CC4)cc32)CC1] |
| 44 | ++++++ | 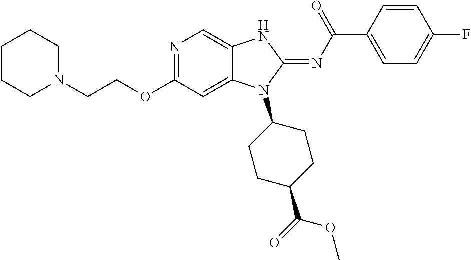 [SMILES: COC(=O)[C@H]1CC[C@@H](n2/c(=N/C(=O)c3ccc(F)cc3)[nH]c3cnc(OCCN4CCCCC4)cc32)CC1] |
| 45 | ++++++ | 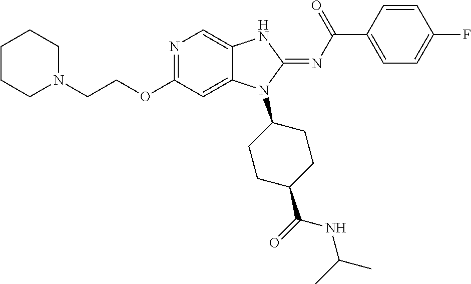 [SMILES: CC(C)NC(=O)[C@H]1CC[C@@H](n2/c(=N/C(=O)c3ccc(F)cc3)[nH]c3cnc(OCCN4CCCCC4)cc32)CC1] |
| 46 | ++++++ | 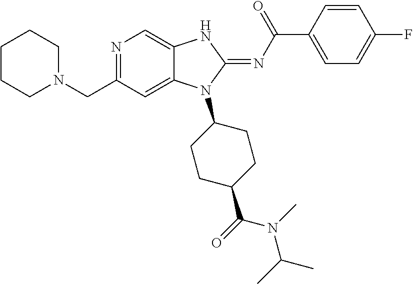 [SMILES: CC(C)N(C)C(=O)[C@H]1CC[C@@H](n2/c(=N/C(=O)c3ccc(F)cc3)[nH]c3cnc(CN4CCCCC4)cc32)CC1] |
| 47 | ++++++ | 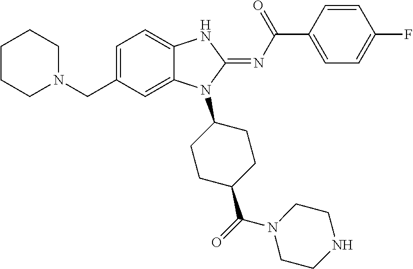 [SMILES: O=C(/N=c1\[nH]c2ccc(CN3CCCCC3)cc2n1[C@H]1CC[C@@H](C(=O)N2CCNCC2)CC1)c1ccc(F)cc1] |
| 48 | ++++++ | 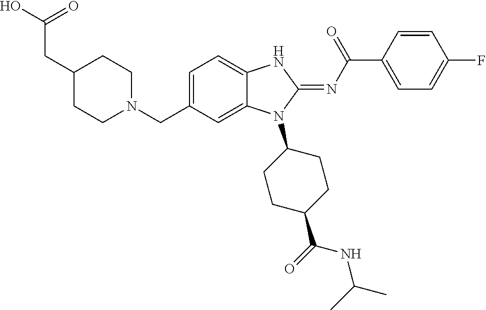 [SMILES: CC(C)NC(=O)[C@H]1CC[C@@H](n2/c(=N/C(=O)c3ccc(F)cc3)[nH]c3ccc(CN4CCC(CC(=O)O)CC4)cc32)CC1] |
| 49 | +++++ | 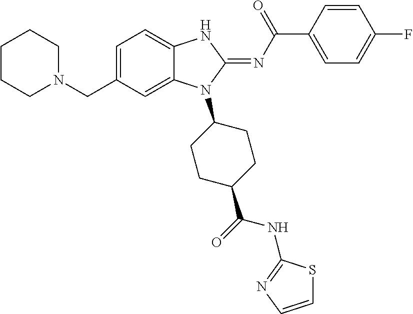 [SMILES: O=C(/N=c1\[nH]c2ccc(CN3CCCCC3)cc2n1[C@H]1CC[C@@H](C(=O)Nc2nccs2)CC1)c1ccc(F)cc1] |
| 50 | ++++++ | 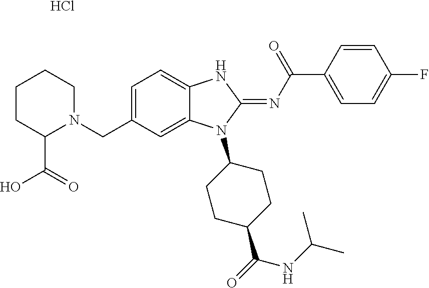 [SMILES: Cl ; CC(C)NC(=O)[C@H]1CC[C@@H](n2/c(=N/C(=O)c3ccc(F)cc3)[nH]c3ccc(CN4CCCCC4C(=O)O)cc32)CC1] |
| 51 | ++++++ | 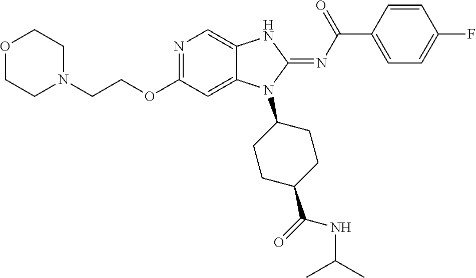 [SMILES: CC(C)NC(=O)[C@H]1CC[C@@H](n2/c(=N/C(=O)c3ccc(F)cc3)[nH]c3cnc(OCCN4CCOCC4)cc32)CC1] |
| 52 | ++++++ | 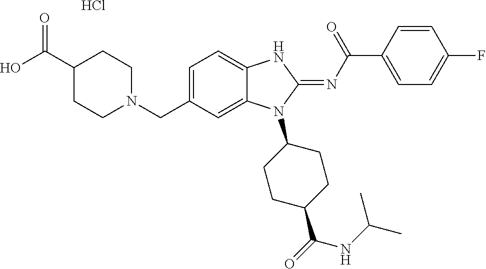 [SMILES: Cl ; CC(C)NC(=O)[C@H]1CC[C@@H](n2/c(=N/C(=O)c3ccc(F)cc3)[nH]c3ccc(CN4CCC(C(=O)O)CC4)cc32)CC1] |
| 53 | ++++++ | 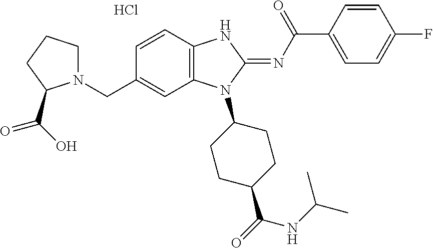 [SMILES: CC(C)NC(=O)[C@H]1CC[C@@H](n2/c(=N/C(=O)c3ccc(F)cc3)[nH]c3ccc(CN4CCC[C@@H]4C(=O)O)cc32)CC1 ; Cl] |
| 54 | ++++++ | 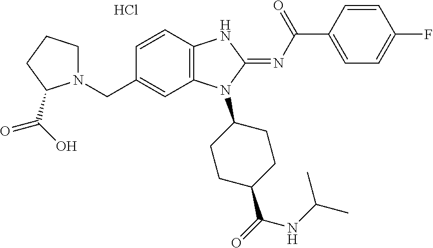 [SMILES: CC(C)NC(=O)[C@H]1CC[C@@H](n2/c(=N/C(=O)c3ccc(F)cc3)[nH]c3ccc(CN4CCC[C@H]4C(=O)O)cc32)CC1 ; Cl] |
| 55 | NA^{b} | |
| 56 | ++++ | 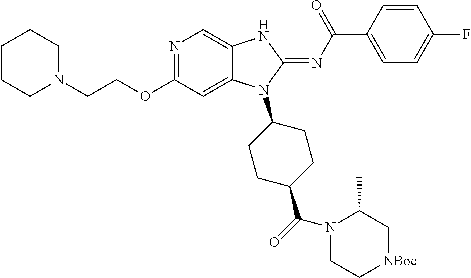 [SMILES: C[C@@H]1CN(C(=O)OC(C)(C)C)CCN1C(=O)[C@H]1CC[C@@H](n2/c(=N/C(=O)c3ccc(F)cc3)[nH]c3cnc(OCCN4CCCCC4)cc32)CC1] |
| 57 | ++++++ | 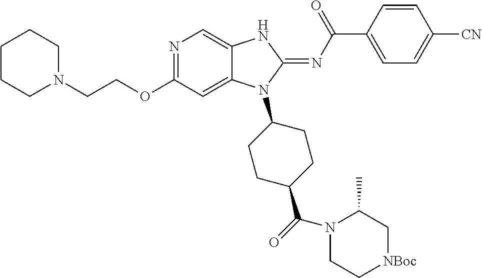 [SMILES: C[C@@H]1CN(C(=O)OC(C)(C)C)CCN1C(=O)[C@H]1CC[C@@H](n2/c(=N/C(=O)c3ccc(C#N)cc3)[nH]c3cnc(OCCN4CCCCC4)cc32)CC1] |
| 58 | ++++++ | 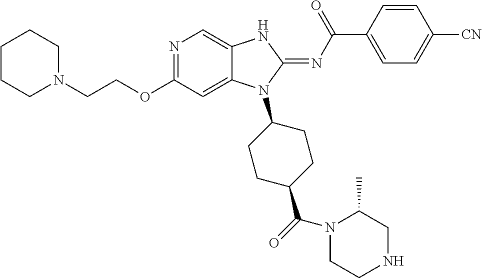 [SMILES: C[C@@H]1CNCCN1C(=O)[C@H]1CC[C@@H](n2/c(=N/C(=O)c3ccc(C#N)cc3)[nH]c3cnc(OCCN4CCCCC4)cc32)CC1] |
| 59 | ++++++ | 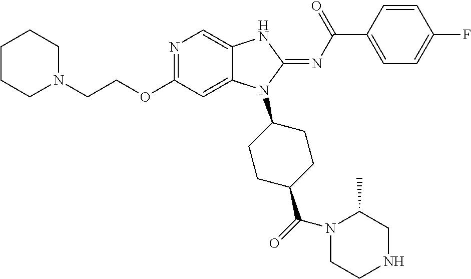 [SMILES: C[C@@H]1CNCCN1C(=O)[C@H]1CC[C@@H](n2/c(=N/C(=O)c3ccc(F)cc3)[nH]c3cnc(OCCN4CCCCC4)cc32)CC1] |
| 60 | +++ | 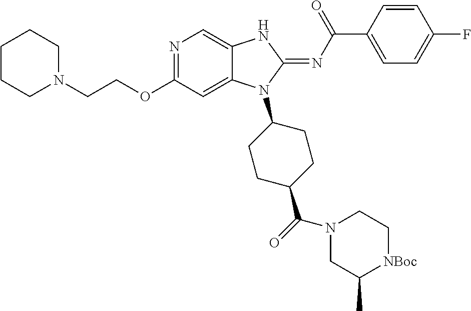 [SMILES: C[C@H]1CN(C(=O)[C@H]2CC[C@@H](n3/c(=N/C(=O)c4ccc(F)cc4)[nH]c4cnc(OCCN5CCCCC5)cc43)CC2)CCN1C(=O)OC(C)(C)C] |
| 61 | ++++++ | 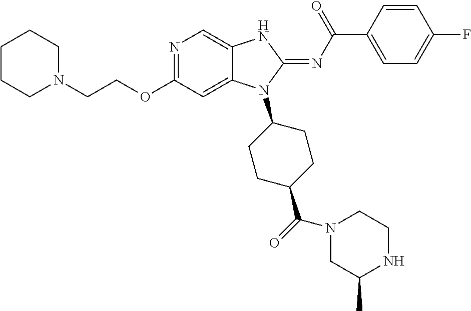 [SMILES: C[C@H]1CN(C(=O)[C@H]2CC[C@@H](n3/c(=N/C(=O)c4ccc(F)cc4)[nH]c4cnc(OCCN5CCCCC5)cc43)CC2)CCN1] |
| 62 | +++++ | 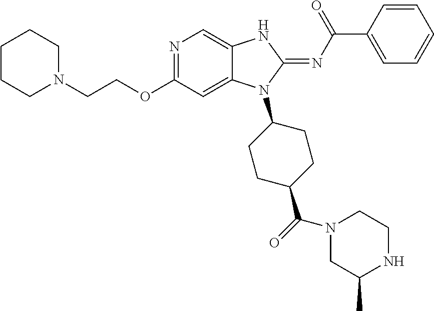 [SMILES: C[C@H]1CN(C(=O)[C@H]2CC[C@@H](n3/c(=N/C(=O)c4ccccc4)[nH]c4cnc(OCCN5CCCCC5)cc43)CC2)CCN1] |
| 63 | +++++ | 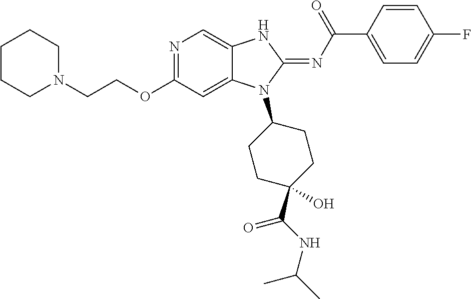 [SMILES: CC(C)NC(=O)[C@]1(O)CC[C@H](n2/c(=N/C(=O)c3ccc(F)cc3)[nH]c3cnc(OCCN4CCCCC4)cc32)CC1] |
| 64 | ++++ | 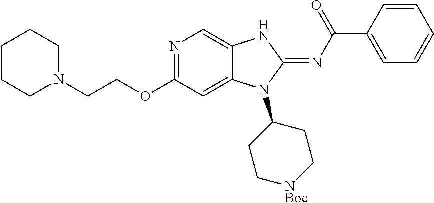 |
| 65 | +++ | 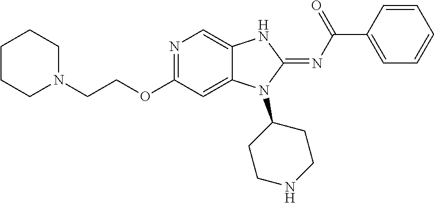 |
| 66 | ++++ | 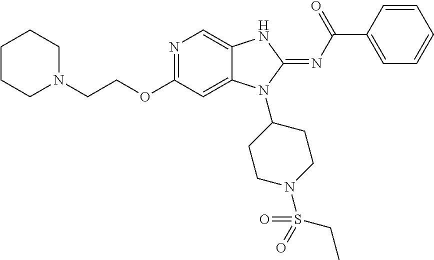 [SMILES: CCS(=O)(=O)N1CCC(n2/c(=N/C(=O)c3ccccc3)[nH]c3cnc(OCCN4CCCCC4)cc32)CC1] |
| 67 | ++++ | 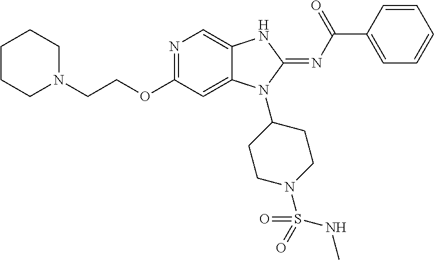 [SMILES: CNS(=O)(=O)N1CCC(n2/c(=N/C(=O)c3ccccc3)[nH]c3cnc(OCCN4CCCCC4)cc32)CC1] |
| 68 | ++ | 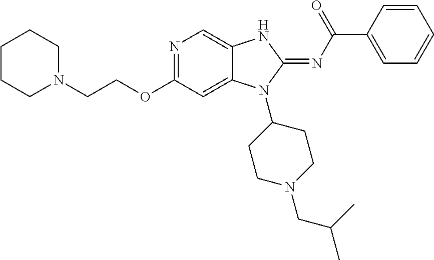 [SMILES: CC(C)CN1CCC(n2/c(=N/C(=O)c3ccccc3)[nH]c3cnc(OCCN4CCCCC4)cc32)CC1] |
| 69 | ++++ | 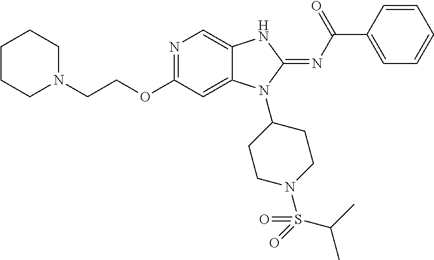 [SMILES: CC(C)S(=O)(=O)N1CCC(n2/c(=N/C(=O)c3ccccc3)[nH]c3cnc(OCCN4CCCCC4)cc32)CC1] |
| 70 | +++ | 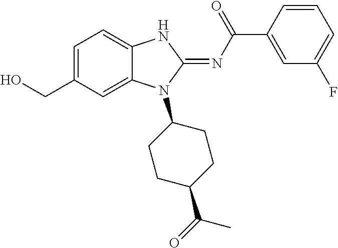 [SMILES: CC(=O)[C@H]1CC[C@@H](n2/c(=N/C(=O)c3cccc(F)c3)[nH]c3ccc(CO)cc32)CC1] |
| 71 | ++++ | 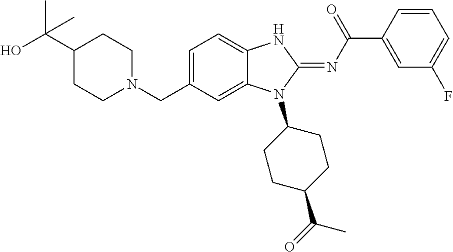 [SMILES: CC(=O)[C@H]1CC[C@@H](n2/c(=N/C(=O)c3cccc(F)c3)[nH]c3ccc(CN4CCC(C(C)(C)O)CC4)cc32)CC1] |
| 72 | ++++++ | 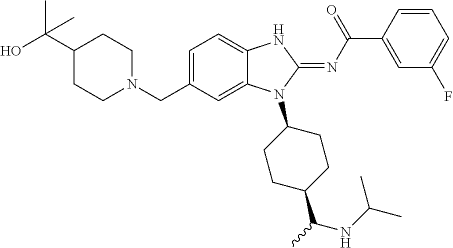 [SMILES: CC(C)NC(C)[C@H]1CC[C@@H](n2/c(=N/C(=O)c3cccc(F)c3)[nH]c3ccc(CN4CCC(C(C)(C)O)CC4)cc32)CC1] |
| 73 | ++++ | 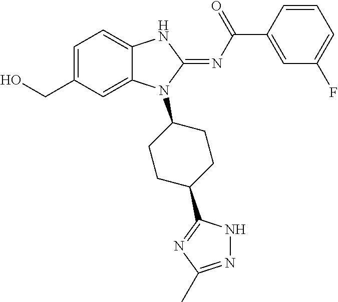 [SMILES: Cc1n[nH]c([C@H]2CC[C@@H](n3/c(=N/C(=O)c4cccc(F)c4)[nH]c4ccc(CO)cc43)CC2)n1] |
| 74 | ++++++ | 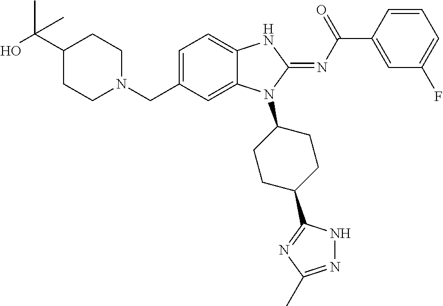 [SMILES: Cc1n[nH]c([C@H]2CC[C@@H](n3/c(=N/C(=O)c4cccc(F)c4)[nH]c4ccc(CN5CCC(C(C)(C)O)CC5)cc43)CC2)n1] |
| 75 | ++++ | 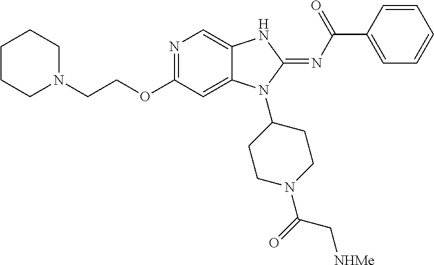 [SMILES: CNCC(=O)N1CCC(n2/c(=N/C(=O)c3ccccc3)[nH]c3cnc(OCCN4CCCCC4)cc32)CC1] |
| 76 | +++ | 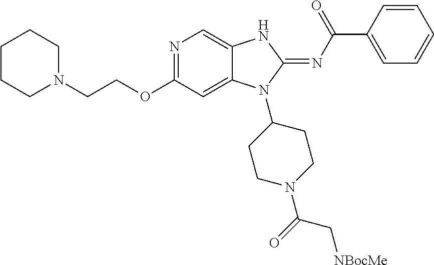 |
| 77 | ++++++ | 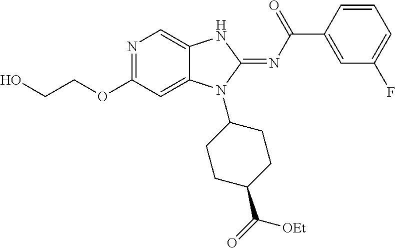 |
| 78 | ++++++ | 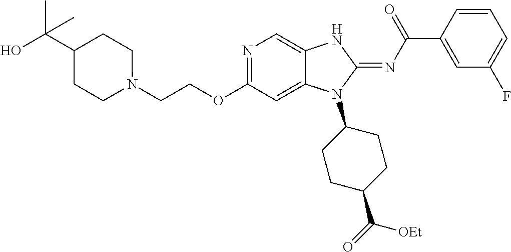 [SMILES: CCOC(=O)[C@H]1CC[C@@H](n2/c(=N/C(=O)c3cccc(F)c3)[nH]c3cnc(OCCN4CCC(C(C)(C)O)CC4)cc32)CC1] |
| 79 | +++ | 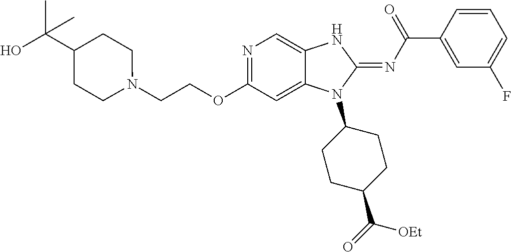 [SMILES: CCOC(=O)[C@H]1CC[C@@H](n2/c(=N/C(=O)c3cccc(F)c3)[nH]c3cnc(OCCN4CCC(C(C)(C)O)CC4)cc32)CC1] |
| 80 | ++++++ | 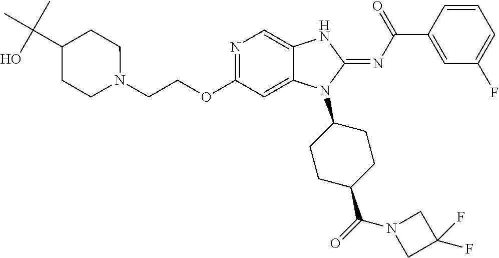 [SMILES: CC(C)(O)C1CCN(CCOc2cc3c(cn2)[nH]/c(=N\C(=O)c2cccc(F)c2)n3[C@H]2CC[C@@H](C(=O)N3CC(F)(F)C3)CC2)CC1] |
| 81 | ++++ | 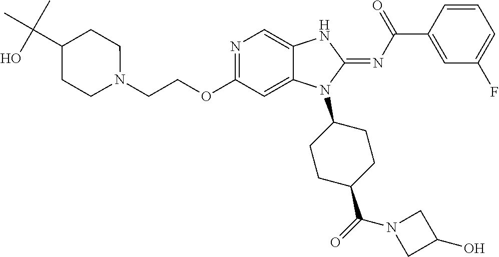 [SMILES: CC(C)(O)C1CCN(CCOc2cc3c(cn2)[nH]/c(=N\C(=O)c2cccc(F)c2)n3[C@H]2CC[C@@H](C(=O)N3CC(O)C3)CC2)CC1] |
| 82 | +++ | 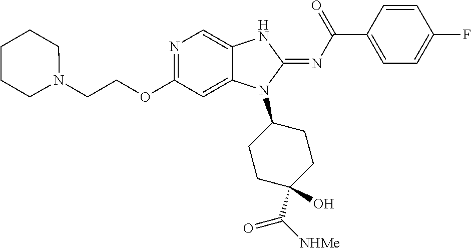 [SMILES: CNC(=O)[C@]1(O)CC[C@@H](n2/c(=N/C(=O)c3ccc(F)cc3)[nH]c3cnc(OCCN4CCCCC4)cc32)CC1] |
| 83 | ++++ | 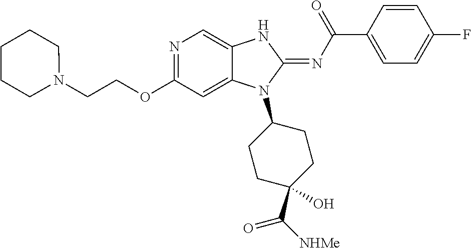 [SMILES: CNC(=O)[C@]1(O)CC[C@H](n2/c(=N/C(=O)c3ccc(F)cc3)[nH]c3cnc(OCCN4CCCCC4)cc32)CC1] |
| 84 | ++++ | 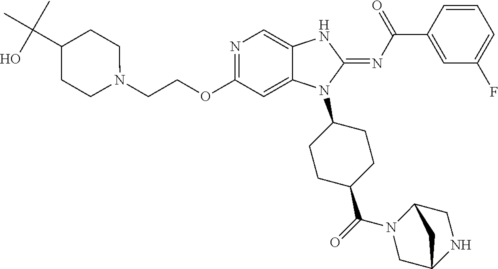 [SMILES: CC(C)(O)C1CCN(CCOc2cc3c(cn2)[nH]/c(=N\C(=O)c2cccc(F)c2)n3[C@H]2CC[C@@H](C(=O)N3C[C@@H]4C[C@H]3CN4)CC2)CC1] |
| 85 | ++++++ | 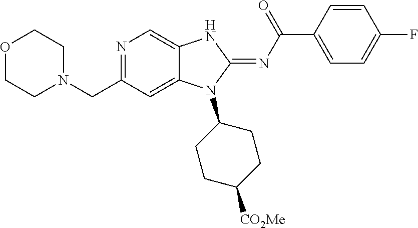 [SMILES: COC(=O)[C@H]1CC[C@@H](n2/c(=N/C(=O)c3ccc(F)cc3)[nH]c3cnc(CN4CCOCC4)cc32)CC1] |
| 86 | ++ | 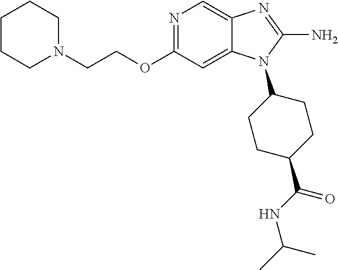 |
| 87 | +++++ | 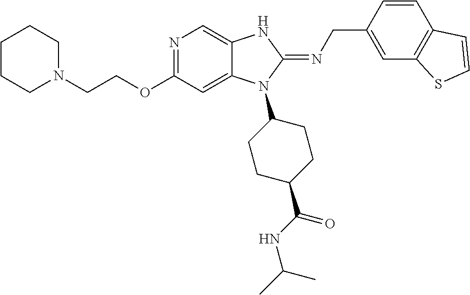 |
| 88 | +++++ | 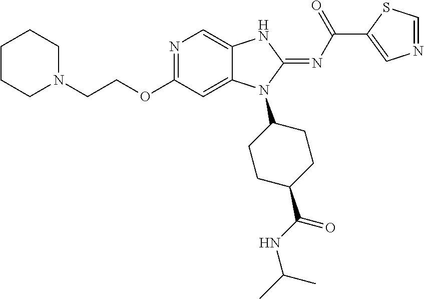 |
| 89 | ++++++ | 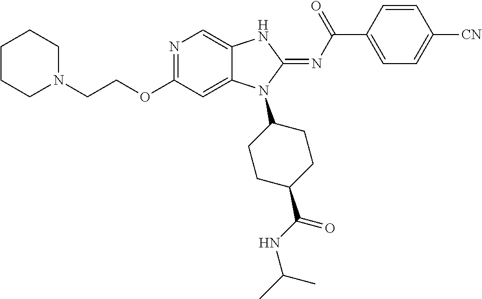 |
| 90 | ++++++ | 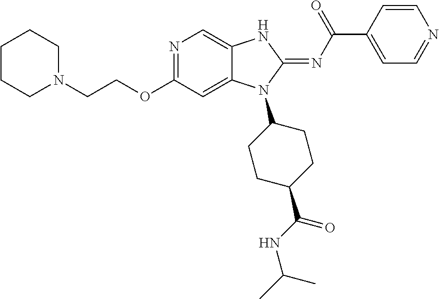 |
| 91 | ++++++ | 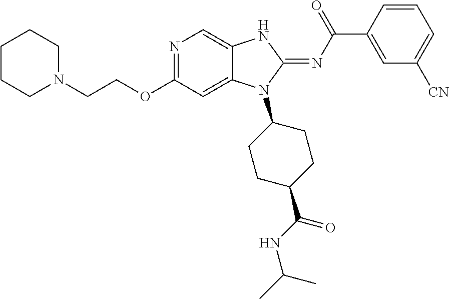 [SMILES: CC(C)NC(=O)[C@H]1CC[C@@H](n2/c(=N/C(=O)c3cccc(C#N)c3)[nH]c3cnc(OCCN4CCCCC4)cc32)CC1] |
| 92 | ++++++ | 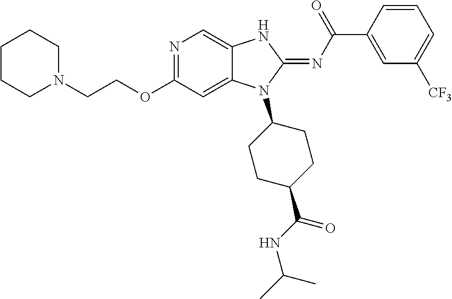 [SMILES: CC(C)NC(=O)[C@H]1CC[C@@H](n2/c(=N/C(=O)c3cccc(C(F)(F)F)c3)[nH]c3cnc(OCCN4CCCCC4)cc32)CC1] |
| 93 | +++ | 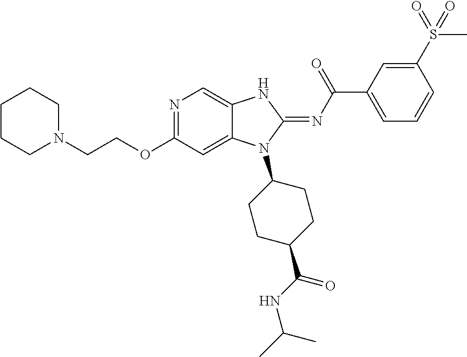 [SMILES: CC(C)NC(=O)[C@H]1CC[C@@H](n2/c(=N/C(=O)c3cccc(S(C)(=O)=O)c3)[nH]c3cnc(OCCN4CCCCC4)cc32)CC1] |
| 94 | +++++ | 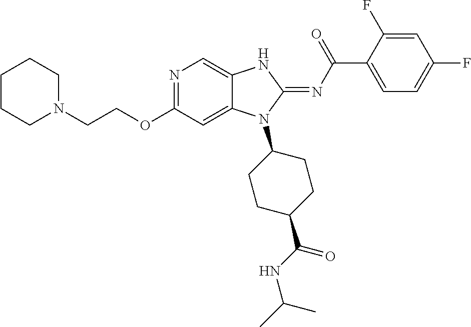 [SMILES: CC(C)NC(=O)[C@H]1CC[C@@H](n2/c(=N/C(=O)c3ccc(F)cc3F)[nH]c3cnc(OCCN4CCCCC4)cc32)CC1] |
| 95 | ++++++ | 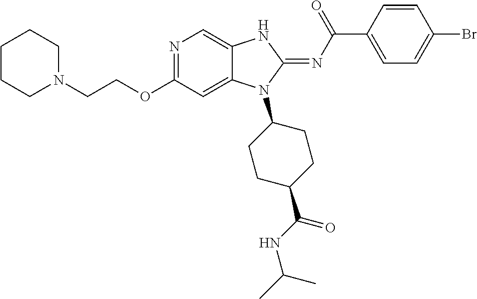 [SMILES: CC(C)NC(=O)[C@H]1CC[C@@H](n2/c(=N/C(=O)c3ccc(Br)cc3)[nH]c3cnc(OCCN4CCCCC4)cc32)CC1] |
| 96 | ++++++ | 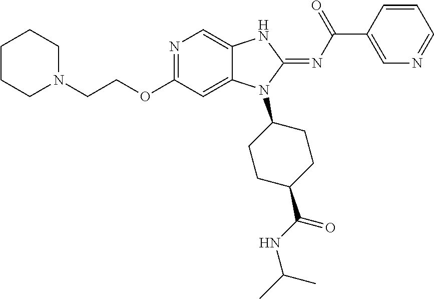 [SMILES: CC(C)NC(=O)[C@H]1CC[C@@H](n2/c(=N/C(=O)c3cccnc3)[nH]c3cnc(OCCN4CCCCC4)cc32)CC1] |
| 97 | +++++ | 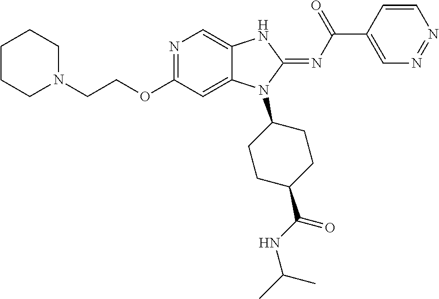 [SMILES: CC(C)NC(=O)[C@H]1CC[C@@H](n2/c(=N/C(=O)c3ccnnc3)[nH]c3cnc(OCCN4CCCCC4)cc32)CC1] |
| 98 | ++++++ | 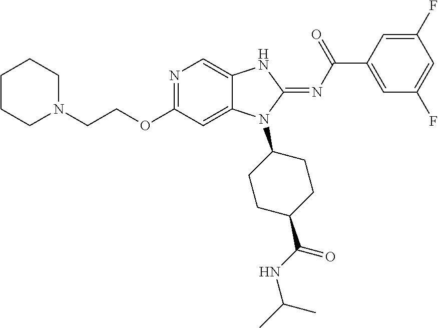 [SMILES: CC(C)NC(=O)[C@H]1CC[C@@H](n2/c(=N/C(=O)c3cc(F)cc(F)c3)[nH]c3cnc(OCCN4CCCCC4)cc32)CC1] |
| 99 | ++++ | 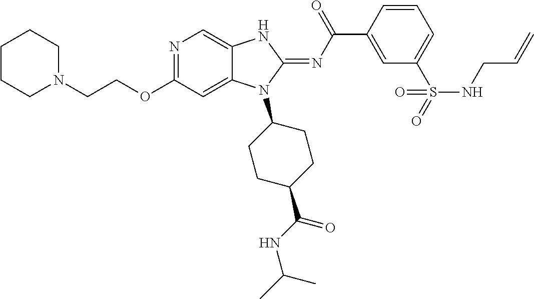 [SMILES: C=CCNS(=O)(=O)c1cccc(C(=O)/N=c2\[nH]c3cnc(OCCN4CCCCC4)cc3n2[C@H]2CC[C@@H](C(=O)NC(C)C)CC2)c1] |
| 100 | +++++ | 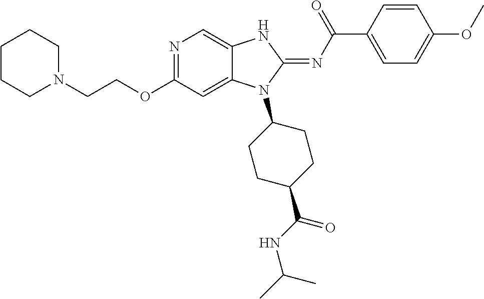 [SMILES: COc1ccc(C(=O)/N=c2\[nH]c3cnc(OCCN4CCCCC4)cc3n2[C@H]2CC[C@@H](C(=O)NC(C)C)CC2)cc1] |
| 101 | ++++++ | 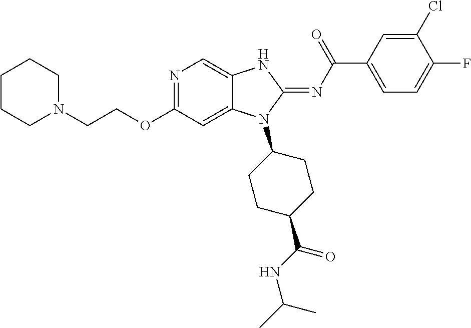 [SMILES: CC(C)NC(=O)[C@H]1CC[C@@H](n2/c(=N/C(=O)c3ccc(F)c(Cl)c3)[nH]c3cnc(OCCN4CCCCC4)cc32)CC1] |
| 102 | ++++ | 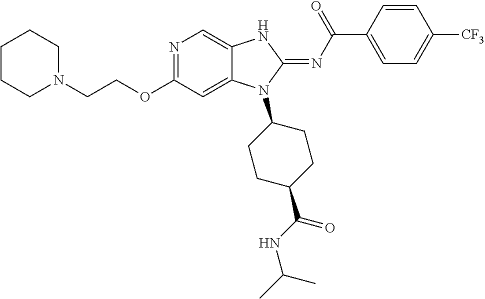 [SMILES: CC(C)NC(=O)[C@H]1CC[C@@H](n2/c(=N/C(=O)c3ccc(C(F)(F)F)cc3)[nH]c3cnc(OCCN4CCCCC4)cc32)CC1] |
| 103 | ++++++ | 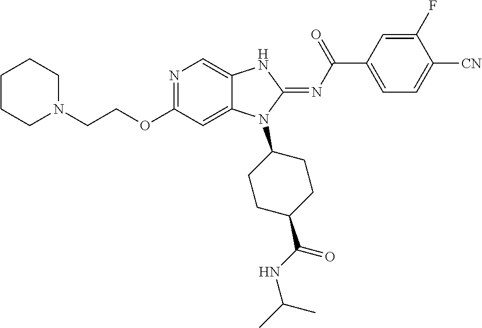 [SMILES: CC(C)NC(=O)[C@H]1CC[C@@H](n2/c(=N/C(=O)c3ccc(C#N)c(F)c3)[nH]c3cnc(OCCN4CCCCC4)cc32)CC1] |
| 104 | ++++ | 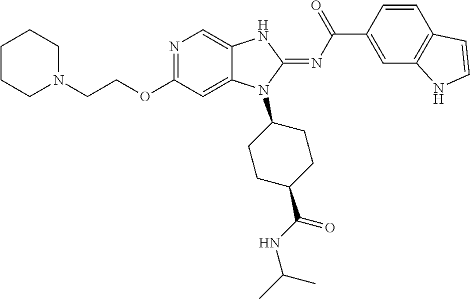 [SMILES: CC(C)NC(=O)[C@H]1CC[C@@H](n2/c(=N/C(=O)c3ccc4cc[nH]c4c3)[nH]c3cnc(OCCN4CCCCC4)cc32)CC1] |
| 105 | +++ | 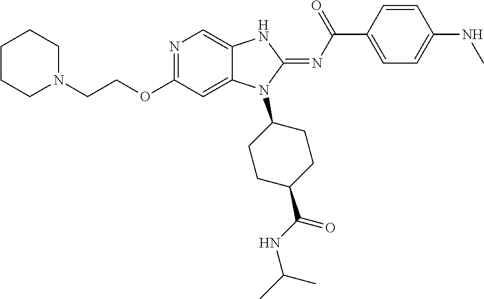 [SMILES: CNc1ccc(C(=O)/N=c2\[nH]c3cnc(OCCN4CCCCC4)cc3n2[C@H]2CC[C@@H](C(=O)NC(C)C)CC2)cc1] |
| 106 | ++++++ | 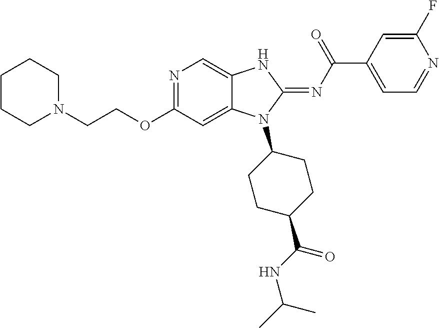 [SMILES: CC(C)NC(=O)[C@H]1CC[C@@H](n2/c(=N/C(=O)c3ccnc(F)c3)[nH]c3cnc(OCCN4CCCCC4)cc32)CC1] |
| 107 | ++++++ | 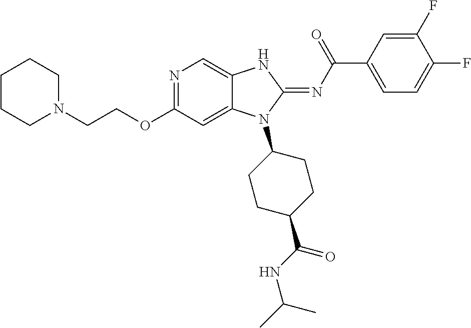 [SMILES: CC(C)NC(=O)[C@H]1CC[C@@H](n2/c(=N/C(=O)c3ccc(F)c(F)c3)[nH]c3cnc(OCCN4CCCCC4)cc32)CC1] |
| 108 | ++++++ | 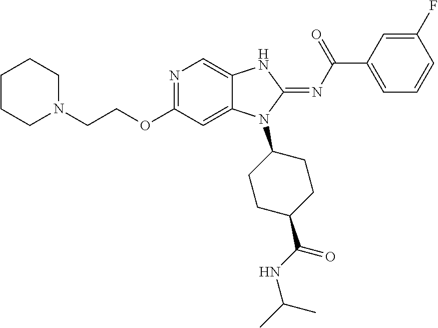 [SMILES: CC(C)NC(=O)[C@H]1CC[C@@H](n2/c(=N/C(=O)c3cccc(F)c3)[nH]c3cnc(OCCN4CCCCC4)cc32)CC1] |
| 109 | +++ | 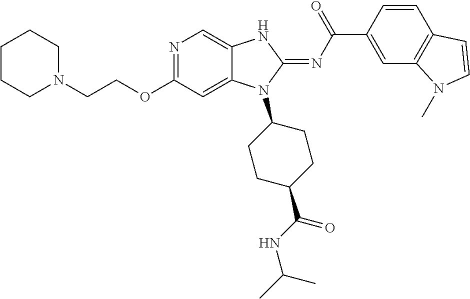 [SMILES: CC(C)NC(=O)[C@H]1CC[C@@H](n2/c(=N/C(=O)c3ccc4ccn(C)c4c3)[nH]c3cnc(OCCN4CCCCC4)cc32)CC1] |
| 110 | +++ | 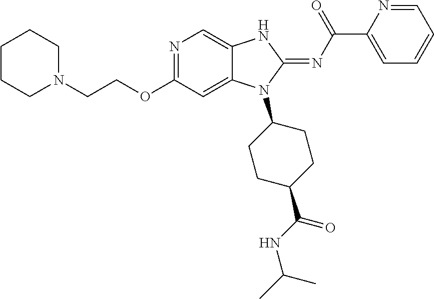 [SMILES: CC(C)NC(=O)[C@H]1CC[C@@H](n2/c(=N/C(=O)c3ccccn3)[nH]c3cnc(OCCN4CCCCC4)cc32)CC1] |
| 111 | ++++++ | 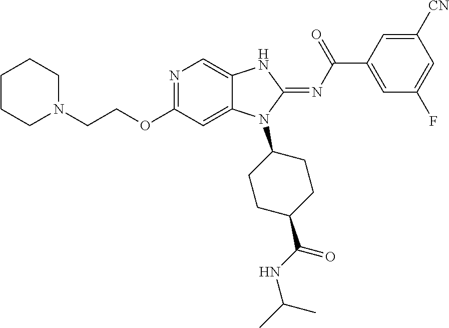 [SMILES: CC(C)NC(=O)[C@H]1CC[C@@H](n2/c(=N/C(=O)c3cc(F)cc(C#N)c3)[nH]c3cnc(OCCN4CCCCC4)cc32)CC1] |
| 112 | ++ | 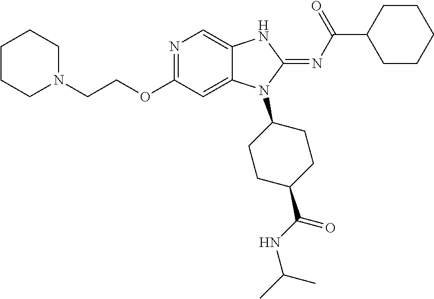 [SMILES: CC(C)NC(=O)[C@H]1CC[C@@H](n2/c(=N/C(=O)C3CCCCC3)[nH]c3cnc(OCCN4CCCCC4)cc32)CC1] |
| 113 | ++++ | 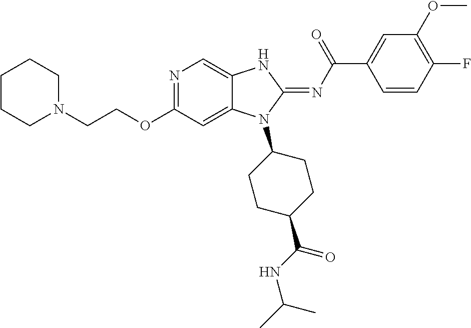 [SMILES: COc1cc(C(=O)/N=c2\[nH]c3cnc(OCCN4CCCCC4)cc3n2[C@H]2CC[C@@H](C(=O)NC(C)C)CC2)ccc1F] |
| 114 | +++++ | 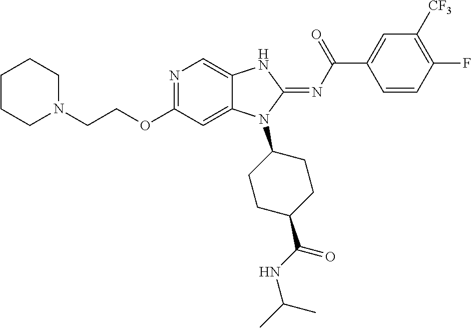 [SMILES: CC(C)NC(=O)[C@H]1CC[C@@H](n2/c(=N/C(=O)c3ccc(F)c(C(F)(F)F)c3)[nH]c3cnc(OCCN4CCCCC4)cc32)CC1] |
| 115 | ++++++ | 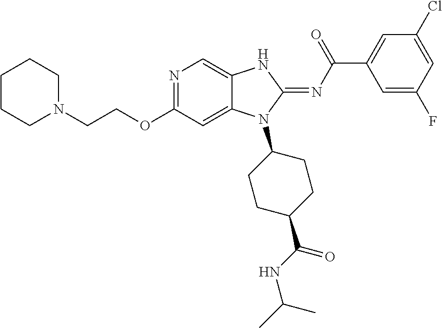 [SMILES: CC(C)NC(=O)[C@H]1CC[C@@H](n2/c(=N/C(=O)c3cc(F)cc(Cl)c3)[nH]c3cnc(OCCN4CCCCC4)cc32)CC1] |
| 116 | +++++ | 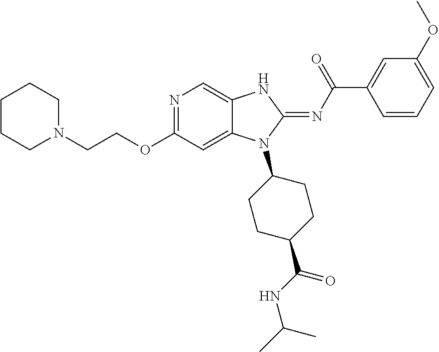 [SMILES: COc1cccc(C(=O)/N=c2\[nH]c3cnc(OCCN4CCCCC4)cc3n2[C@H]2CC[C@@H](C(=O)NC(C)C)CC2)c1] |
| 117 | +++++ | 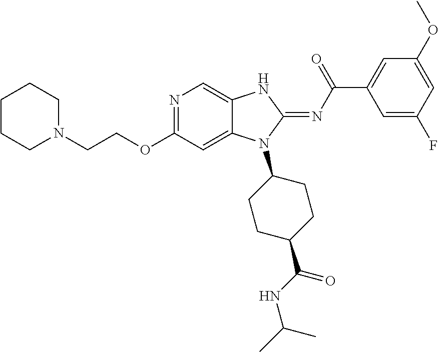 [SMILES: COc1cc(F)cc(C(=O)/N=c2\[nH]c3cnc(OCCN4CCCCC4)cc3n2[C@H]2CC[C@@H](C(=O)NC(C)C)CC2)c1] |
| 118 | ++++++ | 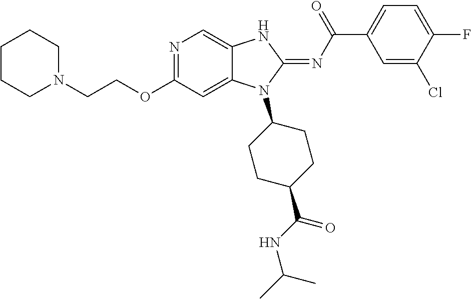 [SMILES: CC(C)NC(=O)[C@H]1CC[C@@H](n2/c(=N/C(=O)c3ccc(F)c(Cl)c3)[nH]c3cnc(OCCN4CCCCC4)cc32)CC1] |
| 119 | ++++++ | 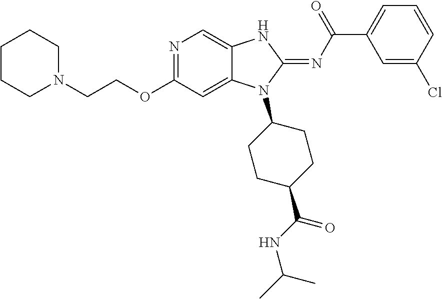 [SMILES: CC(C)NC(=O)[C@H]1CC[C@@H](n2/c(=N/C(=O)c3cccc(Cl)c3)[nH]c3cnc(OCCN4CCCCC4)cc32)CC1] |
| 120 | ++++ | 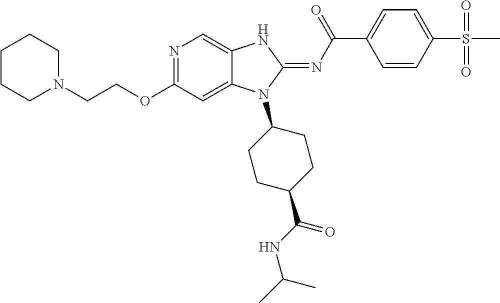 [SMILES: CC(C)NC(=O)[C@H]1CC[C@@H](n2/c(=N/C(=O)c3ccc(S(C)(=O)=O)cc3)[nH]c3cnc(OCCN4CCCCC4)cc32)CC1] |
| 121 | +++++ | 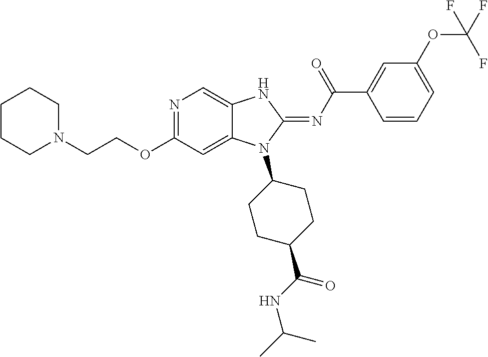 [SMILES: CC(C)NC(=O)[C@H]1CC[C@@H](n2/c(=N/C(=O)c3cccc(OC(F)(F)F)c3)[nH]c3cnc(OCCN4CCCCC4)cc32)CC1] |
| 122 | ++++ | 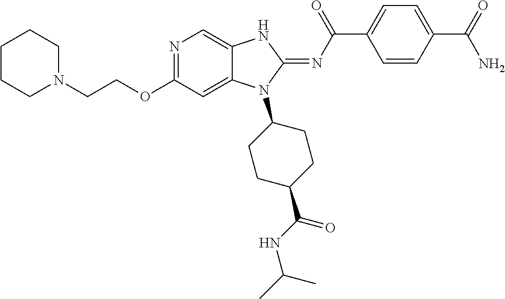 [SMILES: CC(C)NC(=O)[C@H]1CC[C@@H](n2/c(=N/C(=O)c3ccc(C(N)=O)cc3)[nH]c3cnc(OCCN4CCCCC4)cc32)CC1] |
| 123 | ++++++ | 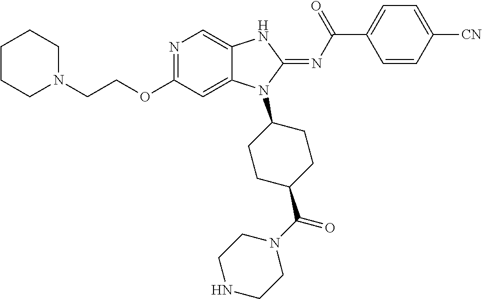 [SMILES: N#Cc1ccc(C(=O)/N=c2\[nH]c3cnc(OCCN4CCCCC4)cc3n2[C@H]2CC[C@@H](C(=O)N3CCNCC3)CC2)cc1] |
| 124 | ++++ | 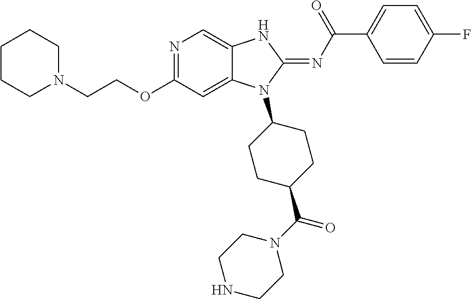 [SMILES: O=C(/N=c1\[nH]c2cnc(OCCN3CCCCC3)cc2n1[C@H]1CC[C@@H](C(=O)N2CCNCC2)CC1)c1ccc(F)cc1] |
| 125 | ++++++ | 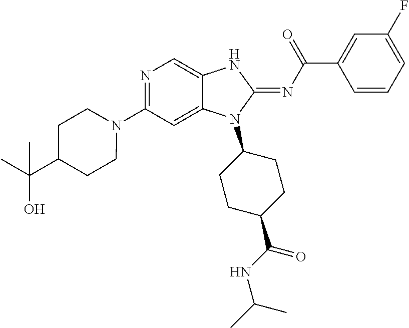 [SMILES: CC(C)NC(=O)[C@H]1CC[C@@H](n2/c(=N/C(=O)c3cccc(F)c3)[nH]c3cnc(N4CCC(C(C)(C)O)CC4)cc32)CC1] |
| 126 | ++++++ | 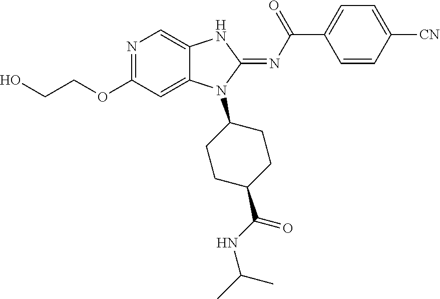 [SMILES: CC(C)NC(=O)[C@H]1CC[C@@H](n2/c(=N/C(=O)c3ccc(C#N)cc3)[nH]c3cnc(OCCO)cc32)CC1] |
| 127 | ++++++ | 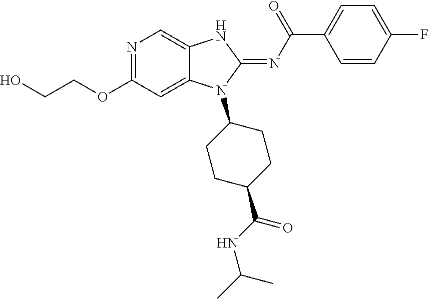 [SMILES: CC(C)NC(=O)[C@H]1CC[C@@H](n2/c(=N/C(=O)c3ccc(F)cc3)[nH]c3cnc(OCCO)cc32)CC1] |
| 128 | ++++++ | 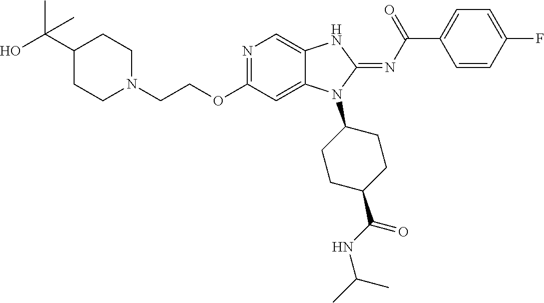 [SMILES: CC(C)NC(=O)[C@H]1CC[C@@H](n2/c(=N/C(=O)c3ccc(F)cc3)[nH]c3cnc(OCCN4CCC(C(C)(C)O)CC4)cc32)CC1] |
| 129 | ++++++ | 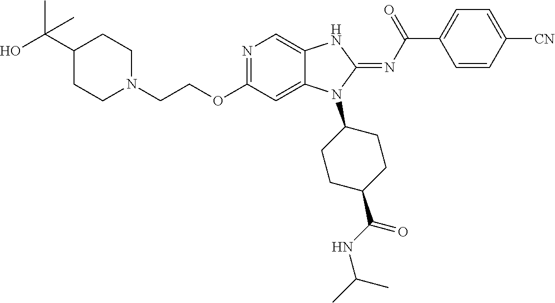 [SMILES: CC(C)NC(=O)[C@H]1CC[C@@H](n2/c(=N/C(=O)c3ccc(C#N)cc3)[nH]c3cnc(OCCN4CCC(C(C)(C)O)CC4)cc32)CC1] |
| 130 | ++++++ | 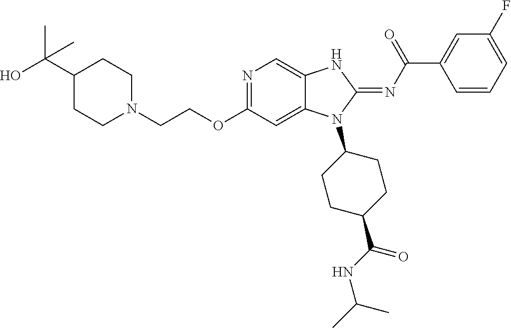 [SMILES: CC(C)NC(=O)[C@H]1CC[C@@H](n2/c(=N/C(=O)c3cccc(F)c3)[nH]c3cnc(OCCN4CCC(C(C)(C)O)CC4)cc32)CC1] |
| 131 | ++++ | 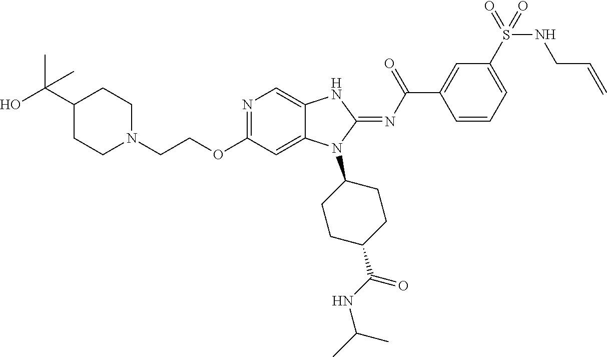 [SMILES: C=CCNS(=O)(=O)c1cccc(C(=O)/N=c2\[nH]c3cnc(OCCN4CCC(C(C)(C)O)CC4)cc3n2[C@H]2CC[C@H](C(=O)NC(C)C)CC2)c1] |
| 132 | ++++ | 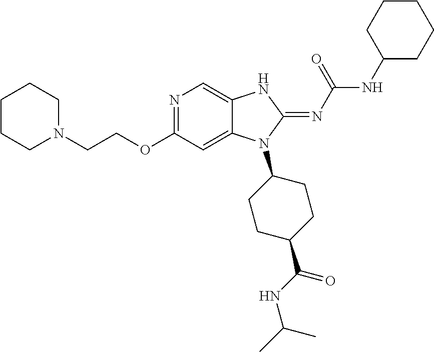 [SMILES: CC(C)NC(=O)[C@H]1CC[C@@H](n2/c(=N/C(=O)NC3CCCCC3)[nH]c3cnc(OCCN4CCCCC4)cc32)CC1] |
| 133 | ++++ | 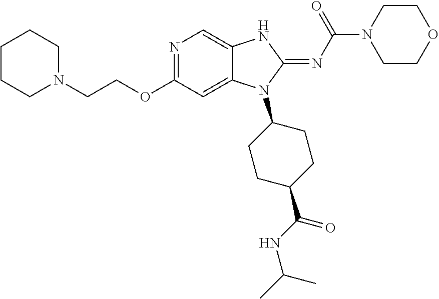 [SMILES: CC(C)NC(=O)[C@H]1CC[C@@H](n2/c(=N/C(=O)N3CCOCC3)[nH]c3cnc(OCCN4CCCCC4)cc32)CC1] |
| 134 | +++++ | 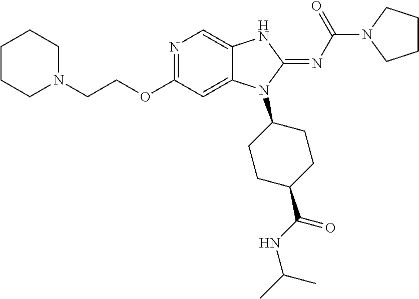 [SMILES: CC(C)NC(=O)[C@H]1CC[C@@H](n2/c(=N/C(=O)N3CCCC3)[nH]c3cnc(OCCN4CCCCC4)cc32)CC1] |
| 135 | ++++++ | 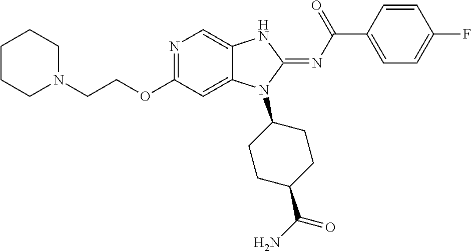 [SMILES: NC(=O)[C@H]1CC[C@@H](n2/c(=N/C(=O)c3ccc(F)cc3)[nH]c3cnc(OCCN4CCCCC4)cc32)CC1] |
| 136 | +++ | 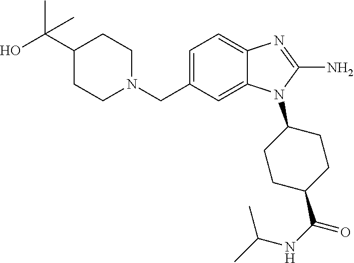 [SMILES: CC(C)NC(=O)[C@H]1CC[C@@H](n2c(N)nc3ccc(CN4CCC(C(C)(C)O)CC4)cc32)CC1] |
| 137 | ++++++ | 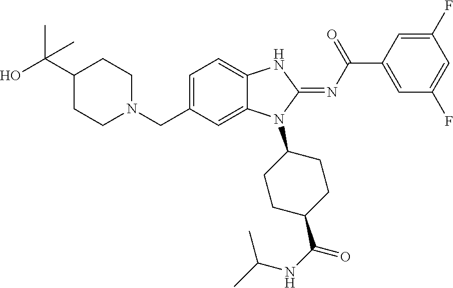 [SMILES: CC(C)NC(=O)[C@H]1CC[C@@H](n2/c(=N/C(=O)c3cc(F)cc(F)c3)[nH]c3ccc(CN4CCC(C(C)(C)O)CC4)cc32)CC1] |
| 138 | ++++++ | 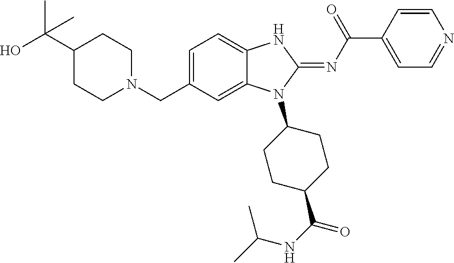 [SMILES: CC(C)NC(=O)[C@H]1CC[C@@H](n2/c(=N/C(=O)c3ccncc3)[nH]c3ccc(CN4CCC(C(C)(C)O)CC4)cc32)CC1] |
| 139 | ++++++ | 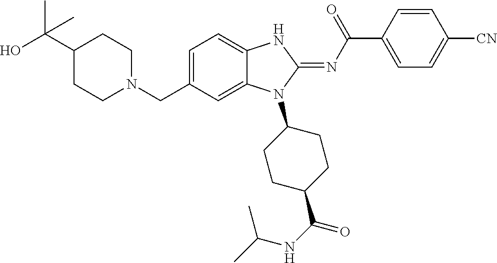 [SMILES: CC(C)NC(=O)[C@H]1CC[C@@H](n2/c(=N/C(=O)c3ccc(C#N)cc3)[nH]c3ccc(CN4CCC(C(C)(C)O)CC4)cc32)CC1] |
| 140 | ++++++ | 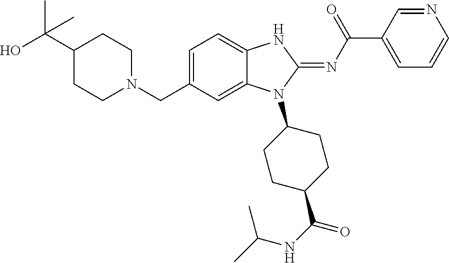 [SMILES: CC(C)NC(=O)[C@H]1CC[C@@H](n2/c(=N/C(=O)c3cccnc3)[nH]c3ccc(CN4CCC(C(C)(C)O)CC4)cc32)CC1] |
| 141 | ++++++ | 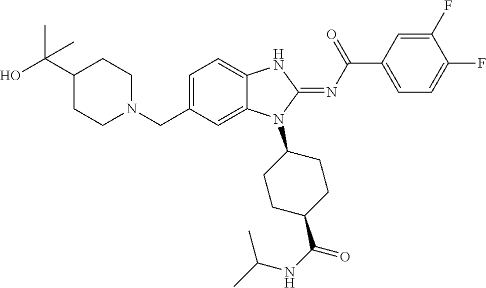 [SMILES: CC(C)NC(=O)[C@H]1CC[C@@H](n2/c(=N/C(=O)c3ccc(F)c(F)c3)[nH]c3ccc(CN4CCC(C(C)(C)O)CC4)cc32)CC1] |
| 142 | ++++++ | 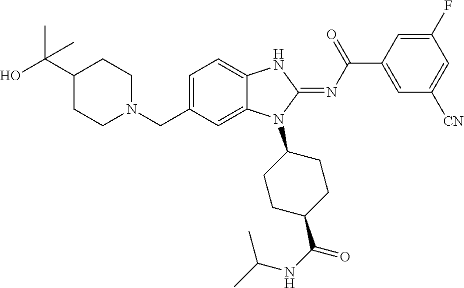 [SMILES: CC(C)NC(=O)[C@H]1CC[C@@H](n2/c(=N/C(=O)c3cc(F)cc(C#N)c3)[nH]c3ccc(CN4CCC(C(C)(C)O)CC4)cc32)CC1] |
| 143 | ++++++ | 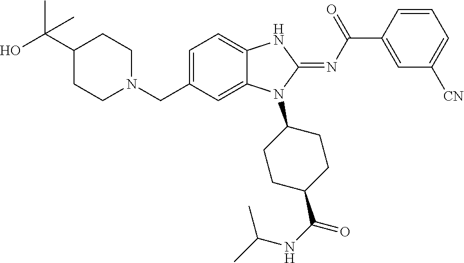 [SMILES: CC(C)NC(=O)[C@H]1CC[C@@H](n2/c(=N/C(=O)c3cccc(C#N)c3)[nH]c3ccc(CN4CCC(C(C)(C)O)CC4)cc32)CC1] |
| 144 | ++++++ | 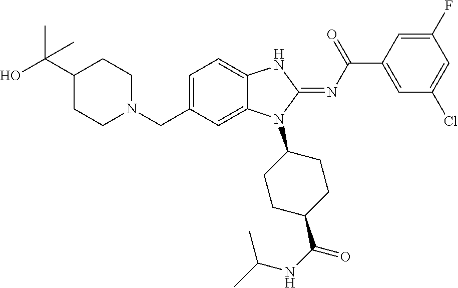 [SMILES: CC(C)NC(=O)[C@H]1CC[C@@H](n2/c(=N/C(=O)c3cc(F)cc(Cl)c3)[nH]c3ccc(CN4CCC(C(C)(C)O)CC4)cc32)CC1] |
| 145 | ++++++ | 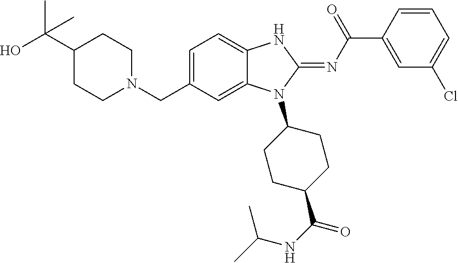 [SMILES: CC(C)NC(=O)[C@H]1CC[C@@H](n2/c(=N/C(=O)c3cccc(Cl)c3)[nH]c3ccc(CN4CCC(C(C)(C)O)CC4)cc32)CC1] |
| 146 | ++++++ | 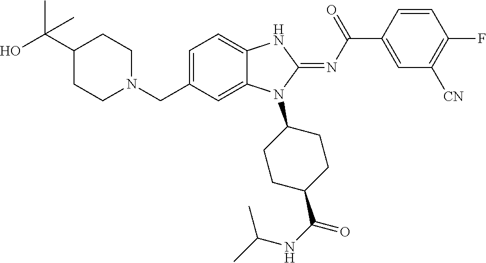 [SMILES: CC(C)NC(=O)[C@H]1CC[C@@H](n2/c(=N/C(=O)c3ccc(F)c(C#N)c3)[nH]c3ccc(CN4CCC(C(C)(C)O)CC4)cc32)CC1] |
| 147 | ++++++ | 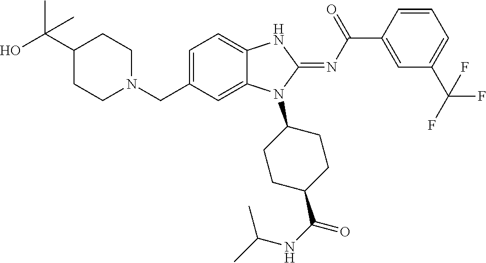 [SMILES: CC(C)NC(=O)[C@H]1CC[C@@H](n2/c(=N/C(=O)c3cccc(C(F)(F)F)c3)[nH]c3ccc(CN4CCC(C(C)(C)O)CC4)cc32)CC1] |
| 148 | ++++++ | 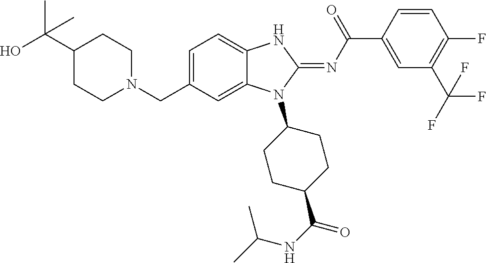 [SMILES: CC(C)NC(=O)[C@H]1CC[C@@H](n2/c(=N/C(=O)c3ccc(F)c(C(F)(F)F)c3)[nH]c3ccc(CN4CCC(C(C)(C)O)CC4)cc32)CC1] |
| 149 | ++++++ | 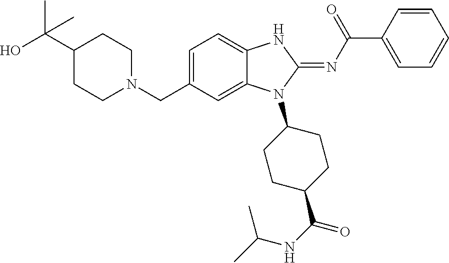 [SMILES: CC(C)NC(=O)[C@H]1CC[C@@H](n2/c(=N/C(=O)c3ccccc3)[nH]c3ccc(CN4CCC(C(C)(C)O)CC4)cc32)CC1] |
| 150 | ++++++ | 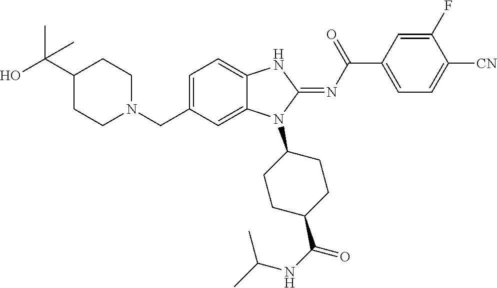 [SMILES: CC(C)NC(=O)[C@H]1CC[C@@H](n2/c(=N/C(=O)c3ccc(C#N)c(F)c3)[nH]c3ccc(CN4CCC(C(C)(C)O)CC4)cc32)CC1] |
| 151 | ++++++ | 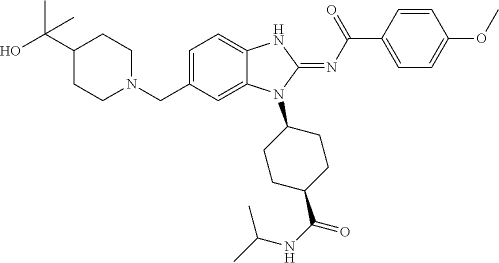 [SMILES: COc1ccc(C(=O)/N=c2\[nH]c3ccc(CN4CCC(C(C)(C)O)CC4)cc3n2[C@H]2CC[C@@H](C(=O)NC(C)C)CC2)cc1] |
| 152 | ++++++ | 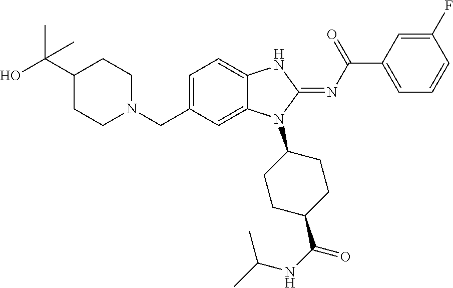 [SMILES: CC(C)NC(=O)[C@H]1CC[C@@H](n2/c(=N/C(=O)c3cccc(F)c3)[nH]c3ccc(CN4CCC(C(C)(C)O)CC4)cc32)CC1] |
| 153 | ++++++ | 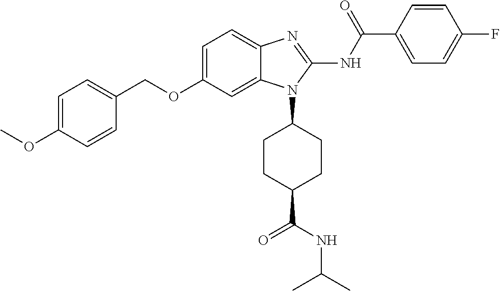 [SMILES: COc1ccc(COc2ccc3nc(NC(=O)c4ccc(F)cc4)n([C@H]4CC[C@@H](C(=O)NC(C)C)CC4)c3c2)cc1] |
| 154 | ++++++ | 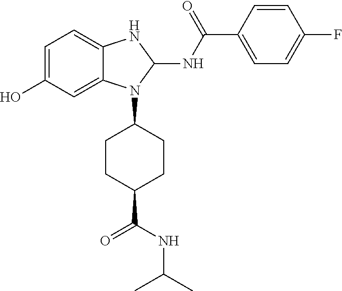 [SMILES: CC(C)NC(=O)[C@H]1CC[C@@H](N2c3cc(O)ccc3NC2NC(=O)c2ccc(F)cc2)CC1] |
| 155 | ++++++ | 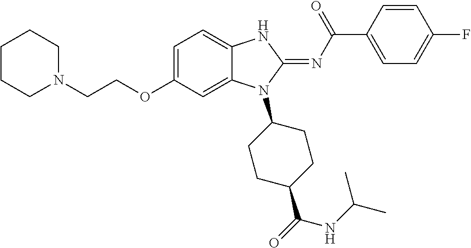 [SMILES: CC(C)NC(=O)[C@H]1CC[C@@H](n2/c(=N/C(=O)c3ccc(F)cc3)[nH]c3ccc(OCCN4CCCCC4)cc32)CC1] |
| 156 | ++++++ | 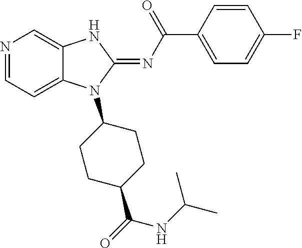 [SMILES: CC(C)NC(=O)[C@H]1CC[C@@H](n2/c(=N/C(=O)c3ccc(F)cc3)[nH]c3cnccc32)CC1] |
| 157 | ++++++ | 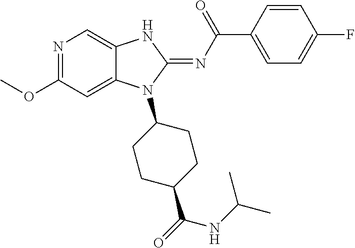 [SMILES: COc1cc2c(cn1)[nH]/c(=N\C(=O)c1ccc(F)cc1)n2[C@H]1CC[C@@H](C(=O)NC(C)C)CC1] |
| 158 | ++++++ | 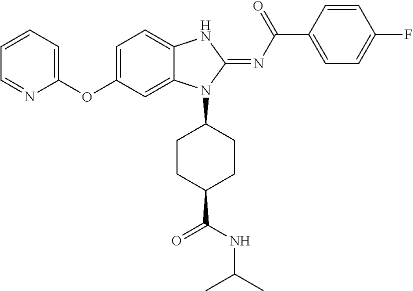 [SMILES: CC(C)NC(=O)[C@H]1CC[C@@H](n2/c(=N/C(=O)c3ccc(F)cc3)[nH]c3ccc(Oc4ccccn4)cc32)CC1] |
| 159 | ++++++ | 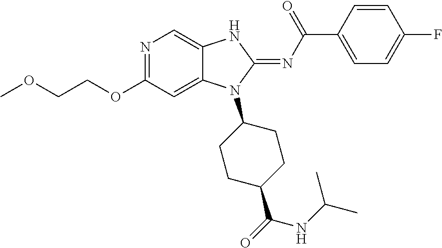 [SMILES: COCCOc1cc2c(cn1)[nH]/c(=N\C(=O)c1ccc(F)cc1)n2[C@H]1CC[C@@H](C(=O)NC(C)C)CC1] |
| 160 | ++++++ | 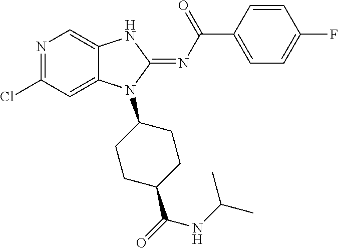 [SMILES: CC(C)NC(=O)[C@H]1CC[C@@H](n2/c(=N/C(=O)c3ccc(F)cc3)[nH]c3cnc(Cl)cc32)CC1] |
| 161 | ++++++ | 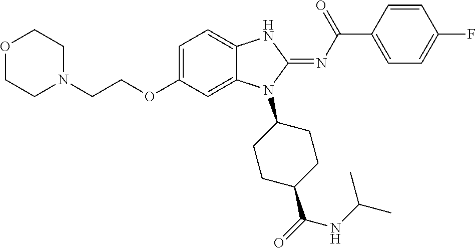 [SMILES: CC(C)NC(=O)[C@H]1CC[C@@H](n2/c(=N/C(=O)c3ccc(F)cc3)[nH]c3ccc(OCCN4CCOCC4)cc32)CC1] |
| 162 | ++++++ | 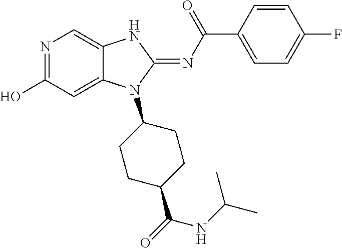 [SMILES: CC(C)NC(=O)[C@H]1CC[C@@H](n2/c(=N/C(=O)c3ccc(F)cc3)[nH]c3cnc(O)cc32)CC1] |
| 163 | ++++++ | 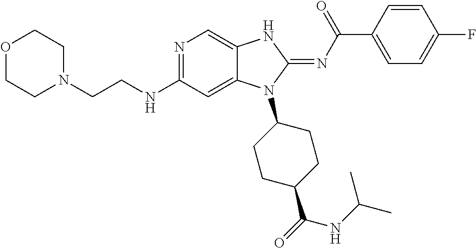 [SMILES: CC(C)NC(=O)[C@H]1CC[C@@H](n2/c(=N/C(=O)c3ccc(F)cc3)[nH]c3cnc(NCCN4CCOCC4)cc32)CC1] |
| 164 | ++++++ | 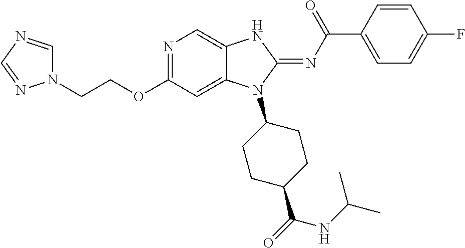 [SMILES: CC(C)NC(=O)[C@H]1CC[C@@H](n2/c(=N/C(=O)c3ccc(F)cc3)[nH]c3cnc(OCCn4cncn4)cc32)CC1] |
| 165 | ++++++ | 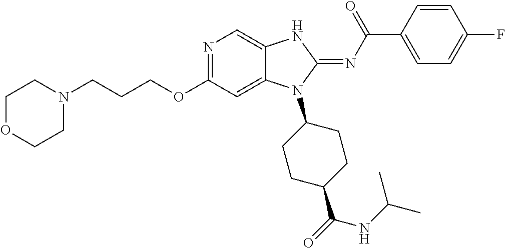 [SMILES: CC(C)NC(=O)[C@H]1CC[C@@H](n2/c(=N/C(=O)c3ccc(F)cc3)[nH]c3cnc(OCCCN4CCOCC4)cc32)CC1] |
| 166 | ++++++ | 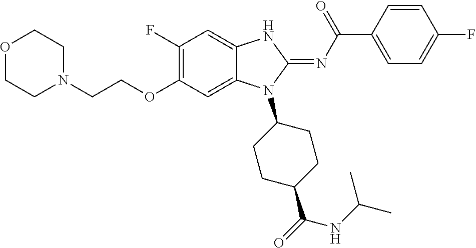 [SMILES: CC(C)NC(=O)[C@H]1CC[C@@H](n2/c(=N/C(=O)c3ccc(F)cc3)[nH]c3cc(F)c(OCCN4CCOCC4)cc32)CC1] |
| 167 | ++++++ | 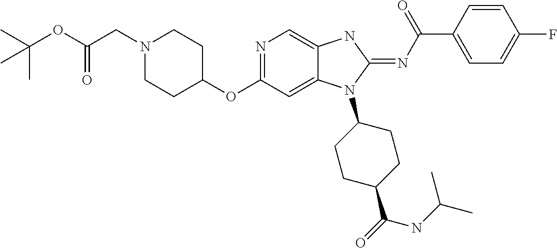 [SMILES: CC(C)NC(=O)[C@H]1CC[C@@H](n2/c(=N/C(=O)c3ccc(F)cc3)[nH]c3cnc(OC4CCN(CC(=O)OC(C)(C)C)CC4)cc32)CC1] |
| 168 | ++++++ | 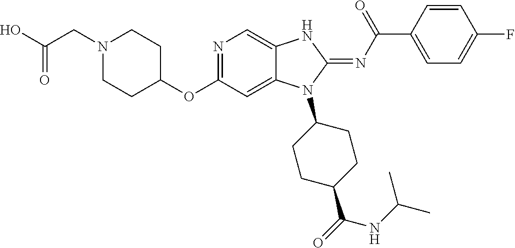 [SMILES: CC(C)NC(=O)[C@H]1CC[C@@H](n2/c(=N/C(=O)c3ccc(F)cc3)[nH]c3cnc(OC4CCN(CC(=O)O)CC4)cc32)CC1] |
| 169 | ++++++ | 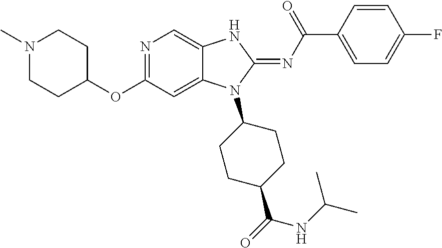 [SMILES: CC(C)NC(=O)[C@H]1CC[C@@H](n2/c(=N/C(=O)c3ccc(F)cc3)[nH]c3cnc(OC4CCN(C)CC4)cc32)CC1] |
| 170 | ++++++ | 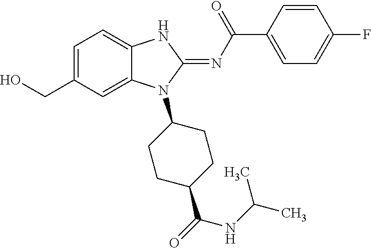 [SMILES: CC(C)NC(=O)[C@H]1CC[C@@H](n2/c(=N/C(=O)c3ccc(F)cc3)[nH]c3ccc(CO)cc32)CC1] |
| 171 | ++++++ | 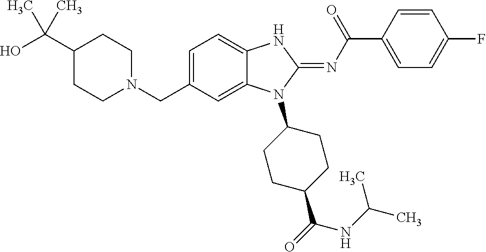 [SMILES: CC(C)NC(=O)[C@H]1CC[C@@H](n2/c(=N/C(=O)c3ccc(F)cc3)[nH]c3ccc(CN4CCC(C(C)(C)O)CC4)cc32)CC1] |
| 172 | ++++++ | 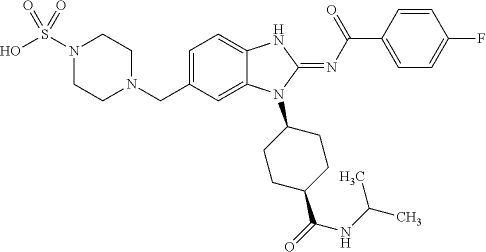 [SMILES: CC(C)NC(=O)[C@H]1CC[C@@H](n2/c(=N/C(=O)c3ccc(F)cc3)[nH]c3ccc(CN4CCN(S(=O)(=O)O)CC4)cc32)CC1] |
| 173 | ++++++ | 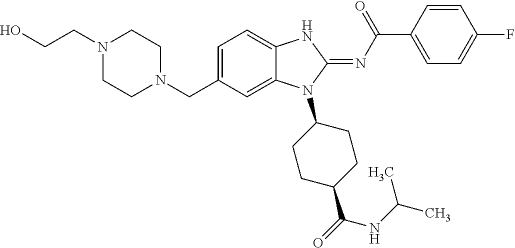 [SMILES: CC(C)NC(=O)[C@H]1CC[C@@H](n2/c(=N/C(=O)c3ccc(F)cc3)[nH]c3ccc(CN4CCN(CCO)CC4)cc32)CC1] |
| 174 | ++++++ | 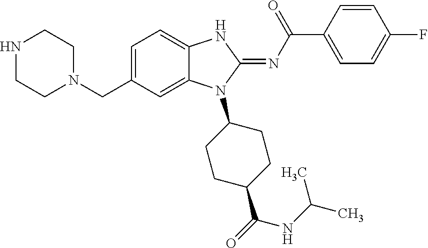 [SMILES: CC(C)NC(=O)[C@H]1CC[C@@H](n2/c(=N/C(=O)c3ccc(F)cc3)[nH]c3ccc(CN4CCNCC4)cc32)CC1] |
| 175 | ++++++ | 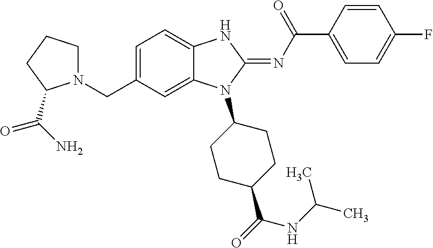 [SMILES: CC(C)NC(=O)[C@H]1CC[C@@H](n2/c(=N/C(=O)c3ccc(F)cc3)[nH]c3ccc(CN4CCC[C@H]4C(N)=O)cc32)CC1] |
| 176 | ++++++ | 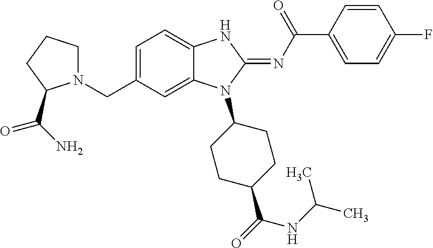 [SMILES: CC(C)NC(=O)[C@H]1CC[C@@H](n2/c(=N/C(=O)c3ccc(F)cc3)[nH]c3ccc(CN4CCC[C@@H]4C(N)=O)cc32)CC1] |
| 177 | ++++++ | 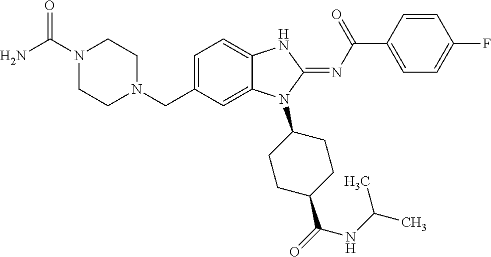 [SMILES: CC(C)NC(=O)[C@H]1CC[C@@H](n2/c(=N/C(=O)c3ccc(F)cc3)[nH]c3ccc(CN4CCN(C(N)=O)CC4)cc32)CC1] |
| 178 | ++++++ | 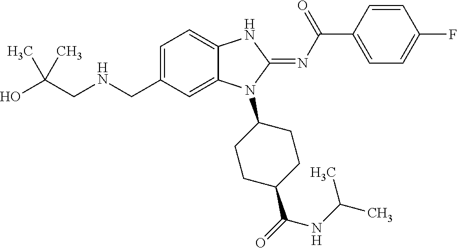 [SMILES: CC(C)NC(=O)[C@H]1CC[C@@H](n2/c(=N/C(=O)c3ccc(F)cc3)[nH]c3ccc(CNCC(C)(C)O)cc32)CC1] |
| 179 | ++++++ | 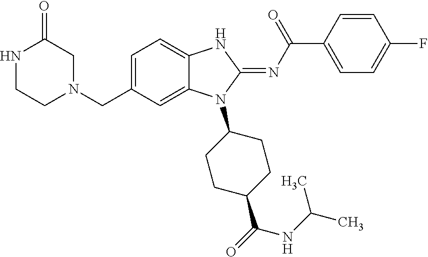 [SMILES: CC(C)NC(=O)[C@H]1CC[C@@H](n2/c(=N/C(=O)c3ccc(F)cc3)[nH]c3ccc(CN4CCNC(=O)C4)cc32)CC1] |
| 180 | ++++++ | 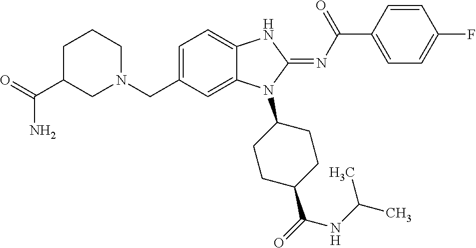 [SMILES: CC(C)NC(=O)[C@H]1CC[C@@H](n2/c(=N/C(=O)c3ccc(F)cc3)[nH]c3ccc(CN4CCCC(C(N)=O)C4)cc32)CC1] |
| 181 | ++++++ | 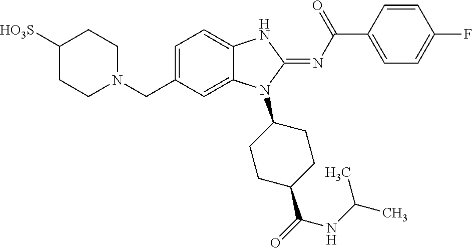 [SMILES: CC(C)NC(=O)[C@H]1CC[C@@H](n2/c(=N/C(=O)c3ccc(F)cc3)[nH]c3ccc(CN4CCC(S(=O)(=O)O)CC4)cc32)CC1] |
| 182 | ++++++ | 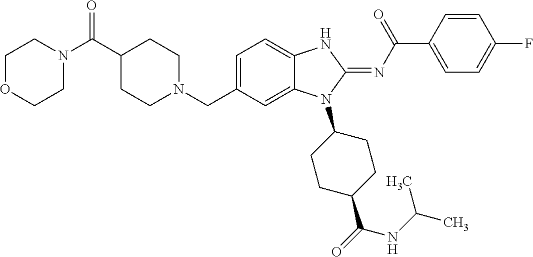 [SMILES: CC(C)NC(=O)[C@H]1CC[C@@H](n2/c(=N/C(=O)c3ccc(F)cc3)[nH]c3ccc(CN4CCC(C(=O)N5CCOCC5)CC4)cc32)CC1] |
| 183 | ++++++ | 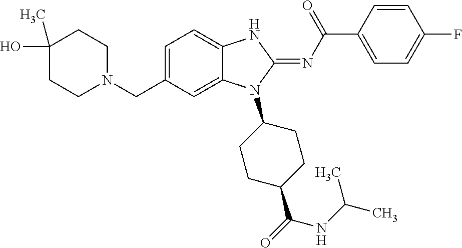 [SMILES: CC(C)NC(=O)[C@H]1CC[C@@H](n2/c(=N/C(=O)c3ccc(F)cc3)[nH]c3ccc(CN4CCC(C)(O)CC4)cc32)CC1] |
| 184 | ++++++ | 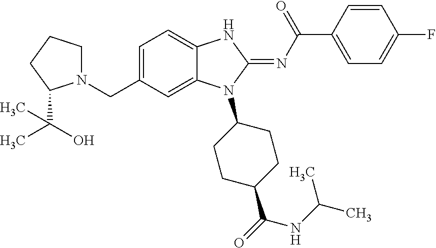 [SMILES: CC(C)NC(=O)[C@H]1CC[C@@H](n2/c(=N/C(=O)c3ccc(F)cc3)[nH]c3ccc(CN4CCC[C@H]4C(C)(C)O)cc32)CC1] |
| 185 | ++++++ | 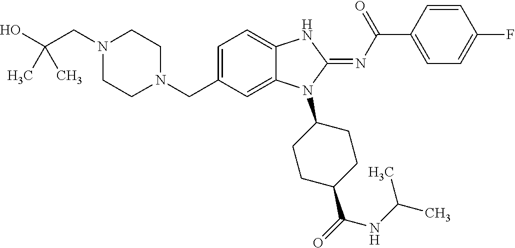 [SMILES: CC(C)NC(=O)[C@H]1CC[C@@H](n2/c(=N/C(=O)c3ccc(F)cc3)[nH]c3ccc(CN4CCN(CC(C)(C)O)CC4)cc32)CC1] |
| 186 | ++++++ | 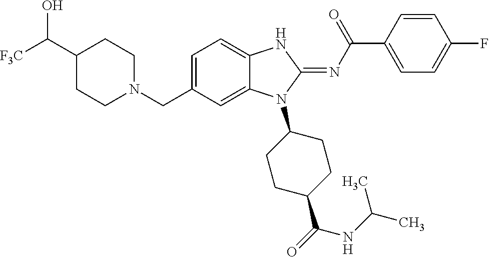 [SMILES: CC(C)NC(=O)[C@H]1CC[C@@H](n2/c(=N/C(=O)c3ccc(F)cc3)[nH]c3ccc(CN4CCC(C(O)C(F)(F)F)CC4)cc32)CC1] |
| 187 | ++++++ | 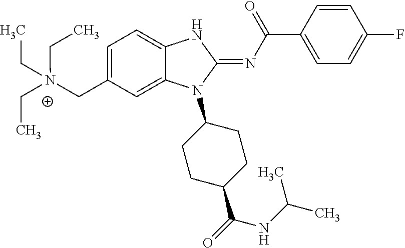 [SMILES: CC[N+](CC)(CC)Cc1ccc2[nH]/c(=N\C(=O)c3ccc(F)cc3)n([C@H]3CC[C@@H](C(=O)NC(C)C)CC3)c2c1] |
| 188 | ++++++ | 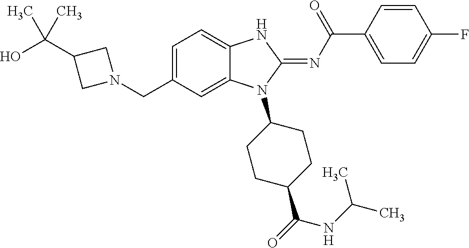 [SMILES: CC(C)NC(=O)[C@H]1CC[C@@H](n2/c(=N/C(=O)c3ccc(F)cc3)[nH]c3ccc(CN4CC(C(C)(C)O)C4)cc32)CC1] |
| 189 | ++++++ | 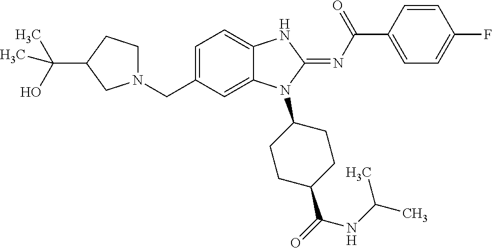 [SMILES: CC(C)NC(=O)[C@H]1CC[C@@H](n2/c(=N/C(=O)c3ccc(F)cc3)[nH]c3ccc(CN4CCC(C(C)(C)O)C4)cc32)CC1] |
| 190 | ++++++ | 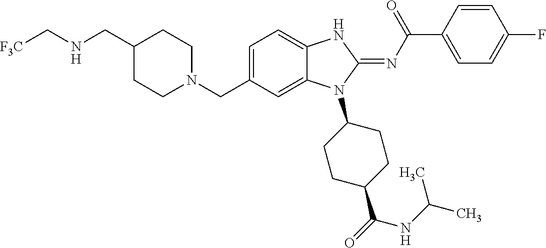 [SMILES: CC(C)NC(=O)[C@H]1CC[C@@H](n2/c(=N/C(=O)c3ccc(F)cc3)[nH]c3ccc(CN4CCC(CNCC(F)(F)F)CC4)cc32)CC1] |
| 191 | ++++++ | 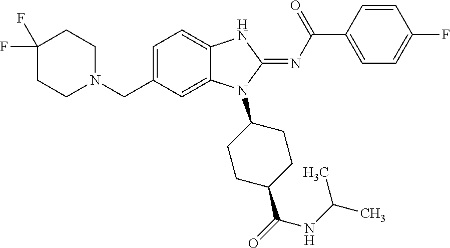 [SMILES: CC(C)NC(=O)[C@H]1CC[C@@H](n2/c(=N/C(=O)c3ccc(F)cc3)[nH]c3ccc(CN4CCC(F)(F)CC4)cc32)CC1] |
| 192 | ++++++ | 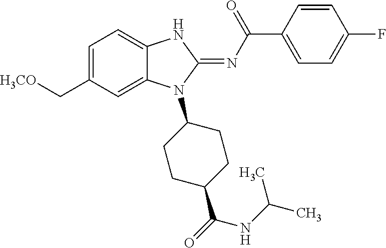 [SMILES: COCc1ccc2[nH]/c(=N\C(=O)c3ccc(F)cc3)n([C@H]3CC[C@@H](C(=O)NC(C)C)CC3)c2c1] |
| 193 | ++++++ | 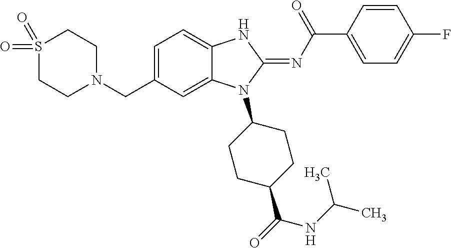 [SMILES: CC(C)NC(=O)[C@H]1CC[C@@H](n2/c(=N/C(=O)c3ccc(F)cc3)[nH]c3ccc(CN4CCS(=O)(=O)CC4)cc32)CC1] |
| 194 | ++++++ | 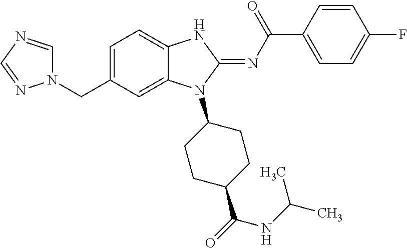 [SMILES: CC(C)NC(=O)[C@H]1CC[C@@H](n2/c(=N/C(=O)c3ccc(F)cc3)[nH]c3ccc(Cn4cncn4)cc32)CC1] |
| 195 | ++++++ | 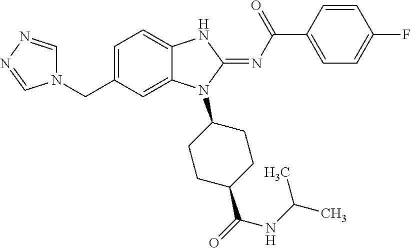 [SMILES: CC(C)NC(=O)[C@H]1CC[C@@H](n2/c(=N/C(=O)c3ccc(F)cc3)[nH]c3ccc(Cn4cnnc4)cc32)CC1] |
| 196 | ++++++ | 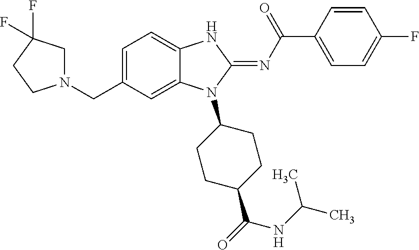 [SMILES: CC(C)NC(=O)[C@H]1CC[C@@H](n2/c(=N/C(=O)c3ccc(F)cc3)[nH]c3ccc(CN4CCC(F)(F)C4)cc32)CC1] |
| 197 | ++++++ | 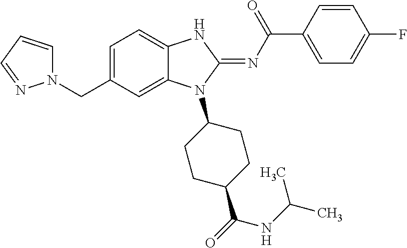 [SMILES: CC(C)NC(=O)[C@H]1CC[C@@H](n2/c(=N/C(=O)c3ccc(F)cc3)[nH]c3ccc(Cn4cccn4)cc32)CC1] |
| 198 | ++++++ | 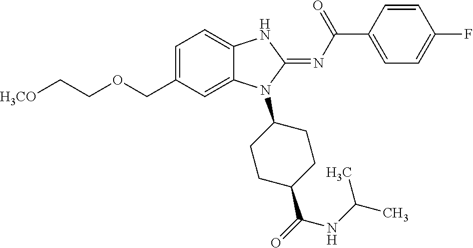 [SMILES: COCCOCc1ccc2[nH]/c(=N\C(=O)c3ccc(F)cc3)n([C@H]3CC[C@@H](C(=O)NC(C)C)CC3)c2c1] |
| 199 | ++++++ | 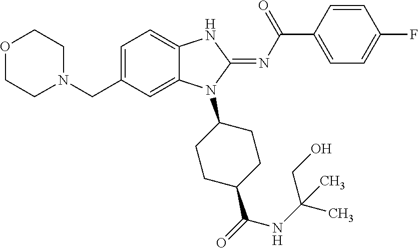 [SMILES: CC(C)(CO)NC(=O)[C@H]1CC[C@@H](n2/c(=N/C(=O)c3ccc(F)cc3)[nH]c3ccc(CN4CCOCC4)cc32)CC1] |
| 200 | ++++++ | 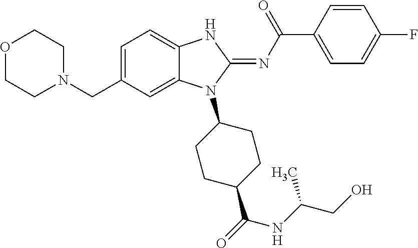 [SMILES: C[C@H](CO)NC(=O)[C@H]1CC[C@@H](n2/c(=N/C(=O)c3ccc(F)cc3)[nH]c3ccc(CN4CCOCC4)cc32)CC1] |
| 201 | ++++++ | 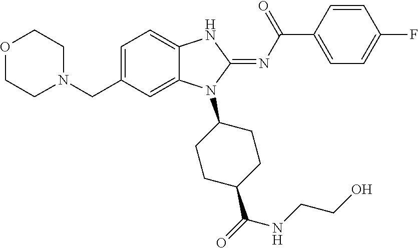 [SMILES: O=C(/N=c1\[nH]c2ccc(CN3CCOCC3)cc2n1[C@H]1CC[C@@H](C(=O)NCCO)CC1)c1ccc(F)cc1] |
| 202 | ++++ | 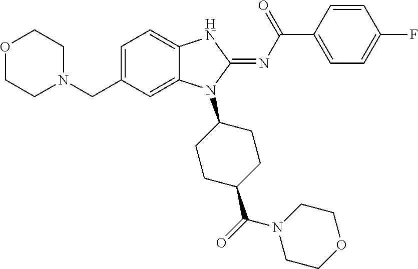 [SMILES: O=C(/N=c1\[nH]c2ccc(CN3CCOCC3)cc2n1[C@H]1CC[C@@H](C(=O)N2CCOCC2)CC1)c1ccc(F)cc1] |
| 203 | ++++ | 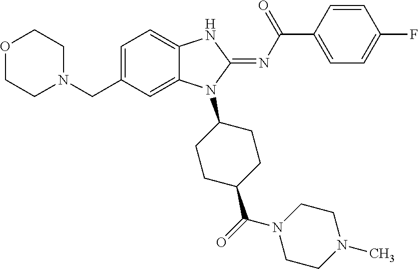 [SMILES: CN1CCN(C(=O)[C@H]2CC[C@@H](n3/c(=N/C(=O)c4ccc(F)cc4)[nH]c4ccc(CN5CCOCC5)cc43)CC2)CC1] |
| 204 | ++++++ | 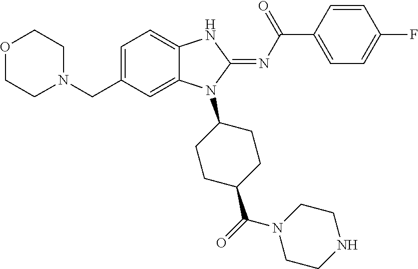 [SMILES: O=C(/N=c1\[nH]c2ccc(CN3CCOCC3)cc2n1[C@H]1CC[C@@H](C(=O)N2CCNCC2)CC1)c1ccc(F)cc1] |
| 205 | ++++++ | 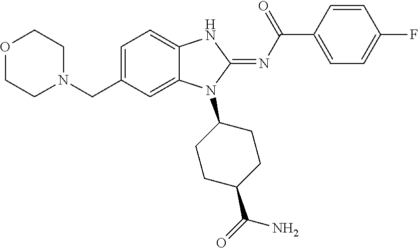 [SMILES: NC(=O)[C@H]1CC[C@@H](n2/c(=N/C(=O)c3ccc(F)cc3)[nH]c3ccc(CN4CCOCC4)cc32)CC1] |
| 206 | +++++ | 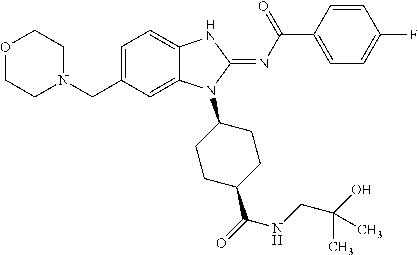 [SMILES: CC(C)(O)CNC(=O)[C@H]1CC[C@@H](n2/c(=N/C(=O)c3ccc(F)cc3)[nH]c3ccc(CN4CCOCC4)cc32)CC1] |
| 207 | +++++ | 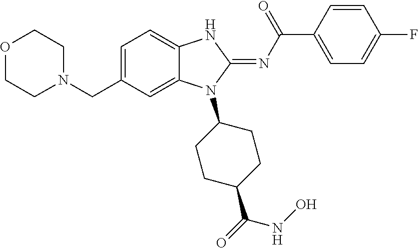 [SMILES: O=C(/N=c1\[nH]c2ccc(CN3CCOCC3)cc2n1[C@H]1CC[C@@H](C(=O)NO)CC1)c1ccc(F)cc1] |
| 208 | ++++++ | 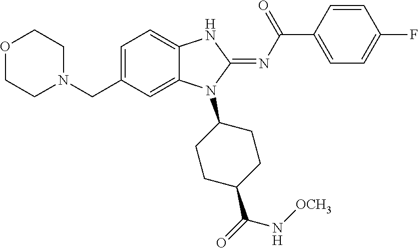 [SMILES: CONC(=O)[C@H]1CC[C@@H](n2/c(=N/C(=O)c3ccc(F)cc3)[nH]c3ccc(CN4CCOCC4)cc32)CC1] |
| 209 | ++++++ | 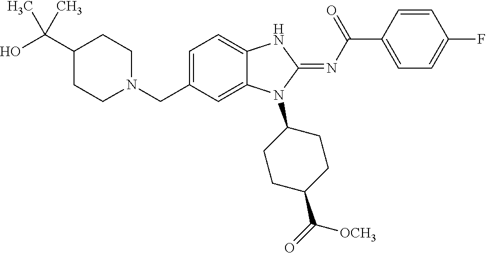 [SMILES: COC(=O)[C@H]1CC[C@@H](n2/c(=N/C(=O)c3ccc(F)cc3)[nH]c3ccc(CN4CCC(C(C)(C)O)CC4)cc32)CC1] |
| 210 | ++++ | 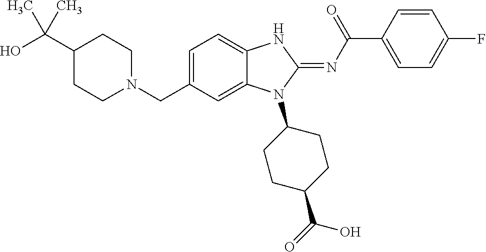 [SMILES: CC(C)(O)C1CCN(Cc2ccc3[nH]/c(=N\C(=O)c4ccc(F)cc4)n([C@H]4CC[C@@H](C(=O)O)CC4)c3c2)CC1] |
| 211 | ++++++ | 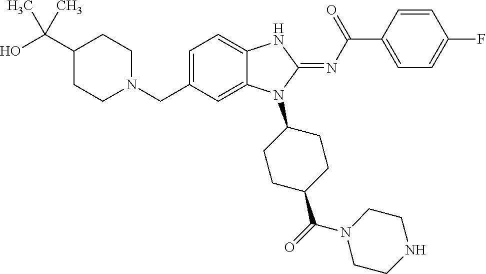 [SMILES: CC(C)(O)C1CCN(Cc2ccc3[nH]/c(=N\C(=O)c4ccc(F)cc4)n([C@H]4CC[C@@H](C(=O)N5CCNCC5)CC4)c3c2)CC1] |
| 212 | ++++++ | 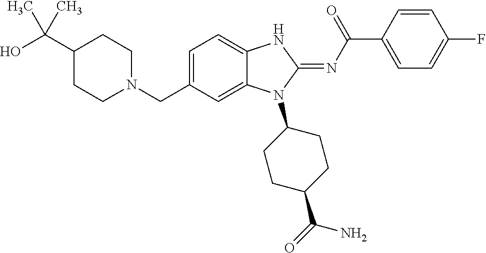 [SMILES: CC(C)(O)C1CCN(Cc2ccc3[nH]/c(=N\C(=O)c4ccc(F)cc4)n([C@H]4CC[C@@H](C(N)=O)CC4)c3c2)CC1] |
| 213 | ++++++ | 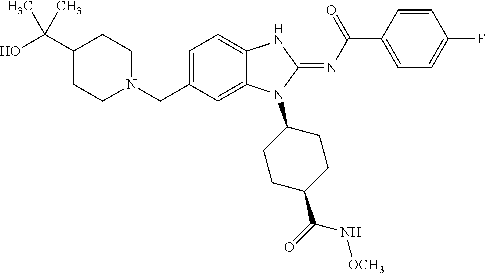 [SMILES: CONC(=O)[C@H]1CC[C@@H](n2/c(=N/C(=O)c3ccc(F)cc3)[nH]c3ccc(CN4CCC(C(C)(C)O)CC4)cc32)CC1] |
| 214 | ++++++ | 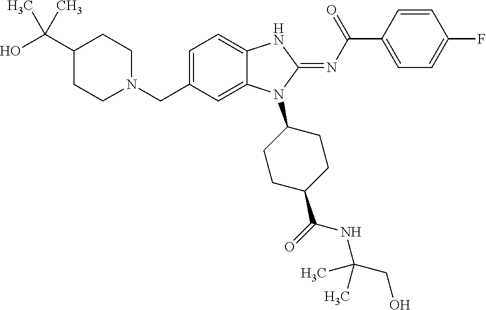 [SMILES: CC(C)(CO)NC(=O)[C@H]1CC[C@@H](n2/c(=N/C(=O)c3ccc(F)cc3)[nH]c3ccc(CN4CCC(C(C)(C)O)CC4)cc32)CC1] |
| 215 | ++++++ | 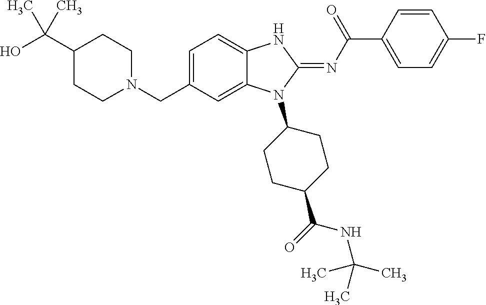 [SMILES: CC(C)(C)NC(=O)[C@H]1CC[C@@H](n2/c(=N/C(=O)c3ccc(F)cc3)[nH]c3ccc(CN4CCC(C(C)(C)O)CC4)cc32)CC1] |
| 216 | ++++++ | 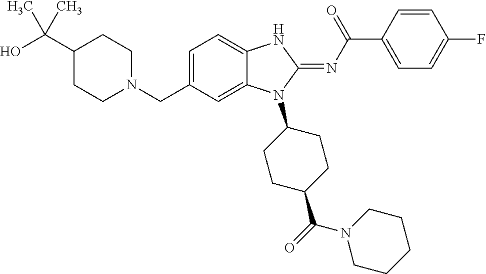 [SMILES: CC(C)(O)C1CCN(Cc2ccc3[nH]/c(=N\C(=O)c4ccc(F)cc4)n([C@H]4CC[C@@H](C(=O)N5CCCCC5)CC4)c3c2)CC1] |
| 217 | ++++++ | 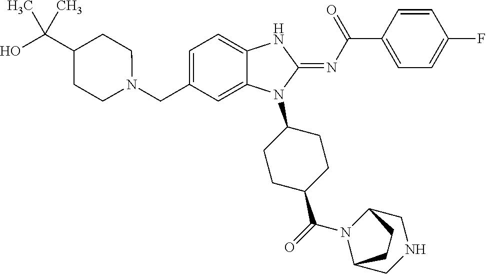 [SMILES: CC(C)(O)C1CCN(Cc2ccc3[nH]/c(=N\C(=O)c4ccc(F)cc4)n([C@H]4CC[C@@H](C(=O)N5[C@@H]6CC[C@H]5CNC6)CC4)c3c2)CC1] |
| 218 | ++++++ | 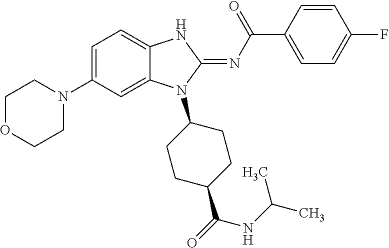 [SMILES: CC(C)NC(=O)[C@H]1CC[C@@H](n2/c(=N/C(=O)c3ccc(F)cc3)[nH]c3ccc(N4CCOCC4)cc32)CC1] |
| 219 | ++++++ | 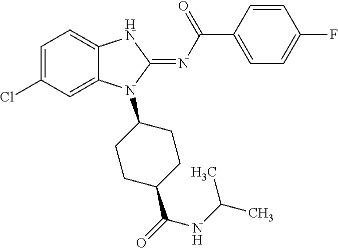 [SMILES: CC(C)NC(=O)[C@H]1CC[C@@H](n2/c(=N/C(=O)c3ccc(F)cc3)[nH]c3ccc(Cl)cc32)CC1] |
| 220 | ++++++ | 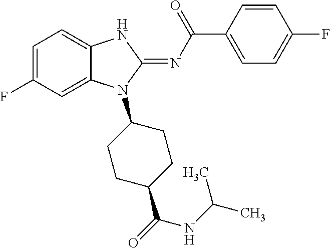 [SMILES: CC(C)NC(=O)[C@H]1CC[C@@H](n2/c(=N/C(=O)c3ccc(F)cc3)[nH]c3ccc(F)cc32)CC1] |
| 221 | ++++++ | 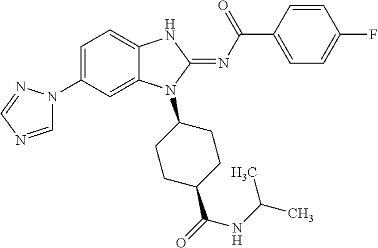 [SMILES: CC(C)NC(=O)[C@H]1CC[C@@H](n2/c(=N/C(=O)c3ccc(F)cc3)[nH]c3ccc(-n4cncn4)cc32)CC1] |
| 222 | ++++++ | 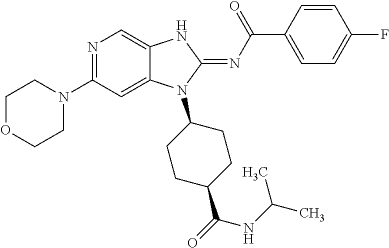 [SMILES: CC(C)NC(=O)[C@H]1CC[C@@H](n2/c(=N/C(=O)c3ccc(F)cc3)[nH]c3cnc(N4CCOCC4)cc32)CC1] |
| 223 | ++++++ | 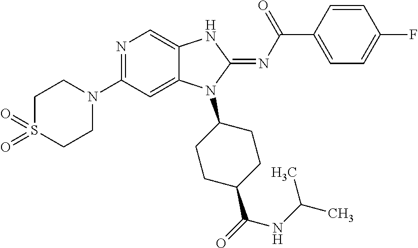 [SMILES: CC(C)NC(=O)[C@H]1CC[C@@H](n2/c(=N/C(=O)c3ccc(F)cc3)[nH]c3cnc(N4CCS(=O)(=O)CC4)cc32)CC1] |
| 224 | ++++++ | 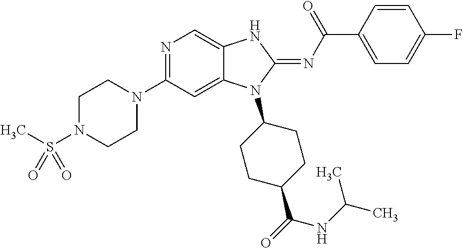 [SMILES: CC(C)NC(=O)[C@H]1CC[C@@H](n2/c(=N/C(=O)c3ccc(F)cc3)[nH]c3cnc(N4CCN(S(C)(=O)=O)CC4)cc32)CC1] |
| 225 | ++++++ | 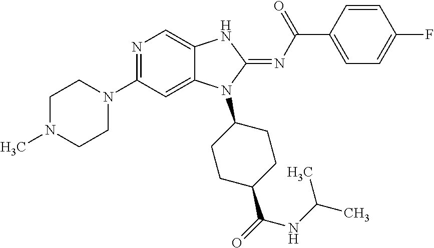 [SMILES: CC(C)NC(=O)[C@H]1CC[C@@H](n2/c(=N/C(=O)c3ccc(F)cc3)[nH]c3cnc(N4CCN(C)CC4)cc32)CC1] |
| 226 | ++++++ | 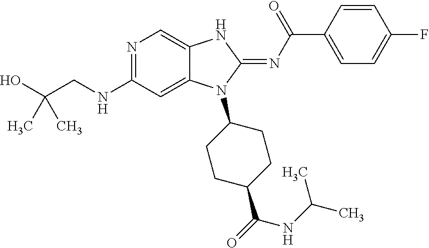 [SMILES: CC(C)NC(=O)[C@H]1CC[C@@H](n2/c(=N/C(=O)c3ccc(F)cc3)[nH]c3cnc(NCC(C)(C)O)cc32)CC1] |
| 227 | +++++ | 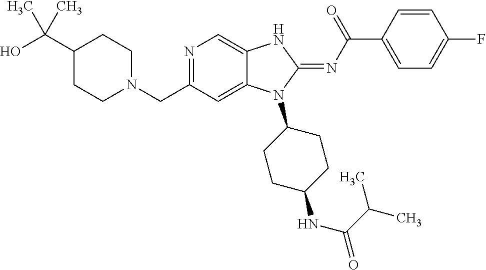 [SMILES: CC(C)C(=O)N[C@H]1CC[C@@H](n2/c(=N/C(=O)c3ccc(F)cc3)[nH]c3cnc(CN4CCC(C(C)(C)O)CC4)cc32)CC1] |
| 228 | ++++ | 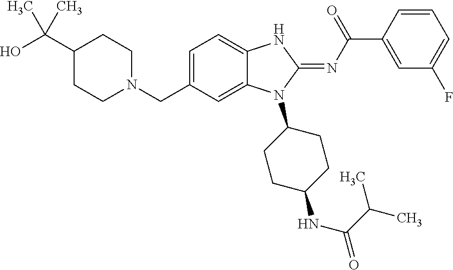 [SMILES: CC(C)C(=O)N[C@H]1CC[C@@H](n2/c(=N/C(=O)c3cccc(F)c3)[nH]c3ccc(CN4CCC(C(C)(C)O)CC4)cc32)CC1] |
| 229 | +++++ | 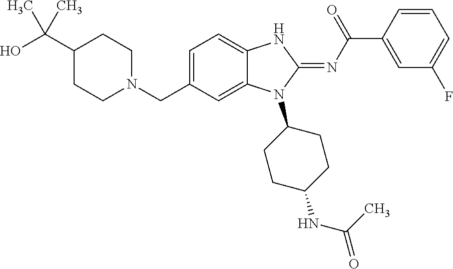 [SMILES: CC(=O)N[C@H]1CC[C@H](n2/c(=N/C(=O)c3cccc(F)c3)[nH]c3ccc(CN4CCC(C(C)(C)O)CC4)cc32)CC1] |
| 230 | ++++++ | 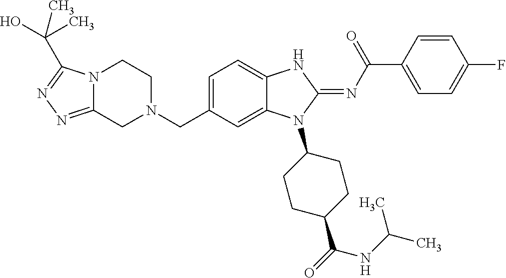 [SMILES: CC(C)NC(=O)[C@H]1CC[C@@H](n2/c(=N/C(=O)c3ccc(F)cc3)[nH]c3ccc(CN4CCn5c(nnc5C(C)(C)O)C4)cc32)CC1] |
| 231 | ++++++ | 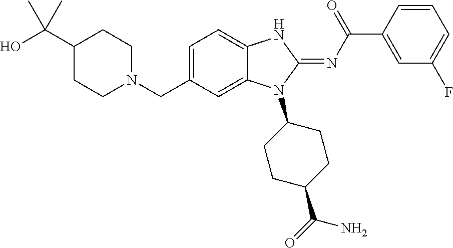 [SMILES: CC(C)(O)C1CCN(Cc2ccc3[nH]/c(=N\C(=O)c4cccc(F)c4)n([C@H]4CC[C@@H](C(N)=O)CC4)c3c2)CC1] |
| 232 | ++++++ | 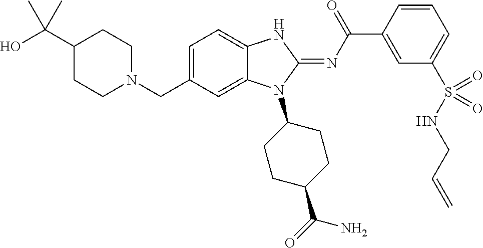 [SMILES: C=CCNS(=O)(=O)c1cccc(C(=O)/N=c2\[nH]c3ccc(CN4CCC(C(C)(C)O)CC4)cc3n2[C@H]2CC[C@@H](C(N)=O)CC2)c1] |
| 233 | +++++ | 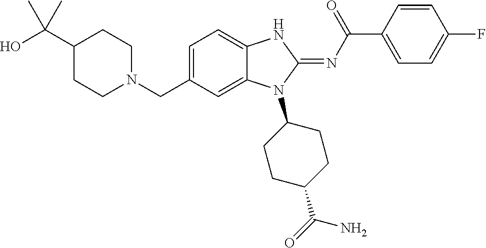 [SMILES: CC(C)(O)C1CCN(Cc2ccc3[nH]/c(=N\C(=O)c4ccc(F)cc4)n([C@H]4CC[C@H](C(N)=O)CC4)c3c2)CC1] |
| 234 | ++++ | 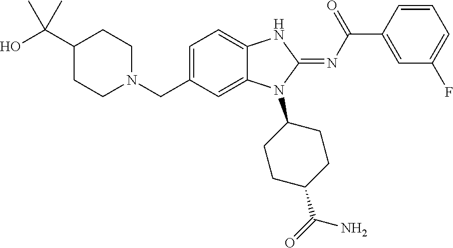 [SMILES: CC(C)(O)C1CCN(Cc2ccc3[nH]/c(=N\C(=O)c4cccc(F)c4)n([C@H]4CC[C@H](C(N)=O)CC4)c3c2)CC1] |
| 235 | ++++++ | 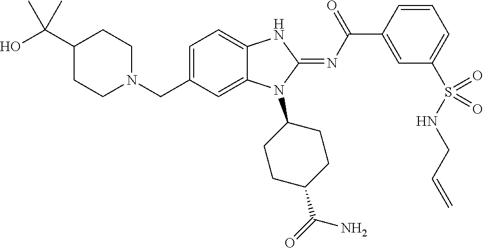 [SMILES: C=CCNS(=O)(=O)c1cccc(C(=O)/N=c2\[nH]c3ccc(CN4CCC(C(C)(C)O)CC4)cc3n2[C@H]2CC[C@H](C(N)=O)CC2)c1] |
| 236 | ++++++ | 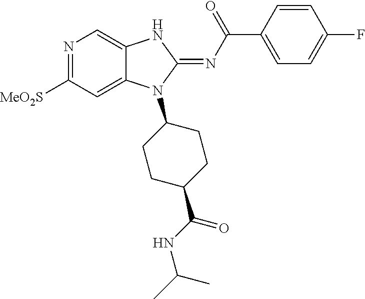 [SMILES: CC(C)NC(=O)[C@H]1CC[C@@H](n2/c(=N/C(=O)c3ccc(F)cc3)[nH]c3cnc(S(C)(=O)=O)cc32)CC1] |
| 237 | ++++ | 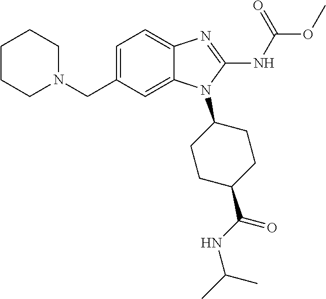 [SMILES: COC(=O)Nc1nc2ccc(CN3CCCCC3)cc2n1[C@H]1CC[C@@H](C(=O)NC(C)C)CC1] |
| 238 | ++++++ | 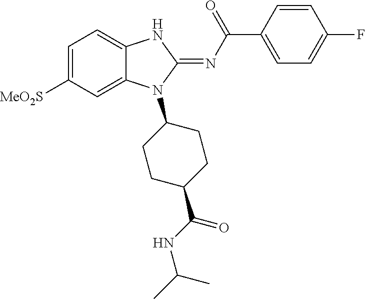 [SMILES: CC(C)NC(=O)[C@H]1CC[C@@H](n2/c(=N/C(=O)c3ccc(F)cc3)[nH]c3ccc(S(C)(=O)=O)cc32)CC1] |
| 239 | ++++++ | 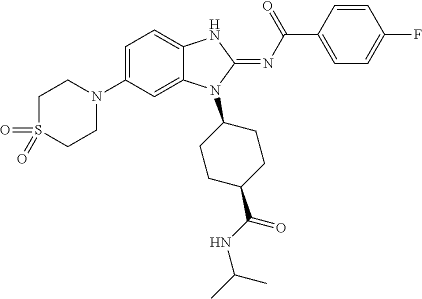 [SMILES: CC(C)NC(=O)[C@H]1CC[C@@H](n2/c(=N/C(=O)c3ccc(F)cc3)[nH]c3ccc(N4CCS(=O)(=O)CC4)cc32)CC1] |
| 240 | +++ | 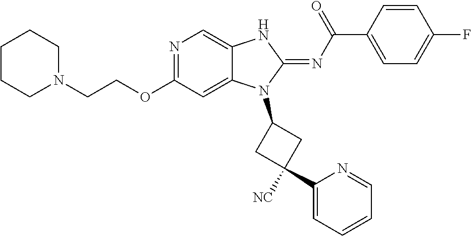 [SMILES: N#C[C@]1(c2ccccn2)C[C@@H](n2/c(=N/C(=O)c3ccc(F)cc3)[nH]c3cnc(OCCN4CCCCC4)cc32)C1] |
| 241 | ++++ | 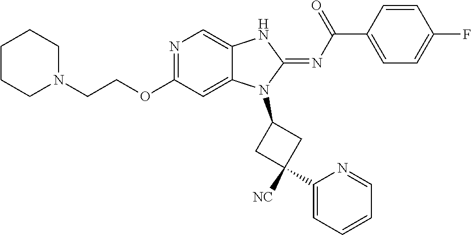 [SMILES: N#C[C@]1(c2ccccn2)C[C@H](n2/c(=N/C(=O)c3ccc(F)cc3)[nH]c3cnc(OCCN4CCCCC4)cc32)C1] |
| 242 | ++++ | 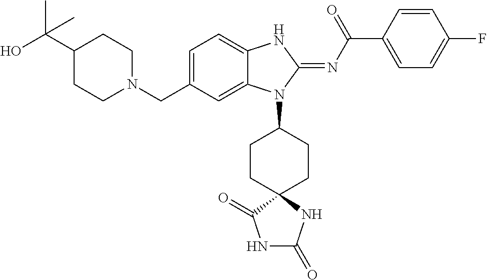 [SMILES: CC(C)(O)C1CCN(Cc2ccc3[nH]/c(=N\C(=O)c4ccc(F)cc4)n([C@H]4CC[C@]5(CC4)NC(=O)NC5=O)c3c2)CC1] |
| 243 | ++++++ | 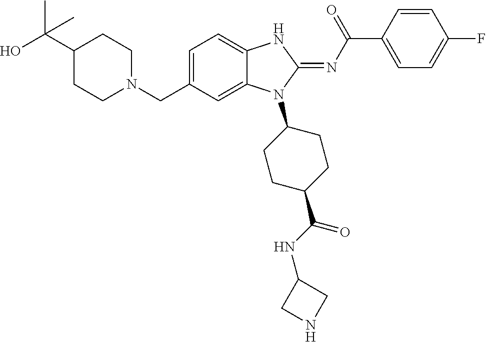 [SMILES: CC(C)(O)C1CCN(Cc2ccc3[nH]/c(=N\C(=O)c4ccc(F)cc4)n([C@H]4CC[C@@H](C(=O)NC5CNC5)CC4)c3c2)CC1] |
| 244 | +++++ | 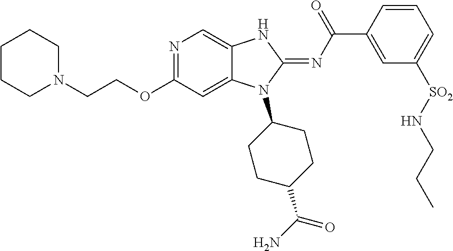 |
| 245 | +++++ | 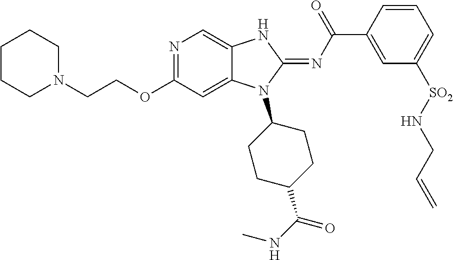 |
| 246 | ++++ | 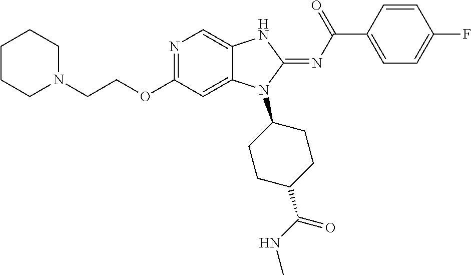 |
| 247 | +++ | 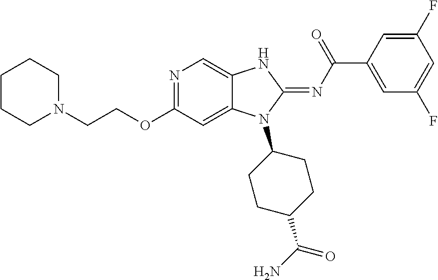 |
| 248 | +++ | 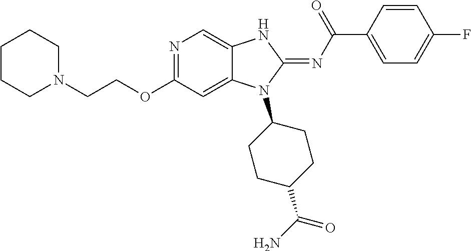 |
| 249 | +++ | 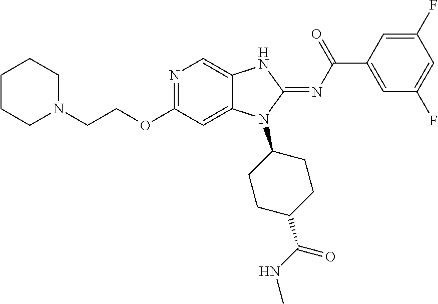 |
| 250 | ++++ | 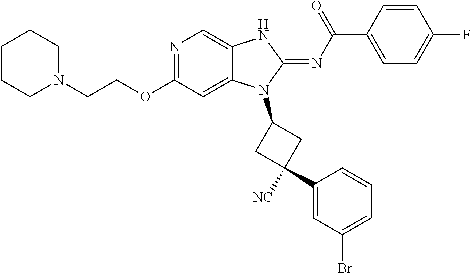 [SMILES: N#C[C@]1(c2cccc(Br)c2)C[C@@H](n2/c(=N/C(=O)c3ccc(F)cc3)[nH]c3cnc(OCCN4CCCCC4)cc32)C1] |
| 251 | +++++ | 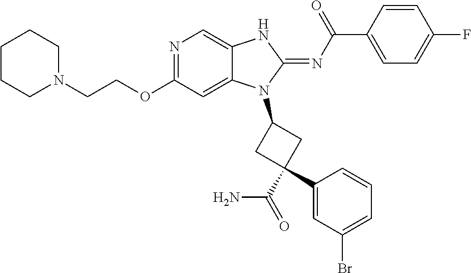 [SMILES: NC(=O)[C@]1(c2cccc(Br)c2)C[C@@H](n2/c(=N/C(=O)c3ccc(F)cc3)[nH]c3cnc(OCCN4CCCCC4)cc32)C1] |
| 252 | +++ | 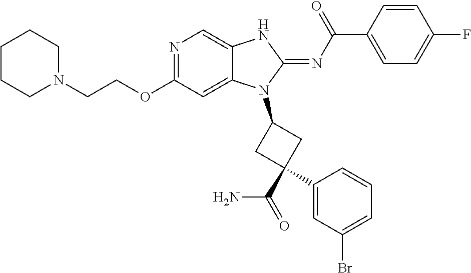 [SMILES: NC(=O)[C@]1(c2cccc(Br)c2)C[C@H](n2/c(=N/C(=O)c3ccc(F)cc3)[nH]c3cnc(OCCN4CCCCC4)cc32)C1] |

^{a}IC₅₀ Ranges:
+ IC₅₀ > 10 μM
++ 5 μM ≦ IC₅₀ ≦ 10 μM
+++ 1 μM ≦ IC₅₀ ≦ 5 μM
++++ 0.1 μM ≦ IC₅₀ < 1 μM
+++++ 0.05 μM < IC₅₀ < 0.1 μM
++++++ IC₅₀ < 0.05 μM
^{b}Not Applicable

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the claims.

We claim:

1. A method comprising steps of:
administering a compound of formula I to a subject suffering from an ALK-associated condition, wherein the subject shows one or more indicia of ALK-inhibitor resistance.

2. The method according to claim 1, wherein the one or more indicia of ALK-inhibitor resistance is selected from L1196M, R1275Q, F1174L, ELM4-ALK, NPM-ALK and combinations thereof.

3. The method according to claim 1 or claim 2, wherein the ALK-inhibitor is crizotinib.

4. The method according to claim 3, wherein the compound of formula I is administered in a dosage amount selected from about 50 mg to about 1200 mg.

5. The method according to claim 4, wherein the compound of formula I is administered once, twice, three or four times daily.

6. A method comprising steps of:
administering to a subject suffering from or susceptible to an ALK-associated condition a compound of formula I in combination with an additional chemotherapeutic agent.

7. The method according to claim 6, wherein the additional chemotherapeutic agent is selected from the group consisting of docetaxel, pemetrexed, carboplatin, paclitaxel and cisplatin.

8. The method according to claim 6 or claim 7, wherein at least one of the compound of formula I and the additional chemotherapeutic agent is administered at a dose lower than when administered as a single agent.

9. The method according to claim 1 or claim 6, wherein the subject has an ALK-associated genetic marker selected from L1196M, R1275Q, F1174L, ELM4-ALK, NPM-ALK and combinations thereof.

10. The method according to claim 9, wherein the ALK-associated genetic marker is detected by fluorescence in situ hybridization.

11. The method according to claim 6, wherein the subject has a crizotinib-resistance associated marker.

12. The method according to claim 11, wherein the crizotinib-resistance associated marker is detected at a level above a threshold correlated with elevated probability of resistance to crizotinib.

13. The method according to claim 12, wherein the crizotinib-resistance associated marker is detected by fluorescence in situ hybridization.

14. The method according to any of claim 11, 12 or 13, wherein the crizotinib-resistance associated marker is L1196M.

15. A method comprising steps of:
i. detecting in a subject an ALK-inhibitor resistance-associated marker; and
ii. determining that the subject is a candidate for therapy with a compound of formula I.

16. A method comprising steps of:
i. detecting in a subject an ALK-inhibitor resistance-associated marker;
ii. determining that the subject is a candidate for therapy with a compound of formula I, and
iii. administering to the patient a therapeutically effective amount of a compound of formula I.

17. The method according to claim 15 or claim 16, wherein the ALK-inhibitor resistance-associated marker is a crizotinib resistance-associated marker.

18. The method according to claim 17, wherein the crizotinib resistance-associated marker is L1196M.

19. The method according to any of claims 15-18, wherein the subject is or has received crizotinib therapy.

20. A method of treating a TRK-associated condition, the method comprising administering to a patient in need thereof a compound of formula I.

21. The method according to claim 20, wherein the TRK-associated condition is cancer.

22. The method according to claim 20, wherein the TRK-associated condition is pain.

23. The method according to claim 22, wherein the TRK-associated condition is cancer pain.

24. A method of treating an ALK-associated condition, the method comprising administering to a patient in need thereof a compound of formula I, wherein the ALK-associated condition is a localized or present in the central nervous system.

25. The method according to claim 24, wherein the ALK-associated condition is localized or present in the brain.

26. The method according to claim 25, wherein the ALK-associated condition is brain cancer.

27. The method according to claim 26, wherein the ALK-associated condition is metastatic brain cancer.

28. The method according to claim 24, wherein the ALK-associated condition is localized or present in the spinal cord.

29. The method according to claim 28, wherein the ALK-associated condition is spinal cancer.
